(19) United States
(12) Patent Application Publication
(10) Pub. No.: US 2017/0210809 A1
(43) Pub. Date: Jul. 27, 2017

(54) BISPECIFIC BINDING AGENTS TARGETING THE IGF-1R AND ERBB3 PATHWAYS AND USES THEREOF

(71) Applicant: Merrimack Pharmaceuticals, Inc., Cambridge, MA (US)

(72) Inventors: Birgit SCHOEBERL, Cambridge, MA (US); Ulrik NIELSEN, Quincy, MA (US); Arthur J. KUDLA, Andover, MA (US); Alexey Alexandrovich LUGOVSKOY, Woburn, MA (US); Jonathan B. FITZGERALD, Arlington, MA (US); Lihui XU, Chestnut Hill, MA (US); Neeraj KOHLI, Brighton, MA (US)

(21) Appl. No.: 15/371,536

(22) Filed: Dec. 7, 2016

Related U.S. Application Data
(62) Division of application No. 13/443,660, filed on Apr. 10, 2012, now abandoned.
Application No. 13/443,660 is a continuation of application No. PCT/US2010/052712, filed on Oct. 14, 2010.
(60) Provisional application No. 61/251,426, filed on Oct. 14, 2009.

(51) Int. Cl. C07K 16/28; A61K 39/395; C07K 16/32

(52) U.S. Cl. CPC C07K 16/2863; C07K 16/32; A61K 39/39558; C07K 2317/31; C07K 2317/622; C07K 2317/54; C07K 2317/92; C07K 2317/76

(57) ABSTRACT

Disclosed are bispecific binding agents that specifically target both of the IGF-1 and the ErbB intracellular signaling pathways. For example, bispecific binding agents that comprise an anti-IGF-1R antibody and an anti-ErbB3 antibody connected by a linker are described herein. These bispecific agents are capable of antagonizing signal transduction by both of the IGF-1 and the ErbB signaling pathways and are useful in inhibiting the proliferation of tumor cells whose growth involves the signaling activity of both pathways.

### RELATED APPLICATIONS

This application is a divisional of U.S. patent application Ser. No. 13/443,660, filed Apr. 10, 2012, which is a continuation of International Application No. PCT/US2010/052712, filed Oct. 14, 2010, which claims priority to U.S. Provisional Application No. 61/251,426, filed Oct. 14, 2009. The entire contents of the aforementioned applications are incorporated herein by reference.

### SEQUENCE LISTING

The instant application contains a Sequence Listing which has been submitted in ASCII format via EFS-Web and is hereby incorporated by reference in its entirety. Said ASCII copy, created on Dec. 1, 2016, is named MMJ_023PCCNDV_Sequence_Listing.txt and is 420,819 bytes in size.

### BACKGROUND

It has been established that tumor cells express receptors for growth factors and cytokines that stimulate proliferation of the cells and, moreover, that antibodies to such receptors can be effective in blocking the stimulation of cell proliferation mediated by growth factors and cytokines to inhibit tumor cell growth. Commercially available therapeutic antibodies that target receptors on cancer cells include, for example, trastuzumab (Herceptin®) for the treatment of breast cancer, which targets the HER2 receptor (also known as ErbB2), and cetuximab (Erbitux®) for the treatment of colorectal cancer and head and neck cancer, which targets the epidermal growth factor receptor (EGFR, also known as HER1 or ErbB1).

While this approach of administering a therapeutic agent comprising only a single therapeutic monoclonal antibody (when administered in the absence of administration of another therapeutic antibody, referred to herein as monotherapy) has shown considerable success in cancer treatment, there are a number of factors that can lead to failure of such treatment or recurrence of tumor growth after initial inhibition. For example, certain tumors rely on more than one growth factor-mediated signal transduction pathway for cell proliferation and thus targeting of a single pathway may prove insufficient to significantly affect tumor cell growth. Alternatively, even in cases where one pathway is the only or predominant growth-stimulatory pathway, certain tumors cells are capable of activating another signaling pathway for growth stimulation when the original one is blocked by antibody (innate resistance to treatment). Still further, some tumors exhibit initial responsiveness to antibody monotherapy but later develop resistance to treatment by switching to use of another signaling pathway (acquired resistance to treatment).

Accordingly, additional therapeutic approaches for cancer treatment are needed to overcome limitations of antibody monotherapy and to provide other benefits.

### SUMMARY

Provided herein are bispecific binding agents (BBAs) that target two signaling pathways used by tumor cells for activation of proliferation, the insulin growth factor 1 receptor (IGF-1R) pathway and the ErbB pathway, and in particular the ErbB3 (also known as HER3) pathway. The BBAs comprise a binding moiety (module) that targets IGF-1R and a binding moiety (module) that targets ErbB3 covalently linked together via a linker moiety (module) in between. As described herein, these BBAs have been shown to be more effective in inhibiting proliferation of tumor cells than use of a single binding agent targeting either the IGF-1 pathway or the ErbB3 pathway alone.

Accordingly, in one aspect, a BBA comprising a first binding moiety that specifically binds the IGF-1 receptor (IGF-1R) and a second binding moiety that specifically binds ErbB3 is provided, wherein the first and second binding moiety are covalently linked by a linker moiety. In one embodiment, the linking moiety is monomeric, in that one molecule of such a linking moiety does not form multimers with other linking moiety molecules. This monomeric moiety can comprise human serum albumin (HSA) having the sequence set forth in SEQ ID:18. In another embodiment, the monomeric linking moiety is a mutated form of human serum albumin, having serine at position 34 and glutamine at position 503, having the sequence set forth in SEQ ID NO:19. In another embodiment the monomeric linker moiety is chemically and biologically inert (in that the linker does not have any biologic binding function or catalytic function) as set forth in SEQ ID:32. In another embodiment the linking moiety is constitutively or inducibly capable of dimerization (referred to herein as dimeric). This dimeric linker can, e.g., comprise a fragment of human immunoglobulin as set forth in SEQ ID NOs:20-29. In another embodiment the linking moiety is constitutively or inducibly capable of trimerization (referred to herein as trimeric). This trimeric linking moiety can comprise Tumor Necrosis Factor homology domain or a fragment of Tumor Necrosis Factor homology domain. The examples of constitutive trimeric linker is set forth in SEQ ID NO:31. An example of inducible trimeric linker is set forth in SEQ ID NO: 30.

The glycosylation states of linker moieties can be engineered by means of introduction of amino acid motif that undergoes N-linked glycosylation in eukaryotic expression hosts. In one embodiment, the linking moiety contains asparagine at position 180, serine at position 181 and threonine at position 182 (as set forth in SEQ ID NO:24) and is glycosylated. In another embodiment the linking moiety contains asparagine to glutamine mutation at position 180 (as set forth in SEQ ID NO:25) and is aglycosylated. In another embodiment the linking moiety was engineered to contain a second N-linked glycosylation motif (asparagine at position 78, glutatmine at position 79, and threonine at position 80). This linking moiety set forth in SEQ ID NO: 26 is hyperglucosylated. Additional examples of glycoengineered linking moieties are set forth in SED ID NOs:27-29.

Additional methods of such glycoengeneering are described in US 2006/0269543 and references therein.

In one embodiment, the first binding moiety is an anti-IGF-1R genetically engineered antibody fragment such as single chain antibody (scFv). An exemplary anti-IGF-1R single chain antibody is set forth in SEQ ID NO:1.

In another embodiment the first binding moiety is an anti-IGF-1R antibody fragment such as a Fab. A Fab fragment is composed of a heterodimer of a light chain (LC) and a heavy chain (HC). Exemplary HC and LC sequences for anti-IGF-1R Fab fragments are set forth in SEQ ID NO:8 and SEQ ID NO:10.

In another embodiment, the first binding moiety is an anti-IGF-1R antibody fragment such as a VH domain. An exemplary anti-IGF-1R VH domain is set forth in SEQ ID NO:6.

In another embodiment, the first binding moiety is an anti-IGF-1R antibody fragment such as a VL domain. An exemplary anti-IGF-1R VL domain is set forth in SEQ ID NO:12.

In one embodiment, the first binding moiety is an anti-ErbB3 genetically engineered antibody fragment such as single chain antibody (scFv). Exemplary anti-ErbB3 single chain antibodies are set forth in SEQ ID NO:33, SEQ ID NO:43, and SEQ ID NO:44.

In another embodiment the first binding moiety is an anti-ErbB3 antibody fragment such as a Fab. A Fab fragment is composed of a heterodimer of light chain (LC) and heavy chain (HC). Exemplary HC and LC sequences for anti-ErbB3 Fabs are set forth in SEQ ID NO:37 and SEQ ID NO:39.

In another embodiment, the first binding moiety is an anti-ErbB3 antibody fragment such as VH domain. An exemplary anti-ErbB3 VH domain is set forth in SEQ ID NO:35.

In another embodiment, the first binding moiety is an anti-ErbB3 antibody fragment such as VL domain. An exemplary anti-ErbB3 VL domain is set forth in SEQ ID NO:41.

In another embodiment, the second binding moiety is an anti-ErbB3 antibody, for example a single chain antibody (scFv). Exemplary anti-ErbB3 single chain antibodies are the AB2-3 scFv (comprising the sequence set forth in SEQ ID NO:33), the AB2-6 scFv (comprising the sequence set forth in SEQ ID NO:43) and the AB2-21 scFv (comprising the sequence set forth in SEQ ID NO:44). In one embodiment, the first binding moiety is an anti-ErbB3 genetically engineered antibody fragment such as single chain antibody (scFv). An exemplary anti-ErbB3 single chain antibody is set forth in SEQ ID NOs:33, 43, and 44.

In another embodiment the first binding moiety is an anti-ErbB3 antibody fragment such as Fab. Fab fragment is composed of heterodimer of light chain (LC) and heavy chain (HC). An exemplary HC and LC sequences for anti-ErbB3 Fab fragment are set forth in SEQ ID NO:37 and SEQ ID NO:39.

In another embodiment, the first binding moiety is an anti-ErbB3 antibody fragment such as VH domain. An exemplary anti-ErbB3 VH domain is set forth in SEQ ID NO:35.

In another embodiment, the first binding moiety is an anti-ErbB3 antibody fragment such as VL domain. An exemplary anti-ErbB3 VL domain is set forth in SEQ ID NO:41.

Another embodiment comprises the AB5-7 scFv linked to the N-terminus of the mutated HSA linker and the AB2-3 scFv linked to the C-terminus of the mutated HSA linker (SEQ ID NO:93, coded for by SEQ ID NO:99), the AB5-7 scFv linked to the N-terminus of the mutated HSA linker and the AB2-6 scFv linked to the C-terminus of the mutated HSA linker (SEQ ID NO:94, coded for by SEQ ID NO:100), the AB5-7 scFv linked to the N-terminus of the mutated

HSA linker and the AB2-21 scFv linked to the C-terminus of the mutated HSA linker (SEQ ID NO:95, coded for by SEQ ID NO:108), the AB2-3 scFv linked to the N-terminus of the mutated HSA linker and the AB5-7 scFv linked to the C-terminus of the mutated HSA linker (SEQ ID NO:96, coded for by SEQ ID NO:115), the AB2-6 scFv linked to the N-terminus of the mutated HSA linker and the AB5-7 scFv linked to the C-terminus of the mutated HSA linker (SEQ ID NO:97, coded for by SEQ ID NO:116) and the AB2-21 scFv linked to the N-terminus of the mutated HSA linker and the AB5-7 scFv linked to the C-terminus of the mutated HSA linker (SEQ ID NO:98, coded for by SEQ ID NO:117).

Other embodiments comprise an anti-ErbB3 moiety N-terminally fused to a linker moiety that is in turn fused to C-terminal anti-IGF-1R moiety. Such molecules can conform to the formula A-L-B as set forth below, and may have particular combinations of moieties as set forth below in Table 11. These moieties are fused continuously without intervening sequences. The coexpressed moiety, if present, is expressed in the same cell as separate polypeptide chain. The folding of these polypeptide chains gives rise to bispecific molecules of ELI topology.

Other embodiments comprise an anti-IGF-1R moiety N-terminally fused to a linker moiety that is in turn fused to C-terminal anti-ErbB3 moiety. Such molecules can conform to the formula A-L-B as set forth below, and may have particular combinations of moieties as set forth in Table 12. These moieties are fused continuously without intervening sequences. The coexpressed moiety, if present, is expressed in the same cell as separate polypeptide chain. The folding of these polypeptide chains gives rise to bispecific molecules of ILE topology.

The C-terminal lysine variation is commonly observed in biopharmaceutical antibodies and antibody-like molecules. The C-terminal lysine can be cleaved by basic carboxypeptidase, such as carboxypeptidise B. This processing is known to be sensitive to the production process and incomplete cleavage can result in increased heterogeneity of biopharmaceutical drug product.

In one embodiment C-terminal anti-IGF-1R moiety is engineered to be homogeneous via removal of C-terminal lysine. An exemplary homogeneous anti-IGF-1R moiety is set forth in SEQ ID NO:3.

In another embodiment C-terminal anti-ErbB3 moiety is engineered to be homogeneous via removal of C-terminal lysine. An exemplary homogeneous anti-ErbB3 moiety is set forth in SEQ ID NO:82.

The methods for engineering of antibody fragments, such as scFv, VH, VL, and Fab with enhanced stability and increased expression are described in US 2006/0127893 US 2009/0048122 and references therein.

In one embodiment anti-ErbB3 moiety is engineered for enhanced stability by such methods. An exemplary stabilized anti-ErbB3 moiety is set forth in SEQ ID NO:34.

In another embodiment anti-IGF-1R moiety is engineered for enhanced stability by such methods. An exemplary stabilized anti-IGF-1R moiety is set forth in SEQ ID NO:2. In another embodiment anti-IGF-1R moiety is engineered for increased expression by such methods. An exemplary expression optimized anti-IGF-1R moiety is set forth in SEQ ID NO:4.

Avidity, in increase in binding strength resulting from a plurality of affinity interactions (typically against a single target), can improve the biologic function of antibodies and antibody-like molecules.

In certain embodiments both of the binding modules comprised by a BBA are capable of only a single affinity interaction, i.e., they are capable of a single affinity interaction with IGF-1R and a single affinity interaction for ErbB3. An exemplary BBA with these characteristics can be constructed by genetic fusion of SEQ ID NO:43 to SEQ ID NO:19 to SEQ ID NO:1, without intervening amino acids, as described in the Example 5.

In other embodiments, one or more of the binding modules of a BBA is capable of a plurality of affinity interactions, yielding avidity binding characteristics. Such binding modules are oligovalent, being capable of two, three, four, five, or more separate affinity interactions with the same target, and are referred to herein as “tandem” modules.

Thus, in certain higher affinity (avidity) embodiments, BBAs are capable of two affinity interactions for IGF-1R and two affinity interactions for ErbB3. An exemplary BBA with these characteristics can be constructed genetic fusion of SEQ ID NO:35 to SEQ ID NO:22 to SEQ ID NO:1 without intervening sequences and co-expression with SEQ ID NO:39 in the same cell as described in the Example 5.

In certain even higher affinity (avidity) embodiments BBAs are capable of three affinity interactions for IGF-1R and three affinity interactions for ErbB3. An exemplary BBA with these characteristics can be constructed by genetic fusion of SEQ ID NO:47 to SEQ ID NO:31 to SEQ ID NO:5 without intervening sequences as described in the Example 5.

In other embodiments a BBA is capable of only a single affinity interaction with IGF-1R and and two affinity interactions for ErbB3. An exemplary BBA with these characteristics can be constructed by genetic fusion of SEQ ID NO:1 to SEQ ID NO:19 to SEQ ID NO:50 without intervening sequences as described in the Example 5.

Also provided is a bispecific binding agent protein, wherein the agent comprises an IGF-1R targeting moiety, a linker moiety, and an ErbB3 targeting moiety, wherein the IGF-1R targeting moiety specifically binds to IGF-1R and the ErbB3 targeting moiety specifically binds to ErbB3 and wherein the targeting moieties are each linked to the linker moiety. In one embodiment, each of the targeting moieties is covalently linked to the linker moiety by a peptide bond to form a single polypeptide and the linker moiety is 2-5, 6-10, 11-25, 26-50, 51-100, 101-250, 251-500, or 501-1000 amino acids long.

Various forms of linker moieties are contemplated. In one embodiment, the linker moiety is chemically and biologically inert. In another embodiment, the linker moiety is composed of one or more protein domains. In another embodiment, the linker moiety binds to one or more receptor, including, for example, Fcγ receptor, neonatal Fc receptor, Tumor Necrosis Factor family receptor, human immunoglobulin, or human serum albumin. In another embodiment, the linker moiety is a human serum albumin. In another embodiment, the linker moiety is an immunoglobulin, or immunoglobulin fragment. In another embodiment, the linker moiety is Tumor Necrosis Factor homology domain, or a fragment of Tumor Necrosis Factor homology domain. In another embodiment, the linker moiety forms a monomer. In another embodiment, the linker moiety forms a homodimer or heterodimer. In another embodiment, the linker moiety forms a homotrimer or heterotrimer. In another embodiment, the linker moiety is glycosylated or aglycosylated (non-glycosylated). In another embodiment, the linker moiety is a mutated form of human serum albumin. In another embodiment, the linker contains CH2 and/or CH3 domain of human immunoglobulin of IgG1, IgG2, IgG3 or IgG4 isotype. In another embodiment, the linker moiety is a fragment of human TRAIL, human LIGHT, human CD40L, human TNFα, human CD95, human BAFF, human TWEAK, human OX40, or human TNFβ and wherein the fragment is constitutively or inducibly capable of dimerization or trimerization. In another embodiment, the linker moiety is glycoengineered to have enhanced solubility. In another embodiment, the linker moiety is engineered to have enhanced stability. In another embodiment, the linker moiety is engineered to provide extended serum half-life. In another embodiment, the linker moiety is engineered to have reduced heterogeneity.

In a particular embodiment, the ErbB3 targeting moiety is linked to the amino terminus of the linker moiety and the IGF-1R targeting moiety is linked to the carboxy terminus of the linker moiety.

In another embodiment, the IGF-1R targeting moiety is linked to the amino terminus of the linker moiety and the ErbB3 targeting moiety is linked to the carboxy terminus of the linker moiety.

In a further embodiment, the IGF-1R targeting moiety comprises one or more anti-IGF-1R antibody (e.g., a single chain antibody or a single domain antibody). In a particular embodiment, the IGF-1R targeting moiety comprises two anti-IGF-1R antibodies and the ErbB3 targeting moiety comprises one anti-ErbB3 antibody.

In another embodiment, the ErbB3 targeting moiety comprises one or more anti-ErbB3 antibody (e.g., a single chain antibody or a single domain antibody).

The targeting moieties provided herein can be engineered to have enhanced stability, reduced heterogeneity, or enhanced expression. For example, in one embodiment, either or both the IGF-1R targeting moiety and the ErbB3 targeting moiety have been engineered to have enhanced stability. In another embodiment, either or both of the IGF-1R targeting moiety and the ErbB3 targeting moiety have been engineered to have reduced heterogeneity. In yet a further embodiment, either or both of the IGF-1R targeting moiety and the ErbB3 targeting moiety have been engineered for enhanced expression.

Another aspect of the invention pertains to nucleic acid molecules, e.g., expression vectors, comprising sequences encoding the bispecific binding agents described herein operatively linked to a promoter, as well as host cells comprising such expression vectors and methods of expressing BBAs comprising culturing such host cells such that a BBA is expressed.

Kits comprising one or more of the BBAs described herein, as well as instructions for use of such agents to treat cancer, are also encompassed.

In another aspect, a method is provided for inhibiting proliferation of a tumor cell expressing IGF-1R and ErbB3 comprising contacting the tumor cell with a BBA described herein such that proliferation of the tumor cell is inhibited. Also provided is a method of treating a tumor expressing IGF-1R and ErbB3 in a patient, the method comprising administering a BBA described herein to the patient such that growth of the tumor is inhibited. Examples of tumors to be treated with BBAs (e.g., according to the methods of treatment disclosed herein) include lung cancer, sarcoma, colorectal cancer, head and neck cancer, pancreatic cancer and breast cancer. In various embodiments, the lung cancer is a non-small cell lung cancer or a gefitinib-resistant lung cancer, the sarcoma is a Ewing's sarcoma or the breast cancer is a tamoxifen-resistant, estrogen receptor-positive breast cancer or a trastuzumab-resistant metastatic breast cancer. The tumor treatment methods provided can further comprise administering a second anti-cancer agent, such as a chemotherapeutic drug, or administering an anti-cancer treatment modality, such as ionizing radiation, to the patient.

Further provided are methods of making bispecific binding agents, as well as methods of inhibiting proliferation of a tumor cell expressing IGF-1R and ErbB3 by contacting the tumor cell with any of the bispecific binding agents described herein, such that proliferation of the tumor cell is inhibited.

Also provided are methods of treating a tumor in a patient (e.g., a tumor comprising tumor cells expressing both IGF-1R and ErbB3), wherein the method comprises administering any one of the bispecific binding agents described herein to the patient in an amount effective to reduce tumor cell proliferation. In one embodiment, the tumor is a lung cancer (e.g., non-small cell lung cancer or a gefitinib-resistant lung cancer), sarcoma (e.g., Ewing's sarcoma), colorectal cancer, head and neck cancer, pancreatic cancer, ovarian cancer, or a breast cancer tumor (e.g., a tamoxifen-resistant, estrogen receptor-positive breast cancer or a trastuzumab-resistant metastatic breast cancer). In another embodiment, the method further comprises administering to the patient, in conjunction with treatment with a BBA, a second anti-cancer agent (e.g., a chemotherapeutic drug) to the patient or administering a second anti-cancer treatment modality to the patient (e.g., ionizing radiation).

Other features and advantages of the invention will be apparent from the following detailed description, and from the claims.

### BRIEF DESCRIPTION OF THE DRAWINGS

FIGS. 1A-1E show the amino acid (SEQ ID NO: 93) and nucleotide sequence (SEQ ID

NO: 99) of the BBA AB5-7N/AB2-3C.

FIGS. 2A-2E show the amino acid (SEQ ID NO: 94) and nucleotide sequence (SEQ ID NO: 100) of the BBA AB5-7N/AB2-6C.

FIGS. 3A-3E show the amino acid (SEQ ID NO: 95) and nucleotide sequence (SEQ ID NO: 108) of the BBA AB5-7N/AB2-21C.

FIGS. 4A-4E show the amino acid (SEQ ID NO: 96) and nucleotide sequence (SEQ ID NO: 115) of the BBA AB2-3N/AB5-7C.

FIGS. 5A-5E show the amino acid (SEQ ID NO: 97) and nucleotide sequence (SEQ ID NO: 116) of the BBA AB2-6N/AB5-7C.

FIGS. 6A-6E show the amino acid (SEQ ID NO: 98) and nucleotide sequence (SEQ ID NO: 117) of the BBA AB2-21N/AB5-7C.

FIGS. 7A-7C are bar graphs showing the inhibitory effect of the BBAs AB2-21N/AB5-7C and AB5-7N/AB2-21C on tumor spheroid growth of ADRr (FIG. 7A), MCF7 (FIG. 7B) and A549 (FIG. 7C) cells, as compared to the effect of anti-IGF-1R IgG alone or anti-ErbB3 IgG alone.

FIGS. 8A-8C are bar graphs showing the inhibitory effect of the BBAs AB2-21N/AB5-7C and AB5-7N/AB2-21C on tumor spheroid growth of ADRr (FIG. 8A), MCF7 (FIG. 8B) and A549 (FIG. 8C) cells, as compared to the effect of anti-IGF-1R IgG alone or anti-ErbB3 IgG alone.

FIG. 9 shows graphs that compare the monomeric BBA ILE-6 (94% monomer) MW 120 kDa to the ELI-7 dimeric BBA (94% monomer) MW 195kDa

FIG. 10 shows SDS page of different lots of the ILE-6 dimeric BBA (MW 195 kDa)

FIG. 11 shows SDS page of different lots of ELI-1 monomeric BBA (MW 120 kDa)

FIG. 12A shows binding on ADRr cells

FIG. 12B shows binding on MC7 cells

FIG. 13 shows comparison among trivalent and HSA bispecific formats

FIG. 14A shows pIGF-1R inhibition by ILE-7 and ELI-7

FIG. 14B shows pErbB3 inhibition by ILE-7 and ELI-7

FIG. 14C shows pAKT inhibition by ILE-7 and ELI-7

FIG. 15 shows effect of ELI-7 in DU145 (CTG)

FIG. 16 shows inhibition of BXPC3 growth in 2D culture by ELI-7

FIG. 17 shows a comparison among trivalent bispecifics (ILE-7 and ILE-9) and control IgG Ab#6

FIG. 18 shows the effect of trivalent bispecific antibody ILE-7 on DU145 spheroid growth

FIG. 19 shows BxPC-3 Tumor Growth Curves

FIG. 20 shows BxPC-3 Final Tumor Volumes on Day 41

FIG. 21A shows DU145 Tumor Growth Curves

FIG. 21B shows DU145 Tumor Volumes on Day 36

FIG. 22 shows HRG-induced pERbB3 by ILE-2 and ILE-3

FIG. 23A shows HRG stimulated pERbB3

FIG. 23B shows HRG-induced pAkt

FIG. 24 shows that the BBA ILE-7 completely inhibited pErbB3 at 10⁻⁷M compared to pErbB3 levels at 10⁻¹¹M ILE-7, whereas the BBA ILE-3 inhibited pErbB3 by no more than 50% at 10⁻⁷M compared to pErbB3 levels at 10⁻¹¹M ILE-3.

FIG. 25 shows that ILE-7 inhibited pAKT by more than 50% at 10⁻⁷M compared to pAkt levels at 10⁻¹¹M ILE-7, whereas ILE-3 inhibited pAkt by no more than 20% at 10⁻⁷M compared to pAkt levels at 10⁻¹¹M ILE-3.

FIG. 26 shows that BBAs inhibit signaling across a broad range of ErbB3 and IGF-1R receptor levels.

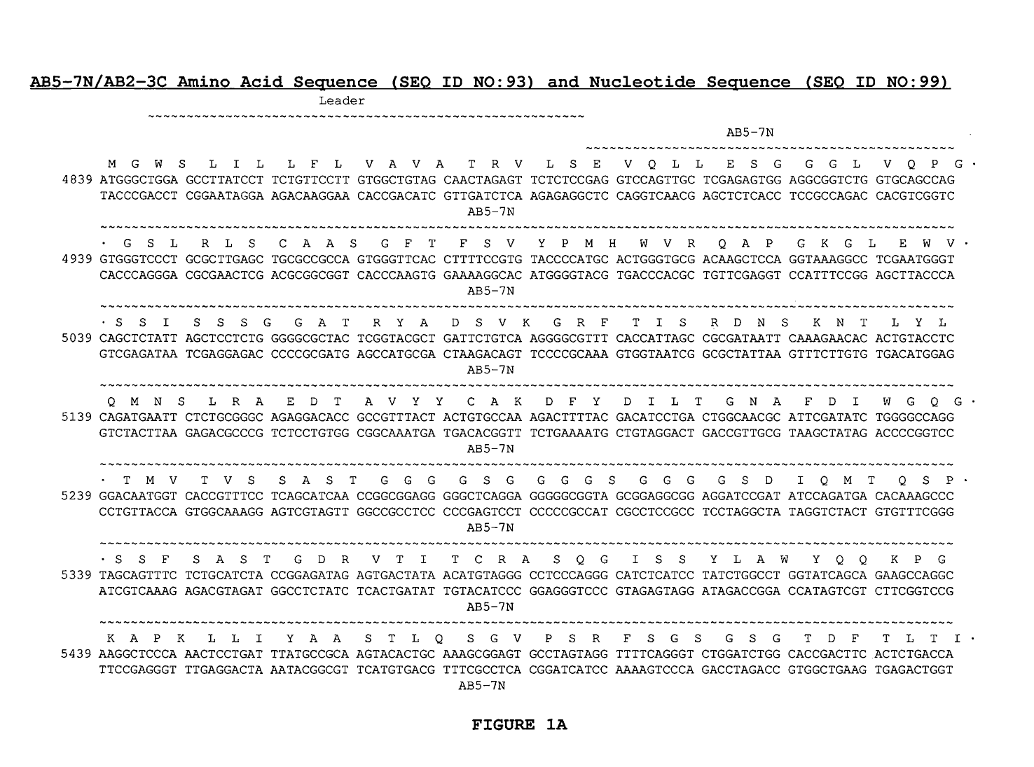

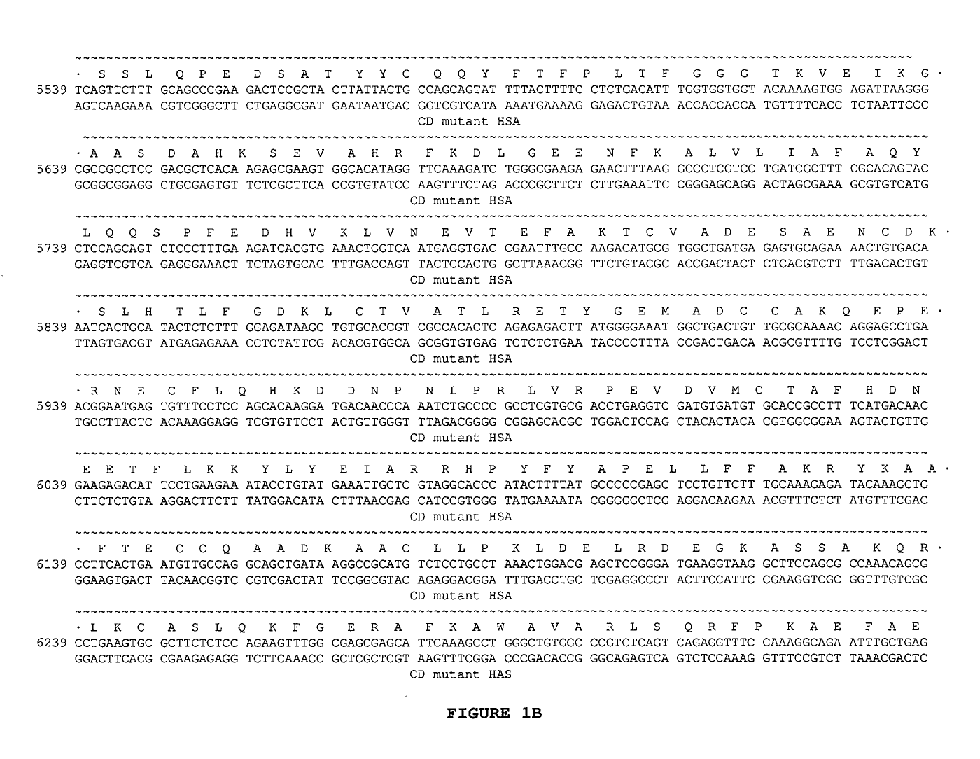

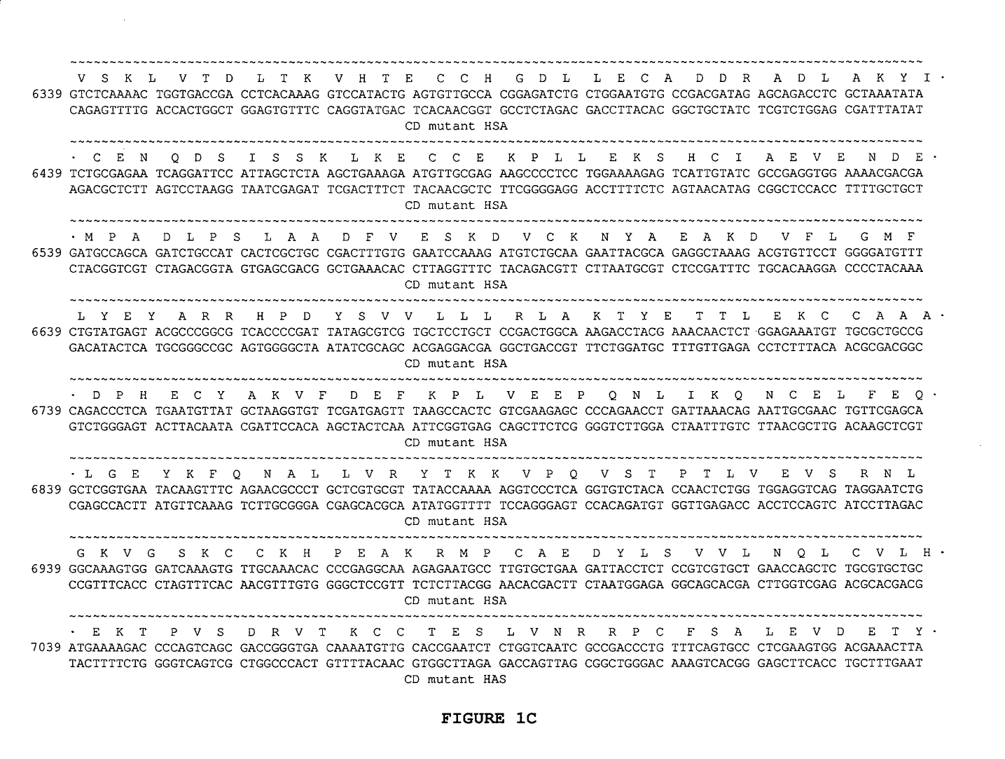

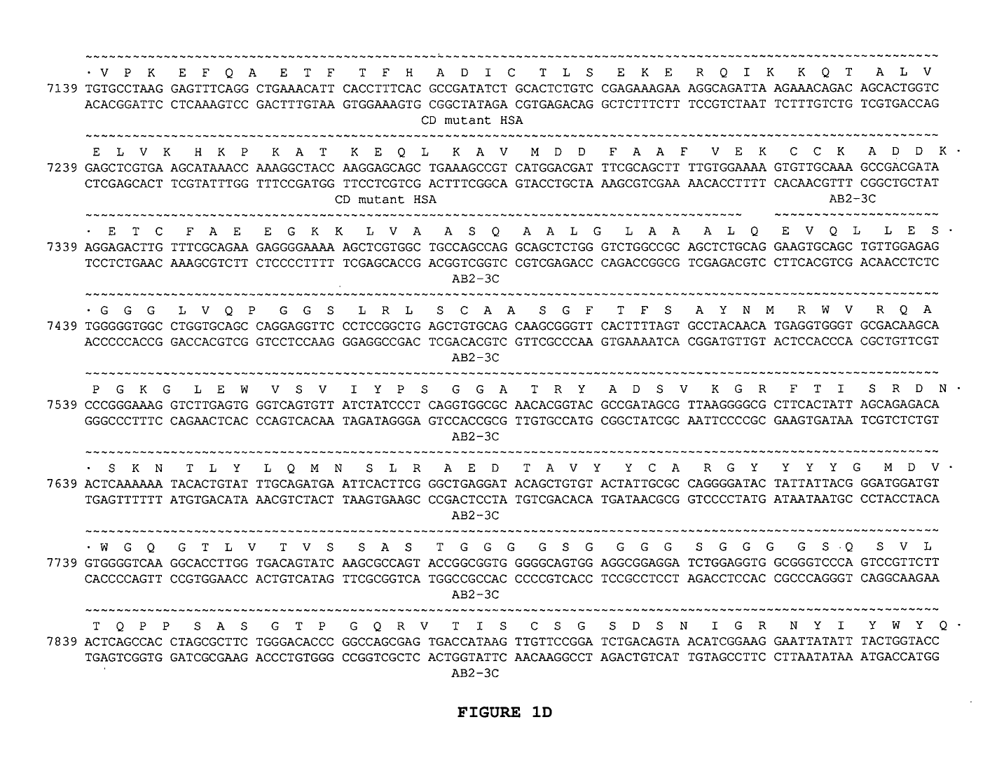

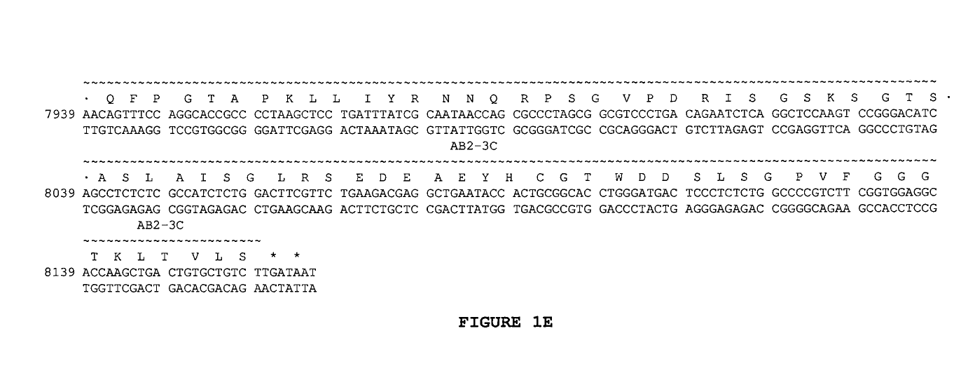

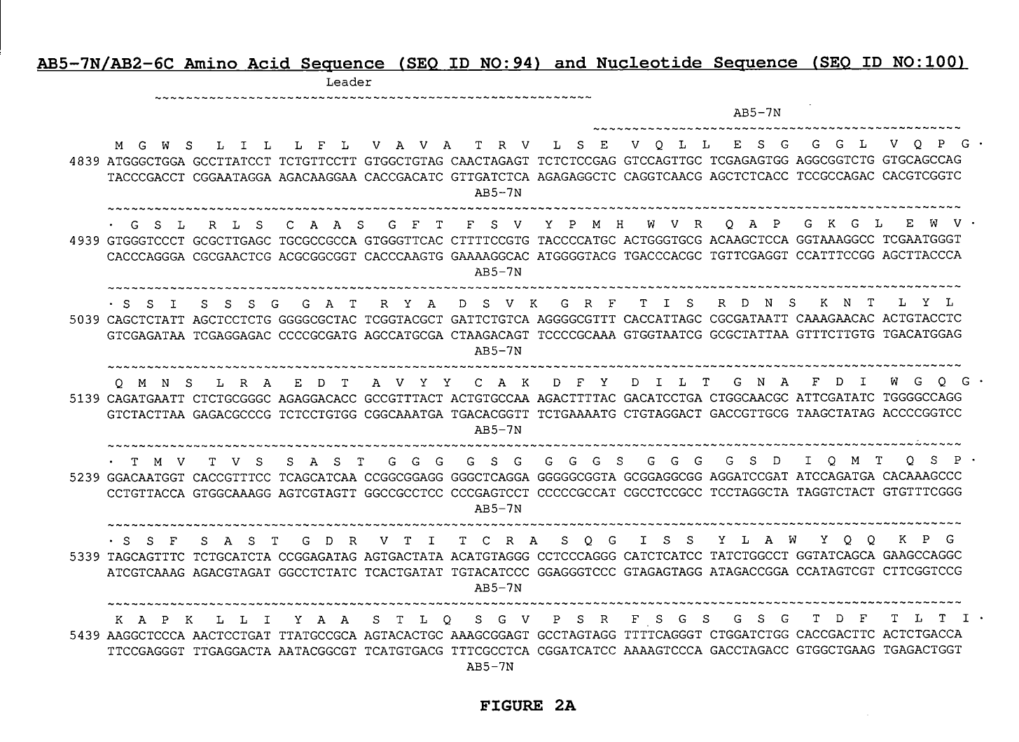

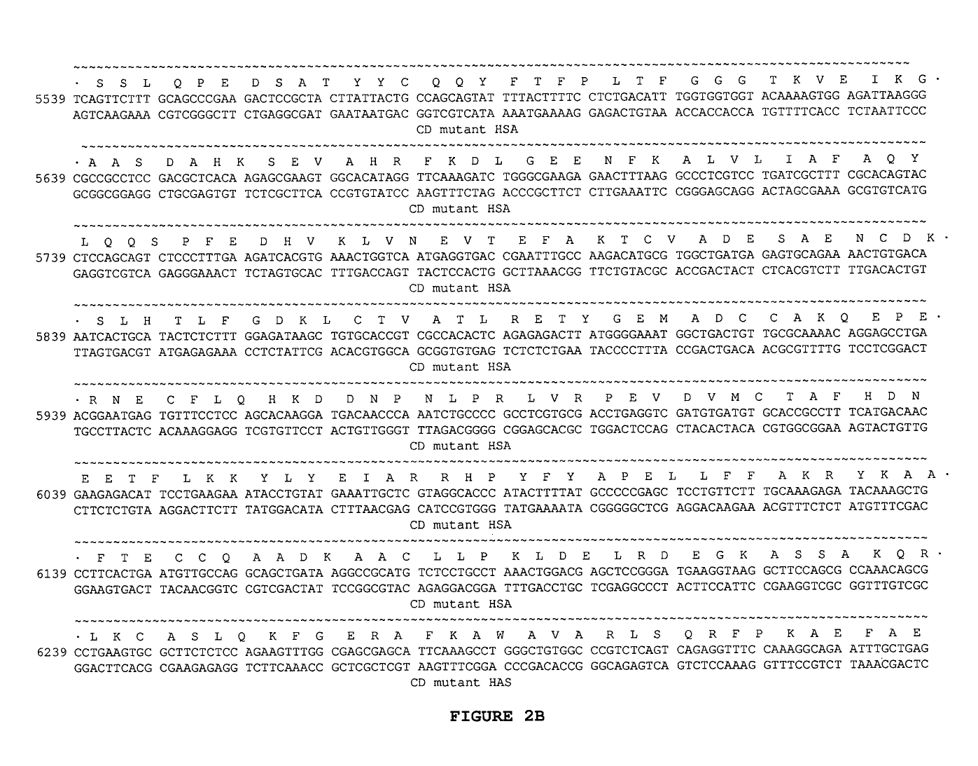

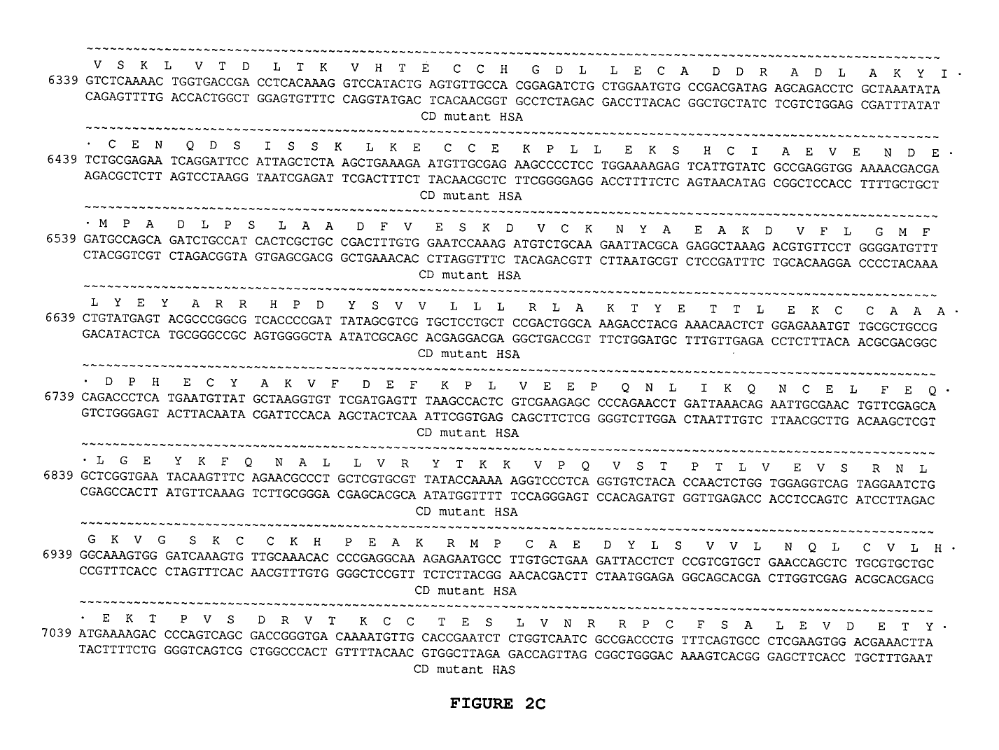

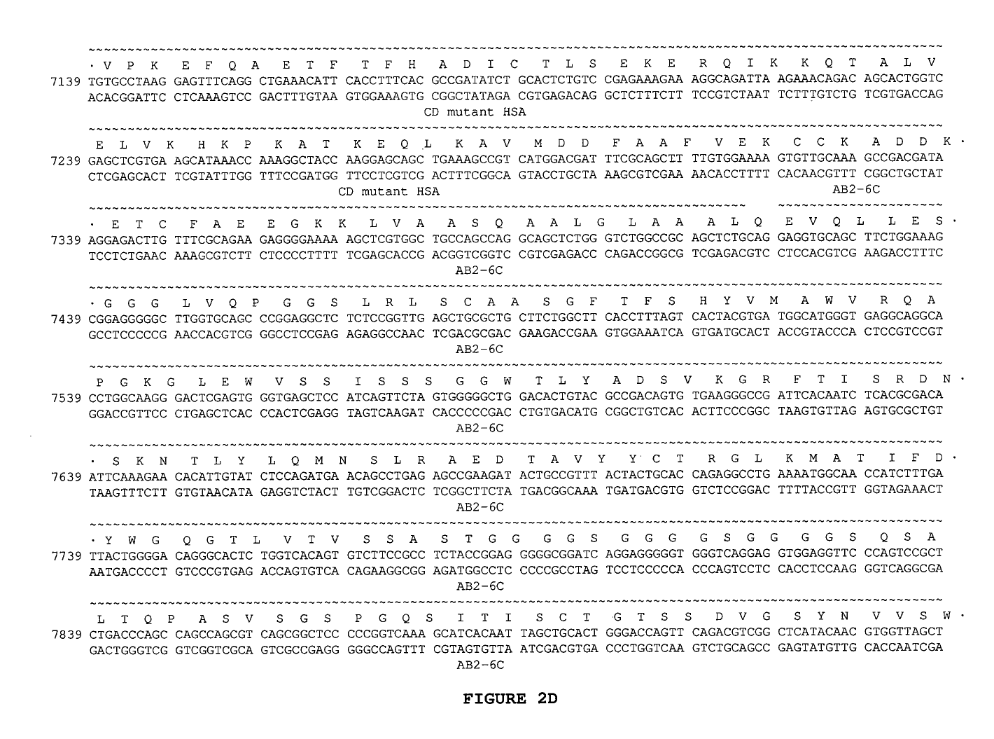

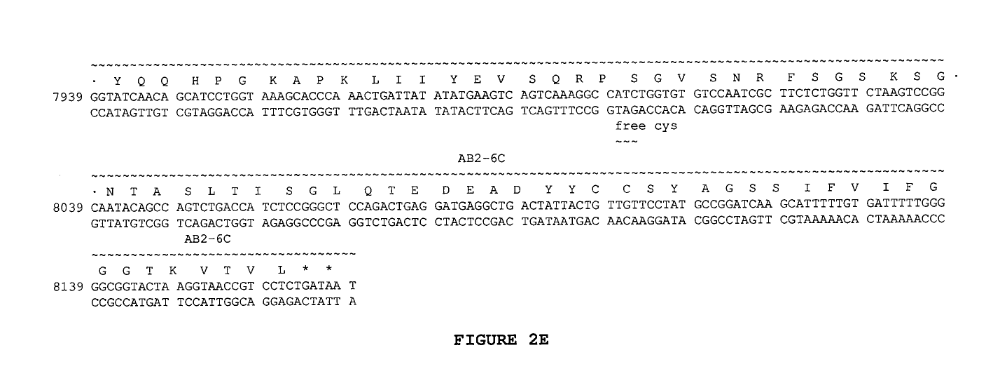

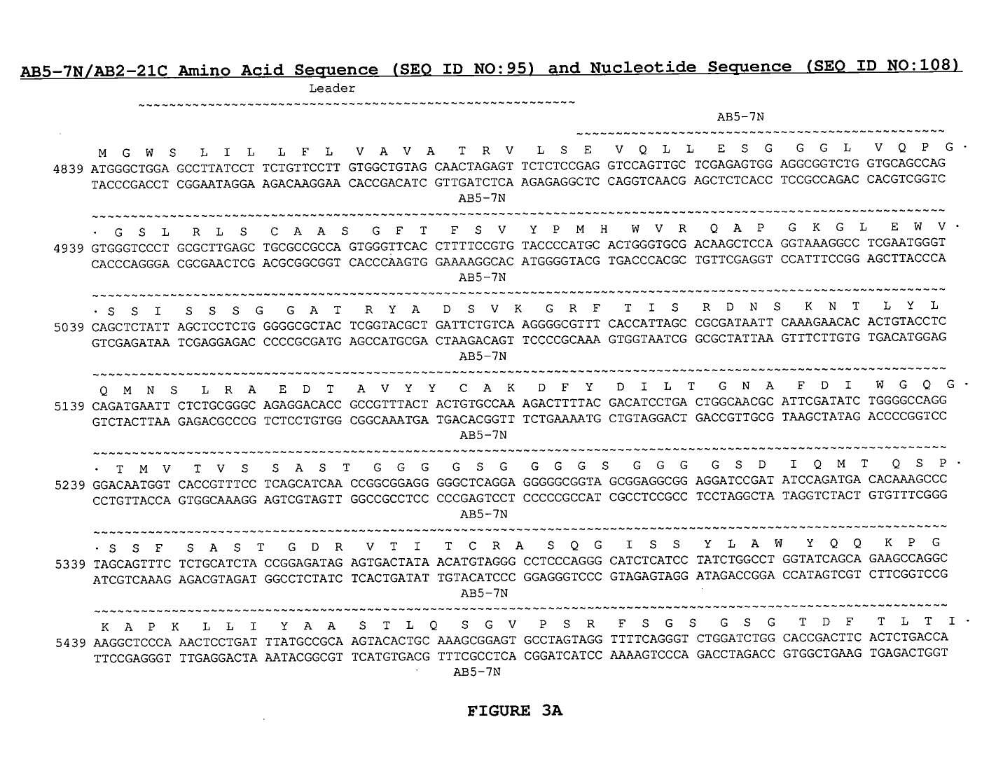

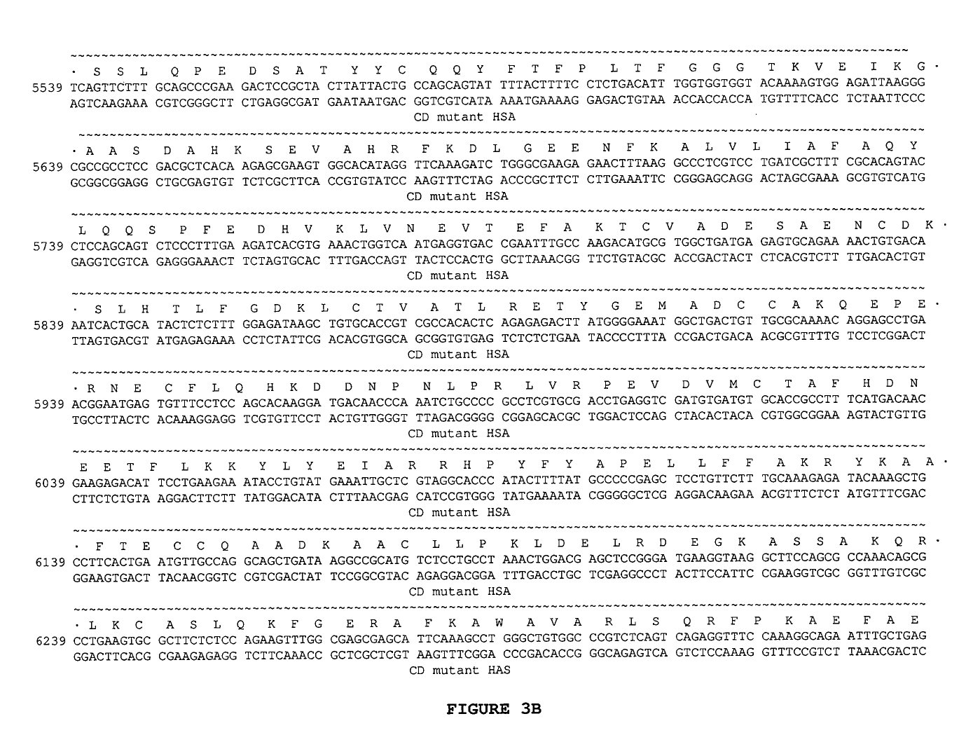

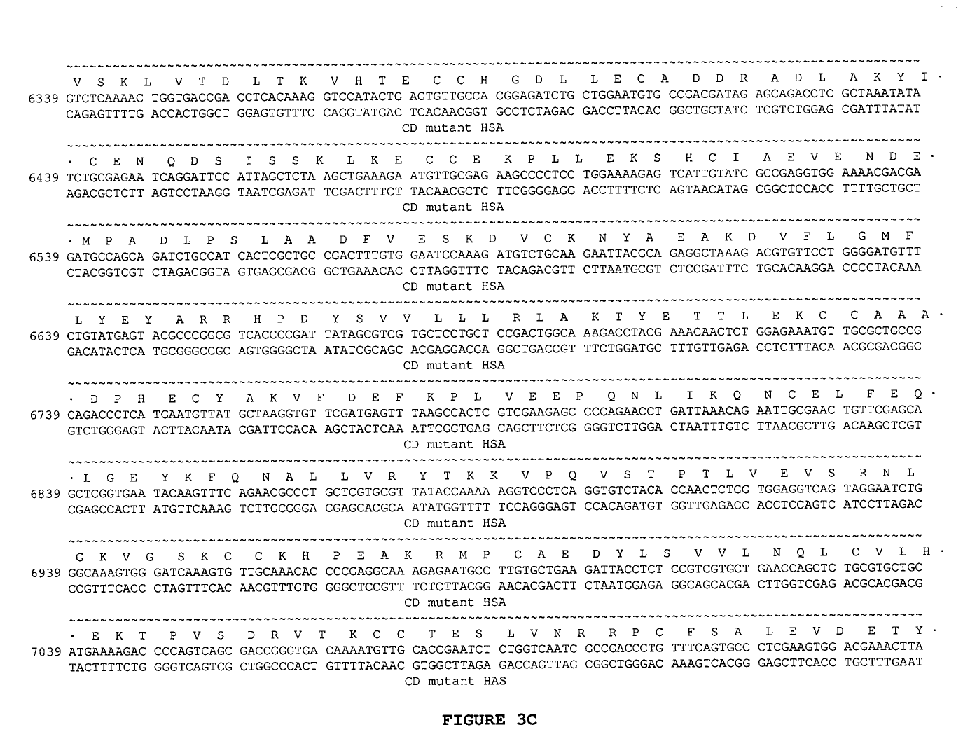

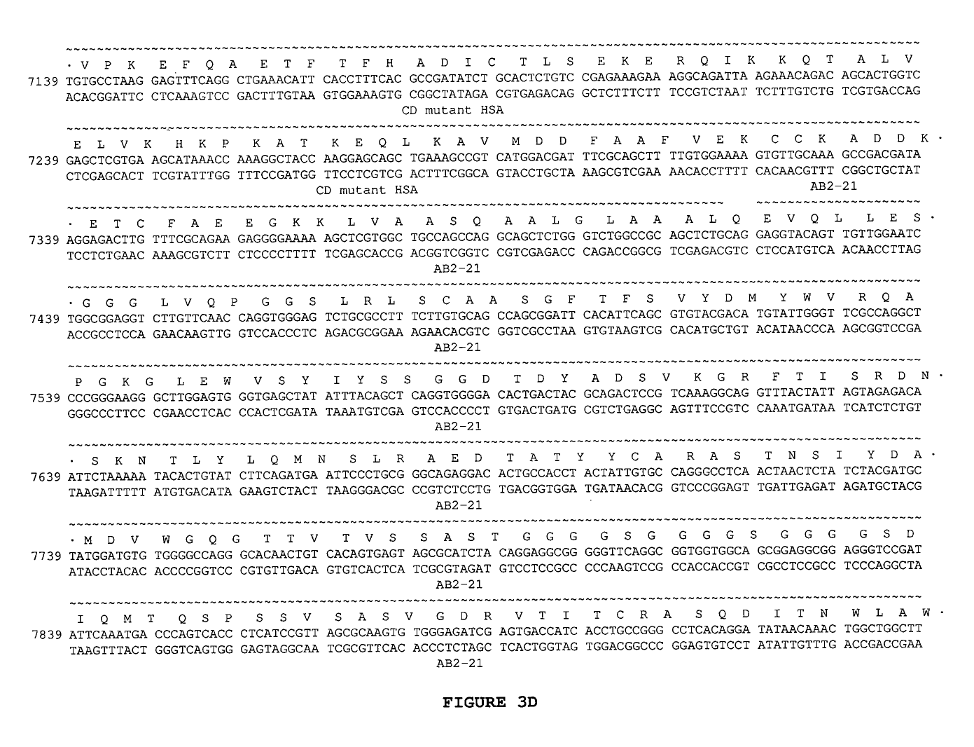

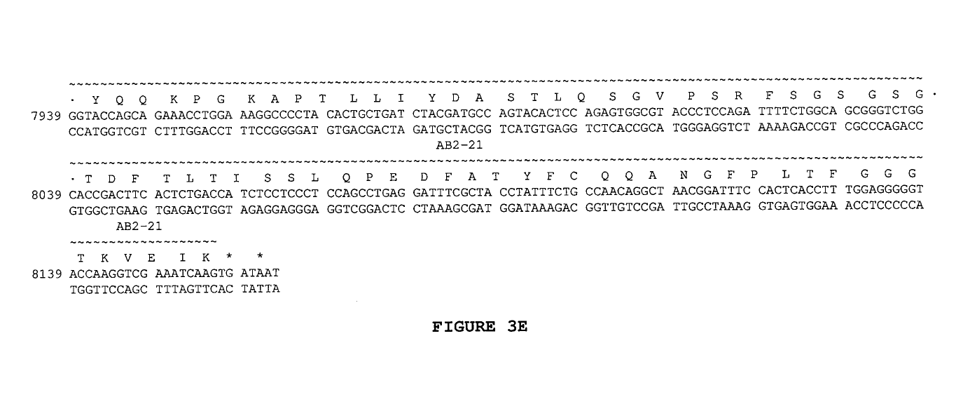

### BRIEF DESCRIPTION OF THE SEQUENCE LISTINGS

SEQ ID NO:1: scFv IGF-1R module 5-7 amino acid sequence

SEQ ID NO:2: stabilized scFv IGF-1R module 5-7 amino acid sequence

SEQ ID NO:3: stabilized homogeneous scFv IGF-1R module 5-7 amino acid sequence

SEQ ID NO:4: stabilized expression optimized scFv IGF-1R module 5-7 amino acid sequence

SEQ ID NO:5: stabilized homogeneous expression optimized scFv IGF-1R module 5-7 amino acid sequence

SEQ ID NO:6: VH IGF-1R module 5-7 amino acid sequence

SEQ ID NO:7: stabilized homogeneous VH IGF-1R module 5-7 amino acid sequence

SEQ ID NO:8: Fab HC IGF-1R module 5-7 amino acid sequence

SEQ ID NO:9: stabilized homogeneous Fab HC IGF-1R module 5-7 amino acid sequence

SEQ ID NO:10: Fab LC IGF-1R module 5-7 amino acid sequence

SEQ ID NO:11: stabilized Fab LC IGF-1R module 5-7 amino acid sequence

SEQ ID NO:12: VL IGF-1R module 5-7 amino acid sequence

SEQ ID NO:13: stabilized VL IGF-1R module 5-7 amino acid sequence

SEQ ID NO:14: scFv IGF-1R module 5-5 amino acid sequence

SEQ ID NO:15: homogeneous scFv IGF-1R module 5-5 amino acid sequence

SEQ ID NO:16: Fab HC IGF-1R module 5-5 amino acid sequence

SEQ ID NO:17: Fab LC IGF-1R module 5-5 amino acid sequence

SEQ ID NO:18: monomeric homogeneous Human albumin-like linker amino acid sequence

SEQ ID NO:19: monomeric homogeneous Human albumin-like linker amino acid sequence with “C345 and N503Q” substitutions in the human albumin sequence

SEQ ID NO:20: dimeric CLkappa-like linker amino acid sequence

SEQ ID NO:21: dimeric CLlambda-like linker amino acid sequence

SEQ ID NO:22: dimeric IgG2-like linker amino acid sequence

SEQ ID NO:23: dimeric IgG2-like short linker amino acid sequence

SEQ ID NO:24: dimeric glycosylated IgG1-like linker amino acid sequence

SEQ ID NO:25: dimeric aglycosylated IgG1-like linker amino acid sequence

SED ID NO:26: dimeric hyperglycosylated IgG1-like linker

SEQ ID NO:27: dimeric glycosylated IgG4-like linker amino acid sequence

SEQ ID NO:28: dimeric aglycosylated IgG4-like linker amino acid sequence

SEQ ID NO:29: dimeric hyperglycosylated IgG4-like linker amino acid sequence

SEQ ID NO:30: trimeric TRAIL-like linker amino acid sequence

SEQ ID NO:31: trimeric LIGHT-like linker amino acid sequence

SEQ ID NO:32: chemically and biologically inert linker amino acid sequence

SEQ ID NO:33: scFv ErbB3 module 2-3 amino acid sequence

SEQ ID NO:34: stabilized scFv ErbB3 module 2-3 amino acid sequence

SEQ ID NO:35: VH ErbB3 module 2-3 amino acid sequence

SEQ ID NO:36: stabilized VH ErbB3 module 2-3 amino acid sequence

SEQ ID NO:37: Fab HC ErbB3 module 2-3 amino acid sequence

SEQ ID NO:38: stabilized Fab HC ErbB3 module 2-3 amino acid sequence

SEQ ID NO:39: Fab LC ErbB3 module 2-3 amino acid sequence

SEQ ID NO:40: stabilized Fab LC ErbB3 module 2-3 amino acid sequence

SEQ ID NO:41: VL ErbB3 module 2-3 amino acid sequence

SEQ ID NO:42: stabilized VL ErbB3 module 2-3 amino acid sequence

SEQ ID NO:43: scFv ErbB3 module 2-6 amino acid sequence

SEQ ID NO:44: scFv ErbB3 module 2-21 amino acid sequence

SEQ ID NO:45: homogeneous scFv ErbB3 module 2-21 amino acid sequence

SEQ ID NO:46: scFv ErbB3 module E3B amino acid sequence

SEQ ID NO:47: scFv stabilized and optimized ErbB3 module E3Bc8 amino acid sequence

SEQ ID NO:48: tandem ErbB3 module A amino acid sequence

SEQ ID NO:49: tandem ErbB3 module B amino acid sequence

SEQ ID NO:50: tandem ErbB3 module C amino acid sequence

SEQ ID NO:51: dimeric IgG2-like Fc linker amino acid sequence

SEQ ID NO:52: dimeric IgG2-like short Fc linker amino acid sequence

SEQ ID NO:53: dimeric glycosylated IgG1-like Fc linker amino acid sequence

SEQ ID NO:54: dimeric aglycosylated IgG1-like Fc linker amino acid sequence

SEQ ID NO:55: dimeric glycosylated IgG4-like Fc linker amino acid sequence

SEQ ID NO:56: dimeric aglycosylated IgG4-like Fc linker amino acid sequence

SEQ ID NO:57: whole chain ErbB3 module 2-3 amino acid sequence

SEQ ID NO:58: whole chain IGF-1R module 5-7 amino acid sequence

SEQ ID NO:59 VH IGF-1R module 5-6 amino acid sequence

SEQ ID NO:60 stabilized VH IGF-1R module 5-6 amino acid sequence

SEQ ID NO:61: Fab HC IGF-1R module 5-6 amino acid sequence

SEQ ID NO:62: stabilized Fab HC IGF-1R module 5-6 amino acid sequence

SEQ ID NO:63: scFv IGF-1R module 5-6 amino acid sequence

SEQ ID NO:64: stabilized scFv IGF-1R module 5-6 amino acid sequence

SEQ ID NO:65: stabilized homogeneous scFv IGF-1R module 5-6 amino acid sequence

SEQ ID NO:66: Fab LC IGF-1R module 5-6 amino acid sequence

SEQ ID NO:67: VL IGF-1R module 5-6 amino acid sequence

SEQ ID NO:68: stabilized scFv IGF-1R module 5-5 amino acid sequence

SEQ ID NO:69: homogeneous stabilized scFv IGF-1R module 5-5 amino acid sequence

SEQ ID NO:70: VH IGF-1R module 5-5 amino acid sequence

SEQ ID NO:71: stabilized VH IGF-1R module 5-5 amino acid sequence

SEQ ID NO:72: stabilized Fab HC IGF-1R module 5-5 amino acid sequence

SEQ ID NO:73: scFv ErbB3 module 2-14 amino acid sequence

SEQ ID NO:74: stabilized scFv ErbB3 module 2-14 amino acid sequence

SEQ ID NO:75: VH ErbB3 module 2-14 amino acid sequence

SEQ ID NO:76: stabilized VH ErbB3 module 2-14 amino acid sequence

SEQ ID NO:77: Fab HC ErbB3 module 2-14 amino acid sequence

SEQ ID NO:78: stabilized Fab HC ErbB3 module 2-14 amino acid sequence

SEQ ID NO:79: Fab LC ErbB3 module 2-14 amino acid sequence

SEQ ID NO:80: VL ErbB3 module 2-14 amino acid sequence

SEQ ID NO:81: stabilized scFv ErbB3 module 2-21 amino acid sequence

SEQ ID NO:82: stabilized homogeneous scFv ErbB3 module 2-21 amino acid sequence

SEQ ID NO:83: VH ErbB3 module 2-21 amino acid sequence

SEQ ID NO:84: stabilized VH ErbB3 module 2-21 amino acid sequence

SEQ ID NO:85: VL ErbB3 module 2-21 amino acid sequence

SEQ ID NO:86: Fab LC ErbB3 module 2-21 amino acid sequence

SEQ ID NO:87: Fab HC ErbB3 module 2-21 amino acid sequence

SEQ ID NO:88: stabilized Fab HC ErbB3 module 2-21 amino acid sequence

SEQ ID NO:89: VH ErbB3 module E3B amino acid sequence

SEQ ID NO:90: VL ErbB3 module E3B amino acid sequence

SEQ ID NO:91: Fab LC ErbB3 module E3B amino acid sequence

SEQ ID NO:92: Fab HC ErbB3 module E3B amino acid sequence

SEQ ID NO:93 AB5-7N/AB2-3C amino acid sequence

SEQ ID NO:94 AB5-7N/AB2-6C amino acid sequence

SEQ ID NO:95 AB5-7N/AB2-21C amino acid sequence

SEQ ID NO:96 AB2-3N/AB5-7C amino acid sequence

SEQ ID NO:97 AB2-6N/AB5-7C amino acid sequence

SEQ ID NO:98 AB2-21N/AB5-7C amino acid sequence

SEQ ID NO:99 AB5-7N/AB2-3C nucleotide sequence

SEQ ID NO:100 AB5-7N/AB2-6C nucleotide sequence

SEQ ID NO:101 scFv IGF-1R module 5-7 nucleotide sequence

SEQ ID NO:102: stabilized scFv IGF-1R module 5-7 nucleotide sequence

SEQ ID NO:103: stabilized homogeneous scFv IGF-1R module 5-7 nucleotide sequence

SEQ ID NO:104: stabilized expression optimized scFv IGF-1R module 5-7 nucleotide sequence

SEQ ID NO:105: stabilized homogeneous expression optimized scFv IGF-1R module 5-7 nucleotide sequence

SEQ ID NO:106: VH IGF-1R module 5-7 nucleotide sequence

SEQ ID NO:107: stabilized homogeneous VH IGF-1R module 5-7

SEQ ID NO:108: AB5-7N/AB2-21C nucleotide sequence

SEQ ID NO:109: stabilized homogeneous Fab HC IGF-1R module 5-7 nucleotide sequence

SEQ ID NO:110: Fab LC IGF-1R module 5-7 nucleotide sequence

SEQ ID NO:111: stabilized Fab LC IGF-1R module 5-7 nucleotide sequence

SEQ ID NO:112: VL IGF-1R module 5-7 nucleotide sequence

SEQ ID NO:113: stabilized VL IGF-1R module 5-7 nucleotide sequence

SEQ ID NO:114: scFv IGF-1R module 5-5 nucleotide sequence

SEQ ID NO:115 AB2-3N/AB5-7C nucleotide sequence

SEQ ID NO:116 AB2-6N/AB5-7C nucleotide sequence

SEQ ID NO:117 AB2-21N/AB5-7C nucleotide sequence

SEQ ID NO:118: monomeric homogeneous Human albumin-like linker nucleotide sequence

SEQ ID NO:119: monomeric homogeneous Human albumin-like linker nucleotide sequence with “C345 and N503Q” substitutions in the human albumin sequence

SEQ ID NO:120: dimeric CLkappa-like linker nucleotide sequence

SEQ ID NO:121: dimeric CLlambda-like linker nucleotide sequence

SEQ ID NO:122: dimeric IgG2-like linker nucleotide sequence

SEQ ID NO:123: dimeric IgG2-like short linker nucleotide sequence

SEQ ID NO:124: dimeric aglycosylated IgG1-like linker nucleotide sequence

SEQ ID NO:125: dimeric hyperglycosylated IgG1-like linker nucleotide sequence

SEQ ID NO:126: trimeric TRAIL-like linker nucleotide sequence

SEQ ID NO:127: trimeric LIGHT-like linker nucleotide sequence

SEQ ID NO:128: scFv ErbB3 module 2-3 nucleotide sequence

SEQ ID NO:129: stabilized scFv ErbB3 module 2-3 nucleotide sequence

SEQ ID NO:130: VH ErbB3 module 2-3 nucleotide sequence

SEQ ID NO:131: Fab LC ErbB3 module 2-3 nucleotide sequence

SEQ ID NO:132: VL ErbB3 module 2-3 nucleotide sequence

SEQ ID NO:133: scFv ErbB3 module 2-6 nucleotide sequence

SEQ ID NO:134: scFv ErbB3 module E3B nucleotide sequence

SEQ ID NO:135: scFv stabilized affinity matured ErbB3 module E3Bc8 nucleotide sequence

SEQ ID NO:136: tandem ErbB3 module A nucleotide sequence

SEQ ID NO:137: tandem ErbB3 module B nucleotide sequence

SEQ ID NO:138: tandem ErbB3 module C nucleotide sequence

SEQ ID NO:139: whole chain ErbB3 module 2-3 nucleotide sequence

SEQ ID NO:140: whole chain IGF-1R module 5-7 nucleotide sequence

SEQ ID NO:141: scFv IGF-1R module 5-6 nucleotide sequence

SEQ ID NO:142: scFv ErbB3 module 2-14 nucleotide sequence

SEQ ID NO:143: (monomeric) Human serum albumin linker amino acid sequence

SEQ ID NO:144: (monomeric) Human serum albumin linker nucleotide sequence

SEQ ID NO:145: (monomeric) Human serum albumin linker amino acid sequence with C34S and N503Q substitutions

SEQ ID NO:146: (monomeric) Human serum albumin linker nucleotide sequence encoding “C345 and N503Q” substitutions

SEQ ID NO:147: control shRNA sequence

SEQ ID NO:148: IGF-1R targeted shRNA sequence

SEQ ID NO:149: ErbB3 targeted shRNA sequence (mod1)

SEQ ID NO:150: ErbB3 targeted shRNA sequence (mod2)

### DETAILED DESCRIPTION

### I. Definitions

The term “BBA” as used herein refers to an artificial hybrid molecule having two different binding moieties and thus two different binding sites (such as two different antibody binding sites). The two different binding moieties are “covalently linked”, meaning that they are chemically bonded together via a “linker moiety”, which refers to a distinct structural component of the BBA that connects the two different binding moieties.

“IGF-1R” refers to the receptor for human insulin-like growth factor 1 (IGF-1, formerly known as somatomedin C). IGF1-R also binds to, and is activated by, insulin-like growth factor 2 (IGF-2). IGF1-R is a receptor tyrosine kinase, meaning that it transmits signals into the cell by catalyzing the addition of phosphate molecule(s) to one or more particular tyrosines of one or more proteins intracellularly. Tyrosine phosphorylation by IGF1-R includes an autocatalytic function: IGFR-1 activation by IGF-1 or IGF-2 results in auto-phosphorylation of IGF1-R. The amino acid sequence of human IGF-1R precursor is provided at Genbank Accession No. NP_000866.

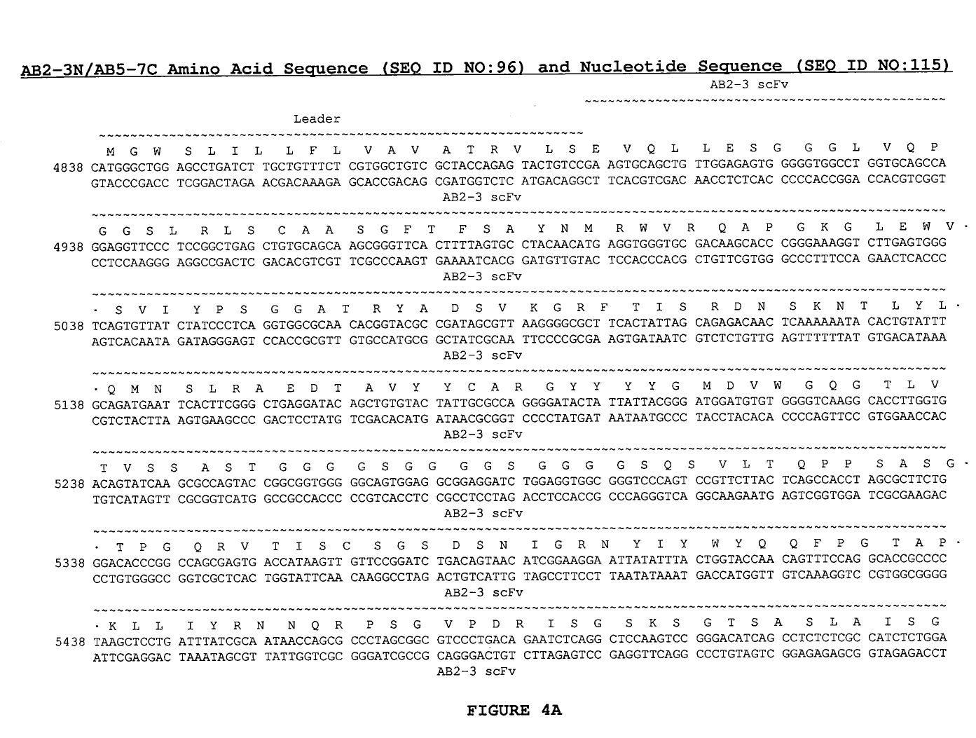

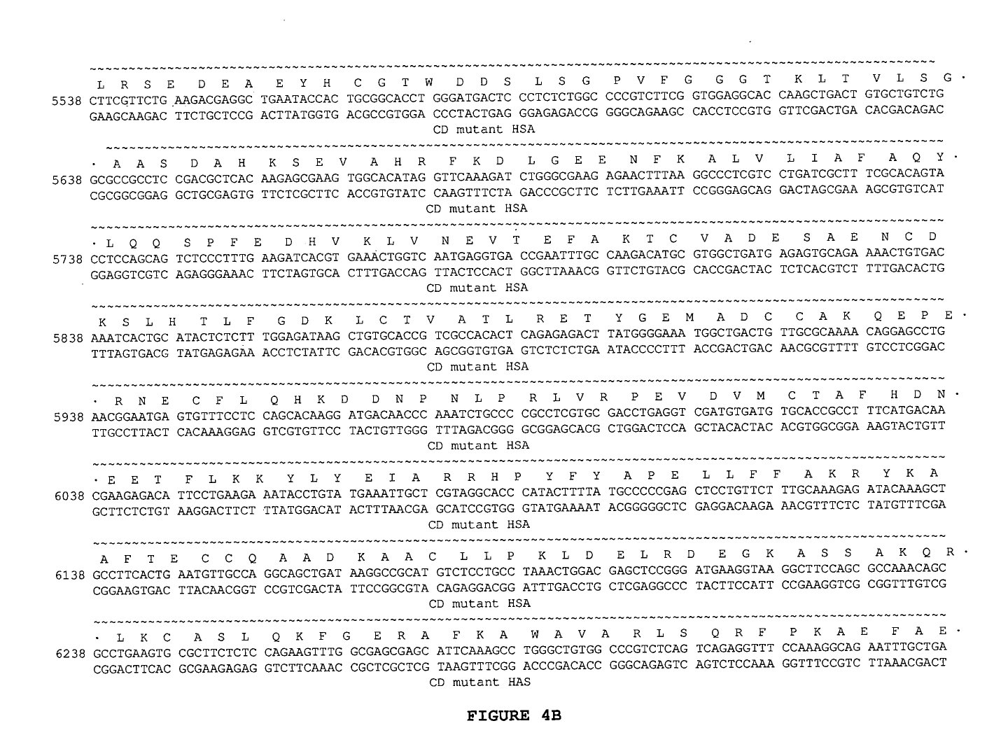

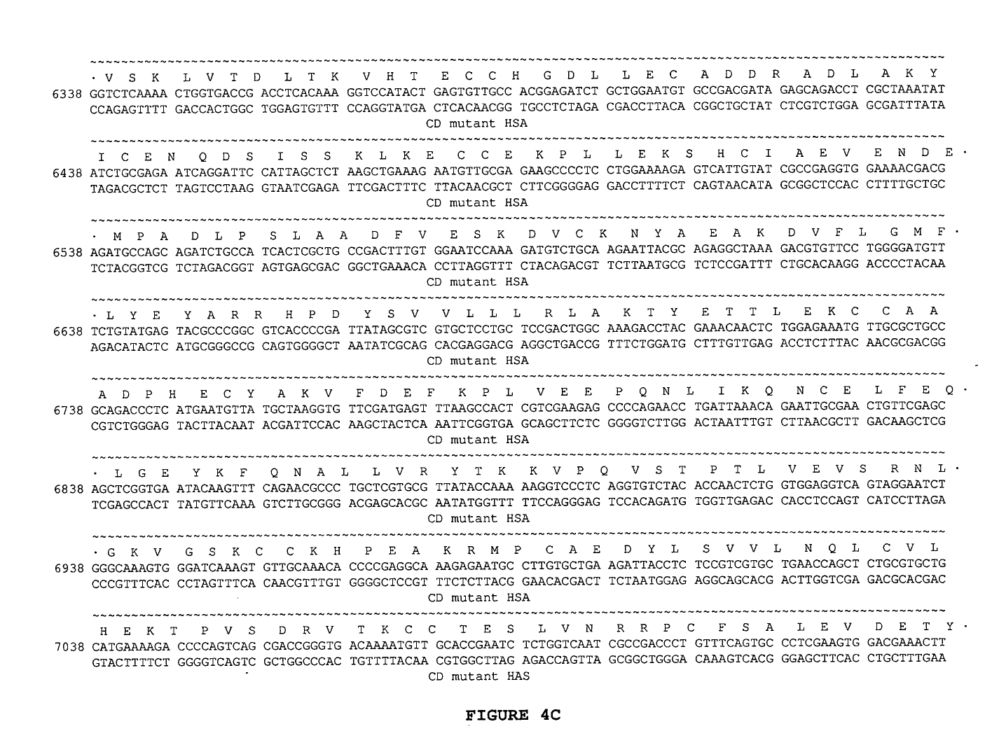

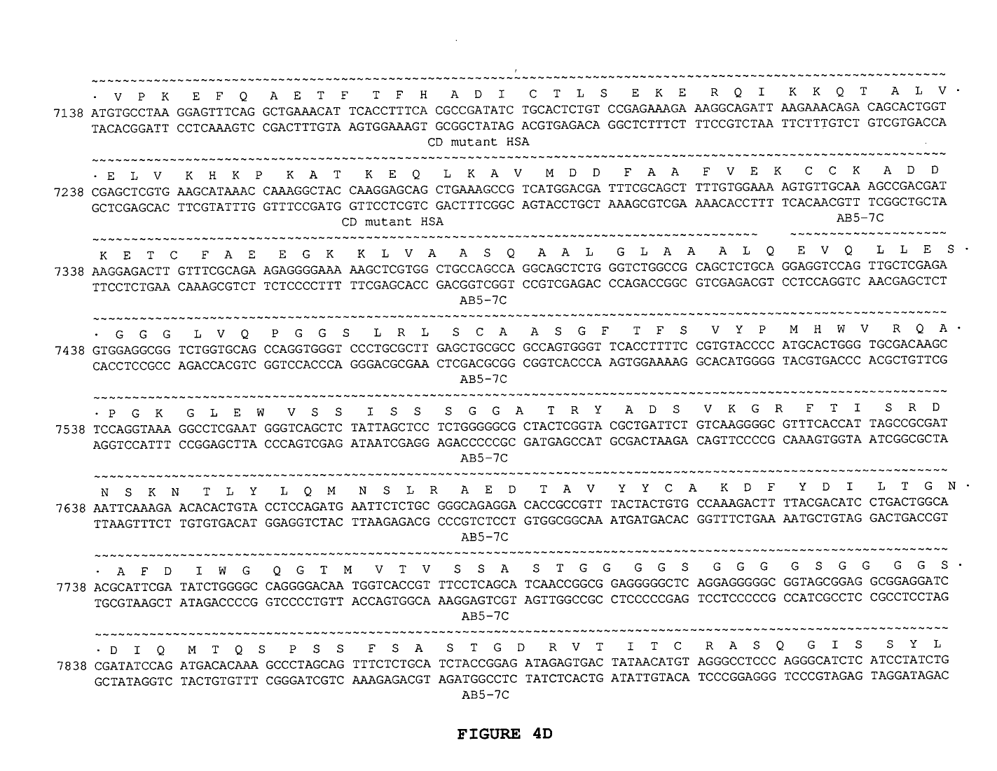

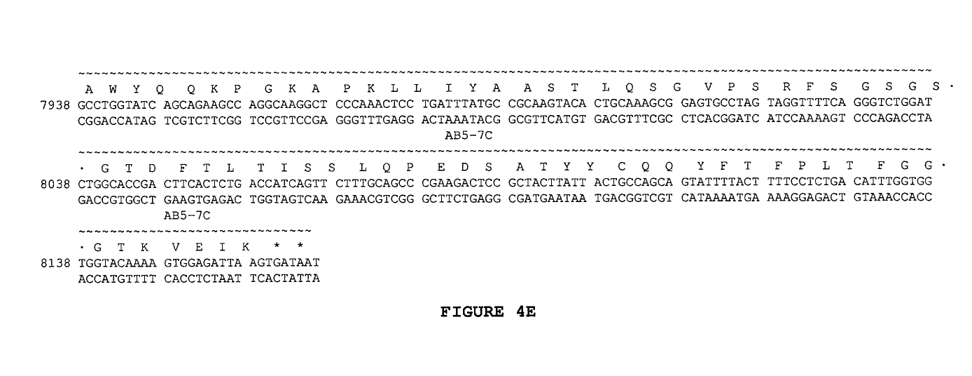

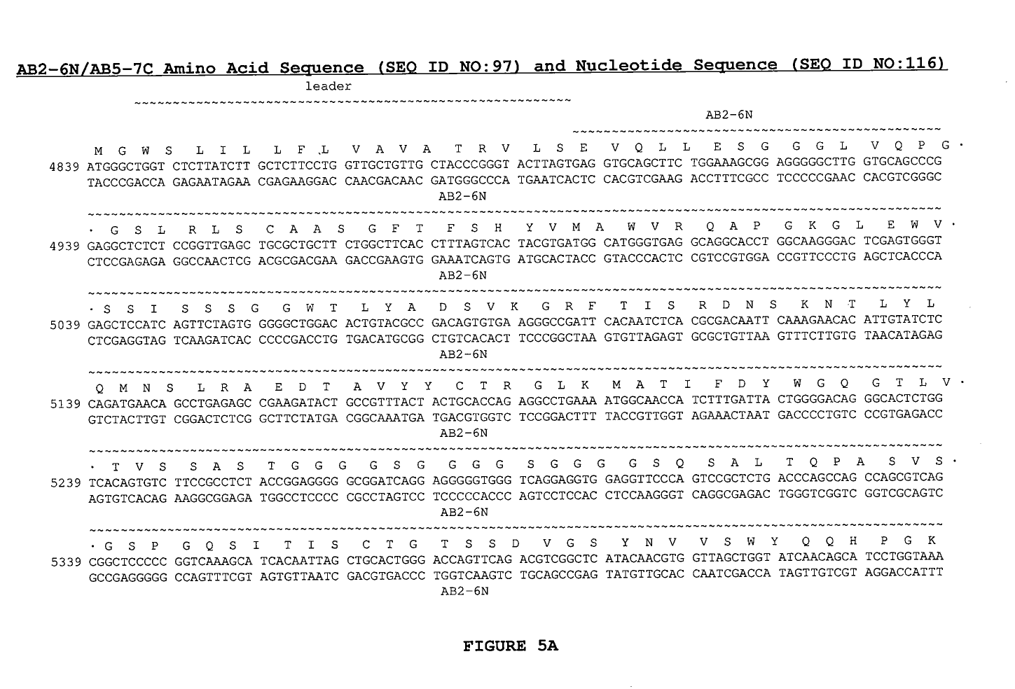

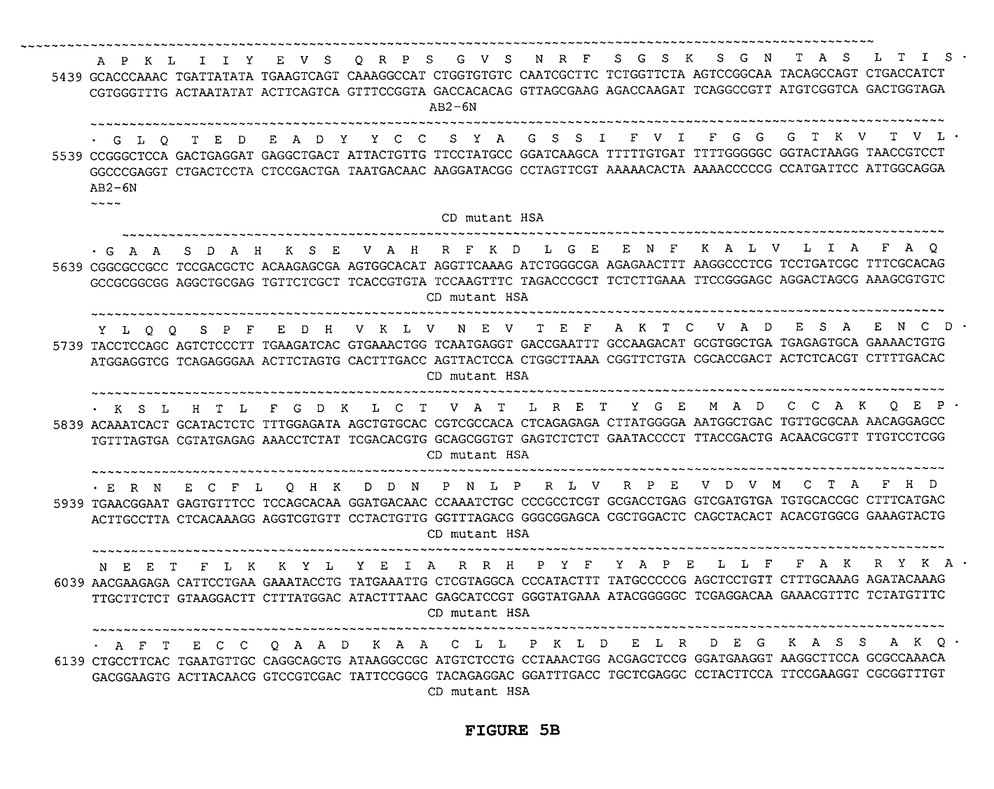

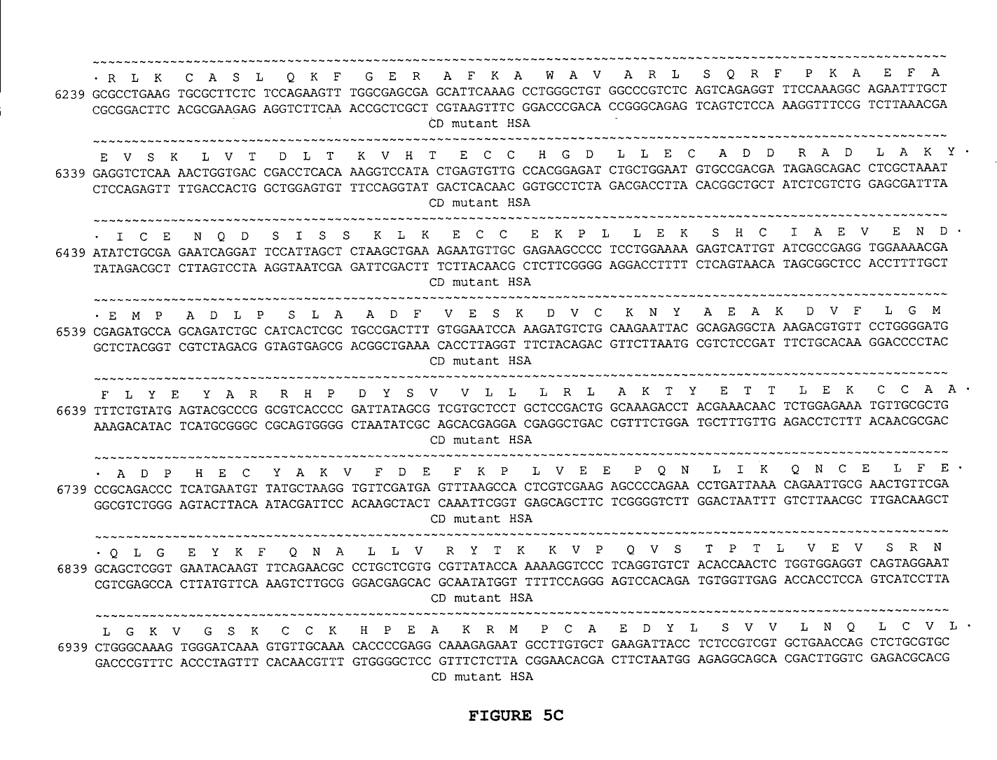

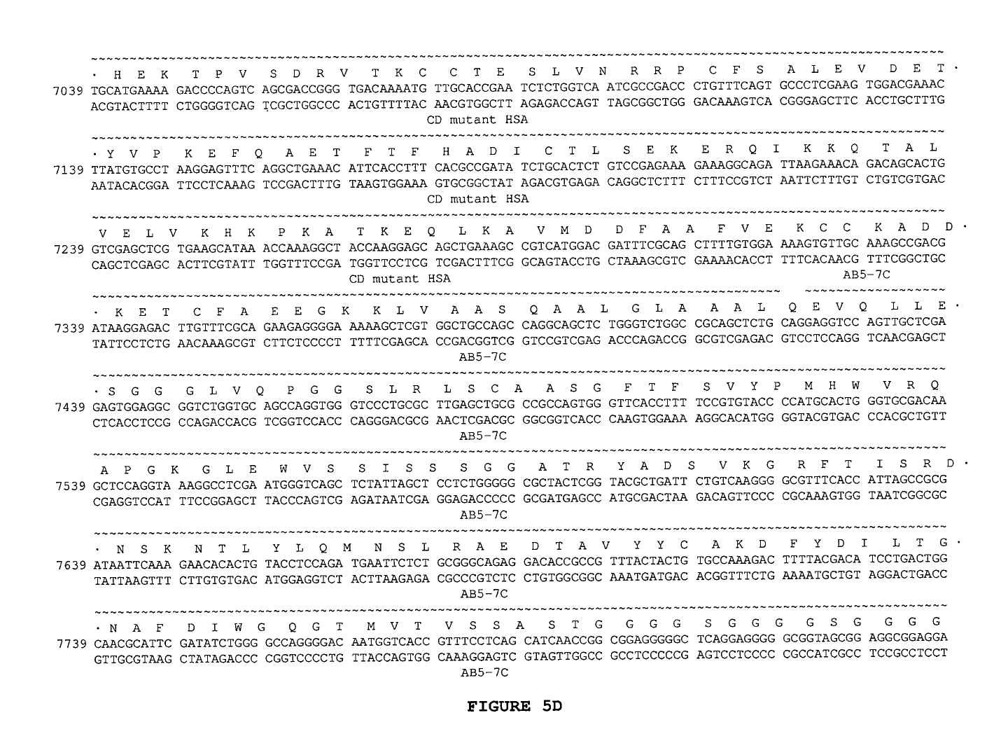

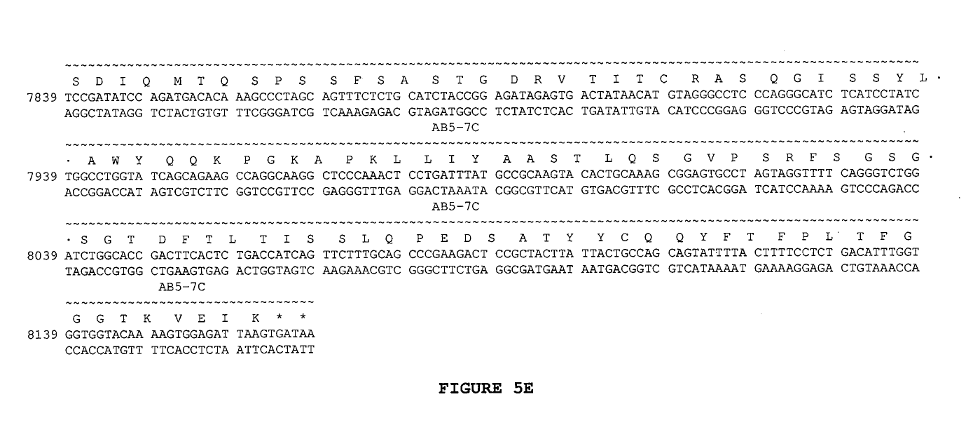

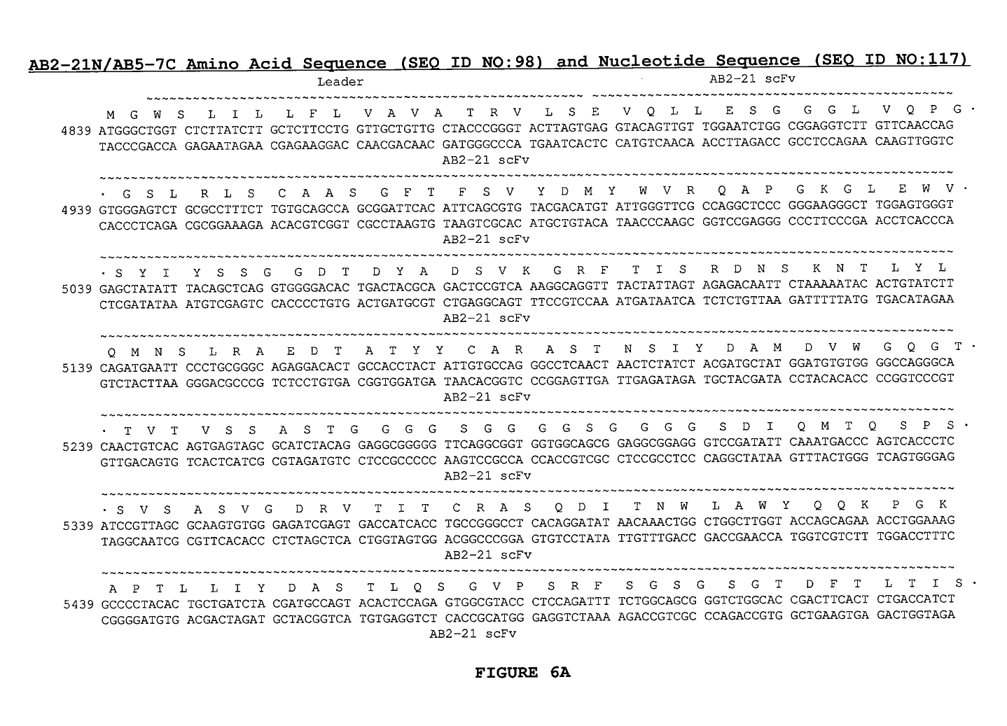

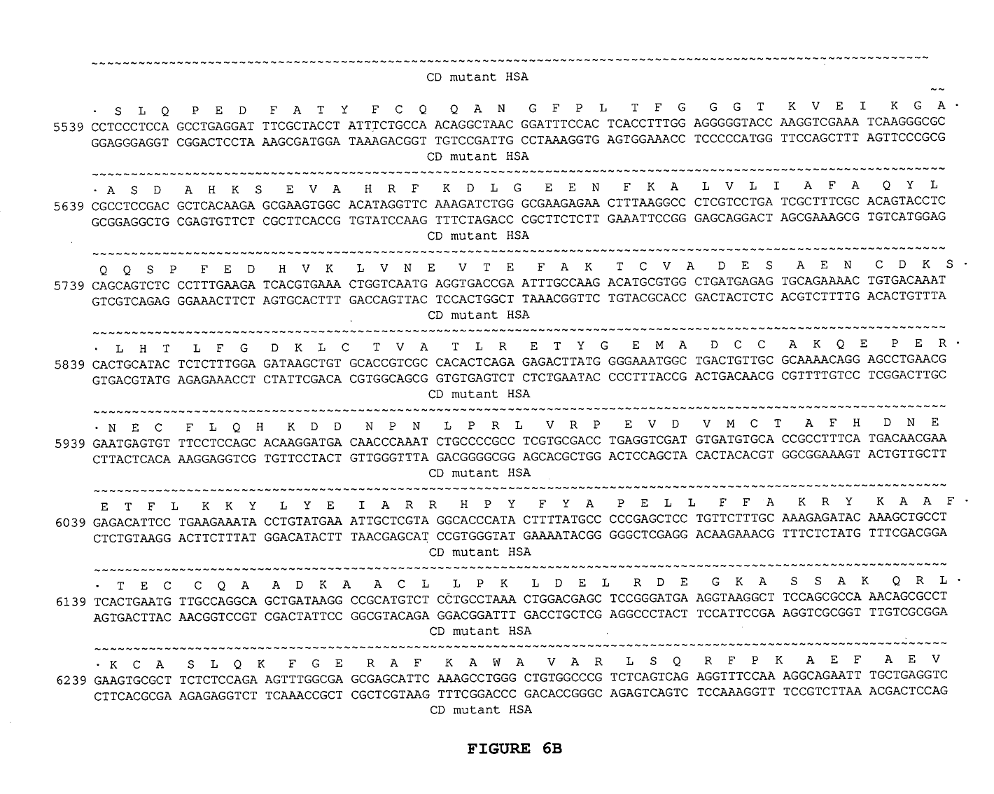

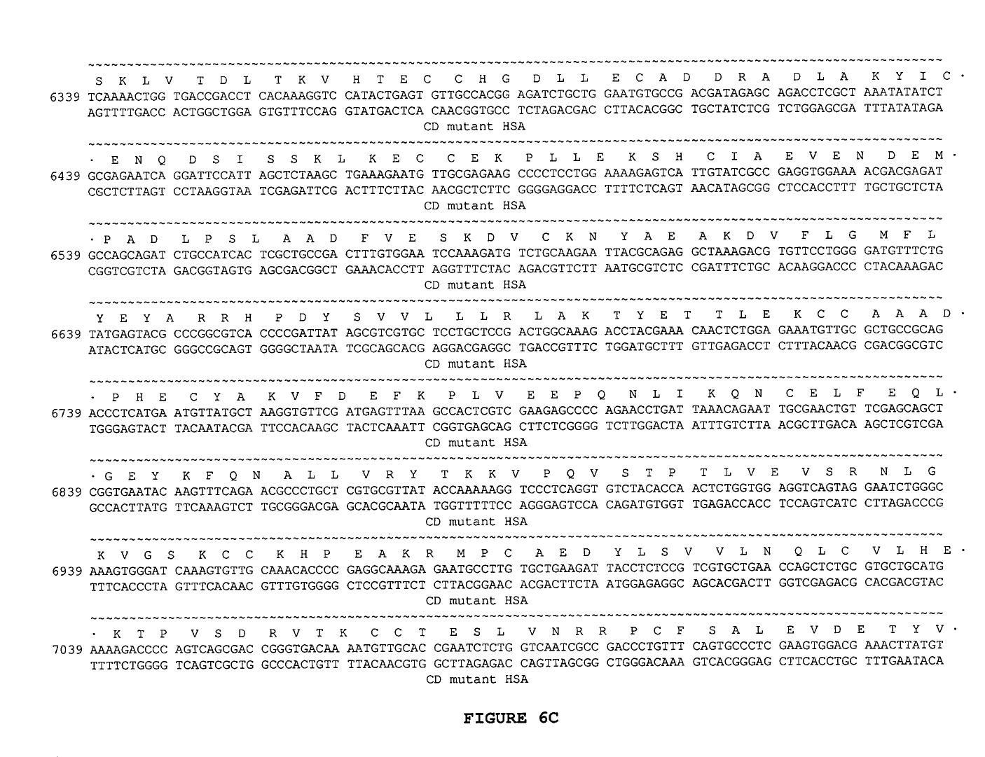

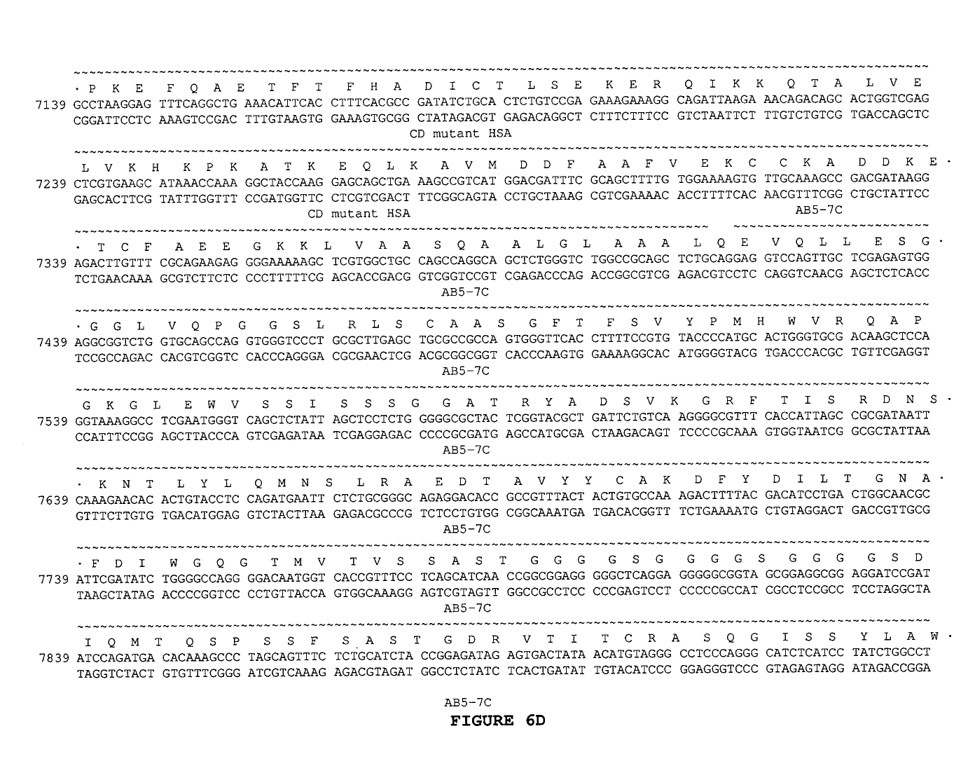

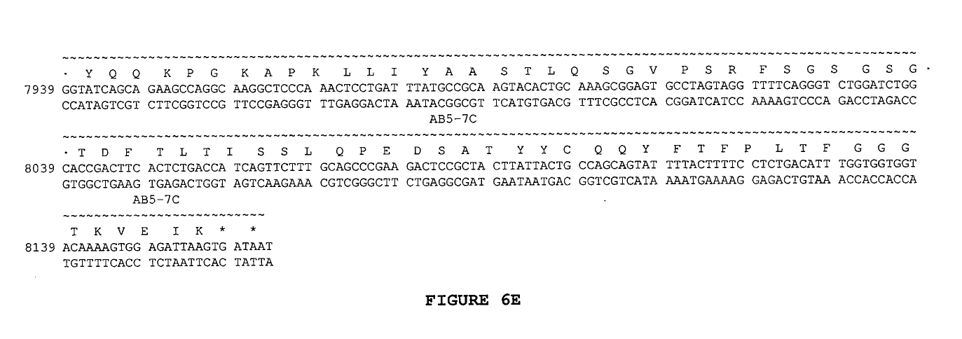

“ErbB3,”and “HER3” refer to human ErbB3 protein, as described in U.S. Pat. No. 5,480,968. The human ErbB3 protein sequence is shown in FIG. 4 and SEQ ID NO:4 of U.S. Pat. No. 5,480,968, wherein the first 19 amino acids correspond to the leader sequence that is cleaved from the mature protein. ErbB3 is a tyrosine kinase substrate and is a member of the ErbB family of receptors, other members of which include ErbB1 (EGFR), ErbB2 (HER2/Neu) and ErbB4. While ErbB3 itself lacks tyrosine kinase activity, ErbB3 is believed to only act in heterodimeric form together with another ErbB family receptor. ErbB 1, ErbB2 and ErbB4 are all receptor tyrosine kinases, and the activation of heterodimeric ErbB3 results in tyrosine phosphorylation of ErbB3. Ligands for the ErbB family include heregulin (HRG), betacellulin (BTC), epidermal growth factor (EGF), heparin-binding epidermal growth factor (HB-EGF), transforming growth factor alpha (TGFα), amphiregulin (AR), epigen (EPG) and epiregulin (EPR).

The term “monomeric linker”, as used herein, refers to a linker moiety used in a bispecifc binding agent (BBA) that results in monomers of the BBA being formed. That is, the complete BBA consist of a single molecule (a monomer) that is composed of the two different binding moieties (one specific for IGF-1R, the other specific for ErbB3) covalently linked together by the linker moiety. Typically, monomeric linkers are derived from proteins that exist as monomers, such as, for example, human serum albumin.

The term “dimeric linker”, as used herein, refers to a linker moiety used in a BBA that results in a dimeric BBA being formed. That is, the complete BBA consists of two molecules (a dimer) or subunits, wherein each subunit of the BBA is composed of at least one, and typically two different binding moieties (one specific for IGF-1R, the other specific for ErbB3) covalently linked together by the linker moiety. Typically, dimeric linkers are derived from proteins that exist as dimers, such as, for example, immunoglobulin molecules, such that these linkers dimerize (e.g., through disulfide bridges) to create dimeric BBAs.

The term “trimeric linker”, as used herein, refers to a linker moiety used in a BBA that results in trimers of the BBA being formed. That is, the complete BBA consists of three molecules (a trimer) or subunits, wherein each subunit of the BBA is composed of the two different binding moieties (one specific for IGF-1R, the other specific for ErbB3) covalently linked together by the linker moiety. Typically, trimeric linkers are derived from proteins that exist as trimers, such as, for example, TRAIL or LIGHT, such that these linkers trimerize (e.g., through disulfide bridges) to create trimeric BBAs.

The term “chemically and biologically inert linker”, as used herein, refers to a linker moiety used in a BBA in which the linker moiety does not itself have any chemical or biological activity, for example when administered to a subject.

As used herein, a “glycosylated” linker or a “glycosylated” BBA refers to a linker or BBA that includes carbohydrate moieties on its structure. For example, the presence of one or more glycosylation sites within the sequence of the linker or BBA results in a “glycosylated” linker or BBA upon expression of the linker or BBA.

As used herein, an “aglycosylated” linker or an “aglycosylated” BBA refers to a linker or BBA that does not include any carbohydrate moieties on its structure. For example, the lack of any glycosylation sites within the sequence of the linker or BBA (either existing naturally or created through site-directed mutagenesis by intentional removal of all glycosylation sites) results in an “aglycosylated” linker or BBA upon expression of the linker or BBA.

As used herein, a “hyperglycosylated” linker or a “hyperglycosylated” BBA refers to a modified form of a linker or BBA that includes a greater number of carbohydrate moieties on its structure as compared to an unmodified form of the linker or BBA. For example, modification of a linker or BBA (e.g., by site-directed mutagenesis) to increase the number of glycosylation sites present within the sequence of the linker or BBA results in a “hyperglycosylated” linker or BBA upon expression of the linker or BBA.

As used herein, a “stabilized” sequence (e.g., of a binding moiety used in a BBA) refers to a sequence that has been modified from its original form in order to enhance the stability of the sequence when it is expressed as a protein. For example, the nucleotide sequence encoding an anti-IGF-1R antibody or an anti-ErbB3 antibody (e.g., the VH and/or VL sequence) can be modified (e.g., by site-directed mutagenesis) at one or more encoded amino acid positions in order to enhance the stability of the encoded antibody when expressed within the BBA. Amino acid modifications that enhance the stability of binding moieties, such as antibodies, without significantly altering their binding affinity/specificity are known in the art and can be incorporated into the binding moieties used in the BBAs described herein through standard recombinant DNA techniques.

As used herein, an “optimized” sequence (e.g., of a binding moiety used in a BBA) refers to a sequence that has been modified from its original form in order to enhance the expression of the sequence as a protein. For example, the nucleotide sequence of an anti-IGF-1R antibody or an anti-ErbB3 antibody (e.g., the VH and/or VL sequence) can be modified (e.g., by site-directed mutagenesis) at one or more codons in order to enhance the expression of the encoded antibody (also known in the art as “codon optimization”). Nucleotide (codon) modifications that enhance the protein expression of the encoded binding moieties, such as antibodies, are known in the art and can be incorporated into the binding moieties described herein through standard recombinant DNA techniques.

As used herein, a “stabilized and optimized” sequence refers to a sequence that has been modified from its original form both to enhance the stability of the sequence when it is expressed as a protein and to enhance the expression of the sequence as a protein.

As used herein, a “homogenous” sequence (e.g., of a binding moiety used in a BBA) refers to a sequence that has been modified from its original form in order to enhance the homogeneity of the sequence when it is expressed as a protein. For example, the nucleotide sequence of an anti-IGF-1R antibody or an anti-ErbB3 antibody (e.g., the VH and/or VL sequence) can be modified (e.g., by site-directed mutagenesis) at one or more encoded amino acid positions in order to enhance the homogeneity of the encoded antibody when expressed within the BBA. Amino acid modifications that enhance the homogeneity of binding moieties, such as antibodies, without significantly altering their binding affinity/specificity are known in the art and can be incorporated into the binding moieties used in the BBAs described herein through standard recombinant DNA techniques.

As used herein, “IGF-1R signaling pathway” is intended to encompass signal transduction pathways that initiate through interaction of a ligand with a receptor of the IGF-1R family. Components within an IGF-1R signaling pathway may include: (i) one or more ligands, examples of which include IGF-1 and IGF-2; (ii) one or more receptors, examples of which include IGF-1R and the insulin receptor; (iii) one or more IGF binding proteins and (iv) intracellular kinases and substrates, examples of which include insulin receptor substrate 2 (IRS2), phosphoinositide 3 kinase (PI3K), AKT, RAS, RAF, MEK and mitogen-activated protein kinase (MAPK).

As used herein, the term “ErbB signaling pathway” is intended to encompass signal transduction pathways that initiate through interaction of a ligand with a receptor of the ErbB family. Components within an ErbB signaling pathway may include: (i) one or more ligands, examples of which include heregulin (HRG), betacellulin (BTC), epidermal growth factor (EGF), heparin-binding epidermal growth factor (HB-EGF), transforming growth factor alpha (TGFα), amphiregulin (AR), epigen (EPG) and epiregulin (EPR); (ii) one or more receptors, examples of which include ErbB 1/EGFR, ErbB2, ErbB3 and ErbB4; and (iii) intracellular kinases and substrates, examples of which include phosphoinositide 3 kinase (PI3K), phosphatidylinositol bisphosphate (PIP2), phosphatidylinositol trisphosphate (PIP3), phosphatase and tensin homolog (PTEN), pyruvate dehydrogenase kinase isozyme 1 (PDK1), AKT, RAS, RAF, MEK, the extracellular signal-regulated kinase (ERK), protein phosphatase 2A (PP2A) and SRC protein tyrosine kinase.

The term “inhibition” as used herein, refers to any reproducibly detectable decrease in biological activity mediated by an antibody or BBA. In some embodiments, inhibition provides a statistically significant decrease in biological activity. For example, “inhibition” can refer to a reproducible decrease of about 5%, 10%, 20%, 30%, 40%, 50%, 60%, 70%, 80%, 90%, or 100% in biological activity.

Accordingly inhibition of a) ligand mediated phosphorylation of ErbB3, and b) IGF-1- or IGF-2-mediated phosphorylation of IGF-1R respectively can be demonstrated by the ability of a BBA to reproducibly decrease the level of phosphorylation of a) ErbB3 induced by an ErbB family ligand, orb) IGF-1R induced by IGF-1 or IGF-2, each relative to the phosphorylation in control cells that are not treated with the BBA. The cell which expresses ErbB3 and/or IGF-1R can be a naturally occurring cell or cell line or can be recombinantly produced by introducing nucleic acid encoding ErbB3 and/or IGF-1R into a host cell. In one embodiment, the BBA inhibits an ErbB family ligand mediated phosphorylation of ErbB3 by at least about 5%, 10%, 20%, 30%, 40%, 50%, 60%, 70%, 80%, 90%, or more, as determined, for example, by Western blotting followed by probing with an anti-phosphotyrosine antibody as described in the Examples infra. In another embodiment, the BBA inhibits IGF-1- or IGF-2-mediated phosphorylation of IGF-1R by at least about 5%, 10%, 20%, 30%, 40%, 50%, 60%, 70%, 80%, 90%, or more, as determined, for example, by Western blotting followed by probing with an anti-phosphotyrosine antibody as described in the Examples infra.

The term “antibody” or “immunoglobulin,” as used interchangeably herein, includes whole antibodies and any antigen binding fragment or single chains thereof. A typical antibody comprises at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, CL. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

It has been shown that the antigen-binding function of an antibody can be performed by antigen binding fragments of a full-length antibody. Examples of such binding fragments include (i) an Fab fragment, a monovalent fragment consisting of the V_{L}, V_{H}, CL and CH1 domains; (ii) an F(ab′)₂ fragment, a bivalent fragment comprising two Fab fragments linked by a disulfide bridge at the hinge region; (iii) an Fd fragment consisting of the V_{H} and CH1 domains; (iv) an Fv fragment consisting of the V_{L} and V_{H} domains of a single arm of an antibody; (v) an antibody fragment including VH and VL domains; (vi) an antibody fragment, which consists of a V_{H} domain; (vii) an antibody fragment which consists of a VH or a VL domain; and (viii) an isolated complementarity determining region (CDR) or (ix) a combination of two or more isolated CDRs which may optionally be joined by a synthetic linker.

The term “single chain antibody” or “single chain Fv” (scFV) refers to an antibody in which both a variable region heavy chain domain and a variable region light chain domain are contained within a single, linear protein. Although these two domains of the Fv fragment, V_{L} and V_{H}, are coded for by separate genes and in natural antibodies are expressed separately on the light chain and heavy chain, respectively, they can be joined, using recombinant methods, by a synthetic linker that enables them to be made as a single protein chain in which the V_{L} and V_{H} regions pair to form monovalent molecules (known as single chain Fv (scFv) or single chain antibody. These single chain antibodies are obtained using conventional techniques known to those with skill in the art, and the fragments are screened for utility in the same manner as are intact antibodies. Single chain antibodies, which also are “antigen binding portions” of antibodies as that term is used herein, can be produced by recombinant DNA techniques, or by enzymatic or chemical cleavage of intact immunoglobulins.

The term “monoclonal antibody” refers to an antibody obtained from or prepared as a population of substantially homogeneous antibodies, i.e., each individual antibody molecule of which the population is comprised is essentially identical to all the others except for e.g., variable glycosylation and/or molecules comprising naturally occurring mutations, that may be present in minor amounts. Monoclonal antibodies are highly specific, being directed against a single antigenic site. Furthermore, in contrast to polyclonal antibody preparations which, even when prepared against a single purified antigen typically include many different antibodies directed against multiple discrete determinants (epitopes) of the antigen, each monoclonal antibody is typically directed against a single determinant on the antigen. Monoclonal antibodies can be prepared using any art recognized technique Monoclonal antibodies include chimeric antibodies, human antibodies and humanized antibodies and may occur naturally or be recombinantly produced.

Antibodies may prepared, expressed, created or isolated by recombinant means, such as (a) antibodies isolated from an animal (e.g., a mouse) that is transgenic or transchromosomal for immunoglobulin genes (e.g., human immunoglobulin genes) or a hybridoma prepared therefrom, (b) antibodies isolated from a host cell transformed to express the antibody, e.g., from a transfectoma, (c) antibodies isolated from a recombinant, combinatorial antibody library (e.g., containing human antibody sequences) using phage display, and (d) antibodies prepared, expressed, created or isolated by any other means that involve splicing of immunoglobulin gene sequences (e.g., human immunoglobulin genes) to other DNA sequences. Such recombinant antibodies may have variable and constant regions derived from human germline immunoglobulin sequences. In certain embodiments, however, such recombinant human antibodies can be subjected to in vitro mutagenesis and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “chimeric immunoglobulin” or “chimeric antibody” refers to an immunoglobulin or antibody whose variable regions derive from a first species and whose constant regions derive from a second species. Chimeric immunoglobulins or antibodies can be constructed, for example by genetic engineering, from immunoglobulin gene segments belonging to different species.

The term “human antibody,” indicates antibodies having variable regions in which both the framework and CDR regions are derived from human germline immunoglobulin sequences. Furthermore, if the human antibody contains a constant region, the constant region also is derived from human germline immunoglobulin sequences. The human antibodies may include amino acid residues not encoded by human germline immunoglobulin sequences (e.g., mutations introduced by random or site-specific mutagenesis in vitro or by somatic mutation in vivo). However, the term “human antibody” does not include antibodies in which CDR sequences derived from the germline of another mammalian species, such as a mouse, have been grafted onto human framework sequences.

The human antibody can have at least one ore more amino acids replaced with an amino acid residue, e.g., an activity enhancing amino acid residue which is not encoded by the human germline immunoglobulin sequence. Typically, the human antibody can have up to twenty positions replaced with amino acid residues which are not part of the human germline immunoglobulin sequence. In a particular embodiment, these replacements are within the CDR regions as described in detail below.

The term “humanized immunoglobulin” or “humanized antibody” refers to an immunoglobulin or antibody that includes at least one humanized immunoglobulin or antibody chain (i.e., at least one humanized light or heavy chain). The term “humanized immunoglobulin chain” or “humanized antibody chain” (i.e., a “humanized immunoglobulin light chain” or “humanized immunoglobulin heavy chain”) refers to an immunoglobulin or antibody chain (i.e., a light or heavy chain, respectively) having a variable region that includes a variable framework region substantially from a human immunoglobulin or antibody and complementarity determining regions (CDRs) (e.g., at least one CDR, preferably two CDRs, more preferably three CDRs) substantially from a non-human immunoglobulin or antibody, and further includes constant regions (e.g., at least one constant region or portion thereof, in the case of a light chain, and e.g., three constant regions in the case of a heavy chain). The term “humanized variable region” (e.g., “humanized light chain variable region” or “humanized heavy chain variable region”) refers to a variable region that includes a variable framework region substantially from a human immunoglobulin or antibody and complementarity determining regions (CDRs) substantially from a non-human immunoglobulin or antibody.

An “isolated BBA” as used herein, is intended to refer to a BBA which is substantially free of other BBA having different antigenic specificities. In addition, an isolated BBA is typically substantially free of cellular materials.

“Isotype” refers to the antibody class that is encoded by heavy chain constant region genes. In one embodiment, an antibody or antigen binding portion thereof is of an isotype selected from an IgG1, an IgG2, an IgG3, an IgG4, an IgM, an IgA1, an IgA2, an IgAsec, an IgD, or an IgE antibody isotype. In some embodiments, an antibody is of the IgG1 isotype. In other embodiments, an antibody is of the IgG2 isotype.

An “antigen” is an entity (e.g., a proteinaceous entity or peptide) to which a binding moiety within a BBA binds. In various embodiments disclosed herein, the antigen is ErbB3 or IGF-1R. In a particular embodiment, the antigen is human ErbB3 or human IGF-1R.

The term “epitope” or “antigenic determinant” refers to a site on an antigen to which an immunoglobulin or antibody specifically binds. Epitopes can be formed both from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, 4, 5, 6, 7, 8, 9, 10, 11, 12, 13, 14 or 15 amino acids in a unique spatial conformation. Methods of determining spatial conformation of epitopes include techniques in the art and those described herein, for example, x-ray crystallography and 2-dimensional nuclear magnetic resonance.

“Specific binding,” “specifically binds,” “selective binding,” and “selectively binds,” mean that an antibody or a binding moiety of a BBA exhibits appreciable affinity for a particular antigen or epitope and, generally, does not exhibit significant cross-reactivity with other antigens and epitopes. “Appreciable” or preferred binding includes binding with a dissociation constant (Ka) of 10⁻⁶, 10⁻⁷, 10⁻⁸, 10⁻⁹ M⁻¹, or 10⁻¹⁰ M or an even lower K_{d} value. Dissociation constants with values of lower than 10⁻⁷M, and preferably lower than 10⁻⁸ M, are more preferred (note that lower values for dissociation constants indicate higher binding affinity, thus a K_{d} of 10⁻⁷ indicates a lower (better) binding affinity than a K_{d} of 10⁻⁸). Values intermediate of those set forth herein are also intended to be within the scope of the disclosure and a preferred binding affinity can be indicated by a range of dissociation constants, for example, 10⁻⁶ to 10⁻¹⁰ M, preferably 10⁻⁷ to 10⁻¹⁰ M, more preferably 10⁻⁸ to 10⁻¹⁰ M or better. A binding moiety that “does not exhibit significant cross-reactivity” is one that will not appreciably bind to the entity with which it does not cross react (e.g., a proteinaceous entity). Specific or selective binding can be determined according to any art-recognized means for determining such binding, including, for example, according to Scatchard analysis and/or competitive binding assays. Dissociation constant (K_{d}), and hence binding affinity, may be conveniently measured using a surface plasmon resonance assay (e.g., as determined in a BIACORE 3000 instrument (GE Healthcare) e.g., using recombinant ErbB3 as the analyte and the antibody as the ligand) or a cell binding assay. One embodiment, the binding moiety of the BBA binds an antigen (either ErbB3 or IGF-1R) with a dissociation constant (K_{d}) of 50 nM or less (i.e., a binding affinity at least as high as that indicated by a K_{d} of 50 nM) (e.g., a K_{d} of 40 nM or 30 nM or 20 nM or 10 nM or less). In a particular embodiment, the binding moiety of the BBA binds an antigen (either ErbB3 or IGF-1R) with K_{d} of 8 nM or better (e.g., 7 nM, 6 nM, 5 nM, 4 nM, 2 nM, 1.5 nM, 1.4 nM, 1.3 nM, 1 nM or less). In other embodiments, the binding moiety binds an antigen (ErbB3 or IGF-1R) with a dissociation constant (K_{d}) of less than approximately 10⁻⁷ M, such as less than approximately 10⁻⁸ M, 10⁻⁹ M or 10⁻¹⁰ M or even lower, and binds to the predetermined antigen with an affinity that is at least two-fold higher (i.e., a K_{d} value that is at least two-fold lower) than its binding affinity for to a non-specific antigen (e.g., BSA, casein—i.e., an antigen other than the predetermined antigen or an antigen closely-related to the predetermined antigen).

The term “IC₅₀,” refers to the concentration of BBA which provides a, 50% inhibition of a maximal response, i.e., reduces the response to a level halfway between the maximal response and the baseline. The IC50 value may be converted to an absolute inhibition constant (Kᵢ) using, e.g., the Cheng-Prusoff equation.

The term “nucleic acid molecule,” as used herein, is intended to include DNA molecules and RNA molecules. A nucleic acid molecule may be single-stranded or double-stranded, but typically is double-stranded DNA.

Nucleic acid molecules may be present in whole cells, in a cell lysate, or in a partially purified or substantially pure form.

The term “operably linked” refers to a nucleic acid sequence placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice. A nucleic acid is “operably linked” when it is placed into a functional relationship with another nucleic acid sequence. For instance, a promoter or enhancer is operably linked to a coding sequence if it is liked so as to affect the transcription of the coding sequence. With respect to transcription regulatory sequences, operably linked means that the DNA sequences being linked are contiguous and, where necessary to join two protein coding regions, contiguous and in reading frame.

The term “vector,” as used herein, is intended to refer to a nucleic acid molecule capable of transporting another nucleic acid to which it has been linked. One type of vector is a “plasmid,” which refers to a circular double stranded DNA loop into which additional DNA segments may be ligated. Another type of vector is a viral vector, (e.g., a replication defective retrovirus, adenovirus and adeno-associated virus) wherein additional DNA segments may be ligated into the viral genome so as to be operatively linked to a promoter (e.g., a viral promoter) that will drive the expression of a protein encoded by the DNA segment. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

The term “host cell,” as used herein, is intended to refer to a cell into which an expression vector has been introduced, which cell is capable of reproducing , and preferably expressing proteins encoded by, the vector. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

The terms “treat,” “treating,” and “treatment,” as used herein, refer to therapeutic or preventative measures described herein. The methods of “treatment” employ administration to a patient, of a BBA disclosed herein, for example, a patient having a disease or disorder associated with ErbB3 and/or IGF-1 dependent signaling or predisposed to having such a disease or disorder, in order to prevent, cure, delay, reduce the severity of, or ameliorate one or more symptoms of the disease or disorder or recurring disease or disorder, or in order to prolong the survival of a patient beyond that expected in the absence of such treatment.

The term “disease or disorder associated with ErbB3 and/or IGF-1R dependent signaling,” as used herein, includes disease states and/or symptoms associated with a disease state, where increased levels of ErbB3 and/or IGF-1R are found and/or activation of cellular cascades involving ErbB3 and/or IGF-1R are found. ErbB3 heterodimerizes with other ErbB proteins such as, EGFR and ErbB2, when increased levels of ErbB3 are found In general, the term “disease or disorder associated with ErbB3 and/or IGF-1R dependent signaling,” refers to any disorder, the onset, progression or the persistence of the symptoms of which requires the participation of ErbB3 and/or IGF-1R. Exemplary ErbB3-mediated and/or IGF-1R mediated disorders include, but are not limited to, for example, cancers.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastric cancer, pancreatic cancer, glial cell tumors such as glioblastoma and neurofibromatosis, cervical cancer, ovarian cancer, liver cancer, bladder cancer, hepatoma, breast cancer, colon cancer, melanoma, colorectal cancer, endometrial carcinoma, salivary gland carcinoma, kidney cancer, renal cancer, prostate cancer, vulval cancer, thyroid cancer, hepatic carcinoma and various types of head and neck cancer. In a particular embodiment, a cancer treated using the methods disclosed herein is selected from melanoma, breast cancer, ovarian cancer, renal carcinoma, gastrointestinal/colon cancer, lung cancer, and prostate cancer. Other cancers for treatment according to the methods disclosed herein are described further in the “Methods of Using BBAs” section below. The term “effective amount,” as used herein, refers to that amount of a BBA that is sufficient to effect treatment, prognosis or diagnosis of a disease or disorder associated with ErbB3 and/or IGF-1 dependent signaling, as described herein, when administered to a patient. A therapeutically effective amount will vary depending upon the patient and disease condition being treated, the weight and age of the patient, the severity of the disease condition, the manner of administration and the like, which can readily be determined by one of ordinary skill in the art. The dosages for administration to a 70 kg patient can range from, for example, about 1 μg to about 5000 mg, about 2 μg to about 4500 mg, about 3 μg to about 4000 mg, about 4 μg to about 3,500 mg, about 5 μg to about 3000 mg, about 6 μg to about 2500 mg, about 7 μg to about 2000 mg, about μg to about 1900 mg, about 9 μg to about 1,800 mg, about 10 μg to about 1,700 mg, about 15 μg to about 1,600 mg, about 20 μg to about 1,575 mg, about 30 μg to about 1,550 mg, about 40 μg to about 1,500 mg, about 50 μg to about 1,475 mg, about 100 μg to about 1,450 mg, about 200 μg to about 1,425 mg, about 300 μg to about 1,000 mg, about 400 μg to about 975 mg, about 500 μg to about 650 mg, about 0.5 mg to about 625 mg, about 1 mg to about 600 mg, about 1.25 mg to about 575 mg, about 1.5 mg to about 550 mg, about 2.0 mg to about 525 mg, about 2.5 mg to about 500 mg, about 3.0 mg to about 475 mg, about 3.5 mg to about 450 mg, about 4.0 mg to about 425 mg, about 4.5 mg to about 400 mg, about 5 mg to about 375 mg, about 10 mg to about 350 mg, about 20 mg to about 325 mg, about 30 mg to about 300 mg, about 40 mg to about 275 mg, about 50 mg to about 250 mg, about 100 mg to about 225 mg, about 90 mg to about 200 mg, about 80 mg to about 175 mg, about 70 mg to about 150 mg, or about 60 mg to about 125 mg, of an antibody or antigen binding portion thereof. Dosage regimen may be adjusted to provide the optimum therapeutic response. An effective amount is also one in which any toxic or detrimental effects (i.e., side effects) of an antibody or antigen binding portion thereof are minimized and/or outweighed by the beneficial effects. Additional dosages regimens are described further below in the section pertaining to pharmaceutical compositions.

The term “patient” indicates a human subject who is or will be receiving either prophylactic or therapeutic treatment. For example, the methods and compositions disclosed herein can be used to treat a patient having cancer.

The term “sample” refers to tissue, body fluid, or a cell from a patient. Normally, the tissue or cell will be removed from the patient, but in vivo diagnosis is also contemplated. In the case of a solid tumor, a tissue sample can be taken from a surgically removed tumor and prepared for testing by conventional techniques. In the case of lymphomas and leukemias, lymphocytes, leukemic cells, or lymph tissues can be obtained and appropriately prepared. Other patient samples, including urine, tear drops, serum, cerebrospinal fluid, feces, sputum, cell extracts etc. can also be useful for particular tumors.

The terms “anti-cancer agent” and “antineoplastic agent” refer to drugs used to treat tumors, cancers, malignancies, and the like. Drug therapy may be used alone, or in combination with other treatments such as surgery or radiation therapy. Several classes of drugs may be used in cancer treatment, depending on the nature of the organ involved. For example, breast cancers are commonly stimulated by estrogens, and may be treated with drugs which inactive the sex hormones. Similarly, prostate cancer may be treated with drugs that inactivate androgens, the male sex hormone. Anti-cancer agents for use in combination with BBAs disclosed herein in certain methods disclosed herein include, among others, those listed in APPENDIX A, which should not be construed as limiting. One or more anti-cancer agents may be administered either simultaneously or before or after administration of a BBA disclosed herein.

The term “anti-cancer treatment modality” refers to a treatment, other than administration of a drug or other form of therapeutic agent, which is effective in the treatment of cancer or inhibition of growth of cancer cells. Non-limiting examples of such anti-cancer modalities include surgery and treatment with heat or ionizing radiation.

Additional definitions may be found throughout this specification.

II. scFvs and BBAs and Preparation Thereof

Provided herein as examples are single chain antibody scFv AB2-21 having or comprising an amino acid sequence corresponding to SEQ ID NO:44 and single chain antibody scFv AB5-7 having or comprising an amino acid sequence corresponding to SEQ ID NO:1.

The BBAs provided herein comprise three functionally distinct components: a first binding moiety that specifically binds IGF-1R, a second binding moiety that specifically binds ErbB3 and a linking moiety that connects the first and second binding moieties together, for example, covalently to form a single molecule. Each of these components is described further below.

A. IGF-1R Binding Moiety One binding moiety in the BBA specifically binds to IGF-1R. Preferably, the binding moiety is an antibody (i.e., an anti-IGF-1R antibody), although other binding moieties that specifically bind IGF-1R are also suitable for use in the BBAs. The antibody can be, for example, a full-length antibody or an antigen binding fragment or portion thereof, such as an Fab or F(ab)′2 fragment. The antibody can be, for example, a human or humanized antibody (or comprise human or humanized variable regions). Furthermore, the antibody can be a chimeric antibody. An exemplary IGF-1R binding moiety is a single chain antibody (scFv), e.g., a human single chain antibody. An example of an anti-IGF-1R scFv suitable for use in the BBA is the AB5-7 scFv, the amino acid sequence of which is shown in SEQ ID NO:1. Alternatively, other anti-IGF-1R antibodies known in the art, such as CP-751,871 (Pfizer), IMC-A12 (Imclone), R1507 (Genmab), MK-0646 (Merck), AMG 479 (Amgen) and AVE-1642 (Sanofi-Aventis), can be adapted for use in the BBA. Moreover, other anti-IGF-1R antibodies for use in the BBAs can be prepared using standard methods for making and selecting antibodies, described in further detail below.

B. ErbB3 Binding Moiety

Another binding moiety in the BBA specifically binds to ErbB3. Preferably, the binding moiety is an antibody (i.e., an anti-ErbB3 antibody), although other binding moieties that specifically bind ErbB3 are also suitable for use in the BBAs. The antibody can be, for example, a full-length antibody or an antigen binding fragment or portion thereof, such as an Fab or F(ab)′2 fragment. The antibody can be, for example, a human or humanized antibody (or comprise human or humanized variable regions). Furthermore, the antibody can be a chimeric antibody. The ErbB3 binding moiety may be a single chain antibody (scFv), e.g., a human single chain antibody. Examples of anti-ErbB3 scFvs suitable for use in the BBA are AB2-3 scFv—SEQ ID NO:33, AB2-6 scFv—SEQ ID NO:43 and AB2-21 scFv—SEQ ID NO:44. Alternatively, other anti-ErbB3 antibodies known in the art, such as the antibodies described in PCT Publication WO 2008/100624 by Merrimack Pharmaceuticals and US Patent Publication 20090291085, assigned to Merrimack Pharmaceuticals, 1B4C3 (U3 Pharma AG) and U3-1287 (U3 Pharma/Amgen), can be used in the BBA. Moreover, other anti-ErbB3 antibodies for use in the BBAs can be prepared using standard methods for making and selecting antibodies, described in further detail below.

Additional monoclonal antibodies that can be used in BBAs (i.e., anti-IGF-1R and anti-ErbB3 antibodies) can be produced using a variety of known techniques. In particular embodiments, the antibodies are fully human monoclonal antibodies.

C. Linking Moiety

The linking moiety of a BBA typically is a proteinaceous molecule, although other chemical linkers known in the art for joining two binding moieties also are suitable for use in BBAs. In one embodiment, the linking moiety comprises human serum albumin (HSA), the amino acid sequence and nucleotide sequence of which are shown in SEQ ID NO:143 and SEQ ID NO:144, respectively. In another embodiment, the linking moiety comprises a mutated form of human serum albumin in which position 34 has been substituted with serine and position 503 has been substituted with glutamine. These substitutions were made to enhance the serum half life of the molecule. The amino acid sequence of this mutated from of HSA (mHSA) is shown in SEQ ID NO:145. The nucleotide sequence of mHSA is shown in SEQ ID NO:146. This mutated form of HSA, and use thereof as a linker in BBAs, is further described in PCT Application No. PCT/US2009/040259 by Merrimack Pharmaceuticals, Inc.

In further embodiments, the linker of SEQ ID NO:143 is extended by four amino acids at the N-terminus and by six amino acids at the C-terminus (SEQ ID NO:18), the linker of SEQ ID NO:144 is extended by twelve nucleotides at the N-terminus and by eighteen nucleotides at the C-terminus (SEQ ID NO:118), the linker of SEQ ID NO:145 is extended by four amino acids at the N-terminus and by six amino acids at the C-terminus (SEQ ID NO:19), and the linker of SEQ ID NO:146 is extended by twelve nucleotides at the N-terminus and by eighteen nucleotides at the C-terminus (SEQ ID NO:119).

In one embodiment, the first binding moiety is attached to the amino terminus (N-terminus or N-terminal end) of the linking moiety and the second binding moiety is attached to the carboxy terminus (C-terminus or C-terminal end) of the linker moiety. In another embodiment, the second binding moiety is attached to the amino terminus (N-terminus or N-terminal end) of the linking moiety and the first binding moiety is attached to the carboxy terminus (C-terminus or C-terminal end) of the linker moiety.

Exemplary BBAs that comprise the mutated HSA linker are AB5-7N/AB2-3C (SEQ ID NO:93), AB5-7N/AB2-6C (SEQ ID NO:94), AB5-7N/AB2-21C (SEQ ID NO:95), AB2-3N/AB5-7C (SEQ ID NO:96), AB2-6N/AB5-7C (SEQ ID NO:97) and AB2-21N/AB5-7C (SEQ ID NO:98). The binding activity, antagonist activity and tumor growth inhibitory activity of such binding agents is described in further detail in the Examples, infra. Preferably, as demonstrated in Examples 3 and 4, a BBA exhibits one or more of the following functional properties: (i) inhibits IGF-1-induced phosphorylation of IGF-1R; (ii) inhibits heregulin (HRG)- or betacellulin (BTC)-induced phosphorylation of ErbB3; (iii) inhibits IGF-1-induced phosphorylation of AKT; (iv) inhibits proliferation of tumor cells; (v) inhibits growth of tumor spheroids. Methods for evaluating each of these functional properties are described below in further detail in Examples 3 and 4.

Nucleic acids encoding the BBAs can be prepared using standard recombinant DNA techniques, e.g., through ligating in-frame a nucleic acid molecule encoding the first binding moiety and a nucleic acid encoding the second binding moiety to a nucleic acid encoding the linker moiety. Further provided herein is a method of expressing a BBA provided herein by introducing a BBA-encoding nucleic acid molecule into an expression vector and introducing the resulting BBA expression vector (e.g., via transfection, transduction, or infection) into a host cell such as a lymphoma cell. The resulting BBA host cells can then be cultured to express the BBA, which can be recovered from the host cells or the culture medium in which the host cells are grown. The construction and expression of BBAs are described further in Example 1. The recombinantly expressed BBAs can be purified using various chromatography approaches, such as those described below in Example 2.

In other embodiments, the BBAs described herein can be represented by the formula:

A-L-B

wherein the order of A, L and B is N-terminal to C-terminal and (i) A is a binding moiety that specifically binds to IGF-1R, L is a linker moiety and B is a binding moiety that specifically binds to ErbB3; or (ii) A is a binding moiety that specifically binds to ErbB3, L is a linker moiety and B is a binding moiety that specifically binds to IGF-1R. In various embodiments, the linker moiety, “L”, can be, e.g., a monomeric linker, a dimeric linker or a trimeric linker. In various embodiments, the binding moiety that specifically binds to IGF-1R (“A” or “B”) can be an antibody, or antibody fragment, such as a scFv, an Fab fragment, a V_{H} fragment, a V_{L} fragment or other antigen-binding fragment as described in detail above. In various embodiments a tandem binding moiety has a valencey of 1, 2, or 3. In various embodiments, the binding moiety that specifically binds to ErbB3 (“A” or “B”) can be an antibody, or antibody fragment, such as a scFv, an Fab fragment, a V_{H} fragment, a V_{L} fragment or other antigen-binding fragment as described in detail above. In another embodiment, the linker moiety, “L”, is chemically and biologically inert. In yet other embodiments, the linker moiety, “L”, or the entire BBA can be, for example, glycosylated, aglycosylated or hyperglycosylated. In yet other embodiments, the sequence(s) encoding “A”, “L” and/or “B” can be stabilized, optimized, stabilized and optimized, and/or homogenous. In various embodiments, the linker moiety can be, for example, a fragment of human serum albumin, human immunoglobulin, human TRAIL, human LIGHT, human CD4OL, human TNFα, human CD95, human BAFF, human TWEAK, human OX40, or human TNFβ.

Accordingly, additional non-limiting examples of embodiments comprising the formula A-L-B are set forth in the 14 Tables below (Tables A-N), wherein all sequences indicated are amino acid sequences. Constructs combining the corresponding nucleotide sequences encoding the indicated combinations of amino acid sequences are also contemplated and such corresponding nucleotide sequences are provided in the Sequence Listing. In each of Tables A-N below, all possible A-L-B combinations of each moiety indicated by each of the individual sequences in each column with any moiety indicated in each of the other columns of that Table are contemplated as novel BBAs.

TABLE A

| A = anti-IGF-1R scFv | L = monomeric linker | B = anti-ErbB3 scFv |
|---|---|---|
| SEQ ID NO: 1 | SEQ ID NO: 18 | SEQ ID NO: 33 |
| SEQ ID NO: 2 | SEQ ID NO: 19 | SEQ ID NO: 34 |
| SEQ ID NO: 3 | SEQ ID NO: 143 | SEQ ID NO: 43 |
| SEQ ID NO: 4 | SEQ ID NO: 145 | SEQ ID NO: 44 |
| SEQ ID NO: 5 | SEQ ID NO: 32 | SEQ ID NO: 45 |
| SEQ ID NO: 14 | | SEQ ID NO: 46 |
| SEQ ID NO: 15 | | SEQ ID NO: 47 |
| SEQ ID NO: 63 | | SEQ ID NO: 73 |
| SEQ ID NO: 64 | | SEQ ID NO: 74 |
| SEQ ID NO: 65 | | SEQ ID NO: 81 |
| SEQ ID NO: 68 | | SEQ ID NO: 82 |
| SEQ ID NO: 69 | | SEQ ID NO: 48 |
| | | SEQ ID NO: 49 |
| | | SEQ ID NO: 50 |

TABLE B

| A = anti-ErbB3 scFv | L = monomeric linker | B = anti-IGF-1R scFv |
|---|---|---|
| SEQ ID NO: 33 | SEQ ID NO: 18 | SEQ ID NO: 1 |
| SEQ ID NO: 34 | SEQ ID NO: 19 | SEQ ID NO: 2 |
| SEQ ID NO: 43 | SEQ ID NO: 143 | SEQ ID NO: 3 |
| SEQ ID NO: 44 | SEQ ID NO: 145 | SEQ ID NO: 4 |
| SEQ ID NO: 45 | SEQ ID NO: 32 | SEQ ID NO: 5 |
| SEQ ID NO: 46 | | SEQ ID NO: 14 |
| SEQ ID NO: 47 | | SEQ ID NO: 15 |
| SEQ ID NO: 73 | | SEQ ID NO: 63 |
| SEQ ID NO: 74 | | SEQ ID NO: 64 |
| SEQ ID NO: 81 | | SEQ ID NO: 65 |
| SEQ ID NO: 82 | | SEQ ID NO: 68 |
| SEQ ID NO: 48 | | SEQ ID NO: 69 |
| SEQ ID NO: 49 | | |
| SEQ ID NO: 50 | | |

TABLE C

| A = anti-IGF-1R scFv | L = dimeric linker | B = anti-ErbB3 scFv |
|---|---|---|
| SEQ ID NO: 1 | SEQ ID NO: 20 | SEQ ID NO: 33 |
| SEQ ID NO: 2 | SEQ ID NO: 21 | SEQ ID NO: 34 |
| SEQ ID NO: 3 | SEQ ID NO: 53 | SEQ ID NO: 43 |
| SEQ ID NO: 4 | SEQ ID NO: 54 | SEQ ID NO: 44 |
| SEQ ID NO: 5 | SEQ ID NO: 55 | SEQ ID NO: 45 |
| SEQ ID NO: 14 | SEQ ID NO: 56 | SEQ ID NO: 46 |
| SEQ ID NO: 15 | SEQ ID NO: 51 | SEQ ID NO: 47 |
| SEQ ID NO: 63 | SEQ ID NO: 52 | SEQ ID NO: 73 |
| SEQ ID NO: 64 | | SEQ ID NO: 74 |
| SEQ ID NO: 65 | | SEQ ID NO: 81 |
| SEQ ID NO: 68 | | SEQ ID NO: 82 |
| SEQ ID NO: 69 | | SEQ ID NO: 48 |
| | | SEQ ID NO: 49 |
| | | SEQ ID NO: 50 |

TABLE D

| A = anti-ErbB3 scFv | L = dimeric linker | B = anti-IGF-1R scFv |
|---|---|---|
| SEQ ID NO: 33 | SEQ ID NO: 20 | SEQ ID NO: 1 |
| SEQ ID NO: 34 | SEQ ID NO: 21 | SEQ ID NO: 2 |
| SEQ ID NO: 43 | SEQ ID NO: 53 | SEQ ID NO: 3 |
| SEQ ID NO: 44 | SEQ ID NO: 54 | SEQ ID NO: 4 |
| SEQ ID NO: 45 | SEQ ID NO: 55 | SEQ ID NO: 5 |
| SEQ ID NO: 46 | SEQ ID NO: 56 | SEQ ID NO: 14 |
| SEQ ID NO: 47 | SEQ ID NO: 51 | SEQ ID NO: 15 |
| SEQ ID NO: 73 | SEQ ID NO: 52 | SEQ ID NO: 63 |
| SEQ ID NO: 74 | | SEQ ID NO: 64 |
| SEQ ID NO: 81 | | SEQ ID NO: 65 |
| SEQ ID NO: 82 | | SEQ ID NO: 68 |
| SEQ ID NO: 48 | | SEQ ID NO: 69 |
| SEQ ID NO: 49 | | |
| SEQ ID NO: 50 | | |

TABLE E

| A = anti-IGF-1R scFv | L = trimeric linker | B = anti-ErbB3 scFv |
|---|---|---|
| SEQ ID NO: 1 | SEQ ID NO: 30 | SEQ ID NO: 33 |
| SEQ ID NO: 2 | SEQ ID NO: 31 | SEQ ID NO: 34 |
| SEQ ID NO: 3 | | SEQ ID NO: 43 |
| SEQ ID NO: 4 | | SEQ ID NO: 44 |
| SEQ ID NO: 5 | | SEQ ID NO: 45 |
| SEQ ID NO: 14 | | SEQ ID NO: 46 |
| SEQ ID NO: 15 | | SEQ ID NO: 47 |
| SEQ ID NO: 63 | | SEQ ID NO: 73 |
| SEQ ID NO: 64 | | SEQ ID NO: 74 |
| SEQ ID NO: 65 | | SEQ ID NO: 81 |
| SEQ ID NO: 68 | | SEQ ID NO: 82 |
| SEQ ID NO: 69 | | SEQ ID NO: 48 |
| | | SEQ ID NO: 49 |
| | | SEQ ID NO: 50 |

TABLE F

| A = anti-ErbB3 scFv | L = trimeric linker | B = anti-IGF-1R scFv |
|---|---|---|
| SEQ ID NO: 33 | SEQ ID NO: 30 | SEQ ID NO: 1 |
| SEQ ID NO: 34 | SEQ ID NO: 31 | SEQ ID NO: 2 |
| SEQ ID NO: 43 | | SEQ ID NO: 3 |
| SEQ ID NO: 44 | | SEQ ID NO: 4 |
| SEQ ID NO: 45 | | SEQ ID NO: 5 |
| SEQ ID NO: 46 | | SEQ ID NO: 14 |
| SEQ ID NO: 47 | | SEQ ID NO: 15 |
| SEQ ID NO: 73 | | SEQ ID NO: 63 |
| SEQ ID NO: 74 | | SEQ ID NO: 64 |
| SEQ ID NO: 81 | | SEQ ID NO: 65 |
| SEQ ID NO: 82 | | SEQ ID NO: 68 |
| SEQ ID NO: 48 | | SEQ ID NO: 69 |
| SEQ ID NO: 49 | | |
| SEQ ID NO: 50 | | |

In another embodiment, the invention provides BBAs in which A=anti-IGF-1R antibody fragment+a co-expressed antibody partner (to form a double-chained antibody molecule), L=a monomeric, dimeric or trimeric linker and B=an anti-ErbB3 scFv antibody. All possible combinations of A, L and B from Tables G, H and I below are contemplated, with the caveat that for the antibody pairings for the “A” moiety, the light chain and heavy chain pairings are maintained from the same antibody (e.g., a fragment from antibody 5-7 is paired with an antibody 5-7 partner, a a fragment from antibody 5-6 is paired with an antibody 5-6 partner and so on as set forth in Tables G, H, and I below).

TABLE G

| A = anti-IGF-1R Pairing | | | |
|---|---|---|---|
| | Coexpressed | | B = |
| V_{H} | Partner (LC) | L = Linker | anti-ErbB3 scFv |
| SEQ ID NO: | SEQ ID NO: | SEQ ID NO: 22 | SEQ ID NO: 33 |
| 6 (5-7) | 10 (5-7) | | |
| SEQ ID NO: | SEQ ID NO: | SEQ ID NO: 23 | SEQ ID NO: 34 |
| 7 (5-7) | 11 (5-7) | | |
| | | SEQ ID NO: 24 | SEQ ID NO: 43 |
| SEQ ID NO: | SEQ ID NO: | SEQ ID NO: 25 | SEQ ID NO: 44 |
| 59 (5-6) | 66 (5-6) | | |
| SEQ ID NO: | | SEQ ID NO: 26 | SEQ ID NO: 45 |
| 60 (5-6) | | | |
| | | SEQ ID NO: 27 | SEQ ID NO: 46 |
| SEQ ID NO: | SEQ ID NO: | SEQ ID NO: 28 | SEQ ID NO: 47 |
| 70 (5-5) | 17 (5-5) | | |
| SEQ ID NO: | | SEQ ID NO: 29 | SEQ ID NO: 73 |
| 71 (5-5) | | | SEQ ID NO: 74 |
| | | | SEQ ID NO: 81 |
| | | | SEQ ID NO: 82 |
| | | | SEQ ID NO: 48 |
| | | | SEQ ID NO: 49 |
| | | | SEQ ID NO: 50 |

TABLE H

| A = anti-IGF-1R Pairing | | | |
|---|---|---|---|
| | Coexpressed | | B = |
| V_{L} | Partner (LC) | L = Linker | anti-ErbB3 scFv |
| SEQ ID NO: | SEQ ID NO: | SEQ ID NO: | SEQ ID NO: 33 |
| 12 (5-7) | 58 (5-7) | 20 (5-7) | |
| SEQ ID NO: | | | SEQ ID NO: 34 |
| 13 (5-7) | | | |
| | | | SEQ ID NO: 43 |
| | | | SEQ ID NO: 44 |
| | | | SEQ ID NO: 45 |
| | | | SEQ ID NO: 46 |
| | | | SEQ ID NO: 47 |
| | | | SEQ ID NO: 73 |
| | | | SEQ ID NO: 74 |
| | | | SEQ ID NO: 81 |
| | | | SEQ ID NO: 82 |
| | | | SEQ ID NO: 48 |
| | | | SEQ ID NO: 49 |
| | | | SEQ ID NO: 50 |

TABLE I

| A = anti-IGF-1R Pairing | | | |
|---|---|---|---|
| | Coexpressed | | B = |
| Fab HC | Partner (LC) | L = Linker | anti-ErbB3 scFv |
| | | dimeric | |
| SEQ ID NO: | SEQ ID NO: | SEQ ID NO: 53 | SEQ ID NO: 33 |
| 8 (5-7) | 10 (5-7) | | |
| SEQ ID NO: | SEQ ID NO: | SEQ ID NO: 54 | SEQ ID NO: 34 |
| 9 (5-7) | 11 (5-7) | | |
| | | SEQ ID NO: 55 | SEQ ID NO: 43 |
| SEQ ID NO: | SEQ ID NO: | SEQ ID NO: 56 | SEQ ID NO: 44 |
| 61 (5-6) | 66 (5-6) | | |
| SEQ ID NO: | | SEQ ID NO: 51 | SEQ ID NO: 45 |
| 62 (5-6) | | | |
| | | SEQ ID NO: 52 | SEQ ID NO: 46 |
| SEQ ID NO: | SEQ ID NO: | monomeric | SEQ ID NO: 47 |
| 72 (5-5) | 17 (5-5) | | |
| | | SEQ ID NO: 18 | SEQ ID NO: 73 |
| | | SEQ ID NO: 19 | SEQ ID NO: 74 |
| | | SEQ ID NO: 143 | SEQ ID NO: 81 |
| | | SEQ ID NO: 145 | SEQ ID NO: 82 |
| | | SEQ ID NO: 32 | SEQ ID NO: 48 |
| | | trimeric | SEQ ID NO: 49 |
| | | SEQ ID NO: 30 | SEQ ID NO: 50 |
| | | SEQ ID NO: 31 | |

In another embodiment, the invention provides BBAs in which A=an anti-ErbB3 scFv antibody, L=a monomeric, dimeric or trimeric linker and B=anti-IGF-1R antibody fragment+a co-expressed antibody partner (to form a double-chained antibody molecule). All possible combinations of A, L and B from the Table J below are intended to be encompassed by the invention, with the caveat that for the antibody pairings for the “B” moiety, the light chain and heavy chain pairings are maintained from the same antibody (e.g., a Fab HC from Ab 5-7 is paired with an Ab 5-7 partner, a Fab HC from Ab 5-6 is paired with an Ab 5-6 partner and so on as set forth in Table J below).

TABLE J

| | | B = anti-IGF-1R Pairing | |
|---|---|---|---|
| A = | | | Coexpressed |
| anti-ErbB3 scFv | L = Linker | Fab HC | Partner (LC) |
| SEQ ID NO: 33 | dimeric | | |
| SEQ ID NO: 34 | SEQ ID NO: 53 | SEQ ID NO: | SEQ ID NO: |
| | | 8 (5-7) | 10 (5-7) |
| SEQ ID NO: 43 | SEQ ID NO: 54 | SEQ ID NO: | SEQ ID NO: |
| | | 9 (5-7) | 11 (5-7) |
| SEQ ID NO: 44 | SEQ ID NO: 55 | | |
| SEQ ID NO: 45 | SEQ ID NO: 56 | SEQ ID NO: | SEQ ID NO: |
| | | 61 (5-6) | 66 (5-6) |
| SEQ ID NO: 46 | SEQ ID NO: 51 | SEQ ID NO: | |
| | | 62 (5-6) | |
| SEQ ID NO: 47 | SEQ ID NO: 52 | | |
| SEQ ID NO: 73 | monomeric | SEQ ID NO: | SEQ ID NO: |
| | | 151 (5-5) | 17 (5-5) |
| SEQ ID NO: 74 | SEQ ID NO: 18 | SEQ ID NO: | |
| | | 72 (5-5) | |
| SEQ ID NO: 81 | SEQ ID NO: 19 | | |
| SEQ ID NO: 82 | SEQ ID NO: 143 | | |
| SEQ ID NO: 48 | SEQ ID NO: 145 | | |
| SEQ ID NO: 49 | SEQ ID NO: 32 | | |
| SEQ ID NO: 50 | trimeric | | |
| | SEQ ID NO: 30 | | |
| | SEQ ID NO: 31 | | |

In another preferred embodiment, the invention provides BBAs in which A=anti-ErbB3 antibody fragment+a co-expressed antibody partner (to form a double-chained antibody molecule), L=a monomeric, dimeric or trimeric linker and B=an anti-IGF-1R scFv antibody. All possible combinations of A, L and B from Tables K and M below are intended to be encompassed by the invention, with the caveat that for the antibody pairings for the “A” moiety, the light chain and heavy chain pairings are maintained from the same antibody (e.g., a fragment from antibody 2-3 is paired with an antibody 2-3 partner, a fragment from antibody 2-14 is paired with an antibody 2-14 partner and so on as set forth in Tables K and M below).

TABLE K

| A = anti-ErbB3B3 Pairing | | | |
|---|---|---|---|
| | Coexpressed | | B = |
| V_{H} | Partner (LC) | L = Linker | anti-IGF-1R scFv |
| SEQ ID NO: | SEQ ID NO: | SEQ ID NO: 22 | SEQ ID NO: 1 |
| 35 (2-3) | 39 (2-3) | | |
| SEQ ID NO: | SEQ ID NO: | SEQ ID NO: 23 | SEQ ID NO: 2 |
| 36 (2-3) | 40 (2-3) | | |
| | | SEQ ID NO: 24 | SEQ ID NO: 3 |
| SEQ ID NO: | SEQ ID NO: | SEQ ID NO: 25 | SEQ ID NO: 4 |
| 75 (2-14) | 79 (2-14) | | |
| SEQ ID NO: | | SEQ ID NO: 26 | SEQ ID NO: 5 |
| 76 (2-14) | | | |
| | | SEQ ID NO: 27 | SEQ ID NO: 14 |
| SEQ ID NO: | SEQ ID NO: | SEQ ID NO: 28 | SEQ ID NO: 15 |
| 83 (2-21) | 86 (2-21) | | |
| SEQ ID NO: | | SEQ ID NO: 29 | SEQ ID NO: 63 |
| 84 (2-21) | | | |
| | | | SEQ ID NO: 64 |
| SEQ ID NO: | SEQ ID NO: | | SEQ ID NO: 65 |
| 89 (E3B) | 91 (E3B) | | |
| | | | SEQ ID NO: 68 |
| | | | SEQ ID NO: 69 |

TABLE M

| A = anti-ErbB3 Pairing | | | |
|---|---|---|---|
| | Coexpressed | | B = |
| Fab HC | Partner (LC) | L = Linker | anti-IGF-1R scFv |
| SEQ ID NO: | SEQ ID NO: | dimeric | SEQ ID NO: 1 |
| 37 (2-3) | 39 (2-3) | | |
| SEQ ID NO: | SEQ ID NO: | SEQ ID NO: 53 | SEQ ID NO: 2 |
| 38 (2-3) | 40 (2-3) | | |
| | | SEQ ID NO: 54 | SEQ ID NO: 3 |
| SEQ ID NO: | SEQ ID NO: | SEQ ID NO: 55 | SEQ ID NO: 4 |
| 77 (2-14) | 79 (2-14) | | |
| SEQ ID NO: | | SEQ ID NO: 56 | SEQ ID NO: 5 |
| 78 (2-14) | | | |
| | | SEQ ID NO: 51 | SEQ ID NO: 14 |
| SEQ ID NO: | SEQ ID NO: | SEQ ID NO: 52 | SEQ ID NO: 15 |
| 87 (2-21) | 86 (2-21) | | |
| SEQ ID NO: | | monomeric | SEQ ID NO: 63 |
| 88 (2-21) | | | |
| | | SEQ ID NO: 18 | SEQ ID NO: 64 |
| SEQ ID NO: | SEQ ID NO: | SEQ ID NO: 19 | SEQ ID NO: 65 |
| 92 (E3B) | 91 (E3B) | | |
| | | SEQ ID NO: 143 | SEQ ID NO: 68 |
| | | SEQ ID NO: 145 | SEQ ID NO: 69 |
| | | SEQ ID NO: 32 | |
| | | trimeric | |
| | | SEQ ID NO: 30 | |
| | | SEQ ID NO: 31 | |

In another preferred embodiment, the invention provides BBAs in which A=an anti-IGF-1R scFv antibody, L=a monomeric, dimeric or trimeric linker and B=anti-ErbB3 antibody fragment +a co-expressed antibody partner (to form a double-chained antibody molecule). All possible combinations of A, L and B from Table N below are intended to be encompassed by the invention, with the caveat that for the antibody pairings for the “B” moiety, the light chain and heavy chain pairings are maintained from the same antibody (e.g., a Fab HC from Ab 2-3 is paired with an Ab 2-3 partner, a Fab HC from Ab 2-14 is paired with an Ab 2-14 partner and so on as set forth in Table N below).

TABLE N

| | | B = anti-ErbB3B3 Pairing | |
|---|---|---|---|
| A = | | Fab Heavy | Coexpressed |
| anti-IGF-1R scFv | L = Linker | Chain (HC) | Partner (LC) |
| SEQ ID NO: 1 | dimeric | SEQ ID NO: | SEQ ID NO: |
| | | 37 (2-3) | 39 (2-3) |
| SEQ ID NO: 2 | SEQ ID NO: 53 | SEQ ID NO: | SEQ ID NO: |
| | | 38 (2-3) | 40 (2-3) |
| SEQ ID NO: 3 | SEQ ID NO: 54 | | |
| SEQ ID NO: 4 | SEQ ID NO: 55 | SEQ ID NO: | SEQ ID NO: |
| | | 77 (2-14) | 79 (2-14) |
| SEQ ID NO: 5 | SEQ ID NO: 56 | SEQ ID NO: | |
| | | 78 (2-14) | |
| SEQ ID NO: 14 | SEQ ID NO: 51 | | |
| SEQ ID NO: 15 | SEQ ID NO: 52 | SEQ ID NO: | SEQ ID NO: |
| | | 87 (2-21) | 86 (2-21) |
| SEQ ID NO: 63 | monomeric | SEQ ID NO: | |
| | | 88 (2-21) | |
| SEQ ID NO: 64 | SEQ ID NO: 18 | | |
| SEQ ID NO: 65 | SEQ ID NO: 19 | SEQ ID NO: | SEQ ID NO: |
| | | 92 (E3B) | 91 (E3B) |
| SEQ ID NO: 68 | SEQ ID NO: 143 | | |
| SEQ ID NO: 69 | SEQ ID NO: 145 | | |
| | SEQ ID NO: 32 | | |
| | trimeric | | |
| | SEQ ID NO: 30 | | |
| | SEQ ID NO: 31 | | |

### III. Pharmaceutical Compositions

In another aspect, a composition, e.g., a pharmaceutical composition, is provided containing one or more of the BBAs or single chain antibodies (e.g., scFvs) disclosed herein, formulated together with a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the BBA or scFv may be coated in a material to protect it from the action of acids and other natural conditions that may inactivate proteins.

Pharmaceutical compositions can be administered alone or in combination therapy, i.e., combined with other agents. For example, the combination therapy can include a BBA of the present disclosure with at least one additional therapeutic agent, such as an anti-cancer agent described infra. Pharmaceutical compositions can also be administered in conjunction with another anti-cancer treatment modality, such as radiation therapy and/or surgery.

A composition of the present disclosure can be administered by a variety of methods known in the art. As will be appreciated by the skilled artisan, the route and/or mode of administration will vary depending upon the desired results.

To administer a composition provided herein by certain routes of administration, it may be necessary to coat the BBA with, or co-administer the BBA with, a material to prevent its inactivation. For example, the BBA may be administered to a patient in an appropriate carrier, for example, in liposomes, or a diluent. Pharmaceutically acceptable diluents include saline and aqueous buffer solutions. Liposomes include water-in-oil-in-water CGF emulsions as well as conventional liposomes.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions provided herein is contemplated. Supplementary active compounds (e.g., anti-cancer agents as disclosed infra) can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be useful to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation include vacuum drying and freeze-drying (lyophilization) that yield a powder of the active ingredient plus any additional desired ingredient from a previously sterile-filtered solution thereof.

Dosage regimens are adjusted to provide the optimum desired response (e.g., a therapeutic response). For example, a single bolus or infusion may be administered, several divided doses may be administered over time or the dose may be proportionally reduced or increased as indicated by the exigencies of the therapeutic situation. For example, the BBAs disclosed herein may be administered once or twice weekly by, for example, intravenous or subcutaneous injection or once or twice monthly by, for example, intravenous or subcutaneous injection.

Non-limiting examples of suitable dosage ranges and regimens include 2-50 mg/kg (body weight of the patient) administered once a week, or twice a week or once every three days, or once every two weeks, or once a month, and 1-100 mg/kg administered once a week, or twice a week or once every three days, or once every two weeks, or once a month. In various embodiments, a BBA is administered at a dosage of 3.2 mg/kg, 6 mg/kg, 10 mg/kg, 15 mg/kg, 20 mg/kg, 25 mg/kg, 30 mg/kg, 35 mg/kg or 40 mg/kg at a timing of once a week, or twice a week or once every three days, or once every two weeks, or once a month. Suitable dosage schedules include once every three days, once every five days, once every seven days (i.e., once a week), once every 10 days, once every 14 days (i.e., once every two weeks), once every 21 days (i.e., once every three weeks), once every 28 days (i.e., once every four weeks) and once a month.

It is especially advantageous to formulate parenteral compositions in unit dosage form for ease of administration and uniformity of dosage. Unit dosage form as used herein refers to physically discrete units suited as unitary dosages (e.g., in vials or ampules) for individual patient treatment; each unit containing a predetermined quantity of BBA calculated to produce the desired therapeutic effect in association with the required pharmaceutical carrier.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Examples of suitable aqueous and nonaqueous carriers which may be employed in pharmaceutical compositions include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain additional agents such as preservatives, wetting agents, emulsifying agents and dispersing agents.

Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

When administered as pharmaceuticals, to humans and animals, BBAs can be given as a pharmaceutical composition containing, for example, 0.001 to 90% or 0.005 to 70%, or 0.01 to 30% of active ingredient in combination with a pharmaceutically acceptable carrier.

Actual dosage levels of the active ingredients (e.g., BBAs) in pharmaceutical compositions may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions employed, or the ester, salt or amide thereof, the route of administration, the time of administration, the rate of excretion of the particular compound being employed, the duration of the treatment, other drugs, compounds and/or materials used in combination with the particular BBA(s) employed, the age, sex, weight, condition, general health and prior medical history of the patient being treated, and like factors well known in the medical arts. A physician having ordinary skill in the art can readily determine and prescribe the effective amount of the pharmaceutical composition required. For example, the physician could start doses at levels lower than that required in order to achieve the desired therapeutic effect and gradually increase the dosage until the desired effect is achieved. In general, a suitable daily dose will be that amount of the active ingredient that is the highest dose effective to reproducibly provide a therapeutic effect without causing any unacceptable adverse effect. Such an effective dose will generally depend upon the factors described above. Administration may, for example, be intravenous, intramuscular, intraperitoneal, or subcutaneous. If desired, the effective daily dose of a therapeutic composition may be administered as two, three, four, five, six or more sub-doses administered separately at appropriate intervals throughout the day, optionally, in unit dosage forms.

Therapeutic compositions can be administered with medical devices known in the art. For example, in certain embodiments, a therapeutic composition provided herein can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants include: U.S. Pat. No. 4,487,603, which discloses an implantable micro-infusion pump for dispensing medication at a controlled rate; U.S. Pat. No. 4,486,194, which discloses a therapeutic device for administering medications through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and the like are known to those skilled in the art.

### IV. Methods of Using BBAs

Also provided are methods of using the BBAs for a variety of ex vivo and in vivo detection, diagnostic and therapeutic applications. Since the BBAs specifically bind IGF-1R and ErbB3, these agents can be used to detect the expression of either or both of these receptors on cells, as well as to purify the proteins by immunoaffinity techniques. Furthermore, the BBAs disclosed herein can be used for treating a disease or disorder associated with ErbB3 and/or IGF-1R dependent signaling, including a variety of cancers.

In one embodiment, a method is provided for inhibiting proliferation of a tumor cell expressing IGF-1R and ErbB3 comprising contacting the tumor cell with a BBA as described herein such that proliferation of the tumor cell is inhibited.

In another embodiment, a method is provided for treating a disease or disorder associated with ErbB3 and/or IGF-1R dependent signaling by administering to a patient a BBA disclosed herein in an amount effective to treat the disease or disorder. Suitable diseases or disorders include, for example, a variety of cancers including, but not limited to, melanoma, breast cancer, ovarian cancer, renal carcinoma, gastrointestinal cancer, colon cancer, lung cancer (e.g., non-small cell lung cancer), and prostate cancer.

Still further, a method is provided for treating a tumor expressing IGF-1R and ErbB3 in a patient, the method comprising administering a BBA as described herein such that the tumor is treated. Preferably, the tumor is selected from the group consisting of lung cancer, sarcoma, colorectal cancer, head and neck cancer, pancreatic cancer and breast cancer. In one embodiment, the tumor is a lung cancer that is a non-small cell lung cancer. In another embodiment, the tumor is a sarcoma that is a Ewing's sarcoma. In another embodiment, the tumor is a breast cancer that is a tamoxifen-resistant, estrogen receptor-positive breast cancer. In another embodiment, the tumor is a lung cancer that is a gefitinib-resistant lung cancer. In another embodiment, the tumor is a breast cancer that is trastuzumab-resistant metastatic breast cancer.

In another aspect, the method of treating a tumor can further comprise administering a second anti-cancer agent in combination with the BBA. Examples of suitable anti-cancer agents that can serve as the second anti-cancer agent in such combinations and methods of treatment are listed in APPENDIX A. Thus novel compositions are contemplated comprising a BBA, e.g., a BBA disclosed herein, together with a second anti-cancer agent, e.g., selected from those listed in Appendix A, typically together with at least one pharmaceutically acceptable excipient. Additionally or alternatively, the method of treating a tumor can further comprise administering a second anti-cancer treatment modality in combination with the BBA. Non-limiting examples of modalities that can serve as the “second anti-cancer treatment modality” in such combination methods include surgery and ionizing radiation.

In certain aspects, BBAs disclosed herein are administered to patients

In another embodiment, a method is provided for diagnosing a disease or disorder (e.g., a cancer) associated with ErbB3 and/or IGF-1R dependent signaling in a human subject, by contacting BBA disclosed herein(e.g., ex vivo or in vivo) with cells from the subject, and measuring the level of binding to ErbB3 and/or IGF-1R on the cells. Abnormally high levels of binding to ErbB3 and/or IGF-1R indicate that the subject probably has a disease or disorder associated with ErbB3 and/or IGF-1R dependent signaling.

Also provided are kits comprising BBAs disclosed herein. The kits may include a label indicating the intended use of the contents of the kit and optionally including instructions for use of the kit in treating or diagnosing a disease or disorder associated with ErbB3 and/or IGF-1R dependent signaling, e.g., diagnosing or treating a tumor. The term label includes any writing, marketing materials or recorded material supplied on or with the kit, or which otherwise accompanies the kit.

### EXAMPLES

The following examples should not be construed as limiting the scope of this disclosure.

### Materials and Methods

Throughout the examples, the following materials and methods are used unless otherwise stated. In general, the practice of the techniques of the present disclosure employs, unless otherwise indicated, conventional techniques of chemistry, molecular biology, recombinant DNA technology, immunology (especially, e.g., antibody technology), pharmacology, pharmacy, and standard techniques in polypeptide preparation.

Heregulin

As used in these Examples and in the Figures, HRG refers to the isoform of heregulin variously known as heregulin 1 beta 1, HRG1-B, HRG-β1, neuregulin 1, NRG1, neuregulin 1 beta 1, NRG1-b1, HRG ECD, and the like. HRG is commercially available, e.g., R&D Systems, 377-HB-050/CF.

IGF-1

As used in these Examples and in the Figures, IGF-1 refers to insulin growth factor 1. Recombinant human IGF-1 is commercially available, e.g., R&D Systems, 291-GI-050/CF.

Cell Lines

All the cell lines for use in the experiments described below may be obtained, as indicated, from American Type Culture Collection (ATCC, Manassas, Va.) or the US National Cancer Institute (NCI) e.g., from the Division of Cancer Treatment and Diagnostics (DCTD).
MCF7—ATCC cat. No. HTB-22
T47D—ATCC cat. No. HTB-133
OVCAR8—NCI
A549—ATCC cat. No. CCL-185
ADRr-NCI
BxPC-3—ATCC cat. No. CRL-1687
DU145—ATCC cat. No. HTB-81

Control Antibodies

Ab #6 (MM-121) as described in US Patent Publication 20090291085, was used as an anti-ErbB3 IgG antibody control. The mouse anti-human-IGF-1R monoclonal antibody mAb391 (IgG1, commercially available from R & D Systems, Catalog. No. MAB391) is used as an anti-IGF-1R IgG antibody control.

### EXAMPLE 1

### Preparation and Expression of Monomeric BBAs

The BBAs may be constructed using standard recombinant DNA techniques to ligate nucleic acid encoding each of the binding moieties to DNA encoding the human serum albumin (HSA) linker. More specifically, nucleic acid encoding a mutated form of the HSA linker, having the amino acid sequence shown in SEQ ID NO:145 and the nucleotide sequence shown in SEQ ID NO:146, is used.

In one set of three different agents, the N-terminal end of the linker is operatively linked to nucleic acid encoding the AB5-7 scFv (anti-IGF-1R) and the C-terminal end of the linker is operatively linked to nucleic acid encoding either the AB2-3, AB-2-6 or AB2-21 scFv (anti-ErbB3). These three agents are referred to herein as AB5-7N/AB2-3C and AB5-7N/AB2-6C, and AB5-7N/AB2-21C (Table 11 and 12)

In another set of three different agents, the N-terminal end of the linker is operatively linked to nucleic acid encoding either the AB2-3, AB2-6 or AB2-21 scFv (anti-ErbB3) and the C-terminal end of the linker is operatively linked to nucleic acid encoding AB5-7 scFv (anti-IGF-1R). These three agents are referred to herein as AB2-3N/AB5-7C AB2-6N/AB5-7C and AB2-21N/AB5-7C (Table 11 and 12).

The nucleic acids encoding HSA-fused BBAs are cloned as single molecules into expression plasmids. An exemplary expression vector is pMP 10K (SELEXIS) and an exemplary cell line is CHO-k1-S (SELEXIS). Expression plasmids are linearized (e.g., with Pvu1) followed by QIAQUICK purification (QIAGEN). Lipofectamine LTX (Invitrogen) is used for transfection into CHO cells in OptiMemI (Gibco). Transfected cells is recovered with F12Hams medium containing 10% FBS for 2 days without selection pressure, then with selection pressure for 4 days, then change to serum-free medium with selection pressure. HyClone® (Thermo Scientific) is used for the HSA-fused BBAs, with HT supplements (GIBCO).

### EXAMPLE 2

### Purification of Monomeric BBAs

BBAs are purified using three chromatography steps: protein A affinity, cation exchange and anion exchange. Each may be carried out in accordance with the manufacturer's instructions. The protein A affinity step is the most critical chromatography step because it selectively and efficiently binds the BBAs in complex solutions such as harvested cell culture fluids (HCCF), and it removes >99.5% of product impurities in a single step with high yields and high throughput. This step also provides significant virus clearance. MABSELECT from GE is used as the Protein A affinity resin. Protein G affinity chromatography may be substituted for protein A affinity chromatography if desired. SPFF (sulphopropyl fast flow) from GE, an agarose based resin, is used as the cation exchange resin in the second chromatography step. This step helps further in the removal of host cell impurities and multimeric forms (aggregates) of the BBAs. QSFF (Quaternary-amine sepharose fast flow) from GE, an agarose based anion exchange resin, helps in the final polishing of products, by removing any trace quantities of viruses, endotoxins, and host cell impurities from the SPFF pool in a third and final chromatography step

### EXAMPLE 3

### Binding and Antagonist Activities of Monomeric BBAs

A. Binding Affinities of BBAs

1×10⁵ MCF7 cells and 1×10⁵ ADRr cells are incubated at room temperature for 2 hours with the BBA at 2 uM, followed by 12 subsequent 3-fold dilutions. Then using goat anti-HSA-Alexa647 conjugated antibody as the detection antibody, cells are incubated on ice for 45 minutes. Cell binding dissociation constants (measures of binding affinities) of the BBAs on MCF7 and ADRr cells are assessed by FACS (fluorescence activated cell sorting) and apparent dissociation constants are determined for each BBA. The following results were obtained:

TABLE 1
Dissociation constants of BBAs

| AB-N-mHSA-AB-C | | K_{d} in nM | K_{d} in nM |
|---|---|---|---|
| N | C | MCF7cells | ADRr cells |

| | | |
|---|---|---|
| AB2-6N/AB5-7C | 5.2 | 1.0 |
| AB2-3N/AB5-7C | 10 | 6 |
| AB2-21N/AB5-7C | 19 | 0.4 |
| AB5-7N/AB2-6C | 12 | 6 |
| AB5-7N/AB2-3C | 19 | 14 |
| AB5-7N/AB 2-21C | 5 | 0.5 |

B. Antagonist Activity of BBAs

(i) Inhibition of pIGF-1R Formation

To determine the ability of the BBAs to antagonize IGF-1R, inhibition of IGF-1R phosphorylation in the presence of the agents is examined. 2.5×10⁴ ADRr cells are pre-incubated for 24 hours with either a BBA or with an anti-IGF-1R IgG at 1 □M, followed by 9 subsequent 3-fold dilutions to give a 10-point curve. Cells are treated with IGF-1 at 13 nM for 10 minutes. IGF-1 induced phosphorylation of IGF-1R to yield phospho-IGF-1R (pIGF-1R) is measured by ELISA (R & D Systems; Cat.# DYC1770) to evaluate the ability of the agents to inhibit pIGF-1R formation. The following results were obtained:

TABLE 2
Inhibition of pIGF-1R Formation by BBAs

| BBA (mHSA-fusions) | IC₅₀(in nM) |
|---|---|
| AB5-7N/AB2-21C | 11 |
| AB5-7N/AB2-3C | 28 |
| AB5-7N/AB2-6C | 3 |
| AB2-21N/AB5-7C | 11 |
| AB2-3N/AB5-7C | 27 |
| AB2-6N/AB5-7C | 6 |
| Anti-IGF-1R IgG1 | |
| mAb391 | 15 |

Thus, similar IC₅₀s were observed, indicating that the BBA is capable of antagonizing IGF-1-induced phosphorylation of IGF-1R as well as the anti-IGF-1R IgG antibody.

(ii) Inhibition of pErbB3 Formation

To determine the ability of the BBAs to antagonize ErbB3, inhibition of ErbB3 phosphorylation in the presence of the agents is examined. (2.5,×10⁴ ADRr cells are pre-incubated for 24 hours with either the BBA or with an anti-ErbB3 IgG at 1 □M, followed by 9 subsequent 3-fold dilutions to give a 10-point curve. Cells are treated with 5 nM of heregulin (HRG) for 15 minutes. HRG-induced phosphorylation of ErbB3 is measured by ELISA (R & D Systems; Cat.#DYC1769) to evaluate the ability of the agents to inhibit pErbB3 formation. A monoclonal IgG2 anti-ErbB3 antibody (AB #6) was used as a control. The following results were obtained:

TABLE 3
Inhibition of HRG-Induced pErbB3 Formation by BBAs
(and IgG comparator)

| BBA (mHSA-fusions) | Kᵢ (in nM) |
|---|---|
| AB2-6N/AB5-7C | 1.1 |
| AB2-3N/AB5-7C | 28 |
| AB2-21N/AB5-7C | 0.6 |
| AB5-7N/AB2-6C | 3 |
| AB5-7N/AB2-3C | 37 |
| AB5-7N/AB2-21C | 1.2 |
| Anti-ErbB3 mAb (IgG2) | (WO 2008/100624) |
| AB #6 | 0.1 |

Kᵢ values are averages from 2 independent experiments.

10-20 fold Kᵢ differences were observed between BBAs and anti-ErbB3 IgG, indicating that the BBA is not capable of antagonizing HRG-induced phosphorylation of ErbB3 as well as the anti-ErbB3 IgG antibody.

In another set of experiments, betacellulin (BTC)-induced phosphorylation of ErbB3 is examined. 2×10⁴ ADRr cells are pre-incubated for 1 hour with either the BBA at 1 uM followed by 9 subsequent 3-fold dilutions to give a 10-point curve or with an anti-ErbB3 IgG at 500 nM followed by 9 subsequent 3-fold dilutions to give a 10-point curve. Cells are treated with 50 nM BTC for 5 mins. BTC-induced phosphorylation of ErbB3 is measured using pErbB3 ELISA kit (R&D Systems, Cat # DYC1769E) to evaluate the ability of the agents to inhibit pErbB3 formation. The following results were obtained:

TABLE 4
Inhibition of BTC-Induced pErbB3 Formation by BBAs
(and IgG comparator)

| BBA | Kᵢ (in nM) |
|---|---|
| AB2-6N/AB5-7C | 6.5 |
| AB2-3N/AB5-7C | 14.2 |
| AB2-21N/AB5-7C | 0.52 |
| AB5-7N/AB2-6C | 5.4 |
| AB5-7N/AB2-3C | 23 |
| AB5-7N/AB2-21C | 0.56 |
| Anti-ErbB3 mAb (IgG2) | 1.1 |
| (WO 2008/100624) AB #6 | |

The results demonstrated that the BTC-stimulated pErbB3 signal could be completely inhibited by the BBAs.

(iii) Inhibition of IGF-1-Induced pAKT Formation

To determine the ability of the BBAs to inhibit intracellular signaling along the IGF-1R pathway, the ability of the agents to inhibit pAKT formation is tested. 1.5×10⁵ MCF7 cells are pre-incubated in 12 well plates for 1 hour with the BBA (starting at a high dose of 1 μM, with a total of nine 3-fold dilutions to give a 10-point curve) and then with IGF-1 at 13.5 nM for 15 minutes. IGF-1 induced phosphorylation of AKT is measured by ELISA using the following antibodies: anti-AKT, clone SKB1 (Millipore, Cat.#05-591); biotinylated anti-phospho-AKT (Ser⁴⁷³-specific; Cell Signaling Technology Cat.#5102). Detection is with streptavidin-HRP (R & D Systems, Cat.#DY998. The following results were obtained:

TABLE 5
Inhibition of pAKT Formation by BBAs

| BBA (mHSA-fusions) | Kᵢ (in nM) |
|---|---|
| AB5-7N/AB2-21C | 2 |
| AB5-7N/2-3C | 25 |
| AB5-7N/2-6C | 6 |
| AB2-21N/AB5-7C | 4 |
| AB2-3N/AB5-7C | 40 |
| AB2-6N/AB5-7C | 14 |

Kᵢ values are averages from 2 independent experiments

The results presented in Table 5, taken together with the results set forth in Table 1, demonstrate that the level of inhibition of IGF-1-induced pAKT correlates with the binding affinities of the BBAs. That is, tighter binding of the agent leads to improved inhibition pAKT formation.

(iv) Inhibition of IGF2-Induced pIGF-1R and pAKT Formation

In another set of experiments, IGF2-induced pIGF-IR and pAKT is examined. 2.5×10⁴ MCF7 cells are pre-incubated for 1 hour with either the BBA at 1 uM, followed by 9 subsequent 3-fold dilutions to give a 10-point curve, or with anti-IGF-IR IgG1 mAb391 at from 500 nM, followed by 9 subsequent 3-fold dilutions to give a 10-point curve. Cells are treated with 13 nM IGF2 for 15 minutes. IGF2-induced pIGF-IR is measured by pIGF-IR ELISA kit (R & D systems, Cat. # DYC1770) to evaluate the ability of the agents to inhibit pIGF-1R formation. IGF2-induced pAKT is measured by Merrimack developed ELISA assay to evaluate the ability of the agents to inhibit pAKT formation. The following results were obtained:

TABLE 6
Inhibition of IGF2-Induced pIGF-IR Formation by BBAs

| BBA (mHSA-fusions) | Kᵢ (in nM) |
|---|---|
| AB2-6N/AB5-7C | 4.1 |
| AB2-3N/AB5-7C | 14.0 |
| AB2-21N/AB5-7C | 0.74 |
| AB5-7N/AB2-6C | 2.3 |
| AB5-7N/AB2-3C | 16.8 |
| AB5-7N/AB2-21C | 0.27 |

TABLE 7
Inhibition of IGF2-Induced pAKT Formation by BBAs

| BBA (mHSA-fusions) | Kᵢ (in nM) |
|---|---|
| AB2-6N/AB5-7C | 10.8 |
| AB2-3N/AB5-7C | 14.1 |
| AB2-21N/AB5-7C | 0.07 |
| AB5-7N/AB2-6C | 1.9 |
| AB5-7 IgG2 | 0.7 |
| Anti-IGF-1R IgG1 | |
| mAb391 | 0.06 |

### EXAMPLE 4

### Anti-Tumor Activities of Monomeric BBAs

A. Tumor Cell Proliferation Assay

The effect of the bispecific agents on tumor cell proliferation is examined in vitro using a CTG assay, which is a luminescence-based assay that measures the amount of cellular ATP present (Promega; Cat.# PR-G7572). Three cells lines are examined, ADRr, BxPC-3 and

MCF7, which express the following levels of IGF-1R and ErbB3:

TABLE 8
Cell Line Receptor Expression

| Cell | | IGF- | ErbB3/ | IGF- |
|---|---|---|---|---|
| Line | Source | 1R/cell | cell | 1R:ErbB |
| ADRr | Ovarian | 22,926 | 33,205 | 0.7 |
| MCF7 | Breast | 45,886 | 28,994 | 1.6 |
| BxPC-3 | Pancreatic | 32,000 | 16,430 | 1.9 |

To determine the optimal growth conditions for carrying out the CTG assay, cell numbers, media/growth factors (IGF-1, HRG) and time points are titrated for the three cells lines. These optimization experiments demonstrated that the ADRr cell line showed a minimal response to the growth factors, the BxPC-3 cell line responded well to IGF-1 and the MCF7 cell line responded well to both IGF-1 and HRG. The growth conditions chosen for carrying out the inhibitor assays are 10% serum or 1% serum plus 100 ng/ml IGF-1 and 135 ng/ml HRG. The following cell numbers are used for the CTG assay: ADRr and MCF7—1250 cells/well (Day 6), 5000 cells/well (Day 3); BxPC-3—2500 cells/well (Day 6), 7500 cells/well (Day 3). Cells are incubated for 3 days or 6 days with the following doses of inhibitor: 1 μM, 250 nM, 62.5 nM, or 15.625 nM. Plates are equilibrated at room temperature for 20 minutes, then CTG reagent is added for 10 minutes at room temperature, and plates are read on an EnVision® plate reader (Perkin-Elmer).

The potency of the BBAs in the CTG assay is summarized below, wherein

(−)=<10% inhibition, (+)=>10% inhibition and (++)>20% inhibition.

TABLE 8A
Inhibition of cell proliferation.

| | ADRr | | | | | |
|---|---|---|---|---|---|---|
| | 1% | | BxPC-3 | | MCF7 | |
| | serum + | | 1% | | 1% | |
| | IGF- | ADRr | serum + | BxPC-3 | serum + | MCF7 |
| | 1 + | 10% | IGF-1 + | 10% | IGF-1 + | 10% |
| | HRG | serum | HRG | serum | HRG | serum |
| AB2-21N/AB5-7C | + | + | + | ++ | + | − |
| AB5-7N/AB2-1C | − | + | ++ | + | − | − |
| AB2-3N/AB5-7C | + | ++ | + | + | + | − |
| AB5-7N/AB2-3C | + | ++ | + | + | − | − |
| AB2-6N/AB5-7C | + | + | ++ | ++ | + | − |
| AB5-7N/AB2-6C | + | ++ | ++ | − | − | − |
| AB5-7 IgG2 | − | + | − | − | + | − |
| mAb391 | + | ++ | + | − | − | − |
| Anti-ErbB3 (IgG2) | − | + | ++ | + | − | − |
| (WO 2008/100624) | | | | | | |
| AB #6 | | | | | | |
| AB5-7 IgG2 + Anti- | − | + | + | − | − | − |
| ErbB3 mAb (IgG2) | | | | | | |
| (WO 2008/100624) | | | | | | |
| AB #6 | | | | | | |
| mAb391 + Anti- | + | ++ | ++ | ++ | + | − |
| ErbB3 mAb (IgG2) | | | | | | |
| (WO 2008/100624) | | | | | | |
| AB #6 | | | | | | |

The results indicate that all of the bispecific binding agents are capable of inhibiting the proliferation of at least some of the tumor cells tested. For the ADRr cell line, AB5-7N/AB2-3C and AB5-7N/AB2-6C were able to inhibit proliferation as well as the combination of anti-IGF-1R IgG and anti-ErbB3 IgG. For the BxPC-3 cell line, AB2-6N/AB5-7C was able to inhibit proliferation as well as the combination of anti-IGF-1R IgG and anti-ErbB3 IgG. For the MCF7 cell line, AB2-21N/AB5-7C, AB2-3N/AB5-7C and AB2-6N/AB5-7C were able to inhibit proliferation as well as the combination of anti-IGF-1R IgG and anti-ErbB3 IgG.

Tumor Spheroid Growth Assay

The effect of BBAs on the growth of tumor spheroids in vitro is examined as a model of tumor growth in vivo. Formation of tumor spheroids, including optimal conditions for such formation using small quantities of the basement membrane extract Matrigel, have been described in the art (see e.g., Ivascu, A. and Kubbies, M. (2006) J. Biomol. Screen. 11:922-932; Lin, R. Z. and Chang, H. Y. (2008) Biotechnol. J. 3:1172-1184). Four cells lines are examined, ADRr, MCF7, A549 and Ovcar 8. Cells (2000 cells/200 μl media with 10% fetal bovine serum) are added per well of a 96 well low attachment plate. On Day 2 or 3, the spheroids are photographed and measured and then treated with BBAs. On Day 9 or 10, the spheroids are photographed and measured again.

For data analysis, the spheroid growth based on area is determined using the following formula: (Day 9 area−Day 2 area)/Day 2 area×100. Percent inhibition is determined by: (control−sample)/control×100.

In a set of initial experiments, cells are treated with either a 0.5 μM anti-IGR-1R IgG alone or a 0.5 μM anti-ErbB3 IgG alone (monotherapy) to identify spheroid growth driven by IGF-1 and/or HRG. The results are summarized below. In Tables 9 and 10 below (+)=>15% (but <30%) inhibition, (++)=>30% (but <50%) inhibition, (+++)=>50% inhibition and (−)=<15% inhibition.

TABLE 9
Tumor Cell Line Spheroid Growth Driven by IGF-1 and/or HRG

| Responsiveness to: | Anti-ErbB3 IgG | Anti-IGF-1R IgG |
|---|---|---|
| A549 | ++ | ++ |
| ADRr | ++ | ++ |
| MCF7 | − | ++ |
| OVCAR8 | + | − |
| T47D | + | − |

Next, the effect of the BBAs (at 0.5 μM) on tumor spheroid growth is determined.

TABLE 10
Effect of BBAs and IgG binding agents on Tumor Spheroid Growth.

| Agent | A549 | ADRr | MCF7 | OVCAR8 |
|---|---|---|---|---|
| AB2-6N/AB5-7C | ++ | + | + | + |
| AB2-3N/AB5-7C | ++ | | ++ | − |
| AB2-21N/AB5-7C | +++ | + | ++ | − |
| AB5-7N/AB2-6C | +++ | + | ++ | − |
| AB5-7N/AB2-3C | ++ | − | +++ | − |
| AB5-7N/AB2-21C | +++ | + | ++ | − |
| Anti-ErbB3 (Ab #6) | ++ | + | − | + |
| Anti-IGF-1R (MAB391) | ++ | − | + | − |

These results demonstrate that all of the BBAs are capable of inhibiting the growth of at least some of the tumor spheroids examined.

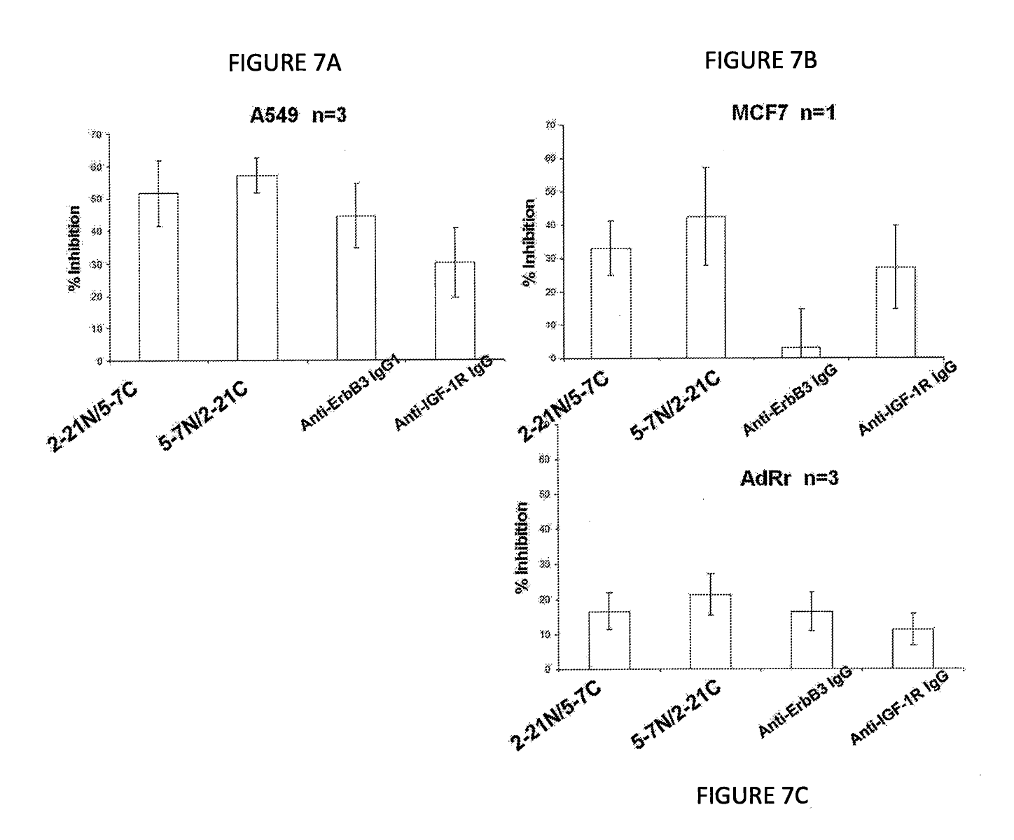

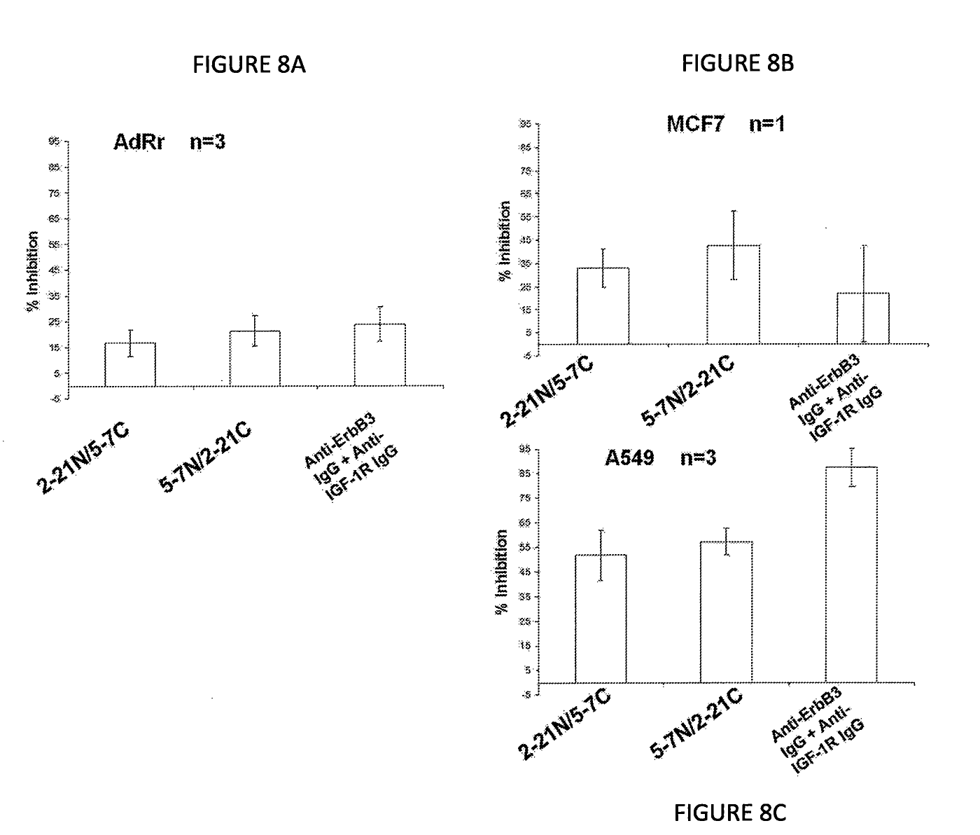

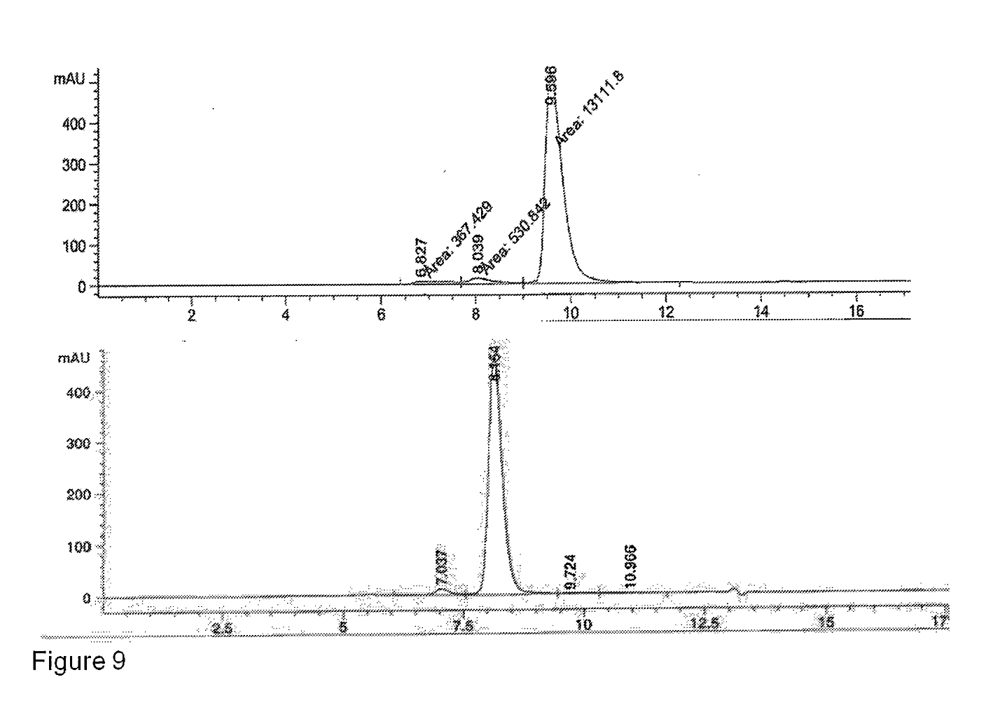

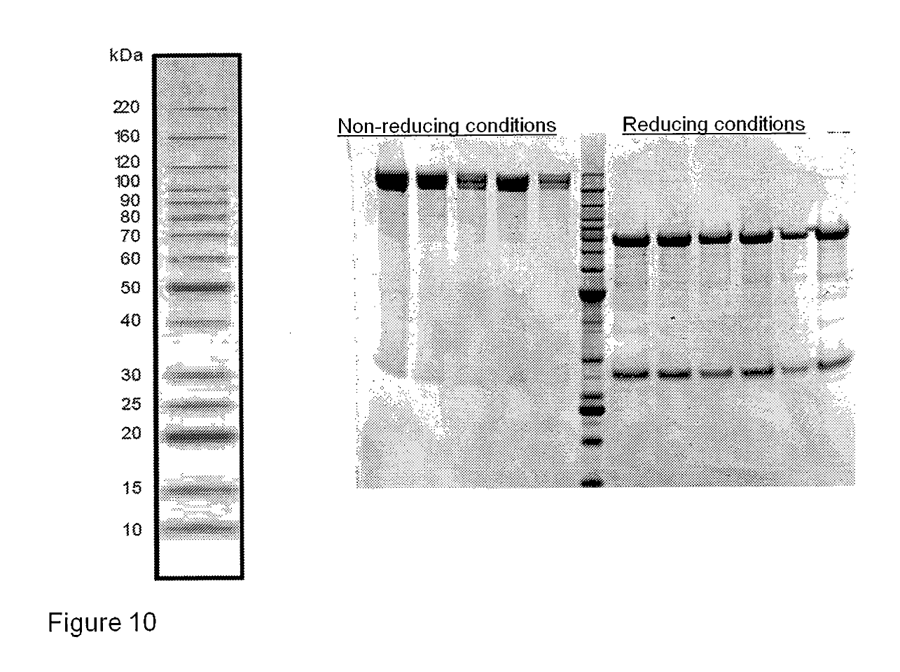

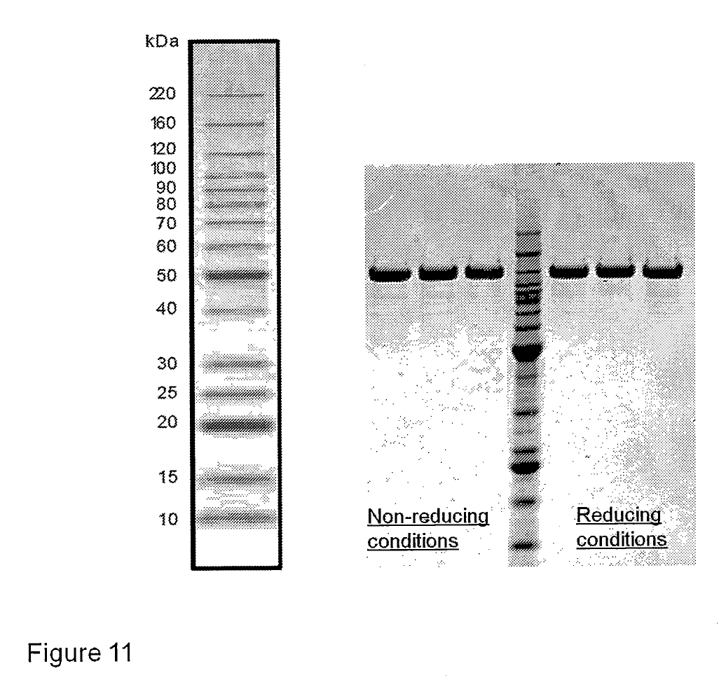

The ability of the BBAs to inhibit tumor spheroid growth also is compared to the effect of anti-IGF-1R IgG monotherapy, anti-ErbB3 IgG monotherapy and anti-IGF-1R IgG +anti-ErbB3 IgG combination therapy. Results for the monotherapy comparison are shown in FIGS. 7A-C, which demonstrates that the DX2-21N/DX5-7C and DX5-7N/DX2-21C BBAs show greater percent inhibition of tumor spheroid growth than either anti-IGF-1R IgG or anti-ErbB3 IgG alone in the A549 and MCF7 cell lines and about comparable inhibition to monotherapy for the ADRr cell line. The results for the combination therapy comparison are shown in FIGS. 8A-C, which demonstrates that the DX2-21N/DX5-7C and DX5-7N/DX2-21C BBAs perform as well as the anti-IGF-1R IgG +anti-ErbB3 IgG combination therapy in the ADRr and MCF7 cell lines, although the combination therapy was more effective in the A549 cell line.

### EXAMPLE 5

### Engineering of BBAs Targeting ErbB3 and IGF-1R

To expand on the therapeutic modalities described in examples 1-4 we engineered a diverse subset of additional BBAs targeting ErbB3 and IGF-1R. Examples of assembly of fusion molecules in anti- ErbB3-linker-anti- IGF-1R (ELI) and anti-IGF-1R-linker-anti-ErbB3 (ILE) N-terminus-to-C-terminus orientations are presented in Tables 11 and 12 respectively. The anti-ErbB3 moiety, linker moiety and anti-IGF-1R moiety of each exemplary molecule set forth in Table 11 are joined together contiguously N-terminus to C-terminus without intervening sequences. Coexpressed moiety, if present, is expressed in the same cell as separate polypeptide chain. The folding of these polypeptide chains gives rise to bispecific molecules of ELI topology.

TABLE 11
Fusion molecules with anti-ErbB3-linker-anti-IGF-1R topology (ELI)

| | | anti-ErbB3 | anti-IGF- | | |
|---|---|---|---|---|---|
| | | moiety | 1R moiety | | |
| | | (N- | (C- | linker | coexpressed |
| molecule | alias | terminal) | terminal) | moiety | moiety |
| ELI-1 | AB2-6N/ | SEQ ID | SEQ ID | SEQ ID | none |
| | AB5-7C | NO: 43 | NO: 1 | NO: 19 | |
| ELI-2 | AB2-6N/ | SEQ ID | SEQ ID | SEQ ID | none |
| | AB5-5C | NO: 43 | NO: 14 | NO: 19 | |
| ELI-3 | AB2-3N/ | SEQ ID | SEQ ID | SEQ ID | none |
| | AB5-7C | NO: 33 | NO: 1 | NO: 19 | |
| ELI-4 | AB2-3N/ | SEQ ID | SEQ ID | SEQ ID | none |
| | AB5-5C | NO: 33 | NO: 14 | NO: 19 | |
| ELI-5 | AB2-21N/ | SEQ ID | SEQ ID | SEQ ID | none |
| | AB5-7C | NO: 44 | NO: 1 | NO: 19 | |
| ELI-6 | AB2-21N/ | SEQ ID | SEQ ID | SEQ ID | none |
| | AB5-5C | NO: 44 | NO: 14 | NO: 19 | |
| ELI-7 | bs5F3 | SEQ ID | SEQ ID | SEQ ID | SEQ ID |
| | huIgG2 | NO: 35 | NO: 1 | NO: 22 | NO: 39 |
| ELI-8 | bs5F2 | SEQ ID | SEQ ID | SEQ ID | SEQ ID |
| | huIgG2 | NO: 35 | NO: 1 | NO: 23 | NO: 39 |
| ELI-9 | bs5W3 | SEQ ID | SEQ ID | SEQ ID | SEQ ID |
| | huIgG2 | NO: 41 | NO: 1 | NO: 21 | NO: 57 |
| ELI-10 | E3Bc8/ | SEQ ID | SEQ ID | SEQ ID | none |
| | THDT/ | NO: 47 | NO: 5 | NO: 30 | |
| | AB5-7 | | | | |
| ELI-11 | E3Bc8/ | SEQ ID | SEQ ID | SEQ ID | none |
| | THDL/ | NO: 47 | NO: 5 | NO: 31 | |
| | AB5-7 | | | | |
| ELI-12 | 5F3 IgG1 | SEQ ID | SEQ ID | SEQ ID | SEQ ID |
| | | NO: 36 | NO: 2 | NO: 24 | NO: 39 |
| ELI-13 | 5F3agly IgG1 | SEQ ID | SEQ ID | SEQ ID | SEQ ID |
| | | NO: 36 | NO: 2 | NO: 25 | NO: 39 |
| ELI-14 | 5F3 | SEQ ID | SEQ ID | SEQ ID | SEQ ID |
| | hyperglyIgG1 | NO: 36 | NO: 2 | NO: 26 | NO: 39 |
| ELI-15 | 5F3 IgG1- | SEQ ID | SEQ ID | SEQ ID | SEQ ID |
| | 11D | NO: 36 | NO: 4 | NO: 24 | NO: 39 |

The anti-IGF-1R moiety, linker moiety and anti-ErbB3 moiety of each exemplary molecule set forth in Table 12 are joined together contiguously N-terminus to C-terminus without intervening sequences. Coexpressed moiety, if present, is expressed in the same cell as separate polypeptide chain. The folding of these polypeptide chains gives rise to bispecific molecules of ILE topology.

TABLE 12
Fusion molecules with anti-IGF-1R-linker-anti-ErbB3 topology (ILE)

| | | anti-IGF-1R | anti-ErbB3 | | |
|---|---|---|---|---|---|
| | | moiety | moiety | linker | coexpressed |
| molecule | Alias | (N-terminal) | (C-terminal) | moiety | moiety |
| ILE-1 | AB5-7N/ | SEQ ID | SEQ ID | SEQ ID | none |
| | AB2-6C | NO: 1 | NO: 43 | NO: 19 | |
| ILE-2 | AB5-7N/ | SEQ ID | SEQ ID | SEQ ID | none |
| | AB2-3C | NO: 1 | NO: 33 | NO: 19 | |
| ILE-3 | AB5-7N/ | SEQ ID | SEQ ID | SEQ ID | none |
| | AB2-21C | NO: 1 | NO: 44 | NO: 19 | |
| ILE-4 | AB5-5N/ | SEQ ID | SEQ ID | SEQ ID | none |
| | AB2-6C | NO: 14 | NO: 43 | NO: 19 | |
| ILE-5 | AB5-5N/ | SEQ ID | SEQ ID | SEQ ID | none |
| | AB2-3C | NO: 14 | NO: 33 | NO: 19 | |
| ILE-6 | AB5-5N/ | SEQ ID | SEQ ID | SEQ ID | none |
| | AB2-21C | NO: 14 | NO: 44 | NO: 19 | |
| ILE-7 | Tvbs15A | SEQ ID | SEQ ID | SEQ ID | none |
| | | NO: 1 | NO: 48 | NO: 19 | |
| ILE-8 | Tvbs15B | SEQ ID | SEQ ID | SEQ ID | none |
| | | NO: 1 | NO: 49 | NO: 19 | |
| ILE-9 | Tvbs15C | SEQ ID | SEQ ID | SEQ ID | none |
| ILE-10 | Bs14F3 | NO: 1 | NO: 50 | NO: 19 | |
| | huIgG2 | SEQ ID | SEQ ID | SEQ ID | SEQ ID |
| ILE-11 | Bs14F2 | NO: 6 | NO: 33 | NO: 22 | NO: 10 |
| | huIgG2 | SEQ ID | SEQ ID | SEQ ID | SEQ ID |
| ILE-12 | Bs15F3 | NO: 6 | NO: 33 | NO: 23 | NO: 10 |
| | huIgG2 | SEQ ID | SEQ ID | SEQ ID | SEQ ID |
| ILE-13 | Bs15F2 | NO: 6 | NO: 44 | NO: 22 | NO: 10 |
| | huIgG2 | SEQ ID | SEQ ID | SEQ ID | SEQ ID |
| ILE-14 | Bs14W3 | NO: 6 | NO: 44 | NO: 23 | NO: 10 |
| | huIgG2 | SEQ ID | SEQ ID | SEQ ID | SEQ ID |
| ILE-15 | 5-7 IgG1- | NO: 12 | NO: 33 | NO: 20 | NO: 58 |
| | E3Bc8 | SEQ ID | SEQ ID | SEQ ID | SEQ ID |
| ILE-16 | 5-7 IgG1- | NO: 7 | NO: 47 | NO: 24 | NO: 11 |
| | AB2-3 | SEQ ID | SEQ ID | SEQ ID | SEQ ID |
| | | NO: 7 | NO: 34 | NO: 24 | NO: 11 |

In summary, fusion molecules ELI-1, ELI-2, ELI-3, ELI-4, ELI-5, ELI-6, ILE-1, ILE-2, ILE-3, ILE-4, ILE-5, ILE-6, ILE-7, ILE-8, ILE-9 were designed to be functionally monomeric. Fusion molecules ELI-7, ELI-8, ELI-9, ELI-12, ELI-13, ELI-14, ELI-15, ILE-10, ILE-11, ILE-12, ILE-13, ILE-14, ILE-15, ILE-16 were designed to be functionally dimeric. Fusion molecules ELI-10 and ELI-11 were designed to be functionally trimeric. Fusion molecules ELI-1, ELI-2, ELI-3, ELI-4, ELI-5, ELI-6, ILE-1, ILE-2, ILE-3, ILE-4, ILE-5, ILE-6, ILE-7, ILE-8, ILE-9, ELI-7, ELI-8, ELI-9, ELI-12, ELI-13, ELI-14, ELI-15, ILE-10, ILE-11, ILE-12, ILE-13, ILE-14, ILE-15, ILE-16 were designed to have enhanced half-life in systemic circulation via active recycling by neonatal Fc receptor (Ghetie et al., Annu. Rev. Immunol., 2000, 18, 739-766; Chaudhury et al., J Exp Med., 2003, 197, 315-22). Fusion molecules ELI-10 and ELI-11 were designed to have enhanced half-life in systemic circulation via the increase of hydrodynamic radius (Tsutsumi et al., J Pharmacol Exp Ther., 1996, 278, 1006-11). Multiple strategies have been employed to improve the drug properties of IGF-1R targeting moieties, ErbB3 targeting moieties, and linker moieties. The stabilities and expression levels of the targeting moieties in ELI-10, ELI-11, ELI-12, ELI-13, ELI-14, ELI-15, ILE-15, and ILE-16 were improved by applying previously reported techniques (Langedijk et al., J. Mol. Biol., 1998, 283, 95-110; Nieba et al., Protein Eng., 1997, 10, 435-444; Ewert at al., Biochemistry, 2003, 42, 1517-1528; Chowdhury et al. J. Mol. Biol., 1998, 281, 917-928; Worn et al., J. Mol. Biol., 305, 989-1010). The heterogeneity of targeting moieties in ELI-10 and ELI-11 was decreased by reengineering of C-terminus for resistance to basic carboxypeptidases (Harris, Journal of Chromatography A, 1995, 23, 129-134). The linker moieties in ELI-13 and ELI-14 were glycoengineered for increased solubility or reduced heterogeneity as previously described (Pepinsky et al., Protein Sci., 2010, 19, 954-66; Lund et al., Mol Immunol., 1993, 30, 741-8). The homogeneity of each of the linker moieties in ELI-1, ELI-2, ELI-3, ELI-4, ELI-5, ELI-6, ILE-1, ILE-2, ILE-3, ILE-4, ILE-5, ILE-6, ILE-7, ILE-8, and ILE-9 was increased.

### EXAMPLE 6

### Preparation, Expression and Purification of BBAs Targeting ErbB3 and IGF-1R.

The nucleic acids encoding fusion molecules described in Example 5 were cloned as single molecules into the expression plasmids using standard recombinant DNA techniques. An expression vector employed was pMP 10K (SELEXIS). Expression plasmids were linearized, purified using QIAquick purification kit (QIAGEN), and co-transfected into CHO cells using Lipofectamine LTX (Invitrogen). Transfected cells were recovered with F12Hams medium containing 10% FBS for 2 days without selection pressure, then with selection pressure for 4 days. After 4 days, they were changed into serum-free medium (Hyclone) containing glutamine with selection pressure. After a week, cells were checked for expression and scaled up to desired volume. All fusion molecules were purified using a combination of three chromatography steps: protein A affinity, cation exchange and anion exchange. Each was carried out in accordance with the manufacturer's instructions. The protein A affinity step was used to selectively and efficiently binds the fusion molecules out of harvested cell culture fluids (HCCF). This step removed >95% of product impurities in a single step with high yields and high throughput. The portion of desired molecular form for fusion molecules after this step was in the range of 60 to 98 percent. MABSELECT from GE was used as the Protein A affinity resin. SPFF (sulphopropyl fast flow) from GE, an agarose based resin, was used as the cation exchange resin in the second chromatography step. The portion of desired molecular form for fusion molecules after this step was in the range of 90 to 99 percent. QSFF (Quaternary-amine sepharose fast flow) from GE, an agarose based anion exchange resin, was used in a third and final chromatography step. The purified material was concentrated and dialyzed into a phosphate buffered saline. The final yield for the fusion molecules after this step was is in the range of 20 mg-100 mg/L.

### EXAMPLE 7

### Binding and Biological Activity of Diverse BBAs Targeting the IGF-1R and ErbB3 Pathways

### A) Binding of BBAs to IGF-1R and ErbB

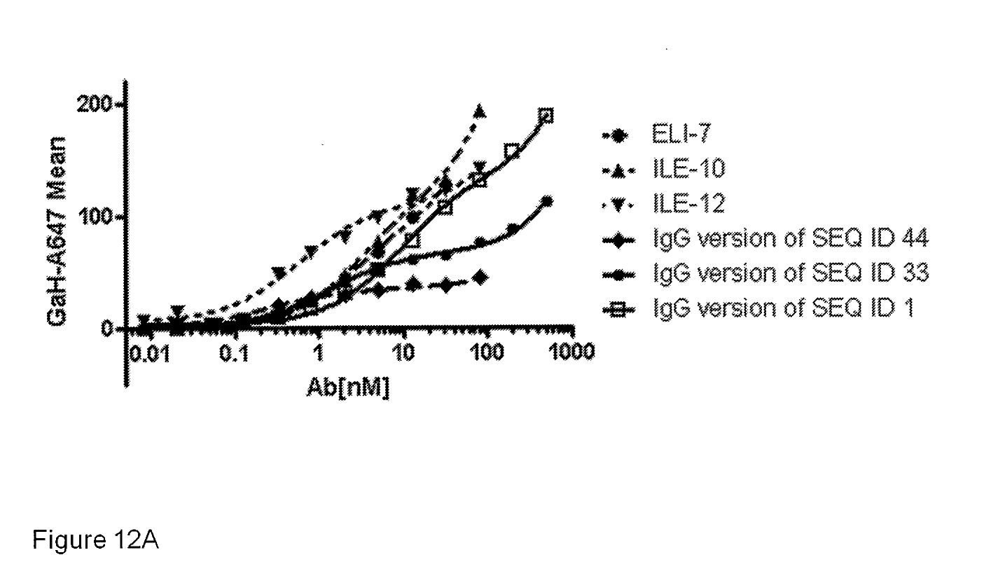

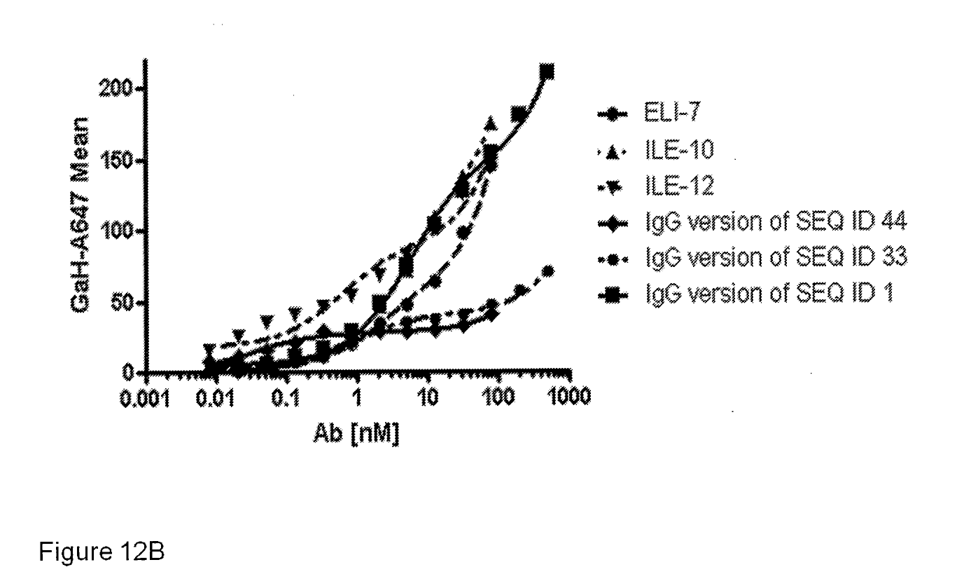

1×10⁵ MCF7 cells and 1×10⁵ ADRr cells are incubated at room temperature for 2 hours with the BBA at 2 uM, followed by 12 subsequent 3-fold dilutions. Then using goat anti-HSA-Alexa647 conjugated antibody as the detection antibody, cells are incubated on ice for 40 minutes. Cell binding dissociation constants (measures of binding affinities) of the BBAs on MCF7 and ADRr cells are assessed by FACS and apparent dissociation constants are determined for each BBA. The following results were obtained (also see FIGS. 12a and 12b):

TABLE 13

| | Kd (nM) | |
|---|---|---|
| | ADRr | MCF7 |
| inhibitor | (n = 3) | (n = 1) |
| ELI-7 | 2.5 | 2.1 |
| ILE-10 | 7.1 | 4.5 |
| ILE-12 | 0.3 | 0.6 |
| IgG version of SEQ ID NO: 44 | 0.4 | 0.04 |
| IgG version of SEQ ID NO: 33 | 1.2 | 0.9 |
| IgG version of SEQ ID NO: 1 | 5.1 | 5.6 |

IgG-bispecifics (i.e. ELI-7, ILE-10, ILE-12) bound to both cell types, in some cases with greater binding at low concentrations indicating avid binding and the ability to bind to each receptor. The IgG-bispecifics had a similar Kd to the equivalent monoclonal antibody component.

2×10⁶ BXPC3 cells are incubated at room temperature for 2 hours with the BBA at 0.5 uM, followed by 11 subsequent 2.5-fold dilutions. Then using goat anti-HSA-Alexa647 conjugated antibody as the detection antibody, cells are incubated on ice for 45 minutes. Cell binding dissociation constants (measures of binding affinities) of the BBAs on BXPC3 cells are assessed by FACS and apparent dissociation constants are determined for each BBA. The following results were obtained:

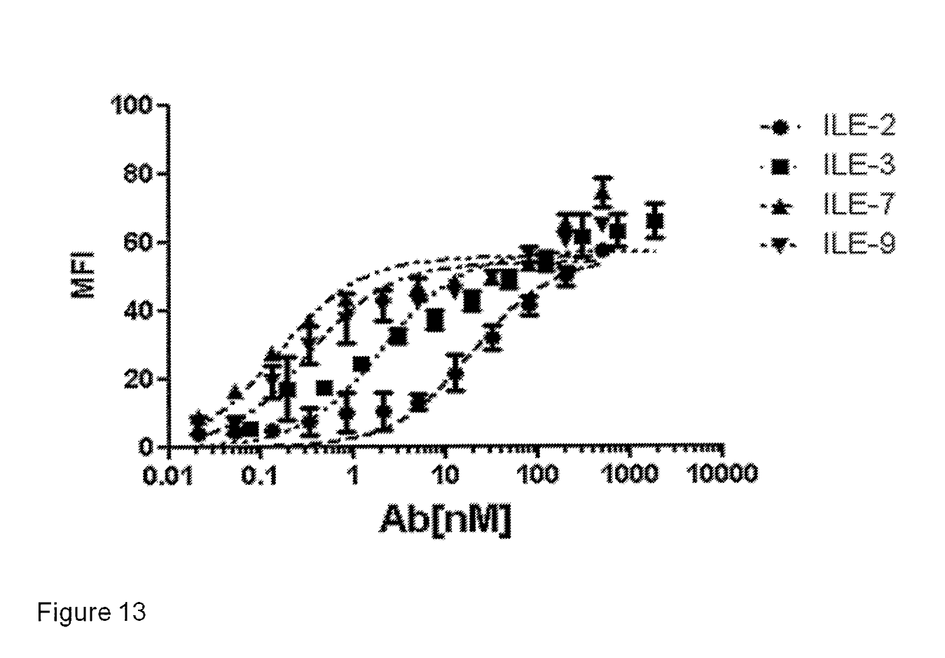

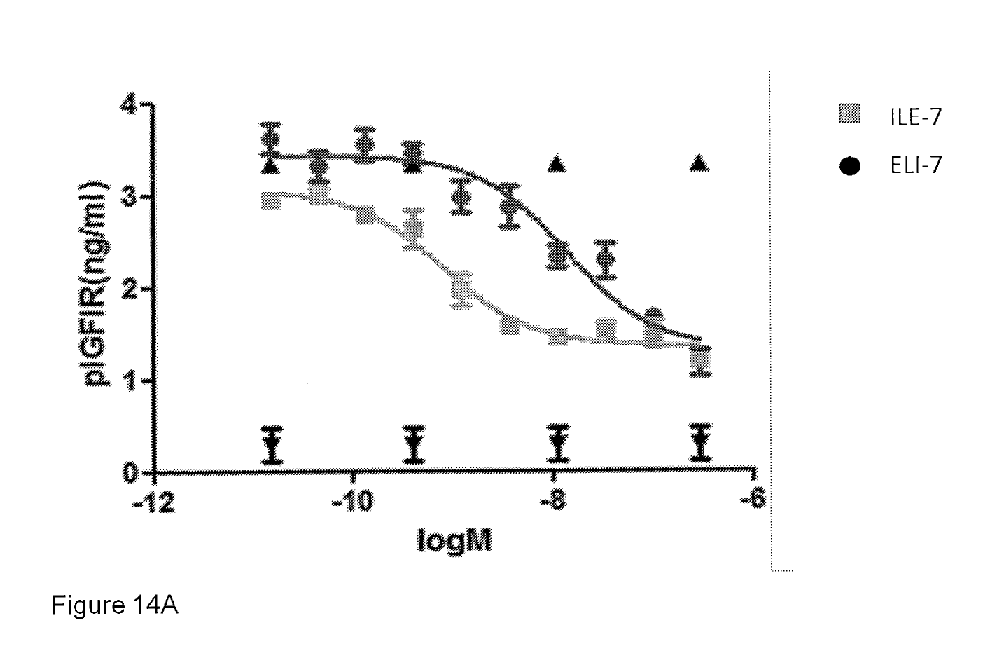

FIG. 13 shows a representative result from three experiments (tabulated below)

TABLE 14

| Kd Apparent | ILE-2 | ILE-3 | ILE-7 | ILE-9 |
|---|---|---|---|---|
| Exp1 | 1.607 | 3.359 | 0.1515 | 0.6337 |
| Exp2 | 38.8 | 2.259 | 0.1372 | 0.5614 |
| Exp3 | 25.41 | 2.463 | 0.1897 | 0.1108 |
| average | 21.94 | 2.69 | 0.16 | 0.44 |

The trivalent bispecifics (ILE-7, ILE-9) bind much more tightly than the control bispecifics (ILE-2, ILE-3), indicating that the addition of a second ErbB3 binding moiety to a non-overlapping epitope significantly improves binding to cells.
B) Signal inhibition of IGF-1R and ErbB3 and Akt by BBAs

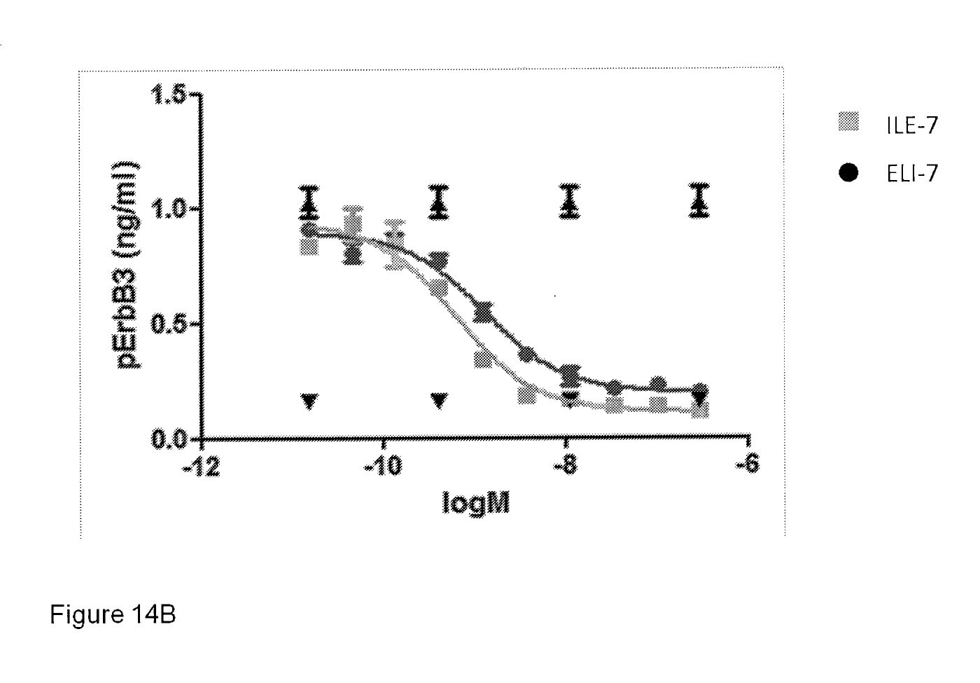

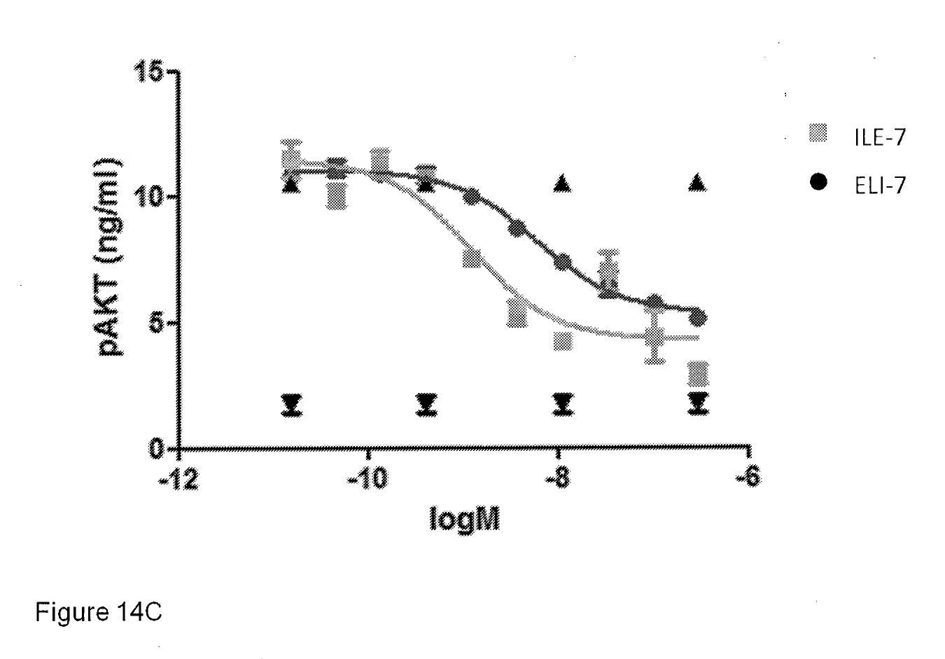

To determine the ability of the BBAs to antagonize IGF-1R and ErbB3 and a common downstream component (Akt), inhibition of IGF-1R phosphorylation, ErbB3 phosphorylation and Akt phosphorylation is examined in the presence of the agents. 3.5×10⁴ BXPC3 cells are pre-incubated for 1 hour with a BBA at 0.3 μM, followed by 9 subsequent 3-fold dilutions to give a 10-point curve. Cells are treated with IGF-1 at 80 ng/ml and heregulin at 20 ng/ml for 15 minutes. Phosphorylation of IGF-1R to yield phospho-IGF-1R (pIGF-1R) is measured by ELISA (R & D Systems; Cat.# DYC1770) to evaluate the ability of the agents to inhibit pIGF-1R formation. Phosphorylation of ErbB3 is measured by ELISA (R & D Systems; Cat.#DYC1769) to evaluate the ability of the agents to inhibit pErbB3 formation. Phosphorylation of AKT is measured by ELISA using the following antibodies: anti-AKT, clone SKB1 (Millipore, Cat.#05-591); biotinylated anti-phospho-AKT (Ser⁴⁷³-specific; Cell Signaling Technology Cat.#5102). FIGS. 14A-14C show the results for ILE-7 and ELI-7, the results are also summarized in the table below.

TABLE 15

| | Ki nM | |
|---|---|---|
| | ELI-7 | ILE-7 |
| pAkt | 6.3 | 1.3 |
| pErbB3 | 1.3 | 0.6 |
| pIGF-1R | 14.1 | 0.8 |

ELI-7, an IgG-linked BBA and ILE-7, a trivalent HSA-linked BBA, can inhibit pErbB3, pIGF-1R and pAkt even with simultaneous stimulation with IGF-1 and HRG.

### C) Cell Growth Inhibition by BBAs in Two Dimensional Culture

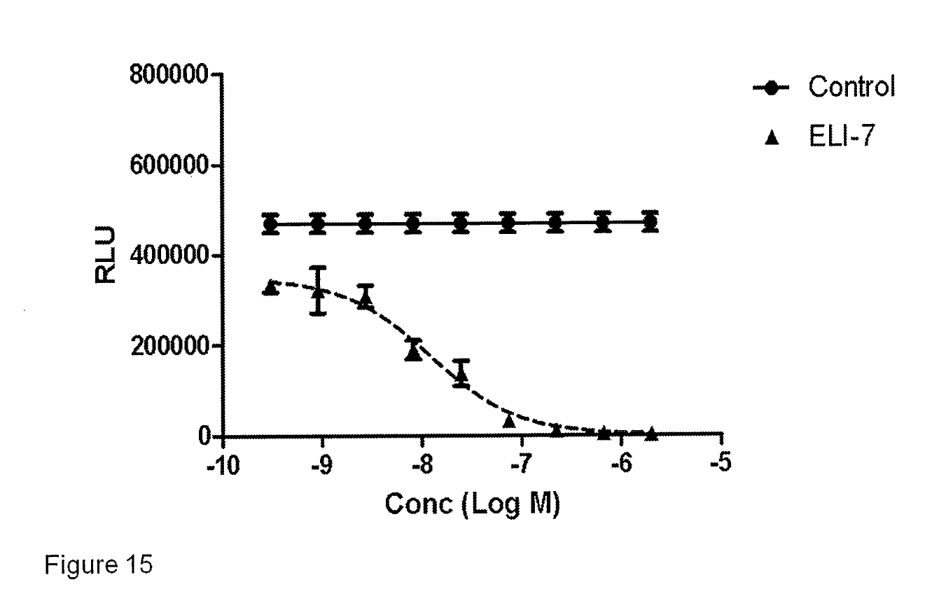

The effect of the bispecific agents on tumor cell proliferation is examined in vitro using a CTG assay, which is a luminescence-based assay that measures the amount of cellular ATP present (Promega; Cat.# PR-G7572), indicated as Relative Light Units (RLU). 500 cells per well of DU145 cells were incubated for 6 days in media with 80 ng/ml IGF-1 and 20 ng/ml heregulin and containing a 3-fold dilution of inhibitors starting at 2uM. The IgG-bispecific BBA ELI-7 inhibited the growth of DU145 cells (Ki=12nM for ELI-7, see FIG. 15. Inhibitors to only IGF-1R or ErbB3 had no effect on cell growth.

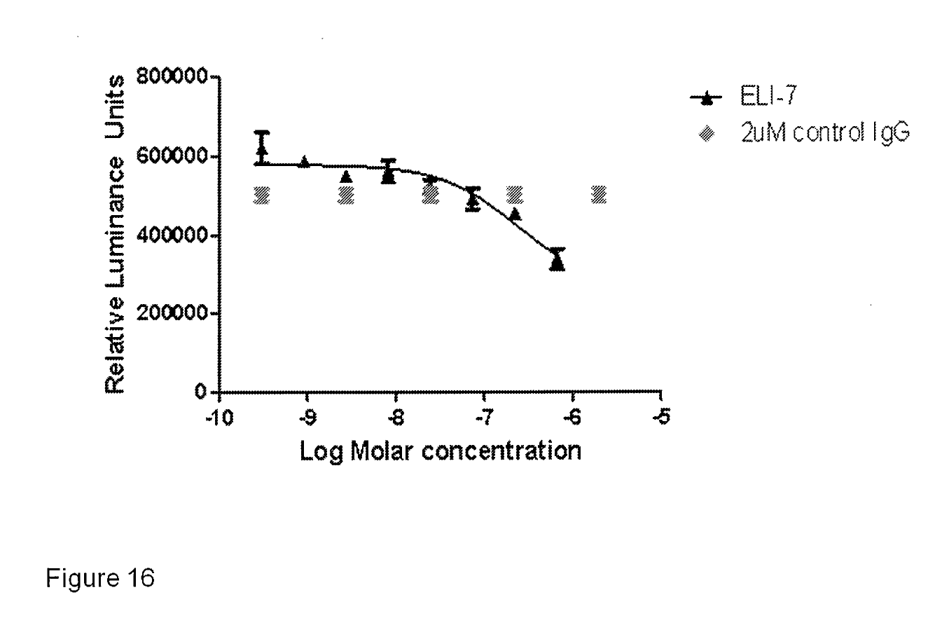

2000 cells per well BXPC3 were incubated for 6 days in media containing a 3-fold dilution of inhibitors starting at 1 uM. The IgG-bispecific BBA ELI-7 inhibited BXPC3 growth by 46% (p <0.001, Student's T-test) (FIG. 16).

### D) Cell Growth Inhibition by BBAs in Three Dimensional Cultures

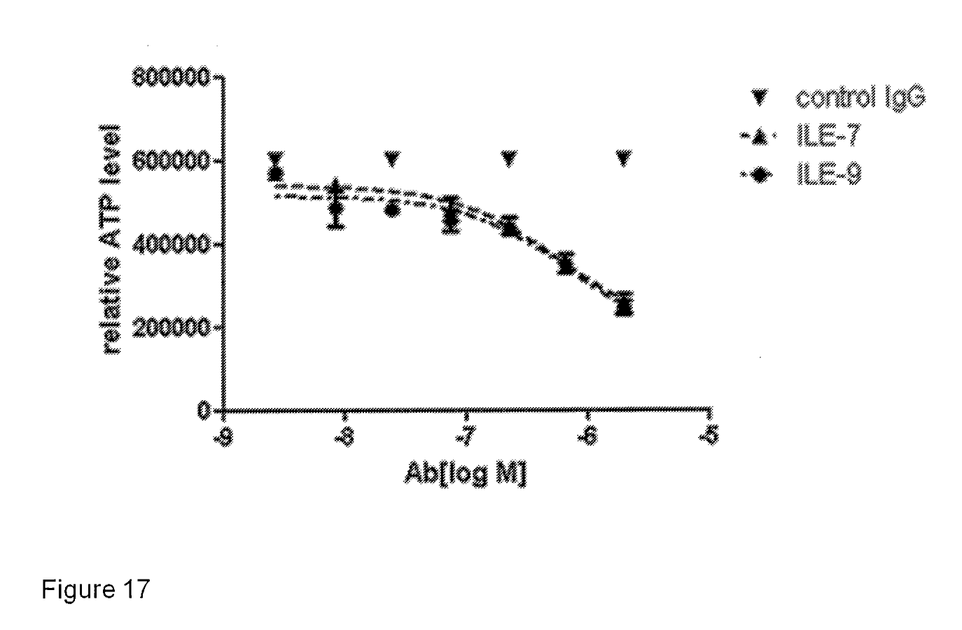

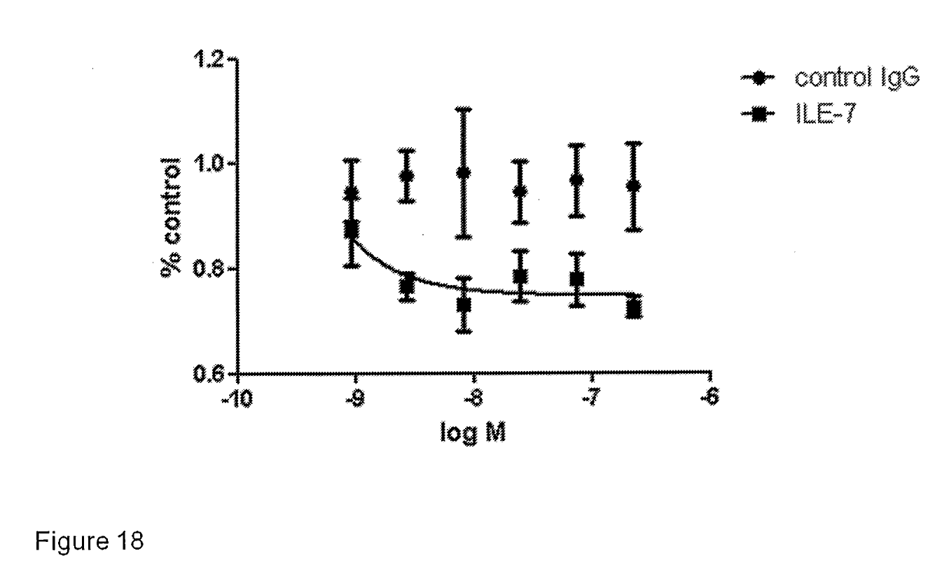

The effect of the BBAs on tumor cell proliferation is examined in vitro using a CTG assay, which is a luminescence-based assay that measures the amount of cellular ATP present (Promega; Cat.# PR-G7572), indicated as Relative Light Units (RLU). In this case specialized nano-culture plates (Scivax: Cat. #NCP-L-MS-96) are used to enable cells to grow in three-dimensions. 10,000 BXPC3 cells are incubated for 10 days in media containing 10% FBS. At the completion of the third day inhibitors are added to various wells using a 3-fold dilution starting at 2uM. The trivalent BBAs ILE-9 and ILE-7 inhibited the growth of BXPC3 cells as measured by CTG by 44% and 48%, respectively (p<0.01 Student's t-test) (FIG. 17). In this case specialized nano-culture plates (Scivax: Cat. #NCP-L-MS-96) are used to enable cells to grow in three-dimensions. 10,000 DU145 cells were incubated for 10 days in media containing 10% FBS. At the completion of the third day inhibitors are added to various wells using a 3-fold dilution starting at 2 uM. The trivalent bispecific, ILE-7, inhibits growth of DU145 cells as measured by CTG by 28% (p=0.02, Student's t-test) (FIG. 18).

### E) Tumor Growth Inhibition by BBAs in Mouse Models of Cancer

The effect of BBAs on tumor growth in mouse models of cancer was assessed by first calculating the pharmacokinetic properties of each BBA. 500 ug of each HSA-linked BBA or 600 ug of each IgG-linked BBA was injected via tail vein into each mouse (4 mice per inhibitor and timepoint), and blood was drawn at various timepoints thereafter (mice were first sacrificed and then blood was drawn by cardiac puncture). Timepoints for BBAs with HSA-linker are: 0.5, 4, 8, 24, 28, 72, and 120 hours. Timepoints for BBAs with IgG-linker are: 0.5, 4, 24, 72, 120, 168 and 240 hours. Concentration in the blood is measured for BBAs with HSA-linker using an in-house ELISA kit that detects IGF-1R and ErbB3 binding. Specifically, plates are coated with His-tagged human IGF-1R, incubated with BBAs, then detected with an human ErbB3-Fc chimera and an anti-Fc-HRP detection reagent. Concentration in the blood is measured for BBAs with IgG-linker using an anti-human IgG ELISA kit (Bethyl labs Cat.# E80-104) according to the manufacturer's instructions. Pharmacokinetic properties (half-life and Cmax) for each BBA is calculated using a one-compartment model. The following results were obtained:

TABLE 16

| | Half life | Cmax |
|---|---|---|
| BBA or antibody | (hours) | (ug/ml) |
| ILE-3 | 15 | 410 |
| ILE-7 | 14 | 516 |
| ILE-9 | 17 | 447 |
| ELI-7 | 48 | 612 |
| matched anti-IGF-1R IgG | 124 | 517 |
| (SEQ ID NO: 1) | | |
| matched anti-ErbB3 IgG | 58 | 645 |
| (SEQ ID NO: 33) | | |

Simulation of drug-specific half-lives led to prediction that the following doses would result in equal exposure (or in the case of ILE-7 50% comparable exposure):

TABLE 17

| | Dose (ug) |
|---|---|
| ILE-7 | 800 |
| ELI-7 | 600 |
| matched anti-IGF-1R IgG | 300 |
| matched anti-ErbB3 IgG | 500 |

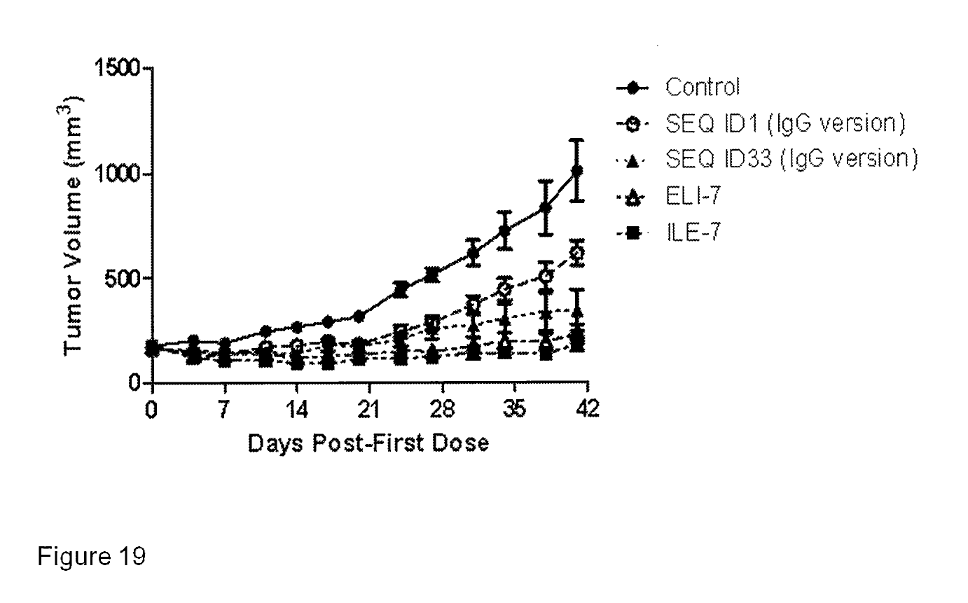

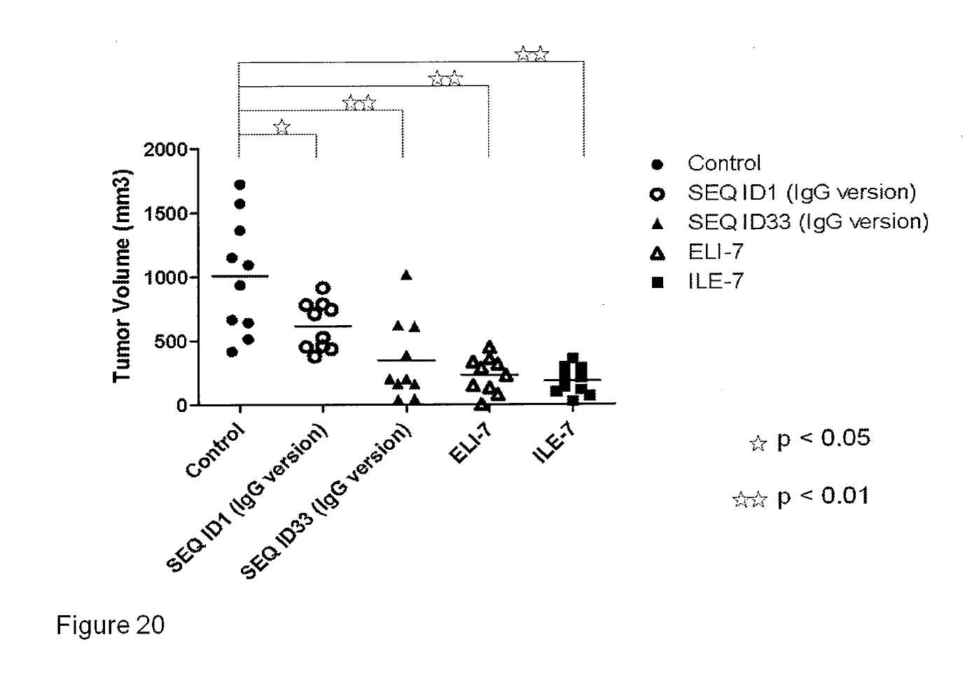

The effect of BBAs on tumor growth in mouse models of pancreatic cancer was then assessed by injecting 5×10⁶ BXPC3 cells (resuspended in a 1:1 mixture of PBS and growth factor-reduced matrigel; BD Biosciences Cat.# 354230) into the subcutaneous space in the flank of each mouse. Tumor were allowed to develop for 7-10 days (until they reached a volume of approximately 100-200 mm³), and then tumor size was measured for each mouse (pi/6×length×widtĥ2, where width is the smallest measurement). Mice were then size-matched and then randomly assigned into the treatment groups. BBAs, anti-IGF-1R antibodies, anti-ErbB3 antibodies, or a PBS control were then injected every 3 days until the completion of the study. FIGS. 19A, 19B, and 20 show that both ILE-7 and ELI-7 significantly inhibited the xenograft tumor growth of BXPC3 cells compared to the PBS control: final tumor volume was 82% lower in ILE-7 treated tumors and 77% lower in ELI-7 treated tumors compared to the PBS control (p values determined by student's T-test). Day 0 refers to the first day of dosing.

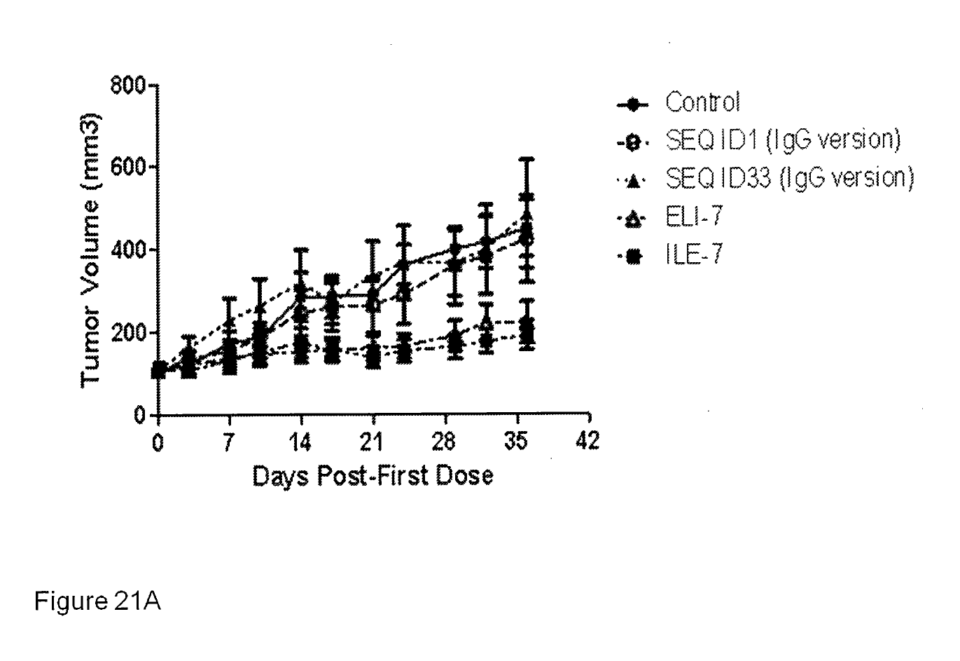

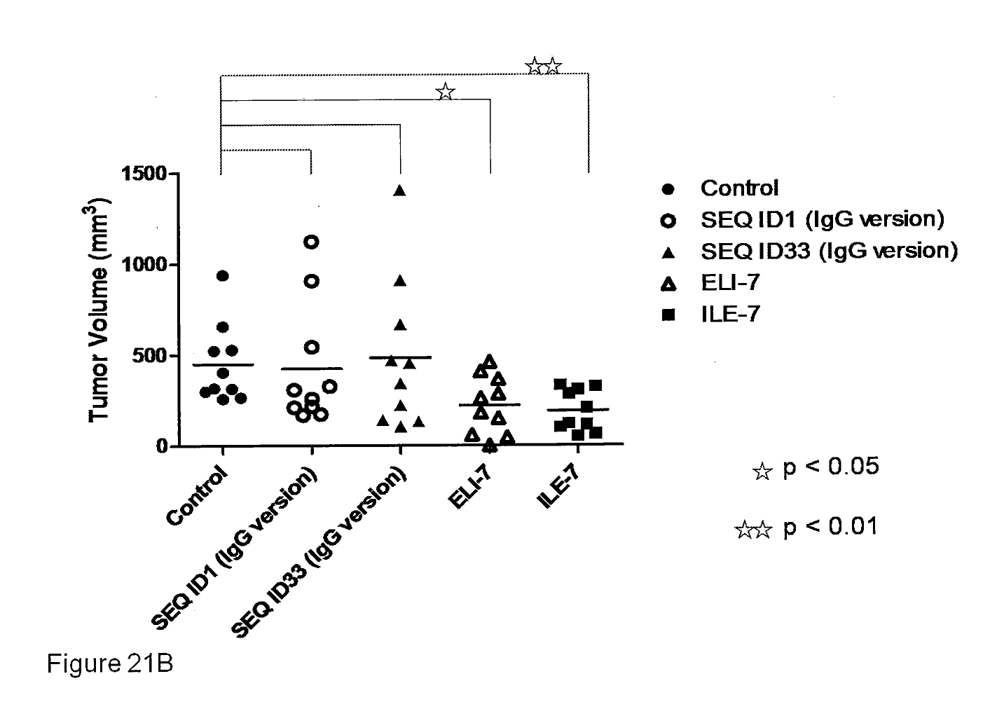

The effect of BBAs on tumor growth in mouse models of prostate cancer was then assessed by injecting 5×10⁶ DU145 cells (resuspended in a 1:1 mixture of PBS and growth factor-reduced matrigel; BD Biosciences Cat.# 354230) into the subcutaneous space in the flank of each mouse. Tumor were allowed to develop for 7-10 days (until they reached a volume of approximately 100-200 mm³), and then tumor size was measured for each mouse (pi/6×length×widtĥ2, where width is the smallest measurement). Mice were then size-matched and then randomly assigned into the treatment groups. BBAs, anti-IGF-1R antibodies, anti-ErbB3 antibodies, or a PBS control were then injected every 3 days until the completion of the study. FIGS. 21A and 21B show that the BBAs ILE-7 and ELI-7 both significantly inhibited xenograft tumor growth of DU145 cells, whereas inhibitors to IGF-1R or ErbB3 did not: final tumor volume was 57% lower in ILE-7 treated tumors and 50% lower in ELI-7 treated tumors compared to the PBS control (p values determined by student's T-test). Day 0 refers to the first day of dosing.

### EXAMPLE 8

### BBAs Have a Novel Mechanism of Action and Display Potency Across a Range of Receptor Profiles

### A) Trivalent-BBA Dual Targeting ErbB3 has Novel Mechanism of Action

a. HSA-Bispecifics Have Limited Signaling Inhibition

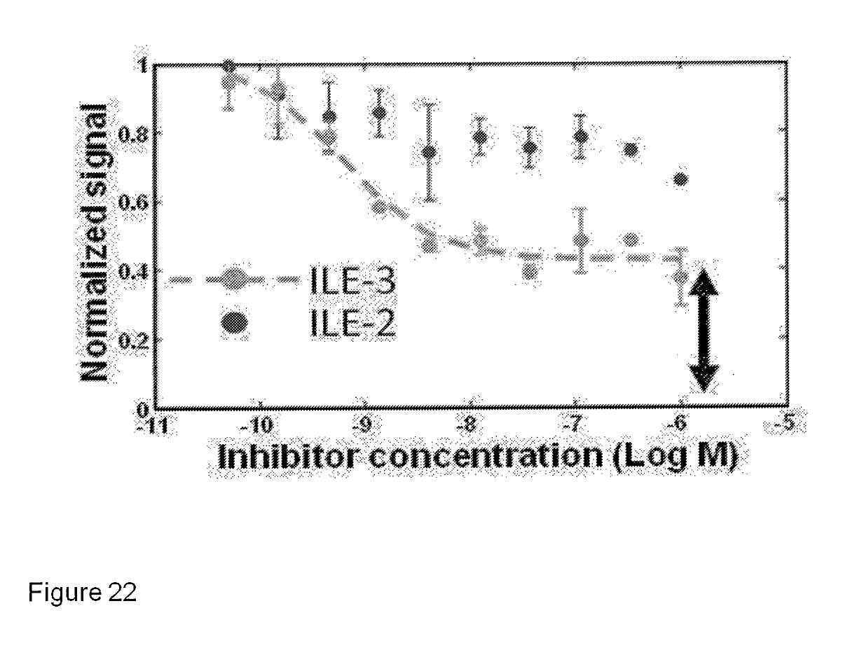

In some cases we observed limited inhibition of pErbB3 by BBAs that utilize only one anti-ErbB3 moiety. For example, 5nM of Heregulin induces pErbB3 in ADRr cells within 15 minutes; however, inhibition with ILE-2 or ILE-3 pre-treatment for 1 hour results in incomplete inhibition (1 uM top dose with 3-fold dilution). In particular ILE-3 is able to reach maximum achievable inhibition at low dosage (<10nM), but cannot inhibit greater than 60% (FIG. 22).

b. Predictions of Combining ErbB3 Antagonists

Competition assays have shown that the anti-ErbB3 moieties, SEQ ID 33 and SEQ ID 44 bind to separate domains of ErbB3, domain 3 and 1, respectively. Therefore it is possible for inhibitors utilizing SEQ ID 33 and SEQ ID 44 to bind simultaneously to ErbB3. This is shown below, where ADRr cells were stimulated with 5nM Heregulin for 15 minutes, and inhibitors were pre-incubated in media for 1 hour.

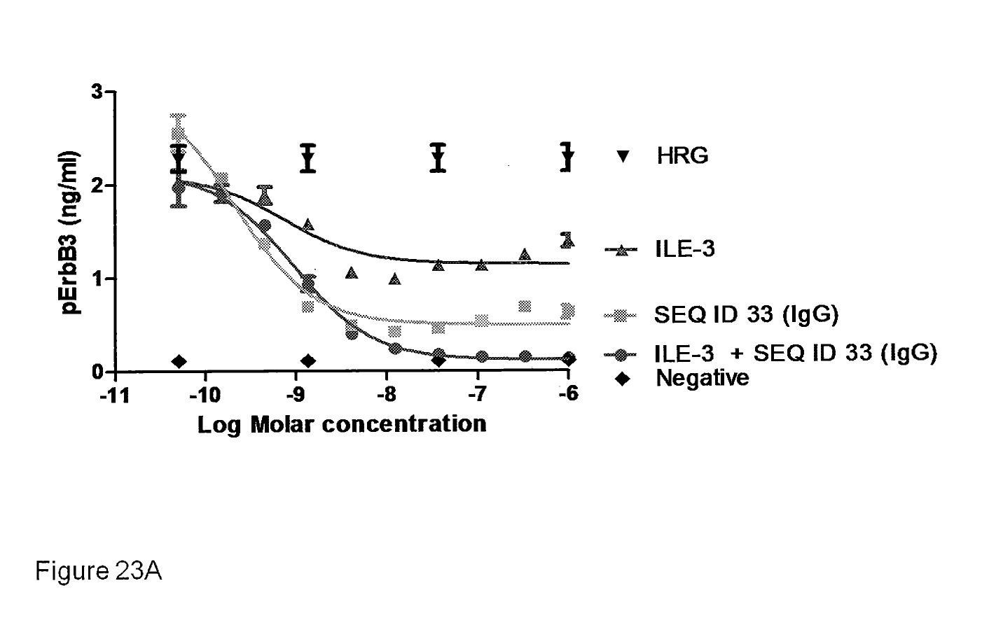

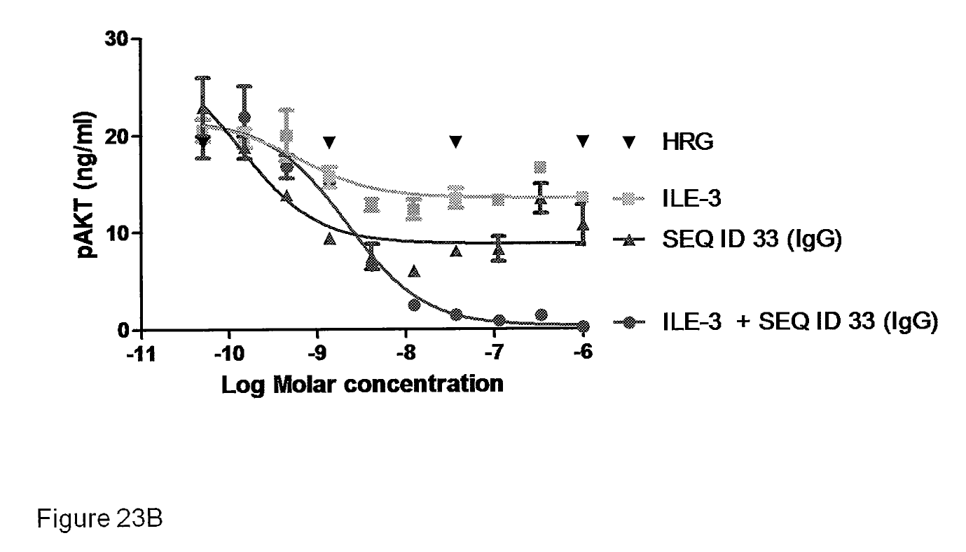

The combination of SEQ ID 33 (as IgG) and ILE-3 is able to achieve completion inhibition of phosphorylated ErbB3, indicating the anti-ErbB3 moieties SEQ ID 33 and SEQ ID 44 have complimentary mechanism of action (FIGS. 23a and 23b).

c. Experimental Confirmation of Full Inhibition of pErbB3 by the Trivalent Bispecific Format

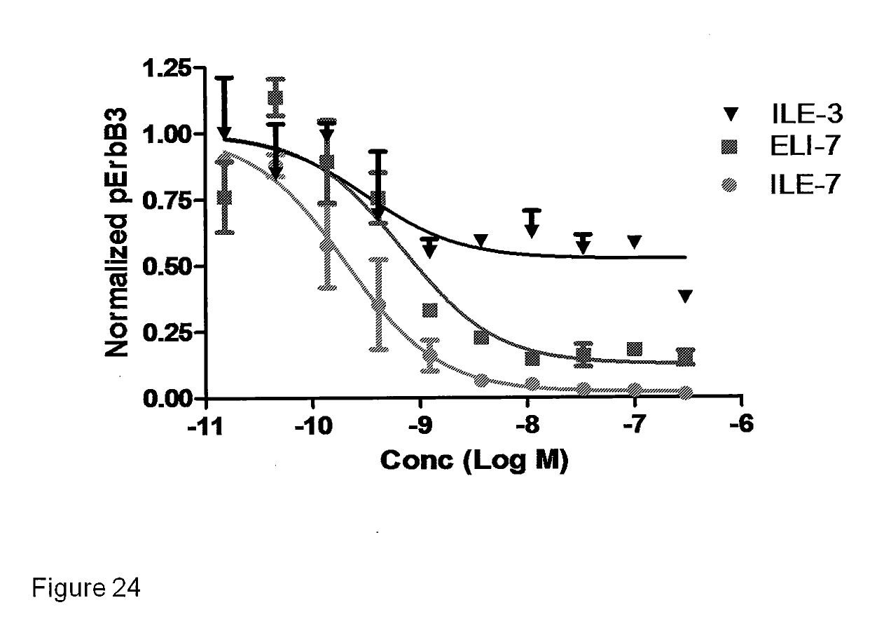

To confirm that ILE-7 (comprised of SEQ ID 1, SEQ ID 33, SEQ ID 44) could indeed completely inhibit phosphorylated ErbB3, 2×10⁴ ADRr cells were treated with 5 nM Heregulin for 15 minutes following 1 hour pre-treatment with BBAs (0.5 nM starting concentration with 10-point 3-fold dilution). ILE-7 completely inhibited pErbB3 whereas ILE-3 did not (FIG. 24).

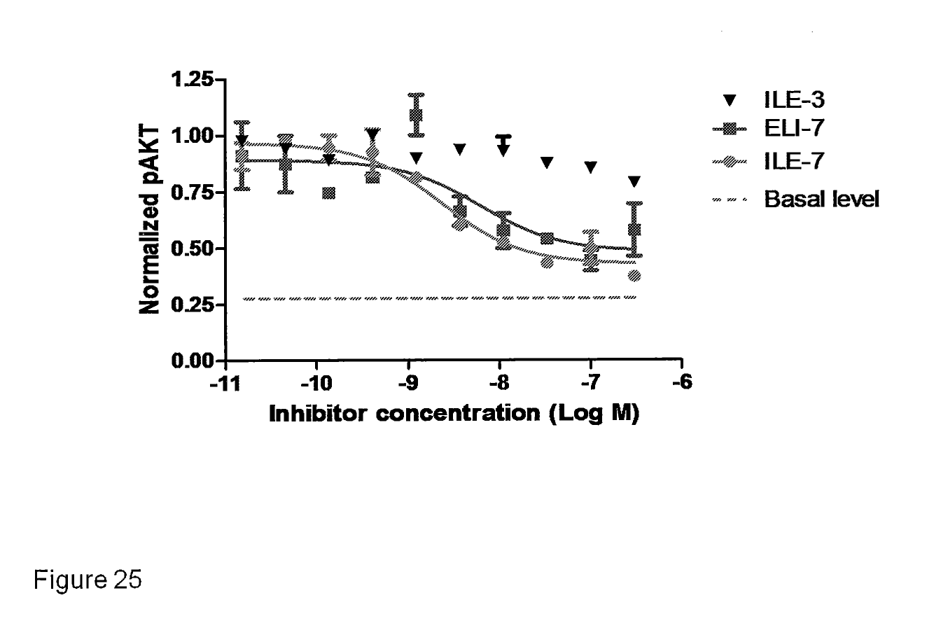

In a similar experiment, 3.5×10⁴ BXPC3 cells were treated with 20 ng/ml heregulin and 80 ng/ml IGF1 for 15 minutes, following pre-treatment with BBAs for 1 hour (starting concentration of 0.5 nM, 10 point 3-fold dilution series). ILE-7 inhibited phosphorylated Akt signaling whereas ILE-3 could not FIG. 25).

B) BBAs inhibit signaling across a broad range of ErbB3 and IGF-1R receptor levels To determine whether dimeric BBAs (e.g., BBAs with four binding moieties—two to each target) can inhibit downstream signaling across a broad range of ErbB3 and IGF-1R receptor levels the following experiment was performed:

BXPC3 cell receptor levels were varied by shRNA-mediated knockdown of IGF-1R or ErbB3 in BxPC-3 cells using the pLKO.1 PURO vector (Sigma). ErbB3 and IGF-1R levels were then measured by quantitative FACS and the mean receptor levels were calculated from the resulting distribution (see Table 1.1 for relative expression levels). To determine the potency of BBAs, cells were serum-starved and pretreated with ELI-7 for 1 hour @ 37oC., followed by a 15-minute stimulation with 20 ng/ml HRG +80ng/ml IGF1. Signal inhibition was assessed by ELISA for pAKT.

Relative receptor levels and pAkt IC50 values for four BXPC3 cell lines:

TABLE 18

| | % of control | | 95% | | |
|---|---|---|---|---|---|
| Engineered | receptor | pAkt | Confidence | shRNA | Sigma-Aldrich |
| BXPC3 cell line | level | IC50 | Interval | sequence | Catalog # |
| BXPC3-non- | IGF-1R and | 3.6 nM | 0.9-14.7 nM | SEQ ID | SHC002V |
| targeted control | ErbB3 levels | | | NO: 147 | |
| | unchanged | | | | |
| BXPC3-IGF-1R- | IGF-1R level | 6.4 nM | 2.9-14.1 nM | SEQ ID | SHCLNV- |
| mod1 | reduced by | | | NO: 148 | NM_000875- |
| | 37% | | | | TRCN0000039673 |
| BXPC3-ErbB3- | ErbB3 level | 3.3 nM | 1.4-8.0 nM | SEQ ID | SHCLNV- |
| mod1 | reduced by | | | NO: 149 | NM_001982- |
| | 48% | | | | TRCN0000230091 |
| BXPC3-ErbB3- | ErbB3 level | 7.6 nM | 1.2-50.0 nM | SEQ ID | SHCLNV- |
| mod2 | reduced by | | | NO: 150 | NM_001982- |
| | 88% | | | | TRCN0000018327 |

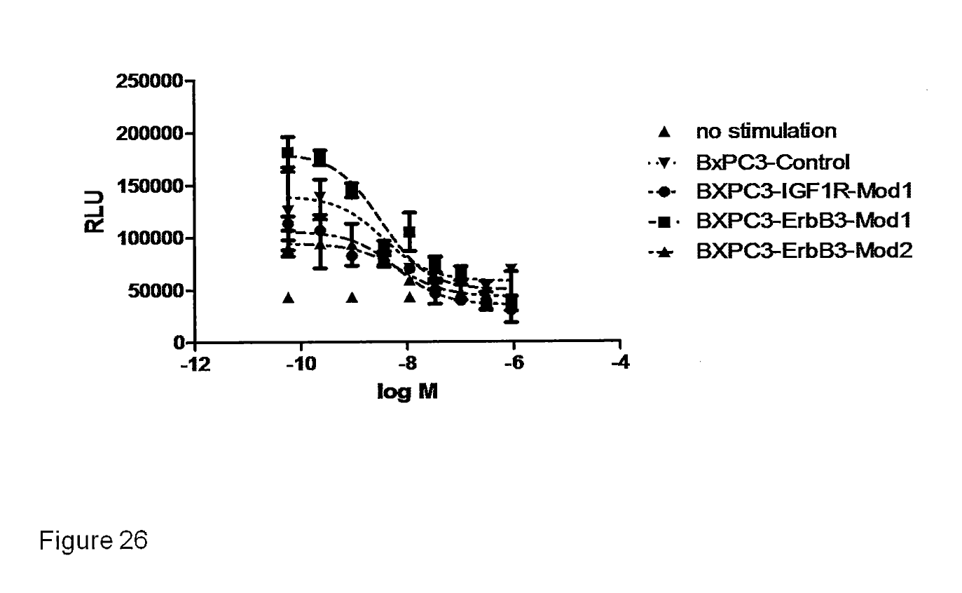

The BBA ELI-7 displayed similar potency across the BXPC3 cells lines with modified receptor levels as indicated by their IC50 values and overlapping confidence intervals (see Table 1.1), indicating that they have broad activity against a range of receptor profiles (FIG. 26).

### EQUIVALENTS

Those skilled in the art will recognize, or be able to ascertain and implement using no more than routine experimentation, many equivalents of the specific embodiments described herein. Such equivalents are intended to be encompassed by the following claims. Any combinations of the embodiments disclosed in the dependent claims are contemplated to be within the scope of the disclosure.

### INCORPORATION BY REFERENCE

The disclosure of each and every US and foreign patent and pending patent application and publication referred to herein is hereby incorporated herein by reference in its entirety.

APPENDIX A
ANTI-CANCER AGENTS

| Anti-Cancer Agent | Comments | Examples |
|---|---|---|
| Antibodies | Antibodies which bind | A12 (fully humanized mAb) |
| (a) antibodies other | IGF-1R (insulin-like | 19D12 (fully humanized mAb) |
| than anti-ErbB3 | growth factor type 1 | CP751-871 (fully humanized mAb) |
| antibodies; and | receptor), which is | H7C10 (humanized mAb) |
| (b) anti-ErbB3 | expressed on the cell | alphaIR3 (mouse) |
| antibodies which | surface of must human | scFV/FC (mouse/human chimera) |
| bind different | cancers | EM/164 (mouse) |
| epitopes | | AMG 479 (fully humanized mAb; Amgen) |
| | | IMCA 12 (fully humanized mAb; Imclone) |
| | | NSC-742460 (Dyax) |
| | | MR-0646, F50035 (Pierre Fabre Medicament, |
| | | Merck) |
| | Antibodies which bind | matuzumab (EMD72000) |
| | EGFR; Mutations | Erbitux ®/cetuximab (Imclone) |
| | affecting EGFR | Vectibix ®/panitumumab (Amgen) |
| | expression or activity can | mAb 806 |
| | result in cancer | nimotuzumab (TheraCIM ®) |
| | | INCB7839 (Incyte) |
| | | panitumumab (Vectibix ®; Amgen) |
| | Antibodies which bind | AV299 (AVEO) |
| | cMET (mesenchymal | AMG102 (Amgen) |
| | epithelial transition | 5D5 (OA-5D5) (Genentech) |
| | factor); a member of the | |
| | MET family of receptor | |
| | tyrosine kinases) | |
| | Anti-ErbB3 antibodies | Ab #14 described in WO 2008/100624 |
| | which bind different | 1B4C3; 2D1D12 (U3 Pharma AG) |
| | epitopes | U3-1287/AMG888 (U3 Pharma/Amgen) |
| | Anti-ErbB2 (HER2) | Herceptin ® (trastuzumab; Genentech/Roche); |
| | antibodies | Omnitarg ® (pertuzumab; 2C4, R1273; |
| | | Genentech/Roche) |
| Small Molecules | IGF-1R (insulin-like | NVP-AEW541-A |
| Targeting IGF1R | growth factor type 1 | BMS-536, 924 (1H-benzoimidazol-2-yl)-1H- |
| | receptor), which is | pyridin-2-one) |
| | expressed on the cell | BMS-554, 417 |
| | surface of must human | Cycloligan |
| | cancers | TAE226 |
| | | PQ401 |
| Small Molecules | EGFR; Mutations | Iressa ®/gefitinib (AstraZeneca) |
| Targeting EGFR | affecting EGFR | CI-1033 (PD 183805) (Pfizer) |
| | expression or activity can | TYVERB/lapatinib (GlaxoSmithKline) |
| | result in cancer | Tykerb ®/lapatinib ditosylate (SmithKline |
| | | Beecham) |
| | | Tarceva ®/Erlotinib HCL (OSI Pharma) |
| | | PKI-166 (Novartis) |
| | | PD-158780 |
| | | EKB-569 |
| | | Tyrphostin AG 1478(4-(3-Chloroanillino)- |
| | | 6,7-dimethoxyquinazoline) |
| Small Molecules | ErbB2, also known as | HKI-272 (neratinib; Wyeth) |
| Targeting ErbB2 | HER2, a member of the | KOS-953 (tanespimycin; Kosan Biosciences) |
| | ErbB family of receptors, | |
| | which is expressed on | |
| | certain cancer cells | |
| Small Molecules | cMET (Mesenchymal | PHA665752 |
| Targeting cMET | epithelial transition | ARQ 197 (ArQule) |
| | factor); a member of the | ARQ-650RP (ArQule) |
| | MET family of receptor | |
| | tyrosine kinases) | |
| Antimetabolites | An antimetabolite is a | flourouracil (5-FU) |
| | chemical with a similar | capecitabine/XELODA ® (HLR Roche) |
| | structure to a substance (a | 5-trifluoromethyl-2′-deoxyuridine |
| | metabolite) required for | methotrexate sodium (Trexall) (Barr) |
| | normal biochemical | raltitrexed/Tomudex ® (AstraZaneca) |
| | reactions, yet different | pemetrexed/Alimta ® (Lilly) |
| | enough to interfere with | tegafur |
| | the normal functions of | cytosine arabinoside (Cytarabine, Ara-C)/ |
| | cells, including cell | tioguanine/Lanvis ® (GlaxoSmithKline) |
| | division. | 5-azacytidine |
| | | 6-mercaptopurine (Mercaptopurine, 6-MP) |
| | | azathioprine/Azasan ® (AAIPHARMA LLC) |
| | | 6-thioguanine (6-TG)/Purinethol ® (TEVA) |
| | | pentostatin/Nipent ® (Hospira Inc.) |
| | | fludarabine phosphate/Fludara ® (Bayer |
| | | Health Care) |
| | | cladribine/Leustatin ® (2-CdA, 2- |
| | | chlorodeoxyadenosine) (Ortho Biotech) |
| | | floxuridine (5-fluoro-2′-deoxyuridine)/ |
| | | FUDR ® (Hospira, Inc,) |
| Alkylating agents | An alkylating | Ribonucleotide Reductase Inhibitor (RNR) |
| | antineoplastic agent is an | cyclophosphamide/Cytoxan ® (BMS)/ |
| | alkylating agent that | Neosar ® (TEVA) |
| | attaches an alkyl group to | ifosfamide/Mitoxana ® (ASTA Medica) |
| | DNA. Since cancer cells | ThioTEPA (Bedford, Abraxis, Teva) |
| | generally proliferate | BCNU→ 1,3-bis(2-chloroethyl)-1-nitosourea |
| | unrestrictively more than | CCNU→ 1,-(2-chloroethyl)-3-cyclohexyl-1- |
| | do healthy cells they are | nitrosourea (methyl CCNU) |
| | more sensitive to DNA | hexamethylmelamine (altretamine, HMM)/ |
| | damage, and alkylating | Hexalen ® (MGI Pharma Inc.) |
| | agents are used clinically | busulfan/Myleran ® (GlaxoSmithKline) |
| | to treat a variety of | procarbazine HCL/Matulane ® (Sigma Tau) |
| | tumors. | Dacarbazine (DTIC ®) |
| | | chlorambucil/Leukaran ® (SmithKline |
| | | Beecham) |
| | | Melphalan/Alkeran ® (GlaxoSmithKline) |
| | | cisplatin (Cisplatinum, CDDP)/Platinol |
| | | (Bristol Myers) |
| | | carboplatin/Paraplatin (BMS) |
| | | oxaliplatin/Eloxitan ® (Sanofi-Aventis US) |
| | | Bendamustine |
| | | carboquone |
| | | carmustine |
| | | chloromethine |
| | | dacarbazine (DTIC) |
| | | fotemustine |
| | | lomustine |
| | | mannosulfan |
| | | nedaplatin |
| | | nimustine |
| | | prednimustine |
| | | ranimustine |
| | | satraplatin |
| | | semustine |
| | | streptozocin |
| | | temozolomide |
| | | treosulfan |
| | | triaziquone |
| | | triethylene melamine |
| | | triplatin tetranitrate |
| | | trofosfamide |
| | | uramustine |
| Topoisomerase | Topoisomerase inhibitors | doxorubicin HCL/Doxil ® (Alza) |
| inhibitors | are chemotherapy agents | daunorubicin citrate/Daunoxome ® (Gilead) |
| | designed to interfere with | mitoxantrone HCL/Novantrone (EMD |
| | the action of | Serono) |
| | topoisomerase enzymes | actinomycin D |
| | (topoisomerase I and II), | etoposide/Vepesid ® (BMS)/Etopophos ® |
| | which are enzymes that | (Hospira, Bedford, Teva Parenteral, Etc.) |
| | control the changes in | topotecan HCL/Hycamtin ® |
| | DNA structure by | (GlaxoSmithKline) |
| | catalyzing the breaking | teniposide (VM-26)/Vumon ® (BMS) |
| | and rejoining of the | irinotecan HCL(CPT-11)/ |
| | phosphodiester backbone | camptosar ® (Pharmacia & Upjohn) |
| | of DNA strands during | camptothecin (CPT) |
| | the normal cell cycle. | belotecan |
| | | rubitecan |
| Microtubule | Microtubules are one of | vincristine/Oncovin ® (Lilly) |
| targeting agents | the components of the | vinblastine sulfate/Velban ®(discontinued) |
| | cytoskeleton. They have | (Lilly) |
| | diameter of | vinorelbine tartrate/Navelbine ® |
| | apporximately 24 nm and | (PierreFabre) |
| | length varying from | vindesine sulphate/Eldisine ® (Lilly) |
| | several micrometers to | paclitaxel/Taxol ® (BMS) |
| | possibly millimeters in | docetaxel/Taxotere ® (Sanofi Aventis US) |
| | axons of nerve cells. | Nanoparticle paclitaxel (ABI-007)/ |
| | Microtubules serve as | Abraxane ® (Abraxis BioScience, Inc.) |
| | structural components | ixabepilone/IXEMPRA ™ (BMS) |
| | within cells and are | larotaxel |
| | involved in many cellular | ortataxel |
| | processes including | tesetaxel |
| | mitosis, cytokinesis, and | vinflunine |
| | vesicular transport. | |
| Kinase inhibitors | Tyrosine kinases are | imatinib mesylate/Gleevec (Novartis) |
| | enzymes within the cell | sunitinib malate/Sutent ® (Pfizer) |
| | that function to attach | sorafenib tosylate/Nexavar ® (Bayer) |
| | phosphate groups to the | nilotinib hydrochloride monohydrate/ |
| | amino acid tyrosine. By | Tasigna ® (Novartis) |
| | blocking the ability of | AMG 386 (Amgen) |
| | protein tyrosine kinases | axitinib (AG-013736; Pfizer, Inc.) |
| | to function, these | bosutinib (SKI-606; Wyeth) |
| | compounds provide a tool | brivanib alalinate (BMS-582664; BMS) |
| | for controlling cancerous | cediranib (AZD2171; Recentin, AstraZeneca) |
| | cell growth. | dasatinib (BMS-354825: Sprycel ®; BMS) |
| | | lestaurtinib (CEP-701; Cephalon) |
| | | motesanib diphosphage (AMG-706; |
| | | Amgen/Takeda) |
| | | pazopanib HCL (GW786034; Armala, GSK) |
| | | semaxanib (SU5416; Pharmacia) |
| | | vandetanib (AZD647; Zactima; AstraZeneca) |
| | | vatalanib (PTK-787; Novartis, Bayer Schering |
| | | Pharma) |
| | | XL184 (NSC718781; Exelixis, GSK) |
| Protein synthesis | Induces cell apoptosis | L-asparaginase/Elspar ® (Merck & Co.) |
| inhibitors | | |
| Immunotherapeutic | Induces cancer patients to | Alpha interferon |
| agents | exhibit immune | Angiogenesis Inhibitor/Avastin ® |
| | responsiveness | (Genentech) |
| | | IL-2→ Interleukin 2 (Aldesleukin)/ |
| | | Proleukin ® (Chiron) |
| | | IL-12→ Interleukin 12 |
| Hormonal therapies | Hormonal therapies | Ttoremifene citrate/Fareston ® (GTX, Inc.) |
| | associated with | fulvestrant/Faslodex ® (AstraZeneca) |
| | menopause and aging | raloxifene HCL/Evista ® (Lilly) |
| | seek to increase the | anastrazole/Arimidex ® (AstraZeneca) |
| | amount of certain | letrozole/Femara ® (Novartis) |
| | hormones in the body to | fadrozole (CGS 16949A) |
| | compensate for age- or | exemestane/Aromasin ® (Pharmacia & |
| | disease-related hormonal | Upjohn) |
| | declines. Hormonal | leuprolide acetate/Eligard ® (QTL USA) |
| | therapy as a cancer | Lupron ® (TAP Pharm.) |
| | treatment generally either | goserelin acetate/Zoladex ® (AstraZeneca) |
| | reduces the level of one | triptorelin pamoate/Trelstar ® (Watson Labs) |
| | or more specific | buserelin/Suprefact ® (Sanofi Aventis) |
| | hormones, blocks a | nafarelin |
| | hormone from interacting | cetrorelix/Cetrotide ® (EMD Serono) |
| | with its cellular receptor | bicalutamide/Casodex ® (AstraZeneca) |
| | or otherwise alters the | nilutamide/Nilandron ® (Aventis Pharm.) |
| | cancer's ability to be | megestrol acetate/Megace ® (BMS) |
| | stimulated by hormones | somatostatin Analogs (e.g., Octreotide acetate/ |
| | to grow and spread. Such | Sandostatin ® (Novartis)) |
| | hormonal therapies thus | abarelix (Plenaxis ™; Amgen) |
| | include hormone | abiraterone acetate (CB7630; BTG plc) |
| | antagonists and hormone | afimoxifene (TamoGel; Ascend Therapeutics, |
| | synthesis inhibitors. In | Inc.) |
| | some instances hormone | aromatase inhibitor (Atamestane plus |
| | agonists may also be used | toremifene; Intarcia Therapeutics, Inc.) |
| | as anticancer hormonal | arzoxifene (Eli Lilly & Co) |
| | therapies. | Asentar ™; DN-101 (Novacea; Oregon Health |
| | | Sciences U) |
| | | flutamide (Eulexin ®, Schering; Prostacur, |
| | | Laboratorios Almirall, S.A) |
| | | letrozole (CG520267) (Femara ®, Chugai; |
| | | Estrochek ®, (Jagsonpal Pharamaceuticals Ltd; |
| | | Delestrogen ®, estradiol valerate (Jagsonpal) |
| | | magestrol acetate/Megace ® |
| | | medroxyprogesteone acetate (Veraplex ®; |
| | | Combiphar) |
| | | MT206 (Medisyn Technologies, Inc.) |
| | | nandrolone decanoate (Zestabolin ®); Mankind |
| | | Pharma Ltd) |
| | | tamoxifen (Taxifen ®, Yung Shin |
| | | Pharmaceutical; Tomifen ®, Alkem |
| | | Laboratories Ltd.) |
| | | tamoxifen citrate (Nolvadex, AstraZeneca; |
| | | soltamox, EUSA Pharma Inc; |
| | | tamoxifen citrate SOPHARMA, Sopharma |
| | | JSCo.) |
| Glucocorticoids | Anti-inflammatory drugs | predinsolone |
| | used to reduce swelling | dexamethasone/Decadron ® (Wyeth) |
| | that causes cancer pain. | prednisone (Deltasone, Orasone, Liquid Pred, |
| | | Sterapred ®) |
| Aromatase inhibitors | Includes imidazoles | ketoconazole |
| mTOR inhibitors | The mTOR signaling | sirolimus (Rapamycin)/Rapamune ® (Wyeth) |
| | pathway was originally | Temsirolimus (CCI-779)/Torisel ® (Wyeth) |
| | discovered during studies | Deforolimus (AP23573) (Ariad Pharm.) |
| | of the | Everolimus (RAD001)/Certican ® (Novartis) |
| | immunosuppressive agent | |
| | rapamycin. This highly | |
| | conserved pathway | |
| | regulates cell | |
| | proliferation and | |
| | metabolism in response | |
| | to environmental factors, | |
| | linking cell growth factor | |
| | receptor signaling via | |
| | phosphoinositide-3- | |
| | kinase (PI-3K) to cell | |
| | growth, proliferation, and | |
| | angiogenesis. | |
| Chemotherapeutic | | adriamycin, 5-fluorouracil, cytoxin, |
| agents | | bleomycin, mitomycin C, daunomycin, |
| | | carminomycin, aminopterin, dactinomycin, |
| | | mitomycins, esperamicins, clofarabine, |
| | | mercaptopurine, pentostatin, thioguanine, |
| | | cytarabine, decitabine, floxuridine, |
| | | gemcitabine (Gemzar), enocitabine, |
| | | sapacitabine |
| Protein Kinase B | | AKT Inhibitor Astex ® (Astex Therapeutics) |
| (PKB) Inhibitors | | AKT Inhibitors NERVIANO (Nerviano |
| | | Medical Sciences) |
| | | AKT Kinase Inhibitor TELIK (Telik Inc) |
| | | AKT DECIPHERA (Deciphera |
| | | Pharmaceuticals, LLC) |
| | | perifosine (KRX0401, D-21266; Keryx |
| | | Biopharmaceuticals Inc, AEterna Zentaris |
| | | Inc) |
| | | perifosine with Docetaxel (Keryx |
| | | Biopharmaceuticals Inc, AEterna Zentaris |
| | | Inc) |
| | | perifosine with Gemcitabine (AEterna |
| | | Zentaris Inc) |
| | | perifosine with paclitaxel (AEterna Zentaris |
| | | Inc) |
| | | protein kinase-B inhibitor DEVELOGEN |
| | | (DeveloGen AG) |
| | | PX316 (Oncothyreon, Inc.) |
| | | RX0183 (Rexahn Pharmaceuticals Inc) |
| | | RX0201 (Rexahn Pharmaceuticals Inc) |
| | | VQD002 (VioQuest Pharmaceuticals Inc) |
| | | XL418 (Exelixis Inc) |
| | | ZEN027 (AEterna Zentaris Inc) |
| Phosphatidylinositol | | BEZ235 (Novartis AG) |
| 3-Kinase (PI3K) | | BGT226 (Novartis AG) |
| Inhibitors | | CAL101 (Calistoga Pharmaceuticals, Inc.) |
| | | CHR4432 (Chroma Therapeutics Ltd) |
| | | Erk/PI3K Inhibitors ETERNA (AEterna |
| | | Zentaris Inc) |
| | | GDC0941 (Genentech Inc/Piramed |
| | | Limited/Roche Holdings Ltd) |
| | | enzastaurin HCL (LY317615; Enzastaurin; |
| | | Eli Lilly) |
| | | LY294002/Wortmannin |
| | | PI3K Inhibitors SEMAFORE (Semafore |
| | | Pharmaceuticals) |
| | | PX866 (Oncothyreon, Inc.) |
| | | SF1126 (Semafore Pharmaceuticals) |
| | | VMD-8000 (VM Discovery, Inc.) |
| | | XL147 (Exelixis Inc) |
| | | XL147 with XL647 (Exelixis Inc) |
| | | XL765 (Exelixis Inc) |
| | | PI-103 (Roche/Piramed) |
| Cyclin Dependent | | CYC200, R-roscovitine (Seliciclib; Cyclacel |
| Kinase Inhibitors | | Pharma) |
| | | NSC-649890, L86-8275, HMR-1275 |
| | | (alvocidib; NCI) |
| TLr9, CD289 | | IMOxine (Merck KGaA) |
| | | HYB2055 (Idera) |
| | | IMO-2055 (Isis Pharma) |
| | | 1018 ISS (Dynavax Technologies/UCSF) |
| | | PF-3512676 (Pfizer) |
| Enzyme Inhibitor | | lonafarnib(SCH66336; Sarasar; SuperGen, U |
| | | Arizona) |
| Anti-TRAIL | | AMG-655 (Aeterna Zentaris, Keryx |
| | | Biopharma) |
| | | Apo2L/TRAIL AMG951 (Genentech, |
| | | Amgen) |
| | | APOMAB (fully humanized mAb; |
| | | Genentech) |
| MEK Inhibitors | [Mitogen-Activated | ARRY162 (Array BioPharma Inc) |
| | Protein Kinase Kinase 1 | ARRY704 (Array BioPharma Inc) |
| | (MAP2K1); Mitogen- | ARRY886 (Array BioPharma Inc) |
| | Activated Protein Kinase | AS703026 (Merck Serono S.A) |
| | Kinase 2 (MAP2K2)] | AZD6244 (AstraZeneca Plc) |
| | | AZD8330 (AstraZeneca Plc) |
| | | RDEA119 (Ardea Biosciences, Inc.) |
| | | RDEA436 (Ardea Biosciences, Inc.) |
| | | XL518 (Exelixis Inc; Genentech Inc) |
| Miscellaneous | | Imprime PGG (Biothera) |
| Inhibitors | | CHR-2797 (AminopeptidaseM1 inhibitor; |
| | | Chroma Therapeutics) |
| | | E7820, NSC 719239 (Integrin-alpha2 |
| | | inhibitor, Eisai) |
| | | INCB007839 (ADAM 17, TACE Inhibitor; |
| | | Incyte) |
| | | CNF2024, BIIB021 (Hsp90 Inhibitor; Biogen |
| | | Idec) |
| | | MP470, HPK-56 (Kit/Mel/Ret Inhibitor; |
| | | Schering-Plough) |
| | | SNDX-275/MS-275 (HDAC Inhibitor; |
| | | Syndax) |
| | | Zarnestra ™, Tipifarnib, R115777 (Ras |
| | | Inhibitor; Janssen Pharma) |
| | | volociximab; Eos 200-4, M200 (alpha581 |
| | | integrin inhibitor; Biogen Idec; Eli |
| | | Lilly/UCSF/PDL BioPharma) |
| | | apricoxib (TP2001; COX-2 Inhibitor, Daiichi |
| | | Sankyo; Tragara Pharma) |

1-45. (canceled)

46. A method of inhibiting proliferation of a tumor cell comprising contacting the tumor cell with a bispecific binding agent, said agent comprising an IGF-1R targeting moiety, a linker moiety, and an ErbB3 targeting moiety, wherein the IGF-1R targeting moiety specifically binds to IGF-1R and the ErbB3 targeting moiety specifically binds to ErbB3, and wherein the targeting moieties are each linked to the linker moiety, such that proliferation of the tumor cell is inhibited.

47. The method of claim 46, wherein the tumor cell expresses IGF-1R and ErbB3.

48. The method of claim 46, wherein the IGF-1R targeting moiety is linked to the amino terminus of the linker moiety and the ErbB3 targeting moiety is linked to the carboxy terminus of the linker moiety.

49. The method of claim 46, wherein the linker moiety is an immunoglobulin or an immunoglobulin fragment.

50. The method of claim 49, wherein the linker moiety is an immunoglobulin fragment comprising the amino acid sequence set forth in SEQ ID NO: 53.

51. The method of claim 46, wherein the IGF-1R targeting moiety comprises one or more anti-IGF-1R antibody.

52. The method of claim 46, wherein the ErbB3 targeting moiety comprises one or more anti-ErbB3 antibody.

53. The method of claim 52, wherein the anti-ErbB3 antibody is an scFv.

54. The method of claim 46, wherein the IGF-1R targeting moiety is an anti-IGF-1R F(ab)′₂.

55. A method of treating a tumor in a patient comprising administering a bispecific binding agent, said agent comprising an IGF-1R targeting moiety, a linker moiety, and an ErbB3 targeting moiety, wherein the IGF-1R targeting moiety specifically binds to IGF-1R and the ErbB3 targeting moiety specifically binds to ErbB3 and wherein the targeting moieties are each linked to the linker moiety, to the patient in an amount effective to reduce tumor cell proliferation.

56. The method of claim 55, wherein the tumor expresses IGF-1R and ErbB3.

57. The method of claim 55, wherein the tumor is a lung cancer, sarcoma, colorectal cancer, head and neck cancer, pancreatic cancer, ovarian cancer, or breast cancer tumor.

58. The method of claim 55, further comprising administering a second anti-cancer agent to the patient or administering a second anti-cancer treatment modality to the patient.

59. The method of claim 58, further comprising administering a second anti-cancer agent that is a chemotherapeutic drug.

60. The method of claim 55, wherein the IGF-1R targeting moiety is linked to the amino terminus of the linker moiety and the ErbB3 targeting moiety is linked to the carboxy terminus of the linker moiety.

61. The method of claim 55, wherein the linker moiety is an immunoglobulin or an immunoglobulin fragment.

62. The method of claim 61, wherein the linker moiety is an immunoglobulin fragment comprising the amino acid sequence set forth in SEQ ID NO: 53.

63. The method of claim 55, wherein the IGF-1R targeting moiety comprises one or more anti-IGF-1R antibody.

64. The method of claim 55, wherein the ErbB3 targeting moiety comprises one or more anti-ErbB3 antibody.

65. The method of claim 64, wherein the anti-ErbB3 antibody is an scFv.

66. The method of claim 55, wherein the IGF-1R targeting moiety is an anti-IGF-1R F(ab)′₂.


SEQUENCE LISTING (150 sequences)

SEQ ID NO: 1 - protein, 247 aa, Artificial Sequence
  source: /note=“Description of Artificial Sequence Synthetic polypeptide”
EVQLLESGGGLVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTMVTVSSASTGGGGSGGGGSGGGGSDIQMTQSPSSFSASTGDRVTITCRASQGISSYLAWYQQKPGKAPKLLIYAASTLQSGVPSRFSGSGSGTDFTLTISSLQPEDSATYYCQQYFTFPLTFGGGTKVEIK

SEQ ID NO: 2 - protein, 252 aa, Artificial Sequence
  source: /note=“Description of Artificial Sequence Synthetic polypeptide”
EVQLLQSGGGLVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTSVTVSSASTGGGGSGGGGSGGGGSGGGGSDIQMTQSPSSLSASSGDRVTITCRASQGISSYLAWYQQKPGKAPKLLIYAASTLQSGVPSRFSGSGSGTDFTLTISSLQPEDSATYYCQQYFTFPLTFGGGTKVEIK

SEQ ID NO: 3 - protein, 254 aa, Artificial Sequence
  source: /note=“Description of Artificial Sequence Synthetic polypeptide”
EVQLLQSGGGLVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTSVTVSSASTGGGGSGGGGSGGGGSGGGGSDIQMTQSPSSLSASSGDRVTITCRASQGISSYLAWYQQKPGKAPKLLIYAASTLQSGVPSRFSGSGSGTDFTLTISSLQPEDSATYYCQQYFTFPLTFGGGTKVEIKRT

SEQ ID NO: 4 - protein, 252 aa, Artificial Sequence
  source: /note=“Description of Artificial Sequence Synthetic polypeptide”
EVQLLQSGGGDVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTSVTVSSASTGGGGSGGGGSGGGGSGGGGSDIQMTQSPSSLSASSGDRVTITCRASQGISSYLAWYQQKPGKAPKLLIYAASTLQSGVPSRFSGSGSGTDFTLTISSLQPEDSATYYCQQYFTFPLTFGGGTKVEIK

SEQ ID NO: 5 - protein, 254 aa, Artificial Sequence
  source: /note=“Description of Artificial Sequence Synthetic polypeptide”
EVQLLQSGGGDVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTSVTVSSASTGGGGSGGGGSGGGGSGGGGSDIQMTQSPSSLSASSGDRVTITCRASQGISSYLAWYQQKPGKAPKLLIYAASTLQSGVPSRFSGSGSGTDFTLTISSLQPEDSATYYCQQYFTFPLTFGGGTKVEIKRT

SEQ ID NO: 6 - protein, 122 aa, Artificial Sequence
  source: /note=“Description of Artificial Sequence Synthetic polypeptide”
EVQLLESGGGLVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTMVTVSS

SEQ ID NO: 7 - protein, 122 aa, Artificial Sequence
  source: /note=“Description of Artificial Sequence Synthetic polypeptide”
EVQLLQSGGGLVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTTVTVSS

SEQ ID NO: 8 - protein, 220 aa, Artificial Sequence
  source: /note=“Description of Artificial Sequence Synthetic polypeptide”
EVQLLESGGGLVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 9 - protein, 220 aa, Artificial Sequence
  source: /note=“Description of Artificial Sequence Synthetic polypeptide”
EVQLLQSGGGLVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 10 - protein, 214 aa, Artificial Sequence
  source: /note=“Description of Artificial Sequence Synthetic polypeptide”
DIQMTQSPSSFSASTGDRVTITCRASQGISSYLAWYQQKPGKAPKLLIYAASTLQSGVPSRFSGSGSGTDFTLTISSLQPEDSATYYCQQYFTFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 214 aa, Artificial Sequence
  source: /note=“Description of Artificial Sequence Synthetic polypeptide”
DIQMTQSPSSLSASTGDRVTITCRASQGISSYLAWYQQKPGKAPKLLIYAASTLQSGVPSRFSGSGSGTDFTLTISSLQPEDFATYYCQQYFTFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 12 - protein, 109 aa, Artificial Sequence
  source: /note=“Description of Artificial Sequence Synthetic polypeptide”
DIQMTQSPSSFSASTGDRVTITCRASQGISSYLAWYQQKPGKAPKLLIYAASTLQSGVPSRFSGSGSGTDFTLTISSLQPEDSATYYCQQYFTFPLTFGGGTKVEIKRT

SEQ ID NO: 13 - protein, 109 aa, Artificial Sequence
  source: /note=“Description of Artificial Sequence Synthetic polypeptide”
DIQMTQSPSSLSASTGDRVTITCRASQGISSYLAWYQQKPGKAPKLLIYAASTLQSGVPSRFSGSGSGTDFTLTISSLQPEDFATYYCQQYFTFPLTFGGGTKVEIKRT

SEQ ID NO: 14 - protein, 245 aa, Artificial Sequence
  source: /note=“Description of Artificial Sequence Synthetic polypeptide”
EVQLLESGGGLVQPGGSLRLSCAASGFTFSVYEMHWVRQAPGKGLEWVSGISSSGGWTPYADSVKGRFTISRDNSKNTLYLQMNSLRAEDTAVYYCARQSQYYYYGMDVWGQGTTVTVSSASTGGGGSGGGGSGGGGSDIQMTQSPSTLSASVGDRVTITCRASQSISSWLAWYQQKPGTAPKLLISKTSTLQSGVPSRFSGSGSGTDFTLTITSLQPEDFATYYCLQDYNDPWTFGQGTKVEIK

SEQ ID NO: 15 - protein, 247 aa, Artificial Sequence
  source: /note=“Description of Artificial Sequence Synthetic polypeptide”
EVQLLESGGGLVQPGGSLRLSCAASGFTFSVYEMHWVRQAPGKGLEWVSGISSSGGWTPYADSVKGRFTISRDNSKNTLYLQMNSLRAEDTAVYYCARQSQYYYYGMDVWGQGTTVTVSSASTGGGGSGGGGSGGGGSDIQMTQSPSTLSASVGDRVTITCRASQSISSWLAWYQQKPGTAPKLLISKTSTLQSGVPSRFSGSGSGTDFTLTITSLQPEDFATYYCLQDYNDPWTFGQGTKVEIKRT

SEQ ID NO: 16 - protein, 218 aa, Artificial Sequence
  source: /note=“Description of Artificial Sequence Synthetic polypeptide”
EVQLLESGGGLVQPGGSLRLSCAASGFTFSVYEMHWVRQAPGKGLEWVSGISSSGGWTPYADSVKGRFTISRDNSKNTLYLQMNSLRAEDTAVYYCARQSQYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 17 - protein, 214 aa, Artificial Sequence
  source: /note=“Description of Artificial Sequence Synthetic polypeptide”
DIQMTQSPSTLSASVGDRVTITCRASQSISSWLAWYQQKPGTAPKLLISKTSTLQSGVPSRFSGSGSGTDFTLTITSLQPEDFATYYCLQDYNDPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 594 aa, Homo sapiens
GAASDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLAAALQ

SEQ ID NO: 19 - protein, 594 aa, Homo sapiens
GAASDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLAAALQ

SEQ ID NO: 20 - protein, 122 aa, Artificial Sequence
  source: /note=“Description of Artificial Sequence Synthetic polypeptide”
VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSGGGGSGGGGSLQ

SEQ ID NO: 21 - protein, 122 aa, Artificial Sequence
  source: /note=“Description of Artificial Sequence Synthetic polypeptide”
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECSGGGGSGGGGSGGGGSLQ

SEQ ID NO: 22 - protein, 343 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSLQ

SEQ ID NO: 23 - protein, 338 aa, Artificial Sequence
  source: /note=“Description of Artificial Sequence Synthetic polypeptide”
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSLQ

SEQ ID NO: 24 - protein, 344 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGS

SEQ ID NO: 25 - protein, 344 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGS

SEQ ID NO: 26 - protein, 344 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGNQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGS

SEQ ID NO: 27 - protein, 341 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGS

SEQ ID NO: 28 - protein, 341 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVESKYGPPCPSCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGS

SEQ ID NO: 29 - protein, 340 aa, Artificial Sequence
  source: /note=“Description of Artificial Sequence Synthetic polypeptide”
ASTKGPSVFPLAPSSKSTSGGTAALGCLVKDYFPEPVTVSWNSGALTSGVHTFPAVLQSSGLYSLSSVVTVPSSSLGNQTYICNVNHKPSNTKVDKKVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGSGGGGSGGGGS

SEQ ID NO: 30 - protein, 226 aa, Artificial Sequence
  source: /note=“Description of Artificial Sequence Synthetic polypeptide”
GGGSGGGGSGGGGSGGGGSTSEETISTVQEKQQNISPLVRERGPQRVAAHITGTRGRSNTLSSPNSKNEKALGRKINSWESSRSGHSFLSNLHLRNGELVIHEKGFYYIYSQTYFRFQEEIKENTKNDKQMVQYIYKYTSYPDPILLMKSARNSCWSKDAEYGLYSIYQGGIFELKENDRIFVSVTNEHLIDMDHEASFFGAFLVGGGGGSGGGGSGGGGSGGGGS

SEQ ID NO: 31 - protein, 187 aa, Artificial Sequence
  source: /note=“Description of Artificial Sequence Synthetic polypeptide”
GGGSGGGGSGGGGSGGGGSNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEEVVVRVLDERLVRLRDGTRSYFGAFMVGGGGSGGGGSGGGGSGGGGS

SEQ ID NO: 32 - protein, 17 aa, Artificial Sequence
  source: /note=“Description of Artificial Sequence Synthetic peptide”
GGGGSGGGGSGGGGSLQ

SEQ ID NO: 33 - protein, 247 aa, Artificial Sequence
  source: /note=“Description of Artificial Sequence Synthetic polypeptide”
EVQLLESGGGLVQPGGSLRLSCAASGFTFSAYNMRWVRQAPGKGLEWVSVIYPSGGATRYADSVKGRFTISRDNSKNTLYLQMNSLRAEDTAVYYCARGYYYYGMDVWGQGTLVTVSSASTGGGGSGGGGSGGGGSQSVLTQPPSASGTPGQRVTISCSGSDSNIGRNYIYWYQQFPGTAPKLLIYRNNQRPSGVPDRISGSKSGTSASLAISGLRSEDEAEYHCGTWDDSLSGPVFGGGTKLTVLS

SEQ ID NO: 34 - protein, 252 aa, Artificial Sequence
  source: /note=“Description of Artificial Sequence Synthetic polypeptide”
EVQLQQSGGGLVQPGGSLRLSCAASGFTFSAYNMRWVRQAPGKGLEWVAVIYPSGGATRYADSVKGRFTISRDNSKNTLYLQMNSLRAEDTAVYYCARGYYYYGMDVWGQGTLVTVSSASTGGGGSGGGGSGGGGSGGGGSQSVLTQPPSASGTPGQSVTISCSGSDSNIGRNYIYWYQQFPGTAPKLLIYRNNQRPSGVPDRFSGSKSGTSASLAISGLQSEDEADYYCGTWDDSLSGPVFGGGTKLTVLG

SEQ ID NO: 35 - protein, 118 aa, Artificial Sequence
  source: /note=“Description of Artificial Sequence Synthetic polypeptide”
EVQLLESGGGLVQPGGSLRLSCAASGFTFSAYNMRWVRQAPGKGLEWVSVIYPSGGATRYADSVKGRFTISRDNSKNTLYLQMNSLRAEDTAVYYCARGYYYYGMDVWGQGTLVTVSS

SEQ ID NO: 36 - protein, 118 aa, Artificial Sequence
  source: /note=“Description of Artificial Sequence Synthetic polypeptide”
EVQLQQSGGGLVQPGGSLRLSCAASGFTFSAYNMRWVRQAPGKGLEWVAVIYPSGGATRYADSVKGRFTISRDNSKNTLYLQMNSLRAEDTAVYYCARGYYYYGMDVWGQGTLVTVSS

SEQ ID NO: 37 - protein, 216 aa, Artificial Sequence
  source: /note=“Description of Artificial Sequence Synthetic polypeptide”
EVQLLESGGGLVQPGGSLRLSCAASGFTFSAYNMRWVRQAPGKGLEWVSVIYPSGGATRYADSVKGRFTISRDNSKNTLYLQMNSLRAEDTAVYYCARGYYYYGMDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 38 - protein, 216 aa, Artificial Sequence
  source: /note=“Description of Artificial Sequence Synthetic polypeptide”
EVQLLQSGGGLVQPGGSLRLSCAASGFTFSAYNMRWVRQAPGKGLEWVSVIYPSGGATRYADSVKGRFTISRDNSKNTLYLQMNSLRAEDTAVYYCARGYYYYGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 39 - protein, 216 aa, Artificial Sequence
  source: /note=“Description of Artificial Sequence Synthetic polypeptide”
QSVLTQPPSASGTPGQRVTISCSGSDSNIGRNYIYWYQQFPGTAPKLLIYRNNQRPSGVPDRISGSKSGTSASLAISGLRSEDEAEYHCGTWDDSLSGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 40 - protein, 216 aa, Artificial Sequence
  source: /note=“Description of Artificial Sequence Synthetic polypeptide”
QSVLTQPPSASGTPGQSVTISCSGSDSNIGRNYIYWYQQFPGTAPKLLIYRNNQRPSGVPDRFSGSKSGTSASLAISGLQSEDEADYYCGTWDDSLSGPVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 111 aa, Artificial Sequence
  source: /note=“Description of Artificial Sequence Synthetic polypeptide”
QSVLTQPPSASGTPGQRVTISCSGSDSNIGRNYIYWYQQFPGTAPKLLIYRNNQRPSGVPDRISGSKSGTSASLAISGLRSEDEAEYHCGTWDDSLSGPVFGGGTKLTVLG

SEQ ID NO: 42 - protein, 111 aa, Artificial Sequence
  source: /note=“Description of Artificial Sequence Synthetic polypeptide”
QSVLTQPPSASGTPGQSVTISCSGSDSNIGRNYIYWYQQFPGTAPKLLIYRNNQRPSGVPDRFSGSKSGTSASLAISGLQSEDEADYYCGTWDDSLSGPVFGGGTKLTVLG

SEQ ID NO: 43 - protein, 249 aa, Artificial Sequence
  source: /note=“Description of Artificial Sequence Synthetic polypeptide”
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTGGGGSGGGGSGGGGSQSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLG

SEQ ID NO: 44 - protein, 246 aa, Artificial Sequence
  source: /note=“Description of Artificial Sequence Synthetic polypeptide”
EVQLLESGGGLVQPGGSLRLSCAASGFTFSVYDMYWVRQAPGKGLEWVSYIYSSGGDTDYADSVKGRFTISRDNSKNTLYLQMNSLRAEDTATYYCARASTNSIYDAMDVWGQGTTVTVSSASTGGGGSGGGGSGGGGSDIQMTQSPSSVSASVGDRVTITCRASQDITNWLAWYQQKPGKAPTLLIYDASTLQSGVPSRFSGSGSGTDFTLTISSLQPEDFATYFCQQANGFPLTFGGGTKVEIK

SEQ ID NO: 45 - protein, 248 aa, Artificial Sequence
  source: /note=“Description of Artificial Sequence Synthetic polypeptide”
EVQLLESGGGLVQPGGSLRLSCAASGFTFSVYDMYWVRQAPGKGLEWVSYIYSSGGDTDYADSVKGRFTISRDNSKNTLYLQMNSLRAEDTATYYCARASTNSIYDAMDVWGQGTTVTVSSASTGGGGSGGGGSGGGGSDIQMTQSPSSVSASVGDRVTITCRASQDITNWLAWYQQKPGKAPTLLIYDASTLQSGVPSRFSGSGSGTDFTLTISSLQPEDFATYFCQQANGFPLTFGGGTKVEIKRT

SEQ ID NO: 46 - protein, 249 aa, Artificial Sequence
  source: /note=“Description of Artificial Sequence Synthetic polypeptide”
QVQLVQSGGGLVQPGRSLRLSCAASGFTFDDYAMHWVRQAPGKGLEWVSGISWNSGSIGYADSVKGRFTISRDNAKNSLYLQMNSLRPEDTAVYYCARDLGAKQWLEGFDYWGQGTLVTVSSASTGGGGSGGGGSGGGGSSYELTQDPAVSVALGQTVRITCQGDSLRSYYASWYQQKPGQAPVLVIYGKNNRPSGIPDRFSGSTSGNSASLTITGAQAEDEADYYCNSRDSSGNHWVFGGGTKVTVLG

SEQ ID NO: 47 - protein, 254 aa, Artificial Sequence
  source: /note=“Description of Artificial Sequence Synthetic polypeptide”
QVQLVQSGGGLVQPGGSLRLSCAASGFTFDDYAMHWVRQAPGKGLEWVAGISWNSGSIGYADSVKGRFTISRDNAKNSLYLQMNSLRPEDTAVYYCARDLGYNQWVEGFDYWGQGTLVTVSSASTGGGGSGGGGSGGGGSGGGGSSYELTQDPAVSVALGQTVRITCQGDSLRSYYASWYQQKPGQAPVLVIYGKNNRPSGIPDRFSGSTSGNSASLTITGAQAEDEADYYCNSRDSSGNHWVFGGGTKVTVLG

SEQ ID NO: 48 - protein, 554 aa, Artificial Sequence
  source: /note=“Description of Artificial Sequence Synthetic polypeptide”
EVQLLESGGGLVQPGGSLRLSCAASGFTFSVYDMYWVRQAPGKGLEWVSYIYSSGGDTDYADSVKGRFTISRDNSKNTLYLQMNSLRAEDTATYYCARASTNSIYDAMDVWGQGTTVTVSSASTGGGGSGGGGSGGGGSDIQMTQSPSSVSASVGDRVTITCRASQDITNWLAWYQQKPGKAPTLLIYDASTLQSGVPSRFSGSGSGTDFTLTISSLQPEDFATYFCQQANGFPLTFGGGTKVEIKTGQEQQSHQPHSEQDDEHPTGQTQAQEQAETEEGHEAQNAEEQNAHTQDEHDQTPNQPGASEVQLLESGGGLVQPGGSLRLSCAASGFTFSAYNMRWVRQAPGKGLEWVSVIYPSGGATRYADSVKGRFTISRDNSKNTLYLQMNSLRAEDTAVYYCARGYYYYGMDVWGQGTLVTVSSASTGGGGSGGGGSGGGGSQSVLTQPPSASGTPGQRVTISCSGSDSNIGRNYIYWYQQFPGTAPKLLIYRNNQRPSGVPDRISGSKSGTSASLAISGLRSEDEAEYHCGTWDDSLSGPVFGGGTKLTVLS

SEQ ID NO: 49 - protein, 554 aa, Artificial Sequence
  source: /note=“Description of Artificial Sequence Synthetic polypeptide”
DIQMTQSPSSVSASVGDRVTITCRASQDITNWLAWYQQKPGKAPTLLIYDASTLQSGVPSRFSGSGSGTDFTLTISSLQPEDFATYFCQQANGFPLTFGGGTKVEIKASTGGGGSGGGGSGGGGSEVQLLESGGGLVQPGGSLRLSCAASGFTFSVYDMYWVRQAPGKGLEWVSYIYSSGGDTDYADSVKGRFTISRDNSKNTLYLQMNSLRAEDTATYYCARASTNSIYDAMDVWGQGTTVTVSSTGQEQQSHQPHSEQDDEHPTGQTQAQEQAETEEGHEAQNAEEQNAHTQDEHDQTPNQPGASEVQLLESGGGLVQPGGSLRLSCAASGFTFSAYNMRWVRQAPGKGLEWVSVIYPSGGATRYADSVKGRFTISRDNSKNTLYLQMNSLRAEDTAVYYCARGYYYYGMDVWGQGTLVTVSSASTGGGGSGGGGSGGGGSQSVLTQPPSASGTPGQRVTISCSGSDSNIGRNYIYWYQQFPGTAPKLLIYRNNQRPSGVPDRISGSKSGTSASLAISGLRSEDEAEYHCGTWDDSLSGPVFGGGTKLTVLS

SEQ ID NO: 50 - protein, 556 aa, Artificial Sequence
  source: /note=“Description of Artificial Sequence Synthetic polypeptide”
DIQMTQSPSSVSASVGDRVTITCRASQDITNWLAWYQQKPGKAPTLLIYDASTLQSGVPSRFSGSGSGTDFTLTISSLQPEDFATYFCQQANGFPLTFGGGTKVEIKGEPAASSPSSNNAAATTAASEVQLLESGGGLVQPGGSLRLSCAASGFTFSVYDMYWVRQAPGKGLEWVSYIYSSGGDTDYADSVKGRFTISRDNSKNTLYLQMNSLRAEDTATYYCARASTNSIYDAMDVWGQGTTVTVSSTGQEQQSHQPHSEQDDEHPTGQTQAQEQAETEEGHEAQNAEEQNAHTQDEHDQTPNQPGASEVQLLESGGGLVQPGGSLRLSCAASGFTFSAYNMRWVRQAPGKGLEWVSVIYPSGGATRYADSVKGRFTISRDNSKNTLYLQMNSLRAEDTAVYYCARGYYYYGMDVWGQGTLVTVSSASTGGGGSGGGGSGGGGSQSVLTQPPSASGTPGQRVTISCSGSDSNIGRNYIYWYQQFPGTAPKLLIYRNNQRPSGVPDRISGSKSGTSASLAISGLRSEDEAEYHCGTWDDSLSGPVFGGGTKLTVLS

SEQ ID NO: 51 - protein, 245 aa, Artificial Sequence
  source: /note=“Description of Artificial Sequence Synthetic polypeptide”
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSGGGGSLQ

SEQ ID NO: 52 - protein, 240 aa, Artificial Sequence
  source: /note=“Description of Artificial Sequence Synthetic polypeptide”
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGKGGGGSGGGGSLQ

SEQ ID NO: 53 - protein, 246 aa, Artificial Sequence
  source: /note=“Description of Artificial Sequence Synthetic polypeptide”
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGS

SEQ ID NO: 54 - protein, 246 aa, Artificial Sequence
  source: /note=“Description of Artificial Sequence Synthetic polypeptide”
EPKSCDKTHTCPPCPAPELLGGPSVFLFPPKPKDTLMISRTPEVTCVVVDVSHEDPEVKFNWYVDGVEVHNAKTKPREEQYQSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGGGGGSGGGGSGGGGS

SEQ ID NO: 55 - protein, 243 aa, Artificial Sequence
  source: /note=“Description of Artificial Sequence Synthetic polypeptide”
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGS

SEQ ID NO: 56 - protein, 243 aa, Artificial Sequence
  source: /note=“Description of Artificial Sequence Synthetic polypeptide”
ESKYGPPCPSCPAPEFLGGPSVFLFPPKPKDTLMISRTPEVTCVVVDVSQEDPEVQFNWYVDGVEVHNAKTKPREEQFQSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGGGGGSGGGGSGGGGS

SEQ ID NO: 57 - protein, 444 aa, Artificial Sequence
  source: /note=“Description of Artificial Sequence Synthetic polypeptide”
EVQLLESGGGLVQPGGSLRLSCAASGFTFSAYNMRWVRQAPGKGLEWVSVIYPSGGATRYADSVKGRFTISRDNSKNTLYLQMNSLRAEDTAVYYCARGYYYYGMDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 58 - protein, 448 aa, Artificial Sequence
  source: /note=“Description of Artificial Sequence Synthetic polypeptide”
EVQLLESGGGLVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 59 - protein, 124 aa, Artificial Sequence
  source: /note=“Description of Artificial Sequence Synthetic polypeptide”
EVQLLESGGGLVQPGGSLRLSCAASGFTFSWYGMYWVRQAPGKGLEWVSGISSSGGYTMYADSVKGRFTISRDNSKNTLYLQMNSLRAEDTAVYYCAKVGTLVVPAAIPYFQHWGQGTLVTVSS

SEQ ID NO: 60 - protein, 124 aa, Artificial Sequence
  source: /note=“Description of Artificial Sequence Synthetic polypeptide”
EVQLLQSGGGLVQPGGSLRLSCAASGFTFSWYGMYWVRQAPGKGLEWVSGISSSGGYTMYADSVKGRFTISRDNSKNTLYLQMNSLRAEDTAVYYCAKVGTLVVPAAIPYFQHWGQGTLVTVSS

SEQ ID NO: 61 - protein, 222 aa, Artificial Sequence
  source: /note=“Description of Artificial Sequence Synthetic polypeptide”
EVQLLESGGGLVQPGGSLRLSCAASGFTFSWYGMYWVRQAPGKGLEWVSGISSSGGYTMYADSVKGRFTISRDNSKNTLYLQMNSLRAEDTAVYYCAKVGTLVVPAAIPYFQHWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKKV

SEQ ID NO: 62 - protein, 222 aa, Artificial Sequence
  source: /note=“Description of Artificial Sequence Synthetic polypeptide”
EVQLLQSGGGLVQPGGSLRLSCAASGFTFSWYGMYWVRQAPGKGLEWVSGISSSGGYTMYADSVKGRFTISRDNSKNTLYLQMNSLRAEDTAVYYCAKVGTLVVPAAIPYFQHWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 63 - protein, 255 aa, Artificial Sequence
  source: /note=“Description of Artificial Sequence Synthetic polypeptide”
EVQLLESGGGLVQPGGSLRLSCAASGFTFSWYGMYWVRQAPGKGLEWVSGISSSGGYTMYADSVKGRFTISRDNSKNTLYLQMNSLRAEDTAVYYCAKVGTLVVPAAIPYFQHWGQGTLVTVSSASTGGGGSGGGGSGGGGSDIQMTQSPDSLAVSLGERATINCKSSQSILYNSNNKNYLAWYQQKPGQPPKLLIYWTSTRESGVPDRFSGSGSGTDFSLTISSLQAEDVAVYYCQQYYSTPPTFGQGTKVEIK

SEQ ID NO: 64 - protein, 255 aa, Artificial Sequence
  source: /note=“Description of Artificial Sequence Synthetic polypeptide”
EVQLLQSGGGLVQPGGSLRLSCAASGFTFSWYGMYWVRQAPGKGLEWVSGISSSGGYTMYADSVKGRFTISRDNSKNTLYLQMNSLRAEDTAVYYCAKVGTLVVPAAIPYFQHWGQGTLVTVSSASTGGGGSGGGGSGGGGSDIQMTQSPDSLAVSLGERATINCKSSQSILYNSNNKNYLAWYQQKPGQPPKLLIYWTSTRESGVPDRFSGSGSGTDFSLTISSLQAEDVAVYYCQQYYSTPPTFGQGTKVEIK

SEQ ID NO: 65 - protein, 257 aa, Artificial Sequence
  source: /note=“Description of Artificial Sequence Synthetic polypeptide”
EVQLLQSGGGLVQPGGSLRLSCAASGFTFSWYGMYWVRQAPGKGLEWVSGISSSGGYTMYADSVKGRFTISRDNSKNTLYLQMNSLRAEDTAVYYCAKVGTLVVPAAIPYFQHWGQGTLVTVSSASTGGGGSGGGGSGGGGSDIQMTQSPDSLAVSLGERATINCKSSQSILYNSNNKNYLAWYQQKPGQPPKLLIYWTSTRESGVPDRFSGSGSGTDFSLTISSLQAEDVAVYYCQQYYSTPPTFGQGTKVEIKRT

SEQ ID NO: 66 - protein, 220 aa, Artificial Sequence
  source: /note=“Description of Artificial Sequence Synthetic polypeptide”
DIQMTQSPDSLAVSLGERATINCKSSQSILYNSNNKNYLAWYQQKPGQPPKLLIYWTSTRESGVPDRFSGSGSGTDFSLTISSLQAEDVAVYYCQQYYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 67 - protein, 115 aa, Artificial Sequence
  source: /note=“Description of Artificial Sequence Synthetic polypeptide”
DIQMTQSPDSLAVSLGERATINCKSSQSILYNSNNKNYLAWYQQKPGQPPKLLIYWTSTRESGVPDRFSGSGSGTDFSLTISSLQAEDVAVYYCQQYYSTPPTFGQGTKVEIKRT

SEQ ID NO: 68 - protein, 245 aa, Artificial Sequence
  source: /note=“Description of Artificial Sequence Synthetic polypeptide”
EVQLLQSGGGLVQPGGSLRLSCAASGFTFSVYEMHWVRQAPGKGLEWVSGISSSGGWTPYADSVKGRFTISRDNSKNTLYLQMNSLRAEDTAVYYCARQSQYYYYGMDVWGQGTTVTVSSASTGGGGSGGGGSGGGGSDIQMTQSPSTLSASVGDRVTITCRASQSISSWLAWYQQKPGTAPKLLISKTSTLQSGVPSRFSGSGSGTDFTLTITSLQPEDFATYYCLQDYNDPWTFGQGTKVEIK

SEQ ID NO: 69 - protein, 247 aa, Artificial Sequence
  source: /note=“Description of Artificial Sequence Synthetic polypeptide”
EVQLLQSGGGLVQPGGSLRLSCAASGFTFSVYEMHWVRQAPGKGLEWVSGISSSGGWTPYADSVKGRFTISRDNSKNTLYLQMNSLRAEDTAVYYCARQSQYYYYGMDVWGQGTTVTVSSASTGGGGSGGGGSGGGGSDIQMTQSPSTLSASVGDRVTITCRASQSISSWLAWYQQKPGTAPKLLISKTSTLQSGVPSRFSGSGSGTDFTLTITSLQPEDFATYYCLQDYNDPWTFGQGTKVEIKRT

SEQ ID NO: 70 - protein, 120 aa, Artificial Sequence
  source: /note=“Description of Artificial Sequence Synthetic polypeptide”
EVQLLESGGGLVQPGGSLRLSCAASGFTFSVYEMHWVRQAPGKGLEWVSGISSSGGWTPYADSVKGRFTISRDNSKNTLYLQMNSLRAEDTAVYYCARQSQYYYYGMDVWGQGTTVTVSS

SEQ ID NO: 71 - protein, 120 aa, Artificial Sequence
  source: /note=“Description of Artificial Sequence Synthetic polypeptide”
EVQLLQSGGGLVQPGGSLRLSCAASGFTFSVYEMHWVRQAPGKGLEWVSGISSSGGWTPYADSVKGRFTISRDNSKNTLYLQMNSLRAEDTAVYYCARQSQYYYYGMDVWGQGTTVTVSS

SEQ ID NO: 72 - protein, 218 aa, Artificial Sequence
  source: /note=“Description of Artificial Sequence Synthetic polypeptide”
EVQLLQSGGGLVQPGGSLRLSCAASGFTFSVYEMHWVRQAPGKGLEWVSGISSSGGWTPYADSVKGRFTISRDNSKNTLYLQMNSLRAEDTAVYYCARQSQYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 73 - protein, 252 aa, Artificial Sequence
  source: /note=“Description of Artificial Sequence Synthetic polypeptide”
EVQLLESGGGLVQPGGSLRLSCAASGFTFSAYGMGWVRQAPGKGLEWVSYISPSGGHTKYADSVKGRFTISRDNSKNTLYLQMNSLRAEDTAVYYCAKVLETGLLVDAFDIWGQGTMVTVSSASTGGGGSGGGGSGGGGSGGGGSSYELTQPPSVSVYPGQTASITCSGDQLGSKFVSWYQQRPGQSPVLVMYKDKRRPSEIPERFSGSNSGNTATLTISGTQAIDEADYYCQAWDSSTYVFGTGTKVTVLG

SEQ ID NO: 74 - protein, 252 aa, Artificial Sequence
  source: /note=“Description of Artificial Sequence Synthetic polypeptide”
EVQLLQSGGGLVQPGGSLRLSCAASGFTFSAYGMGWVRQAPGKGLEWVSYISPSGGHTKYADSVKGRFTISRDNSKNTLYLQMNSLRAEDTAVYYCAKVLETGLLVDAFDIWGQGTMVTVSSASTGGGGSGGGGSGGGGSGGGGSSYELTQPPSVSVYPGQTASITCSGDQLGSKFVSWYQQRPGQSPVLVMYKDKRRPSEIPERFSGSNSGNTATLTISGTQAIDEADYYCQAWDSSTYVFGTGTKVTVLG

SEQ ID NO: 75 - protein, 122 aa, Artificial Sequence
  source: /note=“Description of Artificial Sequence Synthetic polypeptide”
EVQLLESGGGLVQPGGSLRLSCAASGFTFSAYGMGWVRQAPGKGLEWVSYISPSGGHTKYADSVKGRFTISRDNSKNTLYLQMNSLRAEDTAVYYCAKVLETGLLVDAFDIWGQGTMVTVSS

SEQ ID NO: 76 - protein, 122 aa, Artificial Sequence
  source: /note=“Description of Artificial Sequence Synthetic polypeptide”
EVQLLQSGGGLVQPGGSLRLSCAASGFTFSAYGMGWVRQAPGKGLEWVSYISPSGGHTKYADSVKGRFTISRDNSKNTLYLQMNSLRAEDTAVYYCAKVLETGLLVDAFDIWGQGTMVTVSS

SEQ ID NO: 77 - protein, 220 aa, Artificial Sequence
  source: /note=“Description of Artificial Sequence Synthetic polypeptide”
EVQLLESGGGLVQPGGSLRLSCAASGFTFSAYGMGWVRQAPGKGLEWVSYISPSGGHTKYADSVKGRFTISRDNSKNTLYLQMNSLRAEDTAVYYCAKVLETGLLVDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKKV

SEQ ID NO: 78 - protein, 220 aa, Artificial Sequence
  source: /note=“Description of Artificial Sequence Synthetic polypeptide”
EVQLLQSGGGLVQPGGSLRLSCAASGFTFSAYGMGWVRQAPGKGLEWVSYISPSGGHTKYADSVKGRFTISRDNSKNTLYLQMNSLRAEDTAVYYCAKVLETGLLVDAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKKV

SEQ ID NO: 79 - protein, 212 aa, Artificial Sequence
  source: /note=“Description of Artificial Sequence Synthetic polypeptide”
SYELTQPPSVSVYPGQTASITCSGDQLGSKFVSWYQQRPGQSPVLVMYKDKRRPSEIPERFSGSNSGNTATLTISGTQAIDEADYYCQAWDSSTYVFGTGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 80 - protein, 107 aa, Artificial Sequence
  source: /note=“Description of Artificial Sequence Synthetic polypeptide”
SYELTQPPSVSVYPGQTASITCSGDQLGSKFVSWYQQRPGQSPVLVMYKDKRRPSEIPERFSGSNSGNTATLTISGTQAIDEADYYCQAWDSSTYVFGTGTKVTVLG

SEQ ID NO: 81 - protein, 246 aa, Artificial Sequence
  source: /note=“Description of Artificial Sequence Synthetic polypeptide”
EVQLLQSGGGLVQPGGSLRLSCAASGFTFSVYDMYWVRQAPGKGLEWVSYIYSSGGDTDYADSVKGRFTISRDNSKNTLYLQMNSLRAEDTATYYCARASTNSIYDAMDVWGQGTTVTVSSASTGGGGSGGGGSGGGGSDIQMTQSPSSVSASVGDRVTITCRASQDITNWLAWYQQKPGKAPTLLIYDASTLQSGVPSRFSGSGSGTDFTLTISSLQPEDFATYFCQQANGFPLTFGGGTKVEIK

SEQ ID NO: 82 - protein, 248 aa, Artificial Sequence
  source: /note=“Description of Artificial Sequence Synthetic polypeptide”
EVQLLQSGGGLVQPGGSLRLSCAASGFTFSVYDMYWVRQAPGKGLEWVSYIYSSGGDTDYADSVKGRFTISRDNSKNTLYLQMNSLRAEDTATYYCARASTNSIYDAMDVWGQGTTVTVSSASTGGGGSGGGGSGGGGSDIQMTQSPSSVSASVGDRVTITCRASQDITNWLAWYQQKPGKAPTLLIYDASTLQSGVPSRFSGSGSGTDFTLTISSLQPEDFATYFCQQANGFPLTFGGGTKVEIKRT

SEQ ID NO: 83 - protein, 121 aa, Artificial Sequence
  source: /note=“Description of Artificial Sequence Synthetic polypeptide”
EVQLLESGGGLVQPGGSLRLSCAASGFTFSVYDMYWVRQAPGKGLEWVSYIYSSGGDTDYADSVKGRFTISRDNSKNTLYLQMNSLRAEDTATYYCARASTNSIYDAMDVWGQGTTVTVSS

SEQ ID NO: 84 - protein, 121 aa, Artificial Sequence
  source: /note=“Description of Artificial Sequence Synthetic polypeptide”
EVQLLQSGGGLVQPGGSLRLSCAASGFTFSVYDMYWVRQAPGKGLEWVSYIYSSGGDTDYADSVKGRFTISRDNSKNTLYLQMNSLRAEDTATYYCARASTNSIYDAMDVWGQGTTVTVSS

SEQ ID NO: 85 - protein, 80 aa, Artificial Sequence
  source: /note=“Description of Artificial Sequence Synthetic polypeptide”
DITNWLAWYQQKPGKAPTLLIYDASTLQSGVPSRFSGSGSGTDFTLTISSLQPEDFATYFCQQANGFPLTFGGGTKVEIK

SEQ ID NO: 86 - protein, 187 aa, Artificial Sequence
  source: /note=“Description of Artificial Sequence Synthetic polypeptide”
DITNWLAWYQQKPGKAPTLLIYDASTLQSGVPSRFSGSGSGTDFTLTISSLQPEDFATYFCQQANGFPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - protein, 219 aa, Artificial Sequence
  source: /note=“Description of Artificial Sequence Synthetic polypeptide”
EVQLLESGGGLVQPGGSLRLSCAASGFTFSVYDMYWVRQAPGKGLEWVSYIYSSGGDTDYADSVKGRFTISRDNSKNTLYLQMNSLRAEDTATYYCARASTNSIYDAMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKKV

SEQ ID NO: 88 - protein, 219 aa, Artificial Sequence
  source: /note=“Description of Artificial Sequence Synthetic polypeptide”
EVQLLQSGGGLVQPGGSLRLSCAASGFTFSVYDMYWVRQAPGKGLEWVSYIYSSGGDTDYADSVKGRFTISRDNSKNTLYLQMNSLRAEDTATYYCARASTNSIYDAMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKKV

SEQ ID NO: 89 - protein, 122 aa, Artificial Sequence
  source: /note=“Description of Artificial Sequence Synthetic polypeptide”
QVQLVQSGGGLVQPGRSLRLSCAASGFTFDDYAMHWVRQAPGKGLEWVSGISWNSGSIGYADSVKGRFTISRDNAKNSLYLQMNSLRPEDTAVYYCARDLGAKQWLEGFDYWGQGTLVTVSS

SEQ ID NO: 90 - protein, 109 aa, Artificial Sequence
  source: /note=“Description of Artificial Sequence Synthetic polypeptide”
SYELTQDPAVSVALGQTVRITCQGDSLRSYYASWYQQKPGQAPVLVIYGKNNRPSGIPDRFSGSTSGNSASLTITGAQAEDEADYYCNSRDSSGNHWVFGGGTKVTVLG

SEQ ID NO: 91 - protein, 214 aa, Artificial Sequence
  source: /note=“Description of Artificial Sequence Synthetic polypeptide”
SYELTQDPAVSVALGQTVRITCQGDSLRSYYASWYQQKPGQAPVLVIYGKNNRPSGIPDRFSGSTSGNSASLTITGAQAEDEADYYCNSRDSSGNHWVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 92 - protein, 220 aa, Artificial Sequence
  source: /note=“Description of Artificial Sequence Synthetic polypeptide”
QVQLVQSGGGLVQPGRSLRLSCAASGFTFDDYAMHWVRQAPGKGLEWVSGISWNSGSIGYADSVKGRFTISRDNAKNSLYLQMNSLRPEDTAVYYCARDLGAKQWLEGFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKKV

SEQ ID NO: 93 - protein, 1107 aa, Artificial Sequence
  source: /note=“Description of Artificial Sequence Synthetic polypeptide”
MGWSLILLFLVAVATRVLSEVQLLESGGGLVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTMVTVSSASTGGGGSGGGGSGGGGSDIQMTQSPSSFSASTGDRVTITCRASQGISSYLAWYQQKPGKAPKLLIYAASTLQSGVPSRFSGSGSGTDFTLTISSLQPEDSATYYCQQYFTFPLTFGGGTKVEIKGAASDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLAAALQEVQLLESGGGLVQPGGSLRLSCAASGFTFSAYNMRWVRQAPGKGLEWVSVIYPSGGATRYADSVKGRFTISRDNSKNTLYLQMNSLRAEDTAVYYCARGYYYYGMDVWGQGTLVTVSSASTGGGGSGGGGSGGGGSQSVLTQPPSASGTPGQRVTISCSGSDSNIGRNYIYWYQQFPGTAPKLLIYRNNQRPSGVPDRISGSKSGTSASLAISGLRSEDEAEYHCGTWDDSLSGPVFGGGTKLTVLS

SEQ ID NO: 94 - protein, 1108 aa, Artificial Sequence [sequence omitted]

SEQ ID NO: 95 - protein, 1106 aa, Artificial Sequence
  source: /note=“Description of Artificial Sequence Synthetic polypeptide”
MGWSLILLFLVAVATRVLSEVQLLESGGGLVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTMVTVSSASTGGGGSGGGGSGGGGSDIQMTQSPSSFSASTGDRVTITCRASQGISSYLAWYQQKPGKAPKLLIYAASTLQSGVPSRFSGSGSGTDFTLTISSLQPEDSATYYCQQYFTFPLTFGGGTKVEIKGAASDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLAAALQEVQLLESGGGLVQPGGSLRLSCAASGFTFSVYDMYWVRQAPGKGLEWVSYIYSSGGDTDYADSVKGRFTISRDNSKNTLYLQMNSLRAEDTATYYCARASTNSIYDAMDVWGQGTTVTVSSASTGGGGSGGGGSGGGGSDIQMTQSPSSVSASVGDRVTITCRASQDITNWLAWYQQKPGKAPTLLIYDASTLQSGVPSRFSGSGSGTDFTLTISSLQPEDFATYFCQQANGFPLTFGGGTKVEIK

SEQ ID NO: 96 - protein, 1107 aa, Artificial Sequence [sequence omitted]

SEQ ID NO: 97 - protein, 1108 aa, Artificial Sequence
  source: /note=“Description of Artificial Sequence Synthetic polypeptide”
MGWSLILLFLVAVATRVLSEVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTGGGGSGGGGSGGGGSQSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGAASDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLAAALQEVQLLESGGGLVQPGGSLRLSCAASGFTFSVYPMHWVRQAPGKGLEWVSSISSSGGATRYADSVKGRFTISRDNSKNTLYLQMNSLRAEDTAVYYCAKDFYDILTGNAFDIWGQGTMVTVSSASTGGGGSGGGGSGGGGSDIQMTQSPSSFSASTGDRVTITCRASQGISSYLAWYQQKPGKAPKLLIYAASTLQSGVPSRFSGSGSGTDFTLTISSLQPEDSATYYCQQYFTFPLTFGGGTKVEIK

SEQ ID NO: 98 - protein, 1106 aa, Artificial Sequence [sequence omitted]

SEQ ID NO: 99 - DNA, 3328 bp, Artificial Sequence
  source: /note=“Description of Artificial Sequence Synthetic polynucleotide”
atgggctggagccttatccttctgttccttgtggctgtagcaactagagttctctccgaggtccagttgctcgagagtggaggcggtctggtgcagccaggtgggtccctgcgcttgagctgcgccgccagtgggttcaccttttccgtgtaccccatgcactgggtgcgacaagctccaggtaaaggcctcgaatgggtcagctctattagctcctctgggggcgctactcggtacgctgattctgtcaaggggcgtttcaccattagccgcgataattcaaagaacacactgtacctccagatgaattctctgcgggcagaggacaccgccgtttactactgtgccaaagacttttacgacatcctgactggcaacgcattcgatatctggggccaggggacaatggtcaccgtttcctcagcatcaaccggcggagggggctcaggagggggcggtagcggaggcggaggatccgatatccagatgacacaaagccctagcagtttctctgcatctaccggagatagagtgactataacatgtagggcctcccagggcatctcatcctatctggcctggtatcagcagaagccaggcaaggctcccaaactcctgatttatgccgcaagtacactgcaaagcggagtgcctagtaggttttcagggtctggatctggcaccgacttcactctgaccatcagttctttgcagcccgaagactccgctacttattactgccagcagtattttacttttcctctgacatttggtggtggtacaaaagtggagattaagggcgccgcctccgacgctcacaagagcgaagtggcacataggttcaaagatctgggcgaagagaactttaaggccctcgtcctgatcgctttcgcacagtacctccagcagtctccctttgaagatcacgtgaaactggtcaatgaggtgaccgaatttgccaagacatgcgtggctgatgagagtgcagaaaactgtgacaaatcactgcatactctctttggagataagctgtgcaccgtcgccacactcagagagacttatggggaaatggctgactgttgcgcaaaacaggagcctgaacggaatgagtgtttcctccagcacaaggatgacaacccaaatctgccccgcctcgtgcgacctgaggtcgatgtgatgtgcaccgcctttcatgacaacgaagagacattcctgaagaaatacctgtatgaaattgctcgtaggcacccatacttttatgcccccgagctcctgttctttgcaaagagatacaaagctgccttcactgaatgttgccaggcagctgataaggccgcatgtctcctgcctaaactggacgagctccgggatgaaggtaaggcttccagcgccaaacagcgcctgaagtgcgcttctctccagaagtttggcgagcgagcattcaaagcctgggctgtggcccgtctcagtcagaggtttccaaaggcagaatttgctgaggtctcaaaactggtgaccgacctcacaaaggtccatactgagtgttgccacggagatctgctggaatgtgccgacgatagagcagacctcgctaaatatatctgcgagaatcaggattccattagctctaagctgaaagaatgttgcgagaagcccctcctggaaaagagtcattgtatcgccgaggtggaaaacgacgagatgccagcagatctgccatcactcgctgccgactttgtggaatccaaagatgtctgcaagaattacgcagaggctaaagacgtgttcctggggatgtttctgtatgagtacgcccggcgtcaccccgattatagcgtcgtgctcctgctccgactggcaaagacctacgaaacaactctggagaaatgttgcgctgccgcagaccctcatgaatgttatgctaaggtgttcgatgagtttaagccactcgtcgaagagccccagaacctgattaaacagaattgcgaactgttcgagcagctcggtgaatacaagtttcagaacgccctgctcgtgcgttataccaaaaaggtccctcaggtgtctacaccaactctggtggaggtcagtaggaatctgggcaaagtgggatcaaagtgttgcaaacaccccgaggcaaagagaatgccttgtgctgaagattacctctccgtcgtgctgaaccagctctgcgtgctgcatgaaaagaccccagtcagcgaccgggtgacaaaatgttgcaccgaatctctggtcaatcgccgaccctgtttcagtgccctcgaagtggacgaaacttatgtgcctaaggagtttcaggctgaaacattcacctttcacgccgatatctgcactctgtccgagaaagaaaggcagattaagaaacagacagcactggtcgagctcgtgaagcataaaccaaaggctaccaaggagcagctgaaagccgtcatggacgatttcgcagcttttgtggaaaagtgttgcaaagccgacgataaggagacttgtttcgcagaagaggggaaaaagctcgtggctgccagccaggcagctctgggtctggccgcagctctgcaggaagtgcagctgttggagagtgggggtggcctggtgcagccaggaggttccctccggctgagctgtgcagcaagcgggttcacttttagtgcctacaacatgaggtgggtgcgacaagcacccgggaaaggtcttgagtgggtcagtgttatctatccctcaggtggcgcaacacggtacgccgatagcgttaaggggcgcttcactattagcagagacaactcaaaaaatacactgtatttgcagatgaattcacttcgggctgaggatacagctgtgtactattgcgccaggggatactattattacgggatggatgtgtggggtcaaggcaccttggtgacagtatcaagcgccagtaccggcggtgggggcagtggaggcggaggatctggaggtggcgggtcccagtccgttcttactcagccacctagcgcttctgggacacccggccagcgagtgaccataagttgttccggatctgacagtaacatcggaaggaattatatttactggtaccaacagtttccaggcaccgcccctaagctcctgatttatcgcaataaccagcgccctagcggcgtccctgacagaatctcaggctccaagtccgggacatcagcctctctcgccatctctggacttcgttctgaagacgaggctgaataccactgcggcacctgggatgactccctctctggccccgtcttcggtggaggcaccaagctgactgtgctgtcttgataat

SEQ ID NO: 100 - DNA, 3331 bp, Artificial Sequence
  source: /note=“Description of Artificial Sequence Synthetic polynucleotide”
atgggctggagccttatccttctgttccttgtggctgtagcaactagagttctctccgaggtccagttgctcgagagtggaggcggtctggtgcagccaggtgggtccctgcgcttgagctgcgccgccagtgggttcaccttttccgtgtaccccatgcactgggtgcgacaagctccaggtaaaggcctcgaatgggtcagctctattagctcctctgggggcgctactcggtacgctgattctgtcaaggggcgtttcaccattagccgcgataattcaaagaacacactgtacctccagatgaattctctgcgggcagaggacaccgccgtttactactgtgccaaagacttttacgacatcctgactggcaacgcattcgatatctggggccaggggacaatggtcaccgtttcctcagcatcaaccggcggagggggctcaggagggggcggtagcggaggcggaggatccgatatccagatgacacaaagccctagcagtttctctgcatctaccggagatagagtgactataacatgtagggcctcccagggcatctcatcctatctggcctggtatcagcagaagccaggcaaggctcccaaactcctgatttatgccgcaagtacactgcaaagcggagtgcctagtaggttttcagggtctggatctggcaccgacttcactctgaccatcagttctttgcagcccgaagactccgctacttattactgccagcagtattttacttttcctctgacatttggtggtggtacaaaagtggagattaagggcgccgcctccgacgctcacaagagcgaagtggcacataggttcaaagatctgggcgaagagaactttaaggccctcgtcctgatcgctttcgcacagtacctccagcagtctccctttgaagatcacgtgaaactggtcaatgaggtgaccgaatttgccaagacatgcgtggctgatgagagtgcagaaaactgtgacaaatcactgcatactctctttggagataagctgtgcaccgtcgccacactcagagagacttatggggaaatggctgactgttgcgcaaaacaggagcctgaacggaatgagtgtttcctccagcacaaggatgacaacccaaatctgccccgcctcgtgcgacctgaggtcgatgtgatgtgcaccgcctttcatgacaacgaagagacattcctgaagaaatacctgtatgaaattgctcgtaggcacccatacttttatgcccccgagctcctgttctttgcaaagagatacaaagctgccttcactgaatgttgccaggcagctgataaggccgcatgtctcctgcctaaactggacgagctccgggatgaaggtaaggcttccagcgccaaacagcgcctgaagtgcgcttctctccagaagtttggcgagcgagcattcaaagcctgggctgtggcccgtctcagtcagaggtttccaaaggcagaatttgctgaggtctcaaaactggtgaccgacctcacaaaggtccatactgagtgttgccacggagatctgctggaatgtgccgacgatagagcagacctcgctaaatatatctgcgagaatcaggattccattagctctaagctgaaagaatgttgcgagaagcccctcctggaaaagagtcattgtatcgccgaggtggaaaacgacgagatgccagcagatctgccatcactcgctgccgactttgtggaatccaaagatgtctgcaagaattacgcagaggctaaagacgtgttcctggggatgtttctgtatgagtacgcccggcgtcaccccgattatagcgtcgtgctcctgctccgactggcaaagacctacgaaacaactctggagaaatgttgcgctgccgcagaccctcatgaatgttatgctaaggtgttcgatgagtttaagccactcgtcgaagagccccagaacctgattaaacagaattgcgaactgttcgagcagctcggtgaatacaagtttcagaacgccctgctcgtgcgttataccaaaaaggtccctcaggtgtctacaccaactctggtggaggtcagtaggaatctgggcaaagtgggatcaaagtgttgcaaacaccccgaggcaaagagaatgccttgtgctgaagattacctctccgtcgtgctgaaccagctctgcgtgctgcatgaaaagaccccagtcagcgaccgggtgacaaaatgttgcaccgaatctctggtcaatcgccgaccctgtttcagtgccctcgaagtggacgaaacttatgtgcctaaggagtttcaggctgaaacattcacctttcacgccgatatctgcactctgtccgagaaagaaaggcagattaagaaacagacagcactggtcgagctcgtgaagcataaaccaaaggctaccaaggagcagctgaaagccgtcatggacgatttcgcagcttttgtggaaaagtgttgcaaagccgacgataaggagacttgtttcgcagaagaggggaaaaagctcgtggctgccagccaggcagctctgggtctggccgcagctctgcaggaggtgcagcttctggaaagcggagggggcttggtgcagcccggaggctctctccggttgagctgcgctgcttctggcttcacctttagtcactacgtgatggcatgggtgaggcaggcacctggcaagggactcgagtgggtgagctccatcagttctagtgggggctggacactgtacgccgacagtgtgaagggccgattcacaatctcacgcgacaattcaaagaacacattgtatctccagatgaacagcctgagagccgaagatactgccgtttactactgcaccagaggcctgaaaatggcaaccatctttgattactggggacagggcactctggtcacagtgtcttccgcctctaccggagggggcggatcaggagggggtgggtcaggaggtggaggttcccagtccgctctgacccagccagccagcgtcagcggctcccccggtcaaagcatcacaattagctgcactgggaccagttcagacgtcggctcatacaacgtggttagctggtatcaacagcatcctggtaaagcacccaaactgattatatatgaagtcagtcaaaggccatctggtgtgtccaatcgcttctctggttctaagtccggcaatacagccagtctgaccatctccgggctccagactgaggatgaggctgactattactgttgttcctatgccggatcaagcatttttgtgatttttgggggcggtactaaggtaaccgtcctctgataat

SEQ ID NO: 101 - DNA, 741 bp, Artificial Sequence
  source: /note=“Description of Artificial Sequence Synthetic polynucleotide”
gaggtccagttgctcgagagtggaggcggtctggtgcagccaggtgggtccctgcgcttgagctgcgccgccagtgggttcaccttttccgtgtaccccatgcactgggtgcgacaagctccaggtaaaggcctcgaatgggtcagctctattagctcctctgggggcgctactcggtacgctgattctgtcaaggggcgtttcaccattagccgcgataattcaaagaacacactgtacctccagatgaattctctgcgggcagaggacaccgccgtttactactgtgccaaagacttttacgacatcctgactggcaacgcattcgatatctggggccaggggacaatggtcaccgtttcctcagcatcaaccggcggagggggctcaggagggggcggtagcggaggcggaggatccgatatccagatgacacaaagccctagcagtttctctgcatctaccggagatagagtgactataacatgtagggcctcccagggcatctcatcctatctggcctggtatcagcagaagccaggcaaggctcccaaactcctgatttatgccgcaagtacactgcaaagcggagtgcctagtaggttttcagggtctggatctggcaccgacttcactctgaccatcagttctttgcagcccgaagactccgctacttattactgccagcagtattttacttttcctctgacatttggtggtggtacaaaagtggagattaag

SEQ ID NO: 102 - DNA, 756 bp, Artificial Sequence
  source: /note=“Description of Artificial Sequence Synthetic polynucleotide”
gaggtacagctgctgcaatcagggggaggcctggtccaacccggtggatcgttgcgactctcgtgtgccgcctcgggcttcacgttttcagtctatcccatgcattgggtgcgccaggcaccgggcaaaggcctcgagtgggtgagctcaatctcgtcctcgggaggtgccacaagatacgcggattccgtcaagggtaggttcacaattagcagagataactcaaagaatactctctatttgcagatgaactcactccgagcggaagatacagccgtctactattgtgcgaaagacttctatgatattctgacaggaaatgcgttcgacatctggggccaggggacatcggtcactgtctcgtcagcgtcaaccggaggtggtggctcgggcggtggcgggagcggtggaggaggcagcggaggtggaggatcagatatccaaatgactcagtcgccgtcgtccttgagcgcgtcgtccggtgaccgcgtaacgatcacctgtcgcgcgtcgcagggaatttcctcgtatttggcgtggtatcagcagaaacccggaaaagcgccgaaactgctcatctacgcagcatcgactctccaatcgggggtaccgagccggtttagcggatccgggtcgggaacggatttcacactgacgatctcgtcgcttcagcccgaggactcggccacttactattgtcagcaatactttacatttcctcttacgtttggcggaggaacgaaagtggagatcaaa

SEQ ID NO: 103 - DNA, 762 bp, Artificial Sequence
  source: /note=“Description of Artificial Sequence Synthetic polynucleotide”
gaggtacagctgctgcaatcagggggaggcctggtccaacccggtggatcgttgcgactctcgtgtgccgcctcgggcttcacgttttcagtctatcccatgcattgggtgcgccaggcaccgggcaaaggcctcgagtgggtgagctcaatctcgtcctcgggaggtgccacaagatacgcggattccgtcaagggtaggttcacaattagcagagataactcaaagaatactctctatttgcagatgaactcactccgagcggaagatacagccgtctactattgtgcgaaagacttctatgatattctgacaggaaatgcgttcgacatctggggccaggggacatcggtcactgtctcgtcagcgtcaaccggaggtggtggctcgggcggtggcgggagcggtggaggaggcagcggaggtggaggatcagatatccaaatgactcagtcgccgtcgtccttgagcgcgtcgtccggtgaccgcgtaacgatcacctgtcgcgcgtcgcagggaatttcctcgtatttggcgtggtatcagcagaaacccggaaaagcgccgaaactgctcatctacgcagcatcgactctccaatcgggggtaccgagccggtttagcggatccgggtcgggaacggatttcacactgacgatctcgtcgcttcagcccgaggactcggccacttactattgtcagcaatactttacatttcctcttacgtttggcggaggaacgaaagtggagatcaaacgcacc

SEQ ID NO: 104 - DNA, 756 bp, Artificial Sequence
  source: /note=“Description of Artificial Sequence Synthetic polynucleotide”
gaggtacagctgctgcaatcagggggaggcgacgtccaacccggtggatcgttgcgactctcgtgtgccgcctcgggcttcacgttttcagtctatcccatgcattgggtgcgccaggcaccgggcaaaggcctcgagtgggtgagctcaatctcgtcctcgggaggtgccacaagatacgcggattccgtcaagggtaggttcacaattagcagagataactcaaagaatactctctatttgcagatgaactcactccgagcggaagatacagccgtctactattgtgcgaaagacttctatgatattctgacaggaaatgcgttcgacatctggggccaggggacatcggtcactgtctcgtcagcgtcaaccggaggtggtggctcgggcggtggcgggagcggtggaggaggcagcggaggtggaggatcagatatccaaatgactcagtcgccgtcgtccttgagcgcgtcgtccggtgaccgcgtaacgatcacctgtcgcgcgtcgcagggaatttcctcgtatttggcgtggtatcagcagaaacccggaaaagcgccgaaactgctcatctacgcagcatcgactctccaatcgggggtaccgagccggtttagcggatccgggtcgggaacggatttcacactgacgatctcgtcgcttcagcccgaggactcggccacttactattgtcagcaatactttacatttcctcttacgtttggcggaggaacgaaagtggagatcaaa

SEQ ID NO: 105 - DNA, 762 bp, Artificial Sequence
  source: /note=“Description of Artificial Sequence Synthetic polynucleotide”
gaggtacagctgctgcaatcagggggaggcgacgtccaacccggtggatcgttgcgactctcgtgtgccgcctcgggcttcacgttttcagtctatcccatgcattgggtgcgccaggcaccgggcaaaggcctcgagtgggtgagctcaatctcgtcctcgggaggtgccacaagatacgcggattccgtcaagggtaggttcacaattagcagagataactcaaagaatactctctatttgcagatgaactcactccgagcggaagatacagccgtctactattgtgcgaaagacttctatgatattctgacaggaaatgcgttcgacatctggggccaggggacatcggtcactgtctcgtcagcgtcaaccggaggtggtggctcgggcggtggcgggagcggtggaggaggcagcggaggtggaggatcagatatccaaatgactcagtcgccgtcgtccttgagcgcgtcgtccggtgaccgcgtaacgatcacctgtcgcgcgtcgcagggaatttcctcgtatttggcgtggtatcagcagaaacccggaaaagcgccgaaactgctcatctacgcagcatcgactctccaatcgggggtaccgagccggtttagcggatccgggtcgggaacggatttcacactgacgatctcgtcgcttcagcccgaggactcggccacttactattgtcagcaatactttacatttcctcttacgtttggcggaggaacgaaagtggagatcaaacgcacc

SEQ ID NO: 106 - DNA, 366 bp, Artificial Sequence
  source: /note=“Description of Artificial Sequence Synthetic polynucleotide”
gaggtccagttgctcgagagtggaggcggtctggtgcagccaggtgggtccctgcgcttgagctgcgccgccagtgggttcaccttttccgtgtaccccatgcactgggtgcgacaagctccaggtaaaggcctcgaatgggtcagctctattagctcctctgggggcgctactcggtacgctgattctgtcaaggggcgtttcaccattagccgcgataattcaaagaacacactgtacctccagatgaattctctgcgggcagaggacaccgccgtttactactgtgccaaagacttttacgacatcctgactggcaacgcattcgatatctggggccaggggacaatggtcaccgtttcctca

SEQ ID NO: 107 - DNA, 366 bp, Artificial Sequence
  source: /note=“Description of Artificial Sequence Synthetic polynucleotide”
gaggtgcagctcctccaaagcggagggggtttggtccagcccggtggctcgttgcgcctctcatgtgccgcgtcggggtttacattcagcgtctatccgatgcactgggtacgacaggcgccagggaaaggactggagtgggtgtcatcgatcagcagctccggaggtgctacaagatatgccgactcggtgaaaggccgctttacgatcagccgcgataacagcaagaatacgttgtacctccaaatgaactcgctgcgagccgaggacactgcggtgtactattgcgcgaaggatttctatgacatccttactggtaatgcgtttgatatctggggccaggggacaacggtcaccgtgagctcc

SEQ ID NO: 108 - DNA, 3325 bp, Artificial Sequence
  source: /note=“Description of Artificial Sequence Synthetic polynucleotide”
atgggctggagccttatccttctgttccttgtggctgtagcaactagagttctctccgaggtccagttgctcgagagtggaggcggtctggtgcagccaggtgggtccctgcgcttgagctgcgccgccagtgggttcaccttttccgtgtaccccatgcactgggtgcgacaagctccaggtaaaggcctcgaatgggtcagctctattagctcctctgggggcgctactcggtacgctgattctgtcaaggggcgtttcaccattagccgcgataattcaaagaacacactgtacctccagatgaattctctgcgggcagaggacaccgccgtttactactgtgccaaagacttttacgacatcctgactggcaacgcattcgatatctggggccaggggacaatggtcaccgtttcctcagcatcaaccggcggagggggctcaggagggggcggtagcggaggcggaggatccgatatccagatgacacaaagccctagcagtttctctgcatctaccggagatagagtgactataacatgtagggcctcccagggcatctcatcctatctggcctggtatcagcagaagccaggcaaggctcccaaactcctgatttatgccgcaagtacactgcaaagcggagtgcctagtaggttttcagggtctggatctggcaccgacttcactctgaccatcagttctttgcagcccgaagactccgctacttattactgccagcagtattttacttttcctctgacatttggtggtggtacaaaagtggagattaagggcgccgcctccgacgctcacaagagcgaagtggcacataggttcaaagatctgggcgaagagaactttaaggccctcgtcctgatcgctttcgcacagtacctccagcagtctccctttgaagatcacgtgaaactggtcaatgaggtgaccgaatttgccaagacatgcgtggctgatgagagtgcagaaaactgtgacaaatcactgcatactctctttggagataagctgtgcaccgtcgccacactcagagagacttatggggaaatggctgactgttgcgcaaaacaggagcctgaacggaatgagtgtttcctccagcacaaggatgacaacccaaatctgccccgcctcgtgcgacctgaggtcgatgtgatgtgcaccgcctttcatgacaacgaagagacattcctgaagaaatacctgtatgaaattgctcgtaggcacccatacttttatgcccccgagctcctgttctttgcaaagagatacaaagctgccttcactgaatgttgccaggcagctgataaggccgcatgtctcctgcctaaactggacgagctccgggatgaaggtaaggcttccagcgccaaacagcgcctgaagtgcgcttctctccagaagtttggcgagcgagcattcaaagcctgggctgtggcccgtctcagtcagaggtttccaaaggcagaatttgctgaggtctcaaaactggtgaccgacctcacaaaggtccatactgagtgttgccacggagatctgctggaatgtgccgacgatagagcagacctcgctaaatatatctgcgagaatcaggattccattagctctaagctgaaagaatgttgcgagaagcccctcctggaaaagagtcattgtatcgccgaggtggaaaacgacgagatgccagcagatctgccatcactcgctgccgactttgtggaatccaaagatgtctgcaagaattacgcagaggctaaagacgtgttcctggggatgtttctgtatgagtacgcccggcgtcaccccgattatagcgtcgtgctcctgctccgactggcaaagacctacgaaacaactctggagaaatgttgcgctgccgcagaccctcatgaatgttatgctaaggtgttcgatgagtttaagccactcgtcgaagagccccagaacctgattaaacagaattgcgaactgttcgagcagctcggtgaatacaagtttcagaacgccctgctcgtgcgttataccaaaaaggtccctcaggtgtctacaccaactctggtggaggtcagtaggaatctgggcaaagtgggatcaaagtgttgcaaacaccccgaggcaaagagaatgccttgtgctgaagattacctctccgtcgtgctgaaccagctctgcgtgctgcatgaaaagaccccagtcagcgaccgggtgacaaaatgttgcaccgaatctctggtcaatcgccgaccctgtttcagtgccctcgaagtggacgaaacttatgtgcctaaggagtttcaggctgaaacattcacctttcacgccgatatctgcactctgtccgagaaagaaaggcagattaagaaacagacagcactggtcgagctcgtgaagcataaaccaaaggctaccaaggagcagctgaaagccgtcatggacgatttcgcagcttttgtggaaaagtgttgcaaagccgacgataaggagacttgtttcgcagaagaggggaaaaagctcgtggctgccagccaggcagctctgggtctggccgcagctctgcaggaggtacagttgttggaatctggcggaggtcttgttcaaccaggtgggagtctgcgcctttcttgtgcagccagcggattcacattcagcgtgtacgacatgtattgggttcgccaggctcccgggaagggcttggagtgggtgagctatatttacagctcaggtggggacactgactacgcagactccgtcaaaggcaggtttactattagtagagacaattctaaaaatacactgtatcttcagatgaattccctgcgggcagaggacactgccacctactattgtgccagggcctcaactaactctatctacgatgctatggatgtgtggggccagggcacaactgtcacagtgagtagcgcatctacaggaggcgggggttcaggcggtggtggcagcggaggcggagggtccgatattcaaatgacccagtcaccctcatccgttagcgcaagtgtgggagatcgagtgaccatcacctgccgggcctcacaggatataacaaactggctggcttggtaccagcagaaacctggaaaggcccctacactgctgatctacgatgccagtacactccagagtggcgtaccctccagattttctggcagcgggtctggcaccgacttcactctgaccatctcctccctccagcctgaggatttcgctacctatttctgccaacaggctaacggatttccactcacctttggagggggtaccaaggtcgaaatcaagtgataat

SEQ ID NO: 109 - DNA, 660 bp, Artificial Sequence
  source: /note=“Description of Artificial Sequence Synthetic polynucleotide”
gaggtgcagctcctccaaagcggagggggtttggtccagcccggtggctcgttgcgcctctcatgtgccgcgtcggggtttacattcagcgtctatccgatgcactgggtacgacaggcgccagggaaaggactggagtgggtgtcatcgatcagcagctccggaggtgctacaagatatgccgactcggtgaaaggccgctttacgatcagccgcgataacagcaagaatacgttgtacctccaaatgaactcgctgcgagccgaggacactgcggtgtactattgcgcgaaggatttctatgacatccttactggtaatgcgtttgatatctggggccaggggacaacggtcaccgtgagctcggccagcacgaaggggcctagcgtctttccgctcgccccctcgtcaaaatcaacgtcgggtggaacggccgcattgggttgtctcgtaaaggactactttccggaacccgtcaccgtatcgtggaattcaggcgccctgacttccggggtccacacctttcccgctgtcttgcagtcctcgggtttgtattcactgtcgtcagtggtgacagtaccatcatcctcactgggaactcaaacgtacatctgcaacgtgaaccacaagccgagcaacactaaggtggataagaaggta

SEQ ID NO: 110 - DNA, 642 bp, Artificial Sequence
  source: /note=“Description of Artificial Sequence Synthetic polynucleotide”
gatatccagatgacacaaagccctagcagtttctctgcatctaccggagatagagtgactataacatgtagggcctcccagggcatctcatcctatctggcctggtatcagcagaagccaggcaaggctcccaaactcctgatttatgccgcaagtacactgcaaagcggagtgcctagtaggttttcagggtctggatctggcaccgacttcactctgaccatcagttctttgcagcccgaagactccgctacttattactgccagcagtattttacttttcctctgacatttggtggtggtacaaaagtggagattaagcgtacggtggccgctccctccgtctttatctttcctccaagcgacgagcagctgaagtctggcaccgcaagtgtggtgtgtctgctgaacaatttctaccccagggaagccaaagtgcaatggaaagtggataacgctctgcagtcaggaaattcccaggagagcgtcacagaacaagactctaaagatagtacttattcactgtccagcaccctgacactgtctaaggccgattatgagaaacacaaggtgtatgcctgtgaagtcactcatcaggggctgagttcacctgtgaccaaatcctttaacagaggtgagtgc

SEQ ID NO: 111 - DNA, 642 bp, Artificial Sequence
  source: /note=“Description of Artificial Sequence Synthetic polynucleotide”
gacattcagatgactcagagcccttcgtcccttagcgcatcaacaggggatcgagtaacgattacttgcagagcctcacaaggtatttcatcgtatctcgcatggtaccaacaaaagccgggtaaagctccgaaactcttgatctacgccgcgagcactctgcagtccggagtgccctcacgcttctcgggatcggggtcagggactgacttcacgctgaccatctccagcttgcagccagaggattttgcaacatactattgccagcagtacttcacgttccctcttacatttggaggtggaacaaaagtcgaaatcaaaaggacagtggctgcgccctcggtctttatctttccgccgtcggatgagcaactcaagtcggggactgcctcggtggtatgtttgctcaacaatttctatccccgggaagcgaaggtacagtggaaggtcgataatgccctccagagcggcaactcgcaagagtcagtcacagaacaggattccaaggactcgacgtattcactttcctcgacattgaccttgagcaaggccgactacgagaagcacaaagtgtatgcgtgtgaggtcacgcatcagggcctgtcatcgccagtgaccaagtcgtttaacagaggcgaatgc

SEQ ID NO: 112 - DNA, 327 bp, Artificial Sequence
  source: /note=“Description of Artificial Sequence Synthetic polynucleotide”
gatatccagatgacacaaagccctagcagtttctctgcatctaccggagatagagtgactataacatgtagggcctcccagggcatctcatcctatctggcctggtatcagcagaagccaggcaaggctcccaaactcctgatttatgccgcaagtacactgcaaagcggagtgcctagtaggttttcagggtctggatctggcaccgacttcactctgaccatcagttctttgcagcccgaagactccgctacttattactgccagcagtattttacttttcctctgacatttggtggtggtacaaaagtggagattaagcgtacg

SEQ ID NO: 113 - DNA, 327 bp, Artificial Sequence
  source: /note=“Description of Artificial Sequence Synthetic polynucleotide”
gacattcagatgactcagagcccttcgtcccttagcgcatcaacaggggatcgagtaacgattacttgcagagcctcacaaggtatttcatcgtatctcgcatggtaccaacaaaagccgggtaaagctccgaaactcttgatctacgccgcgagcactctgcagtccggagtgccctcacgcttctcgggatcggggtcagggactgacttcacgctgaccatctccagcttgcagccagaggattttgcaacatactattgccagcagtacttcacgttccctcttacatttggaggtggaacaaaagtcgaaatcaaaaggaca

SEQ ID NO: 114 - DNA, 735 bp, Artificial Sequence
  source: /note=“Description of Artificial Sequence Synthetic polynucleotide”
gaggtgcagctcctggagagtggcggtggcctcgtgcaaccaggaggatctttgcggctgtcatgtgccgctagtgggttcacctttagcgtgtatgaaatgcactgggttcgacaggcccccggcaaaggactcgagtgggtgagcggtatcagtagctccggcggttggaccccttatgccgactccgtaaagggcagattcacaattagccgggataattccaaaaacactctgtaccttcagatgaattctctgcgcgctgaagacacagccgtgtattattgcgcaaggcaaagtcagtattactactacggaatggacgtctggggacaaggcacaactgtaactgtgagttccgcatccaccggcggggggggcagcggtggtggtgggtctggaggagggggaagcgatatccagatgacccagtccccatctactctttccgcctccgtcggagatcgcgtgacaatcacttgtagggcctcacagtccatctcatcttggctggcttggtaccagcaaaagcccgggaccgctccaaagctgctgatttcaaagacttcaacattgcagagtggcgttcctagccgtttctctggtagcggctctgggaccgactttaccctgacaataacatctctccagcctgaagattttgcaacctactattgcttgcaggattacaacgacccctggacatttggccagggcacaaaggtggagattaaa

SEQ ID NO: 115 - DNA, 3329 bp, Artificial Sequence
  source: /note=“Description of Artificial Sequence Synthetic polynucleotide”
catgggctggagcctgatcttgctgtttctcgtggctgtcgctaccagagtactgtccgaagtgcagctgttggagagtgggggtggcctggtgcagccaggaggttccctccggctgagctgtgcagcaagcgggttcacttttagtgcctacaacatgaggtgggtgcgacaagcacccgggaaaggtcttgagtgggtcagtgttatctatccctcaggtggcgcaacacggtacgccgatagcgttaaggggcgcttcactattagcagagacaactcaaaaaatacactgtatttgcagatgaattcacttcgggctgaggatacagctgtgtactattgcgccaggggatactattattacgggatggatgtgtggggtcaaggcaccttggtgacagtatcaagcgccagtaccggcggtgggggcagtggaggcggaggatctggaggtggcgggtcccagtccgttcttactcagccacctagcgcttctgggacacccggccagcgagtgaccataagttgttccggatctgacagtaacatcggaaggaattatatttactggtaccaacagtttccaggcaccgcccctaagctcctgatttatcgcaataaccagcgccctagcggcgtccctgacagaatctcaggctccaagtccgggacatcagcctctctcgccatctctggacttcgttctgaagacgaggctgaataccactgcggcacctgggatgactccctctctggccccgtcttcggtggaggcaccaagctgactgtgctgtctggcgccgcctccgacgctcacaagagcgaagtggcacataggttcaaagatctgggcgaagagaactttaaggccctcgtcctgatcgctttcgcacagtacctccagcagtctccctttgaagatcacgtgaaactggtcaatgaggtgaccgaatttgccaagacatgcgtggctgatgagagtgcagaaaactgtgacaaatcactgcatactctctttggagataagctgtgcaccgtcgccacactcagagagacttatggggaaatggctgactgttgcgcaaaacaggagcctgaacggaatgagtgtttcctccagcacaaggatgacaacccaaatctgccccgcctcgtgcgacctgaggtcgatgtgatgtgcaccgcctttcatgacaacgaagagacattcctgaagaaatacctgtatgaaattgctcgtaggcacccatacttttatgcccccgagctcctgttctttgcaaagagatacaaagctgccttcactgaatgttgccaggcagctgataaggccgcatgtctcctgcctaaactggacgagctccgggatgaaggtaaggcttccagcgccaaacagcgcctgaagtgcgcttctctccagaagtttggcgagcgagcattcaaagcctgggctgtggcccgtctcagtcagaggtttccaaaggcagaatttgctgaggtctcaaaactggtgaccgacctcacaaaggtccatactgagtgttgccacggagatctgctggaatgtgccgacgatagagcagacctcgctaaatatatctgcgagaatcaggattccattagctctaagctgaaagaatgttgcgagaagcccctcctggaaaagagtcattgtatcgccgaggtggaaaacgacgagatgccagcagatctgccatcactcgctgccgactttgtggaatccaaagatgtctgcaagaattacgcagaggctaaagacgtgttcctggggatgtttctgtatgagtacgcccggcgtcaccccgattatagcgtcgtgctcctgctccgactggcaaagacctacgaaacaactctggagaaatgttgcgctgccgcagaccctcatgaatgttatgctaaggtgttcgatgagtttaagccactcgtcgaagagccccagaacctgattaaacagaattgcgaactgttcgagcagctcggtgaatacaagtttcagaacgccctgctcgtgcgttataccaaaaaggtccctcaggtgtctacaccaactctggtggaggtcagtaggaatctgggcaaagtgggatcaaagtgttgcaaacaccccgaggcaaagagaatgccttgtgctgaagattacctctccgtcgtgctgaaccagctctgcgtgctgcatgaaaagaccccagtcagcgaccgggtgacaaaatgttgcaccgaatctctggtcaatcgccgaccctgtttcagtgccctcgaagtggacgaaacttatgtgcctaaggagtttcaggctgaaacattcacctttcacgccgatatctgcactctgtccgagaaagaaaggcagattaagaaacagacagcactggtcgagctcgtgaagcataaaccaaaggctaccaaggagcagctgaaagccgtcatggacgatttcgcagcttttgtggaaaagtgttgcaaagccgacgataaggagacttgtttcgcagaagaggggaaaaagctcgtggctgccagccaggcagctctgggtctggccgcagctctgcaggaggtccagttgctcgagagtggaggcggtctggtgcagccaggtgggtccctgcgcttgagctgcgccgccagtgggttcaccttttccgtgtaccccatgcactgggtgcgacaagctccaggtaaaggcctcgaatgggtcagctctattagctcctctgggggcgctactcggtacgctgattctgtcaaggggcgtttcaccattagccgcgataattcaaagaacacactgtacctccagatgaattctctgcgggcagaggacaccgccgtttactactgtgccaaagacttttacgacatcctgactggcaacgcattcgatatctggggccaggggacaatggtcaccgtttcctcagcatcaaccggcggagggggctcaggagggggcggtagcggaggcggaggatccgatatccagatgacacaaagccctagcagtttctctgcatctaccggagatagagtgactataacatgtagggcctcccagggcatctcatcctatctggcctggtatcagcagaagccaggcaaggctcccaaactcctgatttatgccgcaagtacactgcaaagcggagtgcctagtaggttttcagggtctggatctggcaccgacttcactctgaccatcagttctttgcagcccgaagactccgctacttattactgccagcagtattttacttttcctctgacatttggtggtggtacaaaagtggagattaagtgataat

SEQ ID NO: 116 - DNA, 3330 bp, Artificial Sequence
  source: /note=“Description of Artificial Sequence Synthetic polynucleotide”
atgggctggtctcttatcttgctcttcctggttgctgttgctacccgggtacttagtgaggtgcagcttctggaaagcggagggggcttggtgcagcccggaggctctctccggttgagctgcgctgcttctggcttcacctttagtcactacgtgatggcatgggtgaggcaggcacctggcaagggactcgagtgggtgagctccatcagttctagtgggggctggacactgtacgccgacagtgtgaagggccgattcacaatctcacgcgacaattcaaagaacacattgtatctccagatgaacagcctgagagccgaagatactgccgtttactactgcaccagaggcctgaaaatggcaaccatctttgattactggggacagggcactctggtcacagtgtcttccgcctctaccggagggggcggatcaggagggggtgggtcaggaggtggaggttcccagtccgctctgacccagccagccagcgtcagcggctcccccggtcaaagcatcacaattagctgcactgggaccagttcagacgtcggctcatacaacgtggttagctggtatcaacagcatcctggtaaagcacccaaactgattatatatgaagtcagtcaaaggccatctggtgtgtccaatcgcttctctggttctaagtccggcaatacagccagtctgaccatctccgggctccagactgaggatgaggctgactattactgttgttcctatgccggatcaagcatttttgtgatttttgggggcggtactaaggtaaccgtcctcggcgccgcctccgacgctcacaagagcgaagtggcacataggttcaaagatctgggcgaagagaactttaaggccctcgtcctgatcgctttcgcacagtacctccagcagtctccctttgaagatcacgtgaaactggtcaatgaggtgaccgaatttgccaagacatgcgtggctgatgagagtgcagaaaactgtgacaaatcactgcatactctctttggagataagctgtgcaccgtcgccacactcagagagacttatggggaaatggctgactgttgcgcaaaacaggagcctgaacggaatgagtgtttcctccagcacaaggatgacaacccaaatctgccccgcctcgtgcgacctgaggtcgatgtgatgtgcaccgcctttcatgacaacgaagagacattcctgaagaaatacctgtatgaaattgctcgtaggcacccatacttttatgcccccgagctcctgttctttgcaaagagatacaaagctgccttcactgaatgttgccaggcagctgataaggccgcatgtctcctgcctaaactggacgagctccgggatgaaggtaaggcttccagcgccaaacagcgcctgaagtgcgcttctctccagaagtttggcgagcgagcattcaaagcctgggctgtggcccgtctcagtcagaggtttccaaaggcagaatttgctgaggtctcaaaactggtgaccgacctcacaaaggtccatactgagtgttgccacggagatctgctggaatgtgccgacgatagagcagacctcgctaaatatatctgcgagaatcaggattccattagctctaagctgaaagaatgttgcgagaagcccctcctggaaaagagtcattgtatcgccgaggtggaaaacgacgagatgccagcagatctgccatcactcgctgccgactttgtggaatccaaagatgtctgcaagaattacgcagaggctaaagacgtgttcctggggatgtttctgtatgagtacgcccggcgtcaccccgattatagcgtcgtgctcctgctccgactggcaaagacctacgaaacaactctggagaaatgttgcgctgccgcagaccctcatgaatgttatgctaaggtgttcgatgagtttaagccactcgtcgaagagccccagaacctgattaaacagaattgcgaactgttcgagcagctcggtgaatacaagtttcagaacgccctgctcgtgcgttataccaaaaaggtccctcaggtgtctacaccaactctggtggaggtcagtaggaatctgggcaaagtgggatcaaagtgttgcaaacaccccgaggcaaagagaatgccttgtgctgaagattacctctccgtcgtgctgaaccagctctgcgtgctgcatgaaaagaccccagtcagcgaccgggtgacaaaatgttgcaccgaatctctggtcaatcgccgaccctgtttcagtgccctcgaagtggacgaaacttatgtgcctaaggagtttcaggctgaaacattcacctttcacgccgatatctgcactctgtccgagaaagaaaggcagattaagaaacagacagcactggtcgagctcgtgaagcataaaccaaaggctaccaaggagcagctgaaagccgtcatggacgatttcgcagcttttgtggaaaagtgttgcaaagccgacgataaggagacttgtttcgcagaagaggggaaaaagctcgtggctgccagccaggcagctctgggtctggccgcagctctgcaggaggtccagttgctcgagagtggaggcggtctggtgcagccaggtgggtccctgcgcttgagctgcgccgccagtgggttcaccttttccgtgtaccccatgcactgggtgcgacaagctccaggtaaaggcctcgaatgggtcagctctattagctcctctgggggcgctactcggtacgctgattctgtcaaggggcgtttcaccattagccgcgataattcaaagaacacactgtacctccagatgaattctctgcgggcagaggacaccgccgtttactactgtgccaaagacttttacgacatcctgactggcaacgcattcgatatctggggccaggggacaatggtcaccgtttcctcagcatcaaccggcggagggggctcaggagggggcggtagcggaggcggaggatccgatatccagatgacacaaagccctagcagtttctctgcatctaccggagatagagtgactataacatgtagggcctcccagggcatctcatcctatctggcctggtatcagcagaagccaggcaaggctcccaaactcctgatttatgccgcaagtacactgcaaagcggagtgcctagtaggttttcagggtctggatctggcaccgacttcactctgaccatcagttctttgcagcccgaagactccgctacttattactgccagcagtattttacttttcctctgacatttggtggtggtacaaaagtggagattaagtgataa

SEQ ID NO: 117 - DNA, 3325 bp, Artificial Sequence
  source: /note=“Description of Artificial Sequence Synthetic polynucleotide”
atgggctggtctcttatcttgctcttcctggttgctgttgctacccgggtacttagtgaggtacagttgttggaatctggcggaggtcttgttcaaccaggtgggagtctgcgcctttcttgtgcagccagcggattcacattcagcgtgtacgacatgtattgggttcgccaggctcccgggaagggcttggagtgggtgagctatatttacagctcaggtggggacactgactacgcagactccgtcaaaggcaggtttactattagtagagacaattctaaaaatacactgtatcttcagatgaattccctgcgggcagaggacactgccacctactattgtgccagggcctcaactaactctatctacgatgctatggatgtgtggggccagggcacaactgtcacagtgagtagcgcatctacaggaggcgggggttcaggcggtggtggcagcggaggcggagggtccgatattcaaatgacccagtcaccctcatccgttagcgcaagtgtgggagatcgagtgaccatcacctgccgggcctcacaggatataacaaactggctggcttggtaccagcagaaacctggaaaggcccctacactgctgatctacgatgccagtacactccagagtggcgtaccctccagattttctggcagcgggtctggcaccgacttcactctgaccatctcctccctccagcctgaggatttcgctacctatttctgccaacaggctaacggatttccactcacctttggagggggtaccaaggtcgaaatcaagggcgccgcctccgacgctcacaagagcgaagtggcacataggttcaaagatctgggcgaagagaactttaaggccctcgtcctgatcgctttcgcacagtacctccagcagtctccctttgaagatcacgtgaaactggtcaatgaggtgaccgaatttgccaagacatgcgtggctgatgagagtgcagaaaactgtgacaaatcactgcatactctctttggagataagctgtgcaccgtcgccacactcagagagacttatggggaaatggctgactgttgcgcaaaacaggagcctgaacggaatgagtgtttcctccagcacaaggatgacaacccaaatctgccccgcctcgtgcgacctgaggtcgatgtgatgtgcaccgcctttcatgacaacgaagagacattcctgaagaaatacctgtatgaaattgctcgtaggcacccatacttttatgcccccgagctcctgttctttgcaaagagatacaaagctgccttcactgaatgttgccaggcagctgataaggccgcatgtctcctgcctaaactggacgagctccgggatgaaggtaaggcttccagcgccaaacagcgcctgaagtgcgcttctctccagaagtttggcgagcgagcattcaaagcctgggctgtggcccgtctcagtcagaggtttccaaaggcagaatttgctgaggtctcaaaactggtgaccgacctcacaaaggtccatactgagtgttgccacggagatctgctggaatgtgccgacgatagagcagacctcgctaaatatatctgcgagaatcaggattccattagctctaagctgaaagaatgttgcgagaagcccctcctggaaaagagtcattgtatcgccgaggtggaaaacgacgagatgccagcagatctgccatcactcgctgccgactttgtggaatccaaagatgtctgcaagaattacgcagaggctaaagacgtgttcctggggatgtttctgtatgagtacgcccggcgtcaccccgattatagcgtcgtgctcctgctccgactggcaaagacctacgaaacaactctggagaaatgttgcgctgccgcagaccctcatgaatgttatgctaaggtgttcgatgagtttaagccactcgtcgaagagccccagaacctgattaaacagaattgcgaactgttcgagcagctcggtgaatacaagtttcagaacgccctgctcgtgcgttataccaaaaaggtccctcaggtgtctacaccaactctggtggaggtcagtaggaatctgggcaaagtgggatcaaagtgttgcaaacaccccgaggcaaagagaatgccttgtgctgaagattacctctccgtcgtgctgaaccagctctgcgtgctgcatgaaaagaccccagtcagcgaccgggtgacaaaatgttgcaccgaatctctggtcaatcgccgaccctgtttcagtgccctcgaagtggacgaaacttatgtgcctaaggagtttcaggctgaaacattcacctttcacgccgatatctgcactctgtccgagaaagaaaggcagattaagaaacagacagcactggtcgagctcgtgaagcataaaccaaaggctaccaaggagcagctgaaagccgtcatggacgatttcgcagcttttgtggaaaagtgttgcaaagccgacgataaggagacttgtttcgcagaagaggggaaaaagctcgtggctgccagccaggcagctctgggtctggccgcagctctgcaggaggtccagttgctcgagagtggaggcggtctggtgcagccaggtgggtccctgcgcttgagctgcgccgccagtgggttcaccttttccgtgtaccccatgcactgggtgcgacaagctccaggtaaaggcctcgaatgggtcagctctattagctcctctgggggcgctactcggtacgctgattctgtcaaggggcgtttcaccattagccgcgataattcaaagaacacactgtacctccagatgaattctctgcgggcagaggacaccgccgtttactactgtgccaaagacttttacgacatcctgactggcaacgcattcgatatctggggccaggggacaatggtcaccgtttcctcagcatcaaccggcggagggggctcaggagggggcggtagcggaggcggaggatccgatatccagatgacacaaagccctagcagtttctctgcatctaccggagatagagtgactataacatgtagggcctcccagggcatctcatcctatctggcctggtatcagcagaagccaggcaaggctcccaaactcctgatttatgccgcaagtacactgcaaagcggagtgcctagtaggttttcagggtctggatctggcaccgacttcactctgaccatcagttctttgcagcccgaagactccgctacttattactgccagcagtattttacttttcctctgacatttggtggtggtacaaaagtggagattaagtgataat

SEQ ID NO: 118 - DNA, 1755 bp, Homo sapiens
gatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgtagctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaaatgtgccagtctccaaaaatttggagaaagagctttcaaagcatgggcagtggctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaggattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaactgtgagctttttaagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcacaaaatgctgcacagagtccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagcttgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggctta

SEQ ID NO: 119 - DNA, 1782 bp, Homo sapiens
ggcgccgcctccgacgctcacaagagcgaagtggcacataggttcaaagatctgggcgaagagaactttaaggccctcgtcctgatcgctttcgcacagtacctccagcagtctccctttgaagatcacgtgaaactggtcaatgaggtgaccgaatttgccaagacatgcgtggctgatgagagtgcagaaaactgtgacaaatcactgcatactctctttggagataagctgtgcaccgtcgccacactcagagagacttatggggaaatggctgactgttgcgcaaaacaggagcctgaacggaatgagtgtttcctccagcacaaggatgacaacccaaatctgccccgcctcgtgcgacctgaggtcgatgtgatgtgcaccgcctttcatgacaacgaagagacattcctgaagaaatacctgtatgaaattgctcgtaggcacccatacttttatgcccccgagctcctgttctttgcaaagagatacaaagctgccttcactgaatgttgccaggcagctgataaggccgcatgtctcctgcctaaactggacgagctccgggatgaaggtaaggcttccagcgccaaacagcgcctgaagtgcgcttctctccagaagtttggcgagcgagcattcaaagcctgggctgtggcccgtctcagtcagaggtttccaaaggcagaatttgctgaggtctcaaaactggtgaccgacctcacaaaggtccatactgagtgttgccacggagatctgctggaatgtgccgacgatagagcagacctcgctaaatatatctgcgagaatcaggattccattagctctaagctgaaagaatgttgcgagaagcccctcctggaaaagagtcattgtatcgccgaggtggaaaacgacgagatgccagcagatctgccatcactcgctgccgactttgtggaatccaaagatgtctgcaagaattacgcagaggctaaagacgtgttcctggggatgtttctgtatgagtacgcccggcgtcaccccgattatagcgtcgtgctcctgctccgactggcaaagacctacgaaacaactctggagaaatgttgcgctgccgcagaccctcatgaatgttatgctaaggtgttcgatgagtttaagccactcgtcgaagagccccagaacctgattaaacagaattgcgaactgttcgagcagctcggtgaatacaagtttcagaacgccctgctcgtgcgttataccaaaaaggtccctcaggtgtctacaccaactctggtggaggtcagtaggaatctgggcaaagtgggatcaaagtgttgcaaacaccccgaggcaaagagaatgccttgtgctgaagattacctctccgtcgtgctgaaccagctctgcgtgctgcatgaaaagaccccagtcagcgaccgggtgacaaaatgttgcaccgaatctctggtcaatcgccgaccctgtttcagtgccctcgaagtggacgaaacttatgtgcctaaggagtttcaggctgaaacattcacctttcacgccgatatctgcactctgtccgagaaagaaaggcagattaagaaacagacagcactggtcgagctcgtgaagcataaaccaaaggctaccaaggagcagctgaaagccgtcatggacgatttcgcagcttttgtggaaaagtgttgcaaagccgacgataaggagacttgtttcgcagaagaggggaaaaagctcgtggctgccagccaggcagctctgggtctggccgcagctctgcag

SEQ ID NO: 120 - DNA, 366 bp, Artificial Sequence
  source: /note=“Description of Artificial Sequence Synthetic polynucleotide”
gtggccgctccctccgtctttatctttcctccaagcgacgagcagctgaagtctggcaccgcaagtgtggtgtgtctgctgaacaatttctaccccagggaagccaaagtgcaatggaaagtggataacgctctgcaatcaggaaattcccaggagagcgtcacagaacaagactctaaagatagtacttattcactgtccagcaccctgacactgtctaaggccgattatgagaaacacaaggtgtatgcctgtgaagtcactcatcaggggctgagttcaccggtgaccaaatcctttaacagaggtgagtgcggtggcggaggtagcggtggcggagggagcggaggtggcggtagcctgcag

SEQ ID NO: 121 - DNA, 366 bp, Artificial Sequence
  source: /note=“Description of Artificial Sequence Synthetic polynucleotide”
cagcctaaggccgctccttccgtgaccctgttccctccttcctccgaggagctgcaagccaacaaggctaccctggtgtgcctgatttccgacttctaccctggcgccgtgaccgtggcttggaaggccgactcctcccctgtgaaggccggcgtggagaccaccaccccttccaagcagtccaacaacaagtacgccgcctcctcctacctgtccctgacccctgagcagtggaagtcccaccggtcctacagctgccaggtgacccacgagggctccaccgtggaaaagaccgtggcccctaccgagtgctccggtggcggaggtagcggtggcggagggagcggaggtggcggtagcctgcag

SEQ ID NO: 122 - DNA, 1029 bp, Artificial Sequence
  source: /note=“Description of Artificial Sequence Synthetic polynucleotide”
gctagcactaagggcccttccgtgttccctctggccccttgctcccggtccacctccgagtccaccgccgctctgggctgcctggtgaaggactacttccctgagcctgtgaccgtgagctggaacagcggcgctctgacctccggcgtgcacaccttccctgccgtgctccagtcctccggcctgtactccctgtcctccgtggtgacagtgccttcctccaacttcggcacccagacctacacctgcaacgtggaccacaagccttccaacaccaaggtggacaagaccgtggagcggaagtgctgcgtggagtgccctccttgccctgctccccctgtggctggccctagcgtgttcctgttccctcctaagcctaaggacaccctgatgatctcccggacccctgaggtgacctgcgtggtggtggacgtgtcccacgaggaccctgaggtgcagttcaattggtacgtggacggcgtggaggtgcacaacgctaagaccaagcctcgggaggagcagttcaactccaccttccgggtggtgtccgtgctgaccgtggtgcaccaggactggctgaacggcaaggaatacaagtgcaaggtctccaacaagggcctgcctgcccctatcgagaaaaccatctccaagaccaagggccagccccgggagcctcaggtgtacaccctgcctcccagccgggaggagatgaccaagaaccaggtgtccctgacctgtctggtgaagggcttctacccttccgacatcgccgtggagtgggagtccaacggccagcctgagaacaactacaagaccacccctcctatgctggactccgacggctccttcttcctgtactccaagctgacagtggacaagtcccggtggcagcagggcaacgtgttctcctgctccgtgatgcacgaggccctgcacaaccactacacccagaagtccctgtccctgagccctggcaagggtggcggaggtagcggtggcggagggagcggaggtggcggtagcctgcag

SEQ ID NO: 123 - DNA, 1014 bp, Artificial Sequence
  source: /note=“Description of Artificial Sequence Synthetic polynucleotide”
gctagcactaagggcccttccgtgttccctctggccccttgctcccggtccacctccgagtccaccgccgctctgggctgcctggtgaaggactacttccctgagcctgtgaccgtgagctggaacagcggcgctctgacctccggcgtgcacaccttccctgccgtgctccagtcctccggcctgtactccctgtcctccgtggtgacagtgccttcctccaacttcggcacccagacctacacctgcaacgtggaccacaagccttccaacaccaaggtggacaagaccgtggagcggaagtgctgcgtggagtgccctccttgccctgctccccctgtggctggccctagcgtgttcctgttccctcctaagcctaaggacaccctgatgatctcccggacccctgaggtgacctgcgtggtggtggacgtgtcccacgaggaccctgaggtgcagttcaattggtacgtggacggcgtggaggtgcacaacgctaagaccaagcctcgggaggagcagttcaactccaccttccgggtggtgtccgtgctgaccgtggtgcaccaggactggctgaacggcaaggaatacaagtgcaaggtctccaacaagggcctgcctgcccctatcgagaaaaccatctccaagaccaagggccagccccgggagcctcaggtgtacaccctgcctcccagccgggaggagatgaccaagaaccaggtgtccctgacctgtctggtgaagggcttctacccttccgacatcgccgtggagtgggagtccaacggccagcctgagaacaactacaagaccacccctcctatgctggactccgacggctccttcttcctgtactccaagctgacagtggacaagtcccggtggcagcagggcaacgtgttctcctgctccgtgatgcacgaggccctgcacaaccactacacccagaagtccctgtccctgagccctggcaagggtggcggagggagcggaggtggcggtagcctgcag

SEQ ID NO: 124 - DNA, 1032 bp, Artificial Sequence
  source: /note=“Description of Artificial Sequence Synthetic polynucleotide”
gctagcacaaaaggaccatcggtgtttcctcttgcaccatcatccaaatccacgtcaggtggaacggcggctcttgggtgcttggtcaaggactacttccccgagcctgtaacagtgtcgtggaactcgggggctctgacgtcgggagtgcacacttttcctgccgtcttgcagagctccggattgtattcactgtcgtccgtggtaacagtgccgtcgtcgtcgcttgggacacaaacgtacatctgcaatgtgaaccataagccgagcaataccaaggtggacaagaaggtcgaacccaaatcatgtgacaaaactcatacgtgcccgccttgtcccgctccggaacttctcggtgggccgtcggtctttctcttcccgccgaagccgaaagacacgctcatgattagccgaacccccgaggtcacatgcgtggtggtcgatgtctcgcacgaggaccccgaagtaaagttcaattggtacgtggatggtgtggaggtacacaacgcgaaaaccaaaccccgggaggaacagtaccagtcaacctatcgcgtagtcagcgtgctgacagtcctccatcaagactggttgaacgggaaagagtataagtgcaaagtgtcaaacaaggcgctcccggcacctatcgaaaagactatttcgaaggcgaaggggcagcctcgcgaacctcaagtgtacacccttccaccgagcagagaagagatgaccaaaaaccaggtatcacttacgtgtcttgtgaaggggttctatccttcagacattgcagtcgaatgggaatcaaatgggcagccagagaataactacaagacaacacccccagtactcgactcagatgggagcttctttctctactcaaagctgacggtagataagtcgagatggcagcaggggaacgtgtttagctgctcagtgatgcacgaggccctccataatcactatacacagaaatccttgtcgcttagccccggaggaggagggggttccggaggagggggttccggaggagggggttcc

SEQ ID NO: 125 - DNA, 1032 bp, Artificial Sequence
  source: /note=“Description of Artificial Sequence Synthetic polynucleotide”
gctagcacaaaaggaccatcggtgtttcctcttgcaccatcatccaaatccacgtcaggtggaacggcggctcttgggtgcttggtcaaggactacttccccgagcctgtaacagtgtcgtggaactcgggggctctgacgtcgggagtgcacacttttcctgccgtcttgcagagctccggattgtattcactgtcgtccgtggtaacagtgccgtcgtcgtcgcttgggaaccaaacgtacatctgcaatgtgaaccataagccgagcaataccaaggtggacaagaaggtcgaacccaaatcatgtgacaaaactcatacgtgcccgccttgtcccgctccggaacttctcggtgggccgtcggtctttctcttcccgccgaagccgaaagacacgctcatgattagccgaacccccgaggtcacatgcgtggtggtcgatgtctcgcacgaggaccccgaagtaaagttcaattggtacgtggatggtgtggaggtacacaacgcgaaaaccaaaccccgggaggaacagtacaattcaacctatcgcgtagtcagcgtgctgacagtcctccatcaagactggttgaacgggaaagagtataagtgcaaagtgtcaaacaaggcgctcccggcacctatcgaaaagactatttcgaaggcgaaggggcagcctcgcgaacctcaagtgtacacccttccaccgagcagagaagagatgaccaaaaaccaggtatcacttacgtgtcttgtgaaggggttctatccttcagacattgcagtcgaatgggaatcaaatgggcagccagagaataactacaagacaacacccccagtactcgactcagatgggagcttctttctctactcaaagctgacggtagataagtcgagatggcagcaggggaacgtgtttagctgctcagtgatgcacgaggccctccataatcactatacacagaaatccttgtcgcttagccccggaggaggagggggttccggaggagggggttccggaggagggggttcc

SEQ ID NO: 126 - DNA, 681 bp, Artificial Sequence
  source: /note=“Description of Artificial Sequence Synthetic polynucleotide”
ggaggaggagggagcggtggaggaggctcgggtgggggtgggagcggagggggagggtcgacctccgaagaaacgatctcgacagtgcaagaaaaacagcagaacatttcaccattggtcagggagcgaggtccgcaaagagtagccgcccacatcactgggacgcgcggaagatccaataccctctcatccccgaacagcaagaatgagaaggcgctcggacgcaaaatcaactcgtgggagtcatccagaagcggacattcgttcctctcgaaccttcatcttaggaatggggaacttgtcatccacgagaaagggttttactatatctactcacagacctatttccggttccaagaagagatcaaggagaataccaagaatgacaaacagatggtgcaatacatctacaagtatacctcataccctgatcccattttgcttatgaaatcggcgagaaactcgtgctggtcaaaggacgccgagtatgggctgtactcaatctaccagggaggaatctttgagcttaaagaaaacgatagaatcttcgtatcggtcactaacgagcacctcattgacatggatcacgaggcctccttcttcggggcttttctcgtcggtggtggtggcggatccggtggaggaggatcagggggaggaggaagcgggggaggtggcagc

SEQ ID NO: 127 - DNA, 564 bp, Artificial Sequence
  source: /note=“Description of Artificial Sequence Synthetic polynucleotide”
ggaggaggagggagcggtggaggaggctcgggtgggggtgggagcggagggggagggtcgaaccctgcggcccacttgaccggagcgaactcatcgctcaccggatcgggagggcccctcctttgggaaacgcagctcggtctggcatttctccgcggactgtcgtatcacgacggggcgttggtcgtcacaaaagctggatactactacatctactcaaaagtgcaacttggaggtgtcgggtgcccgcttggtttggcgtcaaccatcacgcacggattgtataagagaactcctcggtaccccgaggaactcgagctgttggtatcacaacagtcgccatgtggaagagccacctcgtcatcgagagtatggtgggattcaagctttctcgggggtgtggtgcatttggaagcaggagaagaggtcgtcgtgcgagtcttggatgagaggcttgtcagattgcgggatggcacaagatcctacttcggggccttcatggtgggaggtgggggttccggtggaggaggatcagggggaggaggaagcgggggaggtggcagc

SEQ ID NO: 128 - DNA, 741 bp, Artificial Sequence
  source: /note=“Description of Artificial Sequence Synthetic polynucleotide”
gaagtgcagctgttggagagtgggggtggcctggtgcagccaggaggttccctccggctgagctgtgcagcaagcgggttcacttttagtgcctacaacatgaggtgggtgcgacaagcacccgggaaaggtcttgagtgggtcagtgttatctatccctcaggtggcgcaacacggtacgccgatagcgttaaggggcgcttcactattagcagagacaactcaaaaaatacactgtatttgcagatgaattcacttcgggctgaggatacagctgtgtactattgcgccaggggatactattattacgggatggatgtgtggggtcaaggcaccttggtgacagtatcaagcgccagtaccggcggtgggggcagtggaggcggaggatctggaggtggcgggtcccagtccgttcttactcagccacctagcgcttctgggacacccggccagcgagtgaccataagttgttccggatctgacagtaacatcggaaggaattatatttactggtaccaacagtttccaggcaccgcccctaagctcctgatttatcgcaataaccagcgccctagcggcgtccctgacagaatctcaggctccaagtccgggacatcagcctctctcgccatctctggacttcgttctgaagacgaggctgaataccactgcggcacctgggatgactccctctctggccccgtcttcggtggaggcaccaagctgactgtgctgtct

SEQ ID NO: 129 - DNA, 756 bp, Artificial Sequence
  source: /note=“Description of Artificial Sequence Synthetic polynucleotide”
gaggtacaattgcagcagtcgggaggcgggcttgtgcaacccggagggtcactgcgcctttcatgtgcggcgagcggtttcacgttctcagcgtacaatatgagatgggtacggcaggcacctggcaaagggttggagtgggtagccgtaatctaccccagcgggggtgcgacacggtatgcggattccgtcaaagggaggtttacaatttcgagggacaattccaagaacacactttatcttcaaatgaattcgctcagggcagaagataccgccgtctactactgcgcgaggggatactactactacgggatggatgtctgggggcaggggactctggtaactgtctcatcggcatcgactggtggtggcgggtccggagggggaggaagcggaggaggcgggagcggaggcggtgggtcccaaagcgtactgacgcagcctccttcggcctccggaacaccggggcagtcggtgaccatctcatgttcagggtcggatagcaacattgggagaaactacatctactggtatcagcaattccccggaacggcacctaaacttctcatctaccggaataaccaaagaccgagcggagtgccggatcggttttcgggttcgaaatccggaacgtcggcgtcgctcgcaatctccggacttcagtcagaggatgaagcggattactattgtggaacgtgggatgactcgttgtcggggcctgtgttcggtgggggtacaaagctcactgtactcggc

SEQ ID NO: 130 - DNA, 354 bp, Artificial Sequence
  source: /note=“Description of Artificial Sequence Synthetic polynucleotide”
gaagtgcagctgttggagagtgggggtggcctggtgcagccaggaggttccctccggctgagctgtgcagcaagcgggttcacttttagtgcctacaacatgaggtgggtgcgacaagcacccgggaaaggtcttgagtgggtcagtgttatctatccctcaggtggcgcaacacggtacgccgatagcgttaaggggcgcttcactattagcagagacaactcaaaaaatacactgtatttgcagatgaattcacttcgggctgaggatacagctgtgtactattgcgccaggggatactattattacgggatggatgtgtggggtcaaggcaccttggtgacagtatcaagc

SEQ ID NO: 131 - DNA, 648 bp, Artificial Sequence
  source: /note=“Description of Artificial Sequence Synthetic polynucleotide”
cagtccgttcttactcagccacctagcgcttctgggacacccggccagcgagtgaccataagttgttccggatctgacagtaacatcggaaggaattatatttactggtaccaacagtttccaggcaccgcccctaagctcctgatttatcgcaataaccagcgccctagcggcgtccctgacagaatctcaggctccaagtccgggacatcagcctctctcgccatctctggacttcgttctgaagacgaggctgaataccactgcggcacctgggatgactccctctctggccccgtcttcggtggaggcaccaagctgactgtcctaggacagcctaaggccgctccttccgtgaccctgttccctccttcctccgaggagctgcaagccaacaaggctaccctggtgtgcctgatttccgacttctaccctggcgccgtgaccgtggcttggaaggccgactcctcccctgtgaaggccggcgtggagaccaccaccccttccaagcagtccaacaacaagtacgccgcctcctcctacctgtccctgacccctgagcagtggaagtcccaccggtcctacagctgccaggtgacccacgagggctccaccgtggaaaagaccgtggcccctaccgagtgctcc

SEQ ID NO: 132 - DNA, 333 bp, Artificial Sequence
  source: /note=“Description of Artificial Sequence Synthetic polynucleotide”
cagtccgttcttactcagccacctagcgcttctgggacacccggccagcgagtgaccataagttgttccggatctgacagtaacatcggaaggaattatatttactggtaccaacagtttccaggcaccgcccctaagctcctgatttatcgcaataaccagcgccctagcggcgtccctgacagaatctcaggctccaagtccgggacatcagcctctctcgccatctctggacttcgttctgaagacgaggctgaataccactgcggcacctgggatgactccctctctggccccgtcttcggtggaggcaccaagctgactgtcctagga

SEQ ID NO: 133 - DNA, 747 bp, Artificial Sequence
  source: /note=“Description of Artificial Sequence Synthetic polynucleotide”
gaggtgcagcttctggaaagcggagggggcttggtgcagcccggaggctctctccggttgagctgcgctgcttctggcttcacctttagtcactacgtgatggcatgggtgaggcaggcacctggcaagggactcgagtgggtgagctccatcagttctagtgggggctggacactgtacgccgacagtgtgaagggccgattcacaatctcacgcgacaattcaaagaacacattgtatctccagatgaacagcctgagagccgaagatactgccgtttactactgcaccagaggcctgaaaatggcaaccatctttgattactggggacagggcactctggtcacagtgtcttccgcctctaccggagggggcggatcaggagggggtgggtcaggaggtggaggttcccagtccgctctgacccagccagccagcgtcagcggctcccccggtcaaagcatcacaattagctgcactgggaccagttcagacgtcggctcatacaacgtggttagctggtatcaacagcatcctggtaaagcacccaaactgattatatatgaagtcagtcaaaggccatctggtgtgtccaatcgcttctctggttctaagtccggcaatacagccagtctgaccatctccgggctccagactgaggatgaggctgactattactgttgttcctatgccggatcaagcatttttgtgatttttgggggcggtactaaggtaaccgtcctcggc

SEQ ID NO: 134 - DNA, 747 bp, Artificial Sequence
  source: /note=“Description of Artificial Sequence Synthetic polynucleotide”
caagttcaattagttcaatctggtgggggtttggttcaaccaggaaggtctttacgactgtcatgcgcagcatctgggttcacattcgatgattacgccatgcactgggtccgtcaggctccaggcaaaggtcttgaatgggtatctgggattagttggaattctggatcaatcgggtatgcagattccgtcaagggtaggttcacaatttccagagataacgccaaaaacagtctgtacttacaaatgaactccttgaggccagaagatactgctgtatactactgtgctagagatttgggcgcgaagcaatggttggagggatttgattactggggccaaggtacgcttgtgacagttagtagtgcttcaacaggtggtggtggttcaggcggtggtgggtcaggaggaggaggctctagttatgagttgactcaagaccctgccgtttctgtcgctcttggtcagaccgtgagaattacatgtcagggtgattccctaagatcatactacgcaagctggtatcagcaaaagcctggacaagctcctgttctcgtgatatacggcaaaaacaacagaccatctggtatcccagacagattctcaggctctactagcggaaattcagcgtccttaactatcactggtgcccaagcagaagatgaagcagactattactgcaattcaagagattcatcaggtaatcattgggtctttggaggcggcactaaggtaaccgtactcggc

SEQ ID NO: 135 - DNA, 763 bp, Artificial Sequence
  source: /note=“Description of Artificial Sequence Synthetic polynucleotide”
caagtgcaattggtacagtcgggaggcgggcttgtgcaacccggagggtcactgcgcctttcatgtgcggcgagcggtttcacgttcgatgattatgcgatgcattgggtacggcaggcacctggcaaagggttggagtgggtagccggaatctcgtggaatagcgggagcatcggttatgcggattccgtcaaagggaggtttacaatttcgagggacaatgcgaagaacagcctgtatctccaaatgaattcgctcaggcccgaagataccgccgtctactactgcgcgagggacttgggatacaaccagtgggtggaagggtttgattactgggggcaggggactctggtaactgtctcatcggcatcgactggtggtggcgggtccggagggggaggaagcggaggaggcgggagcggaggcggtgggtccagctacgaactgactcaagatccggccgtatcggtggccctcggacaaacagtgcgaatcacctgtcagggggatagccttagatcgtactacgcttcgtggtatcagcaaaagcccggacaggcaccagtgcttgtgatctacggaaagaacaatagaccgagcggaattccggatcggttttcgggttcgacttccggaaattcggcgtcgctcacgatcaccggagctcaggcagaggatgaagcggattactattgtaattcccgcgattcgtcggggaatcattgggtgttcggtgggggtacaaaggtaactgtactcggct

SEQ ID NO: 136 - DNA, 1662 bp, Artificial Sequence
  source: /note=“Description of Artificial Sequence Synthetic polynucleotide”
gaggtacagttgttggaatctggcggaggtcttgttcaaccaggtgggagtctgcgcctttcttgtgcagccagcggattcacattcagcgtgtacgacatgtattgggttcgccaggctcccgggaagggcttggagtgggtgagctatatttacagctcaggtggggacactgactacgcagactccgtcaaaggcaggtttactattagtagagacaattctaaaaatacactgtatcttcagatgaattccctgcgggcagaggacactgccacctactattgtgccagggcctcaactaactctatctacgatgctatggatgtgtggggccagggcacaactgtcacagtgagtagcgcatctacaggaggcgggggttcaggcggtggtggcagcggaggcggagggtccgatattcaaatgacccagtcaccctcatccgttagcgcaagtgtgggagatcgagtgaccatcacctgccgggcctcacaggatataacaaactggctggcttggtaccagcagaaacctggaaaggcccctacactgctgatctacgatgccagtacactccagagtggcgtaccctccagattttctggcagcgggtctggcaccgacttcactctgaccatctcctccctccagcctgaggatttcgctacctatttctgccaacaggctaacggatttccactcacctttggagggggtaccaaggtcgaaatcaagaccggtcaagagcaacaatcccaccaaccacattcagagcaagatgacgagcacccaacaggacaaacacaagcacaagaacaagctgaaactgaggagggtcatgaggcacaaaatgctgaagaacagaatgcccatacccaggatgaacacgatcagacaccaaaccaaccaggagctagcgaagtgcagctgttggagagtgggggtggcctggtgcagccaggaggttccctccggctgagctgtgcagcaagcgggttcacttttagtgcctacaacatgaggtgggtgcgacaagcacccgggaaaggtcttgagtgggtcagtgttatctatccctcaggtggcgcaacacggtacgccgatagcgttaaggggcgcttcactattagcagagacaactcaaaaaatacactgtatttgcagatgaattcacttcgggctgaggatacagctgtgtactattgcgccaggggatactattattacgggatggatgtgtggggtcaaggcaccttggtgacagtatcaagcgccagtaccggcggtgggggcagtggaggcggaggatctggaggtggcgggtcccagtccgttcttactcagccacctagcgcttctgggacacccggccagcgagtgaccataagttgttccggatctgacagtaacatcggaaggaattatatttactggtaccaacagtttccaggcaccgcccctaagctcctgatttatcgcaataaccagcgccctagcggcgtccctgacagaatctcaggctccaagtccgggacatcagcctctctcgccatctctggacttcgttctgaagacgaggctgaataccactgcggcacctgggatgactccctctctggccccgtcttcggtggaggcaccaagctgactgtgctgtct

SEQ ID NO: 137 - DNA, 1662 bp, Artificial Sequence
  source: /note=“Description of Artificial Sequence Synthetic polynucleotide”
gatattcaaatgacccagtcaccctcatccgttagcgcaagtgtgggagatcgagtgaccatcacctgccgggcctcacaggatataacaaactggctggcttggtaccagcagaaacctggaaaggcccctacactgctgatctacgatgccagtacactccagagtggcgtaccctccagattttctggcagcgggtctggcaccgacttcactctgaccatctcctccctccagcctgaggatttcgctacctatttctgccaacaggctaacggatttccactcacctttggagggggtaccaaggtcgaaatcaaggcatctacaggaggcgggggttcaggcggtggtggcagcggaggcggagggtccgaggtacagttgttggaatctggcggaggtcttgttcaaccaggtgggagtctgcgcctttcttgtgcagccagcggattcacattcagcgtgtacgacatgtattgggttcgccaggctcccgggaagggcttggagtgggtgagctatatttacagctcaggtggggacactgactacgcagactccgtcaaaggcaggtttactattagtagagacaattctaaaaatacactgtatcttcagatgaattccctgcgggcagaggacactgccacctactattgtgccagggcctcaactaactctatctacgatgctatggatgtgtggggccagggcacaactgtcacagtgagtagcaccggtcaagagcaacaatcccaccaaccacattcagagcaagatgacgagcacccaacaggacaaacacaagcacaagaacaagctgaaactgaggagggtcatgaggcacaaaatgctgaagaacagaatgcccatacccaggatgaacacgatcagacaccaaaccaaccaggagctagcgaagtgcagctgttggagagtgggggtggcctggtgcagccaggaggttccctccggctgagctgtgcagcaagcgggttcacttttagtgcctacaacatgaggtgggtgcgacaagcacccgggaaaggtcttgagtgggtcagtgttatctatccctcaggtggcgcaacacggtacgccgatagcgttaaggggcgcttcactattagcagagacaactcaaaaaatacactgtatttgcagatgaattcacttcgggctgaggatacagctgtgtactattgcgccaggggatactattattacgggatggatgtgtggggtcaaggcaccttggtgacagtatcaagcgccagtaccggcggtgggggcagtggaggcggaggatctggaggtggcgggtcccagtccgttcttactcagccacctagcgcttctgggacacccggccagcgagtgaccataagttgttccggatctgacagtaacatcggaaggaattatatttactggtaccaacagtttccaggcaccgcccctaagctcctgatttatcgcaataaccagcgccctagcggcgtccctgacagaatctcaggctccaagtccgggacatcagcctctctcgccatctctggacttcgttctgaagacgaggctgaataccactgcggcacctgggatgactccctctctggccccgtcttcggtggaggcaccaagctgactgtgctgtct

SEQ ID NO: 138 - DNA, 1668 bp, Artificial Sequence
  source: /note=“Description of Artificial Sequence Synthetic polynucleotide”
gatattcaaatgacccagtcaccctcatccgttagcgcaagtgtgggagatcgagtgaccatcacctgccgggcctcacaggatataacaaactggctggcttggtaccagcagaaacctggaaaggcccctacactgctgatctacgatgccagtacactccagagtggcgtaccctccagattttctggcagcgggtctggcaccgacttcactctgaccatctcctccctccagcctgaggatttcgctacctatttctgccaacaggctaacggatttccactcacctttggagggggtaccaaggtcgaaatcaagggtgaaccagctgcttcttccccatcttcaaataatgcagctgctactacagcagcttctgaggtacagttgttggaatctggcggaggtcttgttcaaccaggtgggagtctgcgcctttcttgtgcagccagcggattcacattcagcgtgtacgacatgtattgggttcgccaggctcccgggaagggcttggagtgggtgagctatatttacagctcaggtggggacactgactacgcagactccgtcaaaggcaggtttactattagtagagacaattctaaaaatacactgtatcttcagatgaattccctgcgggcagaggacactgccacctactattgtgccagggcctcaactaactctatctacgatgctatggatgtgtggggccagggcacaactgtcacagtgagtagcaccggtcaagagcaacaatcccaccaaccacattcagagcaagatgacgagcacccaacaggacaaacacaagcacaagaacaagctgaaactgaggagggtcatgaggcacaaaatgctgaagaacagaatgcccatacccaggatgaacacgatcagacaccaaaccaaccaggagctagcgaagtgcagctgttggagagtgggggtggcctggtgcagccaggaggttccctccggctgagctgtgcagcaagcgggttcacttttagtgcctacaacatgaggtgggtgcgacaagcacccgggaaaggtcttgagtgggtcagtgttatctatccctcaggtggcgcaacacggtacgccgatagcgttaaggggcgcttcactattagcagagacaactcaaaaaatacactgtatttgcagatgaattcacttcgggctgaggatacagctgtgtactattgcgccaggggatactattattacgggatggatgtgtggggtcaaggcaccttggtgacagtatcaagcgccagtaccggcggtgggggcagtggaggcggaggatctggaggtggcgggtcccagtccgttcttactcagccacctagcgcttctgggacacccggccagcgagtgaccataagttgttccggatctgacagtaacatcggaaggaattatatttactggtaccaacagtttccaggcaccgcccctaagctcctgatttatcgcaataaccagcgccctagcggcgtccctgacagaatctcaggctccaagtccgggacatcagcctctctcgccatctctggacttcgttctgaagacgaggctgaataccactgcggcacctgggatgactccctctctggccccgtcttcggtggaggcaccaagctgactgtgctgtct

SEQ ID NO: 139 - DNA, 1332 bp, Artificial Sequence
  source: /note=“Description of Artificial Sequence Synthetic polynucleotide”
gaagtgcagctgttggagagtgggggtggcctggtgcagccaggaggttccctccggctgagctgtgcagcaagcgggttcacttttagtgcctacaacatgaggtgggtgcgacaagcacccgggaaaggtcttgagtgggtcagtgttatctatccctcaggtggcgcaacacggtacgccgatagcgttaaggggcgcttcactattagcagagacaactcaaaaaatacactgtatttgcagatgaattcacttcgggctgaggatacagctgtgtactattgcgccaggggatactattattacgggatggatgtgtggggtcaaggcaccttggtgacagtatcaagcgctagcactaagggcccttccgtgttccctctggccccttgctcccggtccacctccgagtccaccgccgctctgggctgcctggtgaaggactacttccctgagcctgtgaccgtgagctggaacagcggcgctctgacctccggcgtgcacaccttccctgccgtgctccagtcctccggcctgtactccctgtcctccgtggtgacagtgccttcctccaacttcggcacccagacctacacctgcaacgtggaccacaagccttccaacaccaaggtggacaagaccgtggagcggaagtgctgcgtggagtgccctccttgccctgctccccctgtggctggccctagcgtgttcctgttccctcctaagcctaaggacaccctgatgatctcccggacccctgaggtgacctgcgtggtggtggacgtgtcccacgaggaccctgaggtgcagttcaattggtacgtggacggcgtggaggtgcacaacgctaagaccaagcctcgggaggagcagttcaactccaccttccgggtggtgtccgtgctgaccgtggtgcaccaggactggctgaacggcaaggaatacaagtgcaaggtctccaacaagggcctgcctgcccctatcgagaaaaccatctccaagaccaagggccagccccgggagcctcaggtgtacaccctgcctcccagccgggaggagatgaccaagaaccaggtgtccctgacctgtctggtgaagggcttctacccttccgacatcgccgtggagtgggagtccaacggccagcctgagaacaactacaagaccacccctcctatgctggactccgacggctccttcttcctgtactccaagctgacagtggacaagtcccggtggcagcagggcaacgtgttctcctgctccgtgatgcacgaggccctgcacaaccactacacccagaagtccctgtccctgagccctggcaag

SEQ ID NO: 140 - DNA, 1344 bp, Artificial Sequence
  source: /note=“Description of Artificial Sequence Synthetic polynucleotide”
gaggtccagttgctcgagagtggaggcggtctggtgcagccaggtgggtccctgcgcttgagctgcgccgccagtgggttcaccttttccgtgtaccccatgcactgggtgcgacaagctccaggtaaaggcctcgaatgggtcagctctattagctcctctgggggcgctactcggtacgctgattctgtcaaggggcgtttcaccattagccgcgataattcaaagaacacactgtacctccagatgaattctctgcgggcagaggacaccgccgtttactactgtgccaaagacttttacgacatcctgactggcaacgcattcgatatctggggccaggggacaatggtcaccgtttcctcagctagcactaagggcccttccgtgttccctctggccccttgctcccggtccacctccgagtccaccgccgctctgggctgcctggtgaaggactacttccctgagcctgtgaccgtgagctggaacagcggcgctctgacctccggcgtgcacaccttccctgccgtgctccagtcctccggcctgtactccctgtcctccgtggtgacagtgccttcctccaacttcggcacccagacctacacctgcaacgtggaccacaagccttccaacaccaaggtggacaagaccgtggagcggaagtgctgcgtggagtgccctccttgccctgctccccctgtggctggccctagcgtgttcctgttccctcctaagcctaaggacaccctgatgatctcccggacccctgaggtgacctgcgtggtggtggacgtgtcccacgaggaccctgaggtgcagttcaattggtacgtggacggcgtggaggtgcacaacgctaagaccaagcctcgggaggagcagttcaactccaccttccgggtggtgtccgtgctgaccgtggtgcaccaggactggctgaacggcaaggaatacaagtgcaaggtctccaacaagggcctgcctgcccctatcgagaaaaccatctccaagaccaagggccagccccgggagcctcaggtgtacaccctgcctcccagccgggaggagatgaccaagaaccaggtgtccctgacctgtctggtgaagggcttctacccttccgacatcgccgtggagtgggagtccaacggccagcctgagaacaactacaagaccacccctcctatgctggactccgacggctccttcttcctgtactccaagctgacagtggacaagtcccggtggcagcagggcaacgtgttctcctgctccgtgatgcacgaggccctgcacaaccactacacccagaagtccctgtccctgagccctggcaag

SEQ ID NO: 141 - DNA, 765 bp, Artificial Sequence
  source: /note=“Description of Artificial Sequence Synthetic polynucleotide”
gaggtgcaactcctggagtctgggggcggactcgtgcagcctggagggtctttgagactgtcttgcgccgcatctggctttaccttctcctggtacgggatgtattgggttcgccaggcacccgggaagggactggagtgggtgtccggaatttctagttcaggcgggtacactatgtatgccgatagcgtgaaaggaaggtttactatttctcgggacaattccaagaacaccctgtatctccagatgaattccctgagagccgaagacactgctgtctactactgcgctaaggtcggaactctcgttgtgcctgccgctattccttacttccaacactgggggcagggcactcttgtcactgtatcaagcgccagtacaggagggggtggttcaggtggaggcggctctggcggtggcgggagtgatatacagatgacacagagcccagatagtctggccgtgagcttgggtgaaagggctacaatcaactgtaaatctagtcagtccatcttgtacaacagcaataacaaaaattaccttgcatggtatcagcagaagcccggacagccacccaaactcctgatctattggacatccacacgcgaaagcggtgtgcctgatcgattttccggctccggtagtggcaccgacttcagcctgaccatcagcagtctgcaagctgaggacgtcgcagtctactattgtcagcagtattacagtaccccacccacattcggccaaggcaccaaggtggagatcaag

SEQ ID NO: 142 - DNA, 741 bp, Artificial Sequence
  source: /note=“Description of Artificial Sequence Synthetic polynucleotide”
gaggtccagttgcttgaaagtggtggcgggttggtacagcctggcggaagcctgagactctcatgcgcagcctccgggtttacattttctgcttatgggatgggctgggtgagacaagcccccggaaagggcctggagtgggtgtcctacatctcccccagcggtgggcacaccaagtatgccgattcagtgaaagggcgcttcaccattagcagggacaactcaaagaacacattgtaccttcagatgaattccctgcgcgccgaagataccgctgtctattattgcgcaaaggtcctggaaacaggcctcctggttgacgcctttgatatctggggtcagggtactatggtaacagtgagtagtgcaagcacaggtggcggagggtctggagggggaggatctggaggcggcggctcttcctatgagctcactcagccaccttcagtgagtgtgtaccccggacagacagctagtatcacttgttctggcgaccagctgggttctaaattcgtctcatggtaccagcagaggcctggacaaagccccgtgctggttatgtacaaagacaagcgacggccaagtgagattccagaacggttctccggctctaatagtggcaacactgccaccctgactatcagcggtacccaagctatagatgaggcagactactattgtcaggcttgggattccagcacttacgtgttcgggacaggcaccaaagttaccgtgctcggc

SEQ ID NO: 143 - protein, 585 aa, Homo sapiens [sequence omitted]

SEQ ID NO: 144 - DNA, 1755 bp, Homo sapiens; identical to SEQ ID NO: 118

SEQ ID NO: 145 - protein, 585 aa, Homo sapiens
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGL

SEQ ID NO: 146 - DNA, 1755 bp, Homo sapiens
gacgctcacaagagcgaagtggcacataggttcaaagatctgggcgaagagaactttaaggccctcgtcctgatcgctttcgcacagtacctccagcagtctccctttgaagatcacgtgaaactggtcaatgaggtgaccgaatttgccaagacatgcgtggctgatgagagtgcagaaaactgtgacaaatcactgcatactctctttggagataagctgtgcaccgtcgccacactcagagagacttatggggaaatggctgactgttgcgcaaaacaggagcctgaacggaatgagtgtttcctccagcacaaggatgacaacccaaatctgccccgcctcgtgcgacctgaggtcgatgtgatgtgcaccgcctttcatgacaacgaagagacattcctgaagaaatacctgtatgaaattgctcgtaggcacccatacttttatgcccccgagctcctgttctttgcaaagagatacaaagctgccttcactgaatgttgccaggcagctgataaggccgcatgtctcctgcctaaactggacgagctccgggatgaaggtaaggcttccagcgccaaacagcgcctgaagtgcgcttctctccagaagtttggcgagcgagcattcaaagcctgggctgtggcccgtctcagtcagaggtttccaaaggcagaatttgctgaggtctcaaaactggtgaccgacctcacaaaggtccatactgagtgttgccacggagatctgctggaatgtgccgacgatagagcagacctcgctaaatatatctgcgagaatcaggattccattagctctaagctgaaagaatgttgcgagaagcccctcctggaaaagagtcattgtatcgccgaggtggaaaacgacgagatgccagcagatctgccatcactcgctgccgactttgtggaatccaaagatgtctgcaagaattacgcagaggctaaagacgtgttcctggggatgtttctgtatgagtacgcccggcgtcaccccgattatagcgtcgtgctcctgctccgactggcaaagacctacgaaacaactctggagaaatgttgcgctgccgcagaccctcatgaatgttatgctaaggtgttcgatgagtttaagccactcgtcgaagagccccagaacctgattaaacagaattgcgaactgttcgagcagctcggtgaatacaagtttcagaacgccctgctcgtgcgttataccaaaaaggtccctcaggtgtctacaccaactctggtggaggtcagtaggaatctgggcaaagtgggatcaaagtgttgcaaacaccccgaggcaaagagaatgccttgtgctgaagattacctctccgtcgtgctgaaccagctctgcgtgctgcatgaaaagaccccagtcagcgatcgggtgacaaaatgttgcaccgaatctctggtcaatcgccgaccctgtttcagtgccctcgaagtggacgaaacttatgtgcctaaggagtttcaggctgaaacattcacctttcacgccgatatctgcactctgtccgagaaagaaaggcagattaagaaacagacagcactggtcgagctcgtgaagcataaaccaaaggctaccaaggagcagctgaaagccgtcatggacgatttcgcagcttttgtggaaaagtgttgcaaagccgacgataaggagacttgtttcgcagaagaggggaaaaagctcgtggctgccagccaggcagctctgggtctg

SEQ ID NO: 147 - DNA, 57 bp, Artificial Sequence
  source: /note=“Description of Artificial Sequence Synthetic oligonucleotide”
ccggcaacaagatgaagagcaccaactcgagttggtgctcttcatcttgttgttttt

SEQ ID NO: 148 - DNA, 58 bp, Artificial Sequence
  source: /note=“Description of Artificial Sequence Synthetic oligonucleotide”
ccggaacacatttgggatgttcctcctcgaggaggaacatcccaaatgtgtttttttg

SEQ ID NO: 149 - DNA, 58 bp, Artificial Sequence
  source: /note=“Description of Artificial Sequence Synthetic oligonucleotide”
ccggttcgtcatgttgaactataacctcgaggttatagttcaacatgacgaatttttg

SEQ ID NO: 150 - DNA, 58 bp, Artificial Sequence
  source: /note=“Description of Artificial Sequence Synthetic oligonucleotide”
ccggtatatgaatcggcaacgagatctcgagatctcgttgccgattcatatatttttg